(19) United States
(12) Patent Application Publication
(10) Pub. No.: US 2026/0183303 A1
(43) Pub. Date: Jul. 2, 2026

(54) PDK1 REGULATORY COMPOUND AND COMPOSITION CONTAINING THE SAME

(71) Applicant: PROVIBIO CO., LTD., Gimhae-si (KR)

(72) Inventor: Seok Ju PARK, Busan (KR)

(73) Assignee: PROVIBIO CO., LTD., Gimhae-si (KR)

(21) Appl. No.: 19/548,820

(22) Filed: Feb. 24, 2026

Related U.S. Application Data
(63) Continuation of application No. PCT/KR2025/005398, filed on Apr. 21, 2025.

(30) Foreign Application Priority Data
Apr. 19, 2024 (KR) 10-2024-0053033
Apr. 21, 2025 (KR) 10-2025-0051925

(51) Int. Cl. A61K 31/5377; A61K 31/44; A61K 31/4427; A61K 31/443; A61K 31/4439; A61K 31/444; A61K 31/4545; A61K 31/4709; A61K 31/4725; A61K 31/495; A61K 31/496; A61K 31/4985; A61K 31/519; A61K 31/541; A61K 31/55; A61P 35/00; A61P 35/04

(52) U.S. Cl. CPC A61K 31/5377; A61K 31/44; A61K 31/4427; A61K 31/443; A61K 31/4439; A61K 31/444; A61K 31/4545; A61K 31/4709; A61K 31/4725; A61K 31/495; A61K 31/496; A61K 31/4985; A61K 31/519; A61K 31/541; A61K 31/55; A61P 35/00; A61P 35/04

(57) ABSTRACT

Provided is a 3-phosphoinositide-dependent protein kinase 1 (PDK1) modulator compound. The PDK1 modulator compound is provided as a compound represented by chemical formula 1, or a solvate, hydrate, prodrug, stereoisomer or pharmaceutically acceptable salt thereof, and regulates, inhibits or antagonizes the activity of PDK1, and thus can be used for anticancer, antifibrosis, anti-aging and reverse aging, or treatment or prevention of Alzheimer's disease or autoimmune disease. In addition, the present invention provides a composition for anticancer, antifibrosis, anti-aging and reverse aging, or treatment, prevention or alleviation of Alzheimer's disease or autoimmune disease, containing the PDK1 modulator compound and a salt thereof as active ingredients, and provides a method, in which the compound and a salt thereof are administered to a subject, for anticancer, antifibrosis, anti-aging and reverse aging, or treatment, prevention or alleviation of Alzheimer's disease or autoimmune disease.

### TECHNICAL FIELD

The present invention relates to a PDK1 regulator compound and compositions containing the same, and more specifically, to the use of the PDK1 regulator compound for the treatment or prevention of cancer, fibrosis, aging, anti-aging, Alzheimer's disease, or autoimmune diseases by regulating, inhibiting, or antagonizing the activity of PDK1 (3-phosphoinositide-dependent protein kinase 1).

Additionally, the present invention relates to compositions for the prevention or improvement of cancer, antifibrotic, anti-aging, and anti-aging, Alzheimer's disease, or autoimmune diseases, comprising the aforementioned PDK1 regulatory compounds and their salts as active ingredients.

Furthermore, the present invention relates to methods for treating, preventing, or improving cancer or autoimmune diseases by administering the aforementioned compounds and their salts to a subject.

### Priority Information

This application claims priority from Korean Patent Application No. 10-2024-0053033 filed on Apr. 19, 2024 (Prior Application), and Korean Patent Application No. 10-2025-0051925 filed on Apr. 21, 2025, and incorporates the entire contents of the Prior Application herein.

### BACKGROUND ART

PDK1 (3-phosphoinositide-dependent protein kinase 1), also known as PDPK1, is one of the phosphokinases included in the ‘PI3K-PDK1-AKT-mTOR’

pathway, acting as a central regulator of the AGC kinase family, which includes AKT, SGK, PLK, S6K, and RSK, among others, and are essential for cell growth, survival, and metabolism.

PDK1 consists of an N-terminal catalytic domain (Kinase domain, PDK1-PKD) and a C-terminal pleckstrin homology domain (Pleckstrin homology domain, PH domain, PDK1-PHD) and is known to play a central role in regulating the activity of various kinases directly or indirectly within the ‘PI3K-PDK1-AKT-mTOR’ pathway (Belham, Christopher, Shilan Wu, and Joseph Avruch, Curr. Biol., 1999, 9(3):R94-96, FIG. 1).

Activated P13-K (Phosphoinositide 3-kinase) converts PIP2 to PIP3, which sequentially induces the exposure of serine and threonine residues in AKT (Adenosin kinase terminal/Protein Kinase B).

In this state, AKT is activated in the presence of PDK1 and regulates downstream signaling pathways, including mTOR (mammalian Target of rapamycin).

In particular, the phosphorylation of mTOR sequentially activates and inactivates S6K (Ribosomal protein S6 kinase) and 4E-BP1 (Eukaryotic translation initiation factor, 4E-binding protein 1), leading to the initiation of ribosomal protein synthesis and protein translation.

Meanwhile, in T cells, the activation of AKT by PDK1 leads to the activation of mTORC1, a positive regulator of follicular helper T (Tfh) cells, and NFκB, one of the key factors in the T cell signaling pathway, is also activated by this signaling pathway. (Sun Zhen et al., ELife, 2021, 10:e61406, FIG. 2)

Due to its importance, the kinases in the PI3K-PDK1-AKT-mTOR pathway are being utilized as targets for the development of immunosuppressants and anticancer drugs.

Among them, PDK1 is a key phosphokinase that regulates the activity of various phosphokinases involved in inflammatory responses and the onset and growth of cancer, thereby coordinating the overall signaling system, and is attracting attention as a major target for drug development.

By binding to the allosteric site of PDK1, the inhibitor of the present invention effectively blocks the abnormal activation of the PI3K/Akt/mTOR signaling pathway, thereby inducing biological effects that suppress cell survival, growth, or fibrotic pathophysiological responses.

The compounds of the present invention bind to a separate allosteric site of PDK1 rather than its active site, thereby exhibiting non-competitive inhibition. This binding induces changes in the overall structural arrangement or conformational structure of PDK1, preventing enzyme reactions even when substrates bind. Therefore, the compounds of the present invention have the characteristic of effectively blocking downstream signaling pathways by inhibiting PDK1 phosphorylation activity regardless of substrate binding.

Particularly in cancer-related diseases, PDK1 (3-Phosphoinositide-dependent protein kinase-1) is overexpressed in various types of cancer, promoting tumor growth, invasiveness, metastasis, and increasing resistance to cell death, among other factors closely associated with the malignant progression of cancer.

Several preclinical studies have reported that PDK1 inhibition is effective in inhibiting tumor growth, and this effect extends beyond simple intracellular effects to influence the tumor microenvironment (TME).

PDK1 regulates the functions of immune cells, cancer-associated fibroblasts (CAFs), and vascular endothelial cells within the TME and can activate various signaling pathways that induce immune evasion mechanisms.

Inhibiting PDK1 can restore tumor-specific immune responses, enhancing antitumor effects, and mechanisms that regulate metabolic competition between immune cells and tumor cells to induce immune cell activation are also gaining attention.

Additionally, PDK1 plays a key role in angiogenesis.

Through its interaction with the VEGF (vascular endothelial growth factor) signaling pathway, it promotes the formation of new blood vessels, thereby enhancing oxygen and nutrient supply to tumors.

Therefore, PDK1 inhibition can be utilized as a strategy to block angiogenesis and create an unfavorable environment for tumor growth.

From a therapeutic perspective, PDK1 inhibition is known to exhibit particularly potent synergistic effects when used in combination therapy rather than as a monotherapy. Combination with PI3K, AKT, and mTOR inhibitors is a representative example, as PDK1 acts as an upstream regulator of these signaling pathways, enabling more potent antitumor responses through combined inhibition. Additionally, combination with antibody-drug conjugates (ADCs) or immunotherapy strategies are under investigation, with PDK1 inhibition gaining attention as a strategy to overcome resistance to existing therapies.

Meanwhile, PDK1 has been reported to play a role in autophagy regulation, and a pathway through which it inhibits autophagy by interacting with mTOR has been identified. In cancer cells, autophagy can act in either a pro-survival or pro-apoptotic direction, and inhibiting PDK1 to induce autophagy can impair cancer cell survival. Furthermore, PDK1 is involved in histone modification and gene expression regulation, exerting significant influence at the transcriptomic level in cancer cells.

In particular, PDK1 overexpression has been associated with poor prognosis and reduced treatment responsiveness in breast cancer, pancreatic cancer, and lung cancer.

In this regard, PDK1 is considered a key molecule that comprehensively regulates tumor growth, progression, immune evasion, and treatment resistance, and PDK1-targeted combination therapy is evaluated as a highly promising approach for next-generation anticancer strategies.

Additionally, particularly in the context of immunity, PDK1 is essential for activating T cells through NF-κB signaling via TCR and CD28 signaling pathways.

In the absence of PDK1, cell cycle progression and B cell apoptosis increase, while B cell maturation and IgG synthesis are impaired, making PDK1 regulation a potential target for immunotherapy strategies.

In autoimmune diseases such as systemic lupus erythematosus (SLE), which are characterized by abnormal T cells, overactive B cells, and excessive production of autoantibodies, PDK1 inhibitors are expected to be highly useful.

Idiopathic pulmonary fibrosis (IPF) is a progressive fibrotic disease characterized by excessive proliferation of fibroblasts in lung tissue and abnormal accumulation of extracellular matrix (ECM), particularly collagen. This leads to reduced lung elasticity, impaired gas exchange function, and ultimately overall deterioration of respiratory function. The PI3K/Akt signaling pathway, which plays a central role in the pathophysiology of fibrosis, is known to regulate cell survival, proliferation, migration, and metabolism, as well as act as a master regulator of the overall fibrotic response.

In particular, in a bleomycin-induced pulmonary fibrosis animal model, activation of this pathway has been closely associated with increased fibroblast proliferation and expression of collagen I and III, and studies have reported that these responses can be effectively inhibited by PI3K inhibitors. PDK1 is a key factor that directly phosphorylates Akt within the PI3K/Akt pathway and functions as an upstream regulatory point in the pathway.

Therefore, selective inhibition of PDK1 can suppress fibroblast survival and proliferation, thereby blocking fibrosis progression. Furthermore, PDK1 inhibition suppresses cross-talk with TGF-β1 signaling, a major inducer of fibrosis, reducing a-SMA expression and ECM accumulation.

Additionally, it has been reported to inhibit early stages of fibrosis responses such as epithelial-mesenchymal transition (EMT) and fibroblast motility, as well as reduce reactive oxygen species (ROS) production to mitigate inflammatory signals.

These mechanisms are commonly involved not only in pulmonary fibrosis but also in fibrosis in various organs. For example, PDK1-related pathways have been observed to contribute to fibrosis progression in systemic sclerosis-associated interstitial lung disease (SSc-ILD) and radiation-induced pulmonary fibrosis.

In liver fibrosis, chronic inflammation induces collagen accumulation in non-alcoholic steatohepatitis (NASH) and viral hepatitis (hepatitis B and C), and studies have shown that inhibiting PDK1 can mitigate fibrosis in liver tissue.

In renal fibrosis, activation of the TGF-β/PI3K/PDK1 pathway, which is associated with ECM production, has been identified in chronic kidney disease (CKD) and diabetic nephropathy. In cardiac fibrosis, PDK1 is involved in regulating myocardial cell survival and fibroblast activation in heart failure and hypertrophic cardiomyopathy.

Additionally, in retinal fibrosis (PVR), PDK1 inhibition has been suggested as a potential therapeutic approach to mitigate vision loss and blindness risk.

In pancreatic fibrosis and skin fibrosis (e.g., keloids, systemic sclerosis), PDK1 is increasingly recognized as a key signaling pathway involved in fibroblast hyperactivation.

As such, PDK1 inhibitors regulate common pathological fibrosis pathways across various organ fibroses, demonstrating potential as multi-organ antifibrotic therapies.

Additionally, in aging-related contexts, PDK1 activity increases in human dermal fibroblasts (human dermal fibroblasts) during the aging process.

Studies have shown that regulating PDK1 reduces the expression of p21, p16, and SA-β-gal, which are characteristic of senescent cells, thereby partially reversing aging.

PDK1 inhibition has been reported to suppress the activity of the AKT and mTOR pathways, contributing to the reduction of cellular stress responses and the senescence-associated secretory phenotype (SASP).

PDK1 is overly activated in senescent cells, and regulating it has been reported to reduce markers of cellular aging and induce rejuvenation effects.

Therefore, PDK1 is emerging as a promising target for aging control and anti-aging therapy.

Alzheimer's disease (AD) is a representative age-related neurodegenerative disorder characterized by memory loss and cognitive dysfunction, with the number of patients continuing to increase.

The pathophysiology of AD is known to involve complex interactions between amyloid beta (Aβ) accumulation, tau protein hyperphosphorylation, neuronal cell death, and inflammatory responses.

PDK1 is a key regulator of the PI3K/Akt pathway, and its overactivation in AD has been reported to increase the expression of γ-secretase, thereby promoting Aβ production, and to inhibit the activity of α-secretase (TACE), thereby reducing the non-amyloid pathway.

Chronic activation of the PDK1/Akt pathway may act in a direction that induces neuronal cell death rather than survival, and this trend is known to become more pronounced with aging.

Some studies have demonstrated that inhibiting PDK1 activity can mitigate Ap-induced neurotoxicity and enhance TACE activity, thereby promoting the non-amyloid pathway.

Additionally, reductions in neuroinflammatory responses and improvements in cognitive function have been reported, leading to the evaluation that PDK1 possesses potential as a new therapeutic target.

Furthermore, PDK1 (3-Phosphoinositide-dependent protein kinase-1) serves as a central regulatory factor in the PI3K/Akt signaling pathway, inducing the activation of various AGC kinases such as Akt, S6K, and PKC, thereby regulating essential biological processes for tumor progression, including cell survival, proliferation, motility, invasion, and angiogenesis.

Notably, PDK1 induces epithelial-mesenchymal transition (EMT) in tumor cells, thereby increasing the mobility and invasiveness of cancer cells, playing a crucial role in the early stages of cancer metastasis.

In fact, Du et al. reported in a 2016 paper in Oncogene that in a MMTV-PyMT breast cancer mouse model with PDK1 gene deficiency, the number of lung metastases was significantly reduced, cell motility and invasiveness were inhibited, and the expression patterns of EMT-related markers were altered.

Additionally, PDK1 inhibition was found to simultaneously block various pathways involved in metastasis by suppressing the expression of VEGF, a vascular endothelial growth factor, and inhibiting the activity of p-Akt and mTOR signaling pathways.

PDK1 is a key factor that induces angiogenesis through the PI3K/Akt/mTOR/HIF-1a/VEGF axis, and this pathway is closely associated with pathological angiogenesis within the eye.

In fact, in wet age-related macular degeneration (wAMD), choroidal neovascularization, in diabetic retinopathy (DR) and retinal vein occlusion (RVO), retinal ischemic neovascularization, and in proliferative retinal diseases, vascular and fibrotic proliferation act as major pathophysiological mechanisms.

Here, VEGF is utilized as an important therapeutic target.

Therefore, by inhibiting PDK1 to reduce VEGF expression, abnormal vascular proliferation in these ophthalmic diseases can be effectively suppressed, and PDK1 inhibitors can be applied as useful therapeutic agents in the ophthalmic field.

Accordingly, the inventors of this patent application have focused their research on the anticancer, immunosuppressive, antifibrotic, and anti-aging effects of inhibiting PDK1 activity.

### DISCLOSURE

### Technical Problem

The technical problem that the present invention seeks to solve is to provide a compound that exhibits excellent therapeutic, preventive, or improving effects against cancer or autoimmune diseases.

As a result, the compound of chemical formula 1 was developed, and the present invention was completed.

Furthermore, the present invention aims to provide a compound or its salt that exhibits excellent therapeutic, preventive, or improving effects against cancer or autoimmune diseases.

According to the present invention, the compounds of formula 1 or their salts, which have PDK1 inhibitory, regulatory, or antagonistic activity, exhibit excellent effects in inducing apoptosis of cancer cells or inhibiting tumor growth and proliferation, and demonstrate excellent safety for the human body.

Therefore, they can be usefully utilized for the prevention, treatment, or improvement of cancer.

Additionally, the compounds of formula 1 or their salts according to the present invention, which exhibit excellent PDK1 inhibitory activity, not only inhibit T cell proliferation but also induce B cell apoptosis, thereby demonstrating potent immune-suppressive functions. Thus, they can be usefully employed for the treatment or prevention of autoimmune diseases characterized by the formation of autoantibodies, in addition to transplant rejection reactions.

Furthermore, the PDK1 inhibitor of the present invention can effectively block the metastasis process of tumors by inhibiting EMT, reducing mobility and invasiveness, inhibiting angiogenesis, and suppressing Akt/mTOR signaling, thereby serving as a useful therapeutic agent for inhibiting metastasis and treating various solid tumors such as breast cancer, lung cancer, and pancreatic cancer.

### Technical Solution

The present invention provides a compound represented by chemical formula 1, or its solvates, hydrates, prodrugs, stereoisomers, or pharmaceutically acceptable salts thereof, to solve the above-mentioned technical problems.

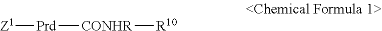

wherein Z1 is —NH2 (or —NHR1, wherein R1 is benzene or a benzene derivative (compound 59)), —SH, methylthio, benzoic acid, benzyloxy group, halogen-substituted alkyl group, benzimidazolyl group, —OH, —SH, halogen, —NO₂—, —CF₃, C1-C6 alkyl, C2-C6 alkenyl, C2-C6 alkynyl, C3-C10 arylalkyl (Ar—(CH2)n—), C5-C10 aryl (Ar—), C3-C10 alkylaryl ((CH₂)n—Ar—), C3-C10 cycloalkyl, C3-C10 heteroaryl, or C3-C10 heterocycloalkyl, or unsubstituted C5-C10 aryl or C3-C10 heteroaryl, where
Prd is pyridine or benzene,
—CONH— is an amide group (amide bond),
R¹⁰ is C8-C14 alkyl, C8-C14 alkenyl, or C8-C14 alkynyl where one, two, or three hydrogen atoms on the terminal carbon are substituted with halogen (fluorine, chlorine, bromine, iodine), morpholine or its analogues, piperazine or its analogues, thiomorpholine or its analogues, pyrolidine or its analogues, piperidine or its analogues, 3,4-dihydro-2H-benzo[b][1,4]oxazine or its analogues, pyrimidine or its analogues, pyrazine or its analogues, or Table 1.

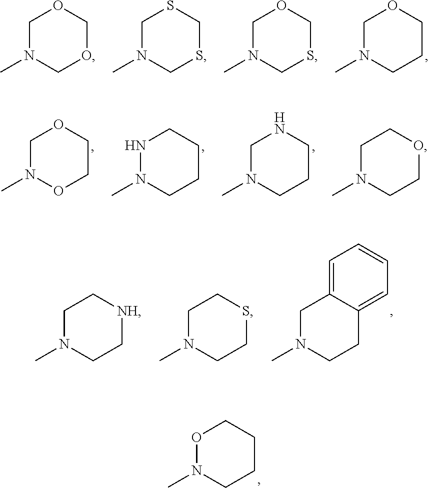

### or Table 1.

TABLE 1

| Derivative | NO. | Functional group |
|---|---|---|
| Morpholine | M1 | 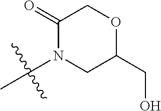 [SMILES: *N1CC(CO)OCC1=O] |
| | M2 | 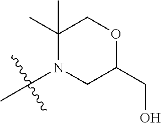 [SMILES: *N1CC(CO)OCC1(C)C] |
| | M3 | 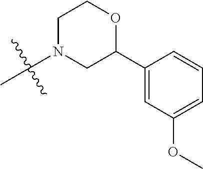 [SMILES: *N1CCOC(c2cccc(OC)c2)C1] |
| | M4 | 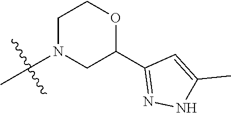 [SMILES: *N1CCOC(c2cc(C)[nH]n2)C1] |
| | M5 | 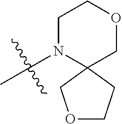 [SMILES: *N1CCOCC12CCOC2] |
| | M6 | 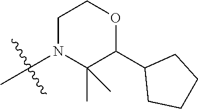 [SMILES: *N1CCOC(C2CCCC2)C1(C)C] |
| | M7 | 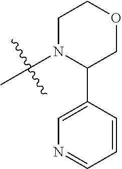 [SMILES: *N1CCOCC1c1cccnc1] |
| | M8 | 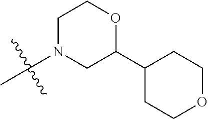 [SMILES: *N1CCOC(C2CCOCC2)C1] |
| | M9 | 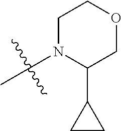 [SMILES: *N1CCOCC1C1CC1] |
| | M10 | 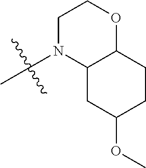 [SMILES: *N1CCOC2CCC(OC)CC21] |
| | M11 | 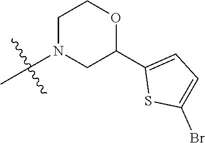 [SMILES: *N1CCOC(c2ccc(Br)s2)C1] |
| | M12 | 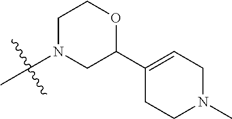 [SMILES: *N1CCOC(C2=CCN(C)CC2)C1] |
| | M13 | 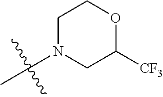 [SMILES: *N1CCOC(C(F)(F)F)C1] |
| | M14 | 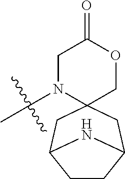 [SMILES: *N1CC(=O)OCC12CC1CCC(C2)N1] |
| | M15 | 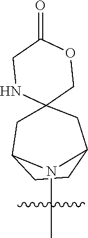 [SMILES: *N1C2CCC1CC1(COC(=O)CN1)C2] |
| | M16 | 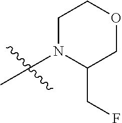 [SMILES: *N1CCOCC1CF] |
| | m17 | 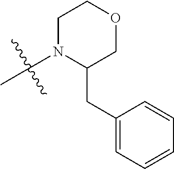 [SMILES: *N1CCOCC1Cc1ccccc1] |
| | M18 | 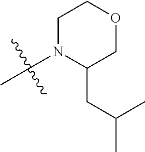 [SMILES: *N1CCOCC1CC(C)C] |
| | M19 | 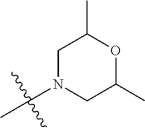 [SMILES: *N1CC(C)OC(C)C1] |
| | M20 | 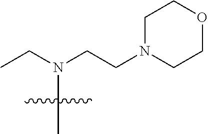 [SMILES: *N(CC)CCN1CCOCC1] |
| | M21 | 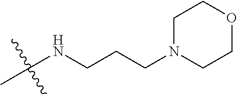 [SMILES: *NCCCN1CCOCC1] |
| | M22 | 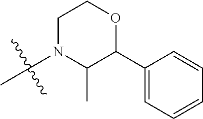 [SMILES: *N1CCOC(c2ccccc2)C1C] |
| | M23 | 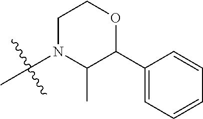 [SMILES: *N1CCOC(c2ccccc2)C1C] |
| | M24 | 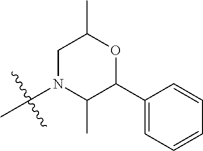 [SMILES: *N1CC(C)OC(c2ccccc2)C1C] |
| | M25 | 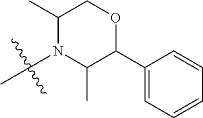 [SMILES: *N1C(C)COC(c2ccccc2)C1C] |
| | M26 | 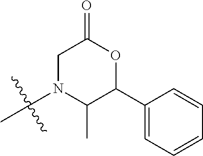 [SMILES: *N1CC(=O)OC(c2ccccc2)C1C] |
| | M27 | 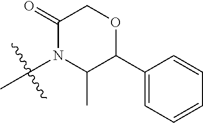 [SMILES: *N1C(=O)COC(c2ccccc2)C1C] |
| | M28 | 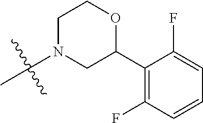 [SMILES: *N1CCOC(c2c(F)cccc2F)C1] |
| | M29 | 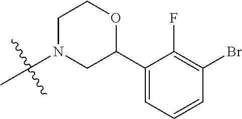 [SMILES: *N1CCOC(c2cccc(Br)c2F)C1] |
| | M30 | 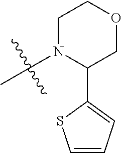 [SMILES: *N1CCOCC1c1cccs1] |
| | M31 | 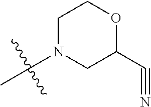 [SMILES: *N1CCOC(C#N)C1] |
| | M32 | 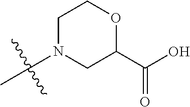 [SMILES: *N1CCOC(C(=O)O)C1] |
| | M33 | 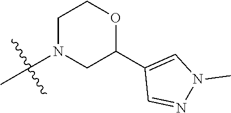 [SMILES: *N1CCOC(c2cnn(C)c2)C1] |
| | M34 | 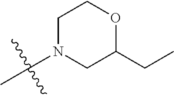 [SMILES: *N1CCOC(CC)C1] |
| | M35 | 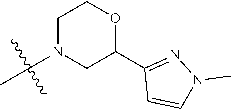 [SMILES: *N1CCOC(c2ccn(C)n2)C1] |
| Piperazine | Pi1 | 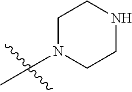 [SMILES: *N1CCNCC1] |
| | Pi2 | 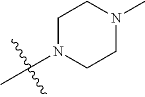 [SMILES: *N1CCN(C)CC1] |
| | Pi3 | 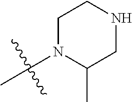 [SMILES: *N1CCNCC1C] |
| | Pi4 | 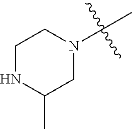 [SMILES: *N1CCNC(C)C1] |
| | Pi5 | 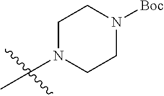 [SMILES: *N1CCN(C(=O)OC(C)(C)C)CC1] |
| | Pi6 | 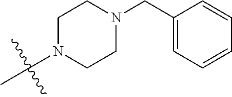 [SMILES: *N1CCN(Cc2ccccc2)CC1] |
| | Pi7 | 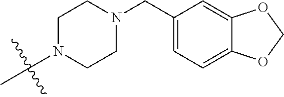 [SMILES: *N1CCN(Cc2ccc3c(c2)OCO3)CC1] |
| | Pi8 | 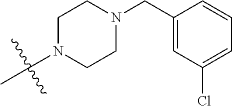 [SMILES: *N1CCN(Cc2cccc(Cl)c2)CC1] |
| | Pi9 | 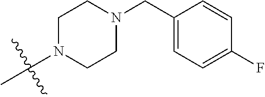 [SMILES: *N1CCN(Cc2ccc(F)cc2)CC1] |
| | Pi10 | 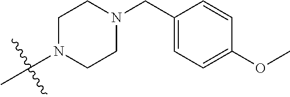 [SMILES: *N1CCN(Cc2ccc(OC)cc2)CC1] |
| | Pi11 | 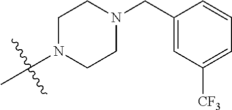 [SMILES: *N1CCN(Cc2cccc(C(F)(F)F)c2)CC1] |
| | Pi12 | 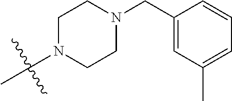 [SMILES: *N1CCN(Cc2cccc(C)c2)CC1] |
| | Pi13 | 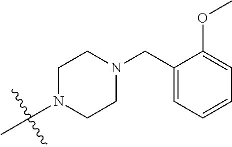 [SMILES: *N1CCN(Cc2ccccc2OC)CC1] |
| | Pi14 | 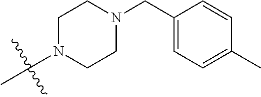 [SMILES: *N1CCN(Cc2ccc(C)cc2)CC1] |
| | Pi15 | 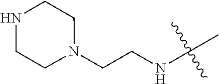 [SMILES: *NCCN1CCNCC1] |
| | Pi16 | 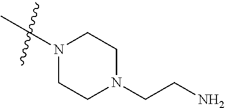 [SMILES: *N1CCN(CCN)CC1] |
| | Pi17 | 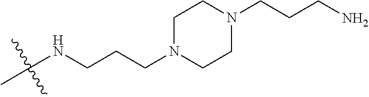 [SMILES: *NCCCN1CCN(CCCN)CC1] |
| | Pi18 | 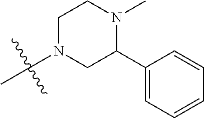 [SMILES: *N1CCN(C)C(c2ccccc2)C1] |
| | Pi19 | 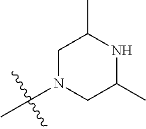 [SMILES: *N1CC(C)NC(C)C1] |
| | Pi20 | 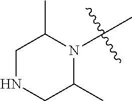 [SMILES: *N1C(C)CNCC1C] |
| | Pi21 | 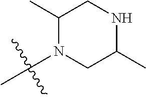 [SMILES: *N1CC(C)NCC1C] |
| | Pi22 | 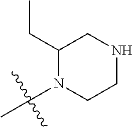 [SMILES: *N1CCNCC1CC] |
| | Pi23 | 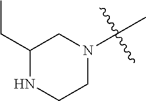 [SMILES: *N1CCNC(CC)C1] |
| | Pi24 | 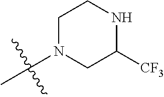 [SMILES: *N1CCNC(C(F)(F)F)C1] |
| | Pi25 | 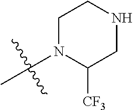 [SMILES: *N1CCNCC1C(F)(F)F] |
| | Pi26 | 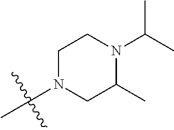 [SMILES: *N1CCN(C(C)C)C(C)C1] |
| | Pi27 | 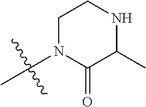 [SMILES: *N1CCNC(C)C1=O] |
| | Pi28 | 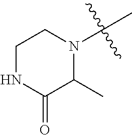 [SMILES: *N1CCNC(=O)C1C] |
| | Pi29 | 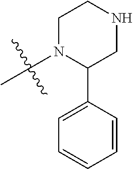 [SMILES: *N1CCNCC1c1ccccc1] |
| | Pi30 | 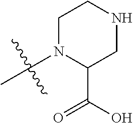 [SMILES: *N1CCNCC1C(=O)O] |
| | Pi31 | 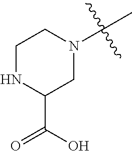 [SMILES: *N1CCNC(C(=O)O)C1] |
| | Pi32 | 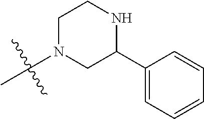 [SMILES: *N1CCNC(c2ccccc2)C1] |
| | Pi33 | 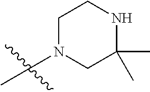 [SMILES: *N1CCNC(C)(C)C1] |
| | Pi34 | 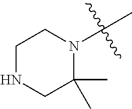 [SMILES: *N1CCNCC1(C)C] |
| | Pi35 | 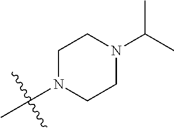 [SMILES: *N1CCN(C(C)C)CC1] |
| | Pi36 | 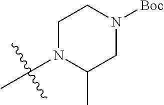 [SMILES: *N1CCN(C(=O)OC(C)(C)C)CC1C] |
| | Pi37 | 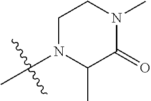 [SMILES: *N1CCN(C)C(=O)C1C] |
| | Pi38 | 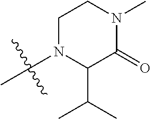 [SMILES: *N1CCN(C)C(=O)C1C(C)C] |
| | Pi39 | 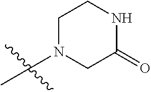 [SMILES: *N1CCNC(=O)C1] |
| | Pi40 | 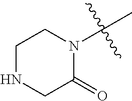 [SMILES: *N1CCNCC1=O] |
| | Pi41 | 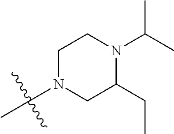 [SMILES: *N1CCN(C(C)C)C(CC)C1] |
| | Pi42 | 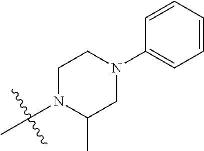 [SMILES: *N1CCN(c2ccccc2)CC1C] |
| | Pi43 | 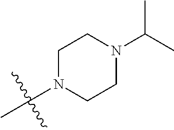 [SMILES: *N1CCN(C(C)C)CC1] |
| Thiomorpholine | Th1 | 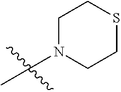 [SMILES: *N1CCSCC1] |
| | Th2 | 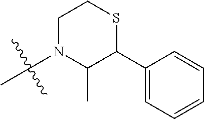 [SMILES: *N1CCSC(c2ccccc2)C1C] |
| | Th3 | 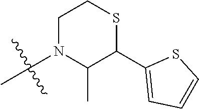 [SMILES: *N1CCSC(c2cccs2)C1C] |
| pyrrolidine | Py1 | 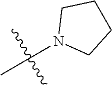 [SMILES: *N1CCCC1] |
| | Py2 | 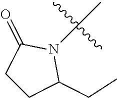 [SMILES: *N1C(=O)CCC1CC] |
| | Py3 | 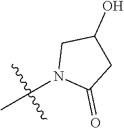 [SMILES: *N1CC(O)CC1=O] |
| | Py4 | 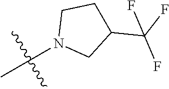 [SMILES: *N1CCC(C(F)(F)F)C1] |
| | Py5 | 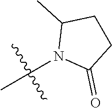 [SMILES: *N1C(=O)CCC1C] |
| | Py6 | 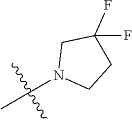 [SMILES: *N1CCC(F)(F)C1] |
| | Py7 | 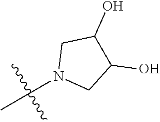 [SMILES: *N1CC(O)C(O)C1] |
| | Py8 | 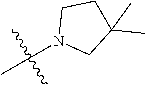 [SMILES: *N1CCC(C)(C)C1] |
| | Py9 | 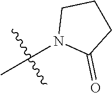 [SMILES: *N1CCCC1=O] |
| | Py10 | 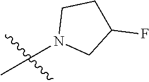 [SMILES: *N1CCC(F)C1] |
| | Py11 | 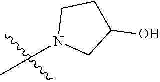 [SMILES: *N1CCC(O)C1] |
| | py12 | 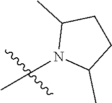 [SMILES: *N1C(C)CCC1C] |
| | py13 | 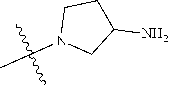 [SMILES: *N1CCC(N)C1] |
| | py14 | 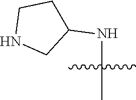 [SMILES: *NC1CCNC1] |
| | Py15 | 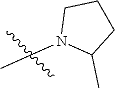 [SMILES: *N1CCCC1C] |
| | Py16 | 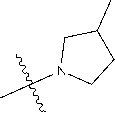 [SMILES: *N1CCC(C)C1] |
| | py17 | 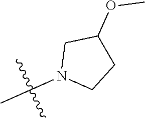 [SMILES: *N1CCC(OC)C1] |
| | py18 | 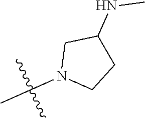 [SMILES: *N1CCC(NC)C1] |
| | py19 | 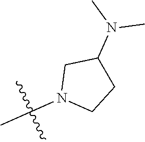 [SMILES: *N1CCC(N(C)C)C1] |
| | Py20 | 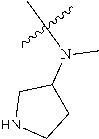 [SMILES: *N(C)C1CCNC1] |
| | Py21 | 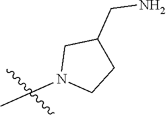 [SMILES: *N1CCC(CN)C1] |
| | Py22 | 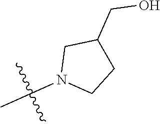 [SMILES: *N1CCC(CO)C1] |
| | Py23 | 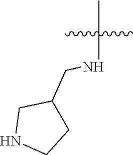 [SMILES: *NCC1CCNC1] |
| | Py24 | 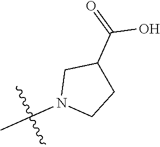 [SMILES: *N1CCC(C(=O)O)C1] |
| | Py25 | 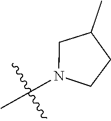 [SMILES: *N1CCC(C)C1] |
| | py26 | 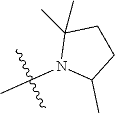 [SMILES: *N1C(C)CCC1(C)C] |
| piperidine | Pd1 | 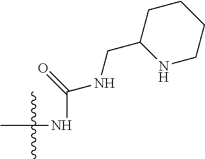 [SMILES: *NC(=O)NCC1CCCCN1] |
| | Pd2 | 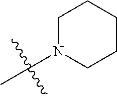 [SMILES: *N1CCCCC1] |
| | Pd3 | 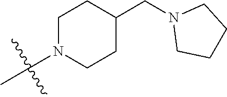 [SMILES: *N1CCC(CN2CCCC2)CC1] |
| | Pd4 | 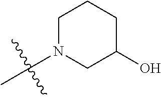 [SMILES: *N1CCCC(O)C1] |
| | Pd5 | 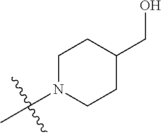 [SMILES: *N1CCC(CO)CC1] |
| | Pd6 | 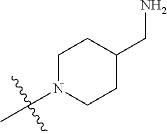 [SMILES: *N1CCC(CN)CC1] |
| | Pd7 | 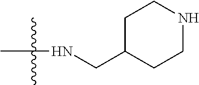 [SMILES: *NCC1CCNCC1] |
| | Pd8 | 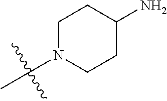 [SMILES: *N1CCC(N)CC1] |
| | Pd9 | 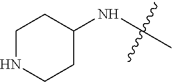 [SMILES: *NC1CCNCC1] |
| | Pd10 | 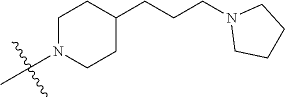 [SMILES: *N1CCC(CCCN2CCCC2)CC1] |
| | Pd11 | 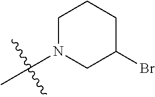 [SMILES: *N1CCCC(Br)C1] |
| | Pd12 | 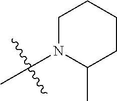 [SMILES: *N1CCCCC1C] |
| | Pd13 | 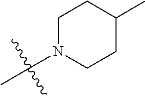 [SMILES: *N1CCC(C)CC1] |
| | Pd14 | 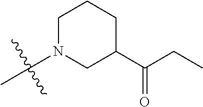 [SMILES: *N1CCCC(C(=O)CC)C1] |
| | Pd15 | 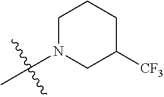 [SMILES: *N1CCCC(C(F)(F)F)C1] |
| | Pd16 | 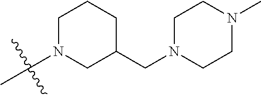 [SMILES: *N1CCCC(CN2CCN(C)CC2)C1] |
| | Pd17 | 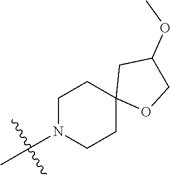 [SMILES: *N1CCC2(CC1)CC(OC)CO2] |
| | Pd18 | 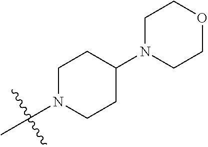 [SMILES: *N1CCC(N2CCOCC2)CC1] |
| | Pd19 | 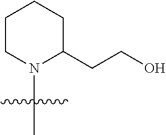 [SMILES: *N1CCCCC1CCO] |
| | Pd20 | 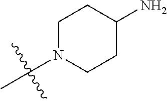 [SMILES: *N1CCC(N)CC1] |
| | Pd21 | 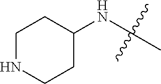 [SMILES: *NC1CCNCC1] |
| | Pd22 | 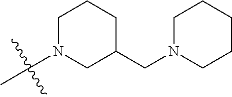 [SMILES: *N1CCCC(CN2CCCCC2)C1] |
| | Pd23 | 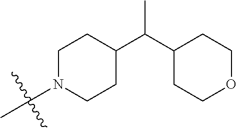 [SMILES: *N1CCC(C(C)C2CCOCC2)CC1] |
| | Pd24 | 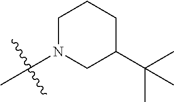 [SMILES: *N1CCCC(C(C)(C)C)C1] |
| | Pd25 | 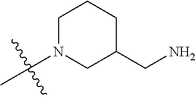 [SMILES: *N1CCCC(CN)C1] |
| | Pd26 | 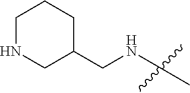 [SMILES: *NCC1CCCNC1] |
| | Pd27 | 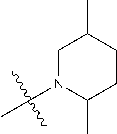 [SMILES: *N1CC(C)CCC1C] |
| | Pd28 | 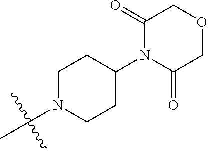 [SMILES: *N1CCC(N2C(=O)COCC2=O)CC1] |
| 3,4-dihydro-2H- benzo[b][1,4]oxazine | Di1 | 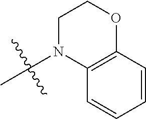 [SMILES: *N1CCOc2ccccc21] |
| | Di2 | 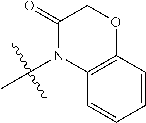 [SMILES: *N1C(=O)COc2ccccc21] |
| | Di3 | 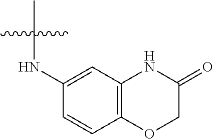 [SMILES: *Nc1ccc2c(c1)NC(=O)CO2] |
| | Di4 | 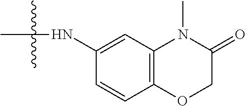 [SMILES: *Nc1ccc2c(c1)N(C)C(=O)CO2] |
| | Di5 | 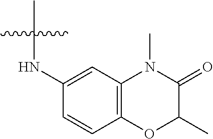 [SMILES: *Nc1ccc2c(c1)N(C)C(=O)C(C)O2] |
| | Di6 | 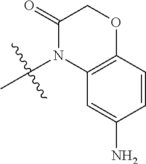 [SMILES: *N1C(=O)COc2ccc(N)cc21] |
| | Di7 | 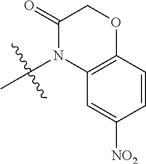 [SMILES: *N1C(=O)COc2ccc([N+](=O)[O-])cc21] |
| Pyrimidine | Pyd1 | 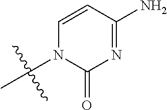 [SMILES: *n1ccc(N)nc1=O] |
| | Pyd2 | 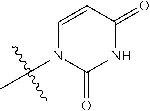 [SMILES: *n1ccc(=O)[nH]c1=O] |
| | Pyd3 | 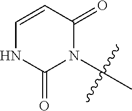 [SMILES: *n1c(=O)cc[nH]c1=O] |
| | Pyd4 | 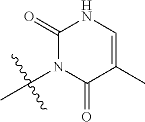 [SMILES: *n1c(=O)[nH]cc(C)c1=O] |
| | Pyd5 | 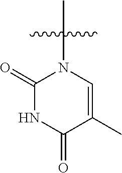 [SMILES: *n1cc(C)c(=O)[nH]c1=O] |
| | Pyd6 | 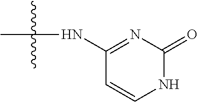 [SMILES: *Nc1cc[nH]c(=O)n1] |
| Pyrazine | Pyz1 | 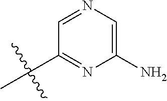 [SMILES: *c1cncc(N)n1] |
| | Pyz2 | 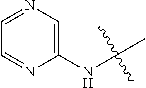 [SMILES: *Nc1cnccn1] |
| | Pyz3 | 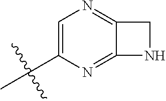 [SMILES: *c1cnc2c(n1)NC2] |
| | Pyz4 | 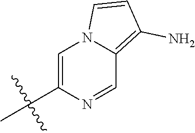 [SMILES: *c1cn2ccc(N)c2cn1] |
| | Pyz5 | 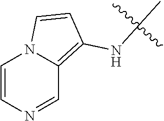 [SMILES: *Nc1ccn2ccncc12] |
| | Pyz6 | 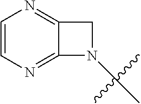 [SMILES: *N1Cc2nccnc21] |
| | Pyz7 | 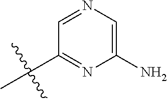 [SMILES: *c1cncc(N)n1] |
| Other | O1 | 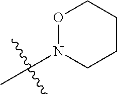 [SMILES: *N1CCCCO1] |
| | O2 | 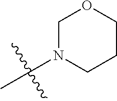 [SMILES: *N1CCCOC1] |
| | O3 | 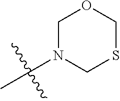 [SMILES: *N1COCSC1] |
| | O4 | 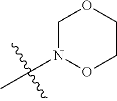 [SMILES: *N1COCCO1] |
| | O5 | 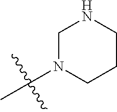 [SMILES: *N1CCCNC1] |
| | O6 | 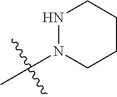 [SMILES: *N1CCCCN1] |
| | O7 | 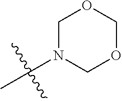 [SMILES: *N1COCOC1] |
| | O8 | 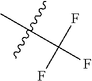 [SMILES: *C(F)(F)F] |
| | O9 | 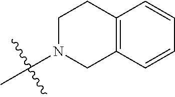 [SMILES: *N1CCc2ccccc2C1] |
| | O10 | 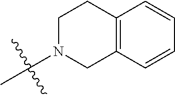 [SMILES: *N1CCc2ccccc2C1] |
| | O11 | 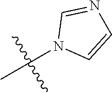 [SMILES: *n1ccnc1] |
| | O12 | 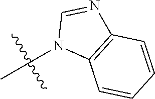 [SMILES: *n1cnc2ccccc21] |
| | O13 | 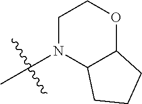 [SMILES: *N1CCOC2CCCC21] |
| | O14 | 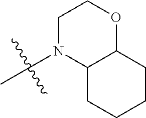 [SMILES: *N1CCOC2CCCCC21] |
| | O15 | 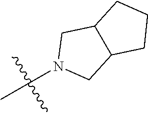 [SMILES: *N1CC2CCCC2C1] |
| | O16 | 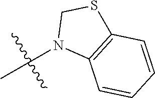 [SMILES: *N1CSc2ccccc21] |
| | O17 | 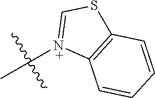 [SMILES: *[n+]1csc2ccccc21] |
| | O18 | 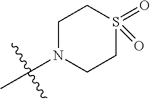 [SMILES: *N1CCS(=O)(=O)CC1] |
| | O19 | 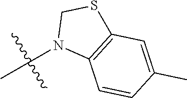 [SMILES: *N1CSc2cc(C)ccc21] |
| | O20 | 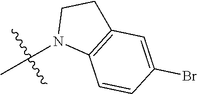 [SMILES: *N1CCc2cc(Br)ccc21] |
| | O21 | 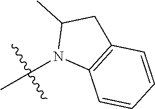 [SMILES: *N1c2ccccc2CC1C] |
| | O22 | 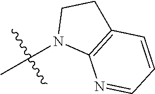 [SMILES: *N1CCc2cccnc21] |
| | O23 | 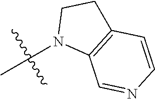 [SMILES: *N1CCc2ccncc21] |
| | O24 | 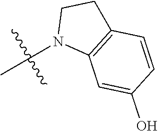 [SMILES: *N1CCc2ccc(O)cc21] |
| | O25 | 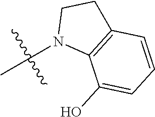 [SMILES: *N1CCc2cccc(O)c21] |
| | O26 | 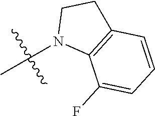 [SMILES: *N1CCc2cccc(F)c21] |
| | O27 | 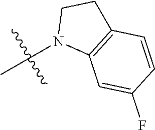 [SMILES: *N1CCc2ccc(F)cc21] |
| | O28 | 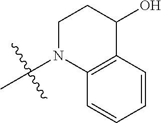 [SMILES: *N1CCC(O)c2ccccc21] |
| | O29 | 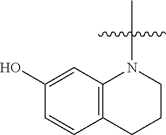 [SMILES: *N1CCCc2ccc(O)cc21] |
| | O30 | 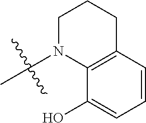 [SMILES: *N1CCCc2cccc(O)c21] |
| | O31 | 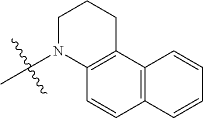 [SMILES: *N1CCCc2c1ccc1ccccc21] |
| | O32 | 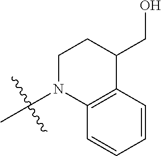 [SMILES: *N1CCC(CO)c2ccccc21] |
| | O33 | 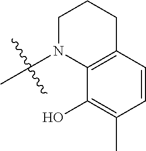 [SMILES: *N1CCCc2ccc(C)c(O)c21] |
| | O34 | 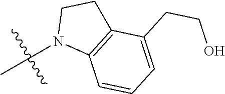 [SMILES: *N1CCc2c(CCO)cccc21] |
| | O35 | 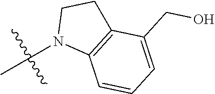 [SMILES: *N1CCc2c(CO)cccc21] |
| | O36 | 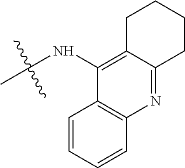 [SMILES: *Nc1c2c(nc3ccccc13)CCCC2] |
| | O37 | 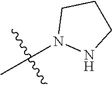 [SMILES: *N1CCCN1] |
| | O38 | 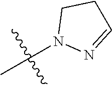 [SMILES: *N1CCC=N1] |
| | O39 | 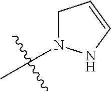 [SMILES: *N1CC=CN1] |
| | O40 | 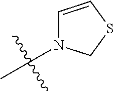 [SMILES: *N1C=CSC1] |
| | O41 | 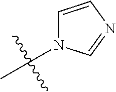 [SMILES: *n1ccnc1] |
| | O42 | 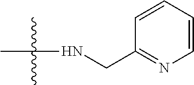 [SMILES: *NCc1ccccn1] |
| | O43 | 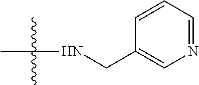 [SMILES: *NCc1cccnc1] |
| | O44 | 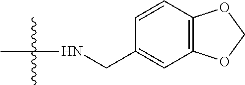 [SMILES: *NCc1ccc2c(c1)OCO2] |
| | O45 | 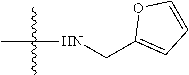 [SMILES: *NCc1ccco1] |
| | O46 | 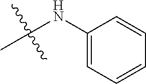 [SMILES: *Nc1ccccc1] |
| | O47 | 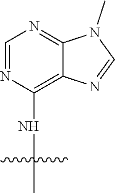 [SMILES: *Nc1ncnc2c1ncn2C] |
| | O48 | 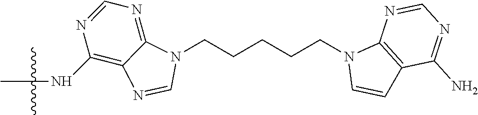 [SMILES: *Nc1ncnc2c1ncn2CCCCCn1ccc2c(N)ncnc21] |
| | O49 | 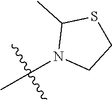 [SMILES: *N1CCSC1C] |
| | O50 | 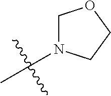 [SMILES: *N1CCOC1] |
| | O51 | 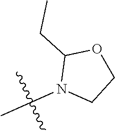 [SMILES: *N1CCOC1CC] |
| | O52 | 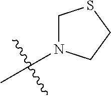 [SMILES: *N1CCSC1] |
| | O53 | 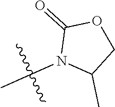 [SMILES: *N1C(=O)OCC1C] |
| | O54 | 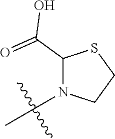 [SMILES: *N1CCSC1C(=O)O] |
| | O55 | 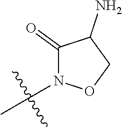 [SMILES: *N1OCC(N)C1=O] |
| | O56 | 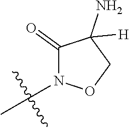 [SMILES: *N1OCC(N)C1=O] |
| | O57 | 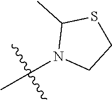 [SMILES: *N1CCSC1C] |
| | O58 | 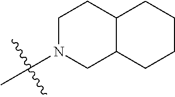 [SMILES: *N1CCC2CCCCC2C1] |
| | O59 | 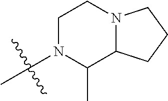 [SMILES: *N1CCN2CCCC2C1C] |
| | O60 | 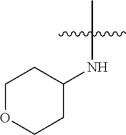 [SMILES: *NC1CCOCC1] |
| | O61 | 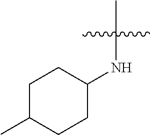 [SMILES: *NC1CCC(C)CC1] |
| | O62 | 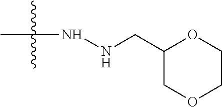 [SMILES: *NNCC1COCCO1] |
| | O63 | 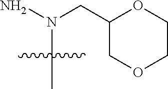 [SMILES: *N(N)CC1COCCO1] |
| | O64 | 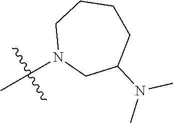 [SMILES: *N1CCCCC(N(C)C)C1] |
| | O65 | 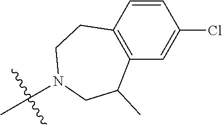 [SMILES: *N1CCc2ccc(Cl)cc2C(C)C1] |
| | O66 | 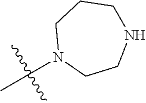 [SMILES: *N1CCCNCC1] |
| | O67 | 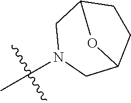 [SMILES: *N1CC2CCC(C1)O2] |
| | O68 | 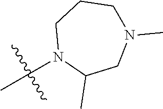 [SMILES: *N1CCCN(C)CC1C] |
| | O69 | 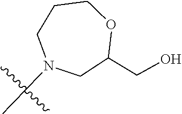 [SMILES: *N1CCCOC(CO)C1] |
| | O70 | 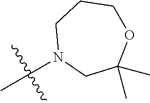 [SMILES: *N1CCCOC(C)(C)C1] |
| | O71 | 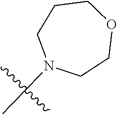 [SMILES: *N1CCCOCC1] |
| | O72 | 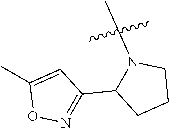 [SMILES: *N1CCCC1c1cc(C)on1] |
| | O73 | 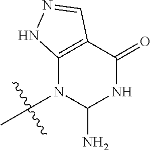 [SMILES: *N1c2[nH]ncc2C(=O)NC1N] |
| | O74 | 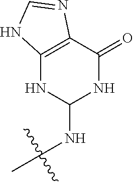 [SMILES: *NC1NC(=O)c2nc[nH]c2N1] |
| | O75 | 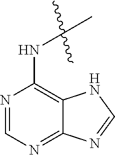 [SMILES: *Nc1ncnc2nc[nH]c12] |
| | O76 | 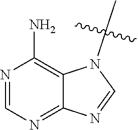 [SMILES: *n1cnc2ncnc(N)c21] |
| | O77 | 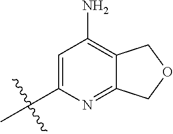 [SMILES: *c1cc(N)c2c(n1)COC2] |
| | O78 | 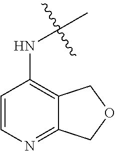 [SMILES: *Nc1ccnc2c1COC2] |
| | O79 | 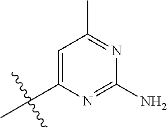 [SMILES: *c1cc(C)nc(N)n1] |
| | O80 | 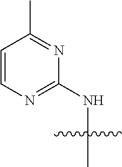 [SMILES: *Nc1nccc(C)n1] |
| | O81 | 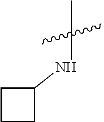 [SMILES: *NC1CCC1] |
| | O82 | 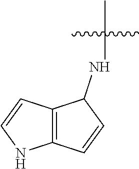 [SMILES: *NC1C=Cc2[nH]ccc21] |
| | O83 | 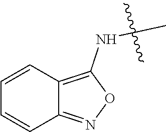 [SMILES: *Nc1onc2ccccc12] |
| | O84 | 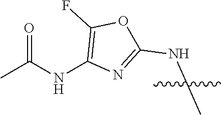 [SMILES: *Nc1nc(NC(C)=O)c(F)o1] |
| | O85 | 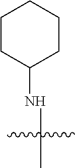 [SMILES: *NC1CCCCC1] |
| | O86 | 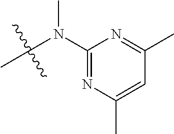 [SMILES: *N(C)c1nc(C)cc(C)n1] |
| | O87 | 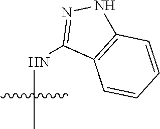 [SMILES: *Nc1n[nH]c2ccccc12] |
| | O88 | 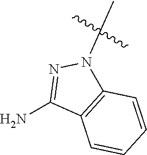 [SMILES: *n1nc(N)c2ccccc21] |

In the present invention, terms such as alkyl, alkenyl, and alkynyl are intended to include both straight chains (also referred to as linear) and side chains (also referred to as branched).

For example, the term “alkyl” refers to a non-branched or branched saturated hydrocarbon chain.

In some embodiments, alkyl may have 1 to 6 carbon atoms ((C1-C6)alkyl) or 8 to 14 carbon atoms ((C8-C14)alkyl).

More specific examples include methyl, ethyl, n-propyl, isopropyl, n-butyl, sec-butyl, tert-butyl, n-pentyl, 2-pentyl, isopentyl, neopentyl, n-hexyl, 2-hexyl, 3-hexyl, and 3-methyl pentyl, but are not limited to these.

When naming alkyl groups with a specific number of carbon atoms, all geometric isomers with that number of carbon atoms may be included.

For example, “butyl” may include n-butyl, sec-butyl, isobutyl, and t-butyl, and “propyl” may include n-propyl and isopropyl.

Unless otherwise specifically stated in this specification, alkyl chains may be arbitrarily substituted with one or more substituents as described herein.

The term “halogen” as used herein means fluorine, chlorine, bromine, or iodine.

The term ‘alkoxy’ as used herein means “oxygen (O)-alkyl.”

The term “hetero” as used herein means a heteroatom selected from oxygen, nitrogen, and sulfur.

The term ‘cycloalkyl’ as used herein means an alkyl forming a ring.

The term “heterocycloalkyl” as used herein means a cycloalkyl containing a heteroatom in the ring.

The term “arylalkyl” used in the present invention refers to an alkyl having an aryl group.

The term “alkylaryl” used in the present invention refers to an aryl having an alkyl group.

The term “analog” as used herein refers to a structure that is chemically similar, such as a structure with a changed ring size, a structure with a heteroatom substitution, a structure with a substituted substituent (such as an alkyl group), or a structure with a change in saturation due to the formation or disappearance of a double bond. For example, an analog of morpholine includes structures where the ring size is changed from 6 atoms to 5 atoms or 7 atoms, or when heteroatoms are substituted, such as oxygen (O) or nitrogen (N) being replaced by sulfur (S), or when substituents (such as alkyl groups) are substituted for nitrogen (N) or oxygen (O).

The term “salt” used in this inventionsalt” refers to compounds that can be manufactured by conventional methods in the relevant technical field, such as salts of inorganic acids like hydrochloric acid, hydrobromic acid, sulfuric acid, sodium bisulfate, phosphoric acid, carbonic acid, or organic acids like formic acid, acetic acid, oxalic acid, benzoic acid, citric acid, tartaric acid, gluconic acid, gastric acid, fumaric acid, lactobionic acid, salicylic acid, or acetylsalicylic acid (aspirin), or by reacting with alkali metal ions such as sodium or potassium to form their metal salts, or by reacting with ammonium ions to form other forms of salts, but is not limited thereto.

The hydrates, isomers, solvates, prodrugs, and crystalline forms of the compounds according to the present invention are included in the scope of the present invention, provided that they exhibit the PDK1 regulatory, inhibitory, or antagonistic properties of the original compound, regardless of differences in terminology.

The term “isomer” as used herein includes stereoisomers, enantiomers, diastereomers, tautomers, and geometric isomers.

The term “solvate” as used herein refers to an aggregate or complex of the compounds of the present invention and one or more solvent molecules, wherein the solvent includes, but is not limited to, water, isopropanol, ethanol, methanol, dimethyl sulfoxide, ethyl acetate, acetic acid, and ethanolamine.

The term “hydrate” as used herein refers to a solvent compound in which the solvent molecule is water.

The term “crystalline form” as used herein refers to a form in which the same molecules form different crystal structures.

According to the present invention, the alkyl, alkenyl, or alkynyl group of R10 having a carbon number of 8 to 14 may be provided to exert an allosteric effect.

According to the present invention, if the number of carbon atoms is 7 or less, it may be difficult to exhibit an immunosuppressive effect, and if it is 15 or more, it may be difficult to form allosteric bonds or may be difficult to absorb in the body.

According to the present invention, the compound of chemical formula 1 according to the present invention may be selected from at least one of the following group:

2-amino-N-(12-fluoro-dodecyl)-nicotinamide, 4′-methyl-biphenyl-2-carboxylic acid (12-fluoro-dodecyl)-amide, 2-amino-N-dodecylnicotinamide, 2-Bromo-N-(12-fluoro-dodecyl)-nicotinamide, 2-Amino-N-[12-(3,4-dihydro-1H-isoquinolin-2-yl)-dodecyl]-nicotinamide, N-(12-fluoro-dodecyl)-3-(3-trifluoromethyl-phenylamino)-isonicotinamide, 4-chloro-N-(12-fluoro-dodecyl)-nicotinamide, 2-amino-N-(12-morpholin-4-yl-dodecyl)-nicotinamide, 2-amino-N-(8-fluoro-octyl)-nicotinamide, N-(12-fluoro-dodecyl)-2-mercapto-nicotinamide, 2-amino-N-(10-morpholin-4-yl-decyl)-nicotinamide, Biphenyl-2-carboxylic acid (12-fluoro-dodecyl)-amide, 1H-Indole-4-carboxylic acid (12-fluoro-dodecyl)-amide, 3-Hydroxy-pyridine-2-carboxylic acid (12-fluoro-dodecyl)-amide, 3-benzoyl-pyridine-2-carboxylic acid (12-fluoro-dodecyl)-amide, isoquinoline-1-carboxylic acid (12-fluoro-dodecyl)-amide, 2-(4-chloro-phenoxy)-N-(12-fluoro-dodecyl)-nicotinamide, N-(12-fluoro-dodecyl)-2-methylsulfonyl-nicotinamide, 2-amino-N-(10-fluoro-decyl)-nicotinamide, 3-(12-fluoro-dodecylcarbamoyl)-pyridine-2-yl-ammonium chloride, 2-Amino-N-(11-fluoro-undecyl)-nicotinamide, 3-(11-fluoro-undecylcarbamoyl)-pyridin-2-yl-ammonium chloride, N-(12-fluoro-dodecyl)-2-hydroxy-nicotinamide, 4-Amino-N-(12-fluoro-dodecyl)-nicotinamide, 3-Amino-N-(12-fluoro-dodecyl)-isonicotinamide, N-(12-fluoro-dodecyl)-2-trifluoromethyl-nicotinamide, N-(12-fluoro-dodecyl)-3-iodo-isonicotinamide, 2-amino-N-(12-thio-morpholin-4-yl-dodecyl)nicotinamide or 2-amino-N-(12-(piperazin-1-yl)dodecyl)nicotinamide.

Furthermore, according to the present invention, the compound may comprise at least one selected from the group consisting of the following chemical formulas: Chemical formulas 2 to 39, 43 to 64, 67 to 71, 73, 76, 88 to 118, and 121 to 217.

The present invention may be a compound characterized by using the aforementioned compound or its pharmaceutically acceptable salt to inhibit, antagonize, or regulate PDK1 (3-phosphoinositide-dependent protein kinase 1).

The present invention provides a method for inhibiting PDK1 (3-phosphoinositide-dependent protein kinase 1) comprising the step of contacting a target body comprising an effective amount of the compound or its pharmaceutically acceptable salt and PDK1 (3-phosphoinositide-dependent protein kinase 1) to inhibit PDK1.

In the above method, the target body may be handled in vivo or in vitro.

The present invention provides a method for inhibiting PDK1, characterized by comprising the step of contacting cells containing an effective amount of the compound or its pharmaceutically acceptable salt and PDK1 (3-phosphoinositide-dependent protein kinase 1) to inhibit PDK1.

In the method, the cells may be handled in vivo or in vitro.

The present invention provides a method for antagonizing PDK1, characterized by comprising the step of contacting a target containing an effective amount of the compound or its pharmaceutically acceptable salt and PDK1 (3-phosphoinositide-dependent protein kinase 1) to antagonize PDK1.

In the aforementioned method, the target may be handled in vivo or in vitro.

The present invention provides a method for antagonizing PDK1, characterized by comprising the step of contacting cells containing an effective amount of the compound or its pharmaceutically acceptable salt and PDK1 (3-phosphoinositide-dependent protein kinase 1) to antagonize PDK1.

In the aforementioned method, the cells may be handled in vivo or in vitro.

The present invention provides a method for treating cancer, comprising administering a therapeutic effective amount of a compound or a pharmaceutically acceptable salt thereof, which is used to inhibit, antagonize, or regulate PDK1 (3-phosphoinositide-dependent protein kinase 1), to a subject having cancer.

According to an embodiment of the present invention, a method for treating autoimmune diseases is provided, comprising administering a therapeutic effective amount of a compound or a pharmaceutically acceptable salt thereof, which is used to inhibit, antagonize, or regulate PDK1 (3-phosphoinositide-dependent protein kinase 1), to a subject suffering from an autoimmune disease.

According to an embodiment of the present invention, the compound or its pharmaceutically acceptable salt may be included as an active ingredient.

According to an embodiment of the present invention, the pharmaceutical composition may non-competitively regulate PDK1 activity.

According to an embodiment of the present invention, the PDK1 regulation may be achieved through allosteric binding.

According to an embodiment of the present invention, the pharmaceutical composition may inhibit the proliferation and survival signals of lymphocytes by blocking the PDK1-involved pathway.

According to an embodiment of the present invention, the pharmaceutical composition may be used for autoimmune diseases, immune hypersensitivity disorders, chronic inflammatory diseases, or lymphocyte-related proliferative disorders occurring in immune cells, macrophages, T cells, B cells, or plasma cells.

According to an embodiment of the present invention, the immune hypersensitivity disorders may include chronic urticaria, asthma, atopic dermatitis, allergic rhinitis, or serum sickness, or at least one thereof.

According to an embodiment of the present invention, the pharmaceutical composition may be used for preventing or treating post-transplant rejection immune responses or for treating lymphocyte proliferative disorders.

According to an embodiment of the present invention, the pharmaceutical composition may exhibit an effect of reducing immunoglobulin production.

According to an embodiment of the present invention, the pharmaceutical composition may exhibit an effect of reducing lymph node enlargement, autoantibodies, and inflammatory cytokine production.

According to an embodiment of the present invention, the pharmaceutical composition may inhibit metastasis of cancer cells.

According to an embodiment of the present invention, the pharmaceutical composition may exert anti-aging or rejuvenating effects.

According to an embodiment of the present invention, the pharmaceutical composition may be characterized by a reduction in intracellular P-galactosidase activity or an increase in Ki-67 expression.

According to an embodiment of the present invention, the pharmaceutical composition may be used for the treatment of fibrotic diseases.

According to an embodiment of the present invention, the fibrotic disease may be pulmonary fibrosis, idiopathic pulmonary fibrosis (IPF), systemic sclerosis-associated interstitial lung disease (SSc-ILD), radiation-induced pulmonary fibrosis, hepatic fibrosis, renal fibrosis, cardiac fibrosis, retinal fibrosis, pancreatic fibrosis, or cutaneous fibrosis.

According to an embodiment of the present invention, the pharmaceutical composition may exhibit an effect of reducing α-SMA expression and collagen accumulation.

According to an embodiment of the present invention, the pharmaceutical composition may be used for inhibiting or antagonizing PDK1 (3-phosphoinositide-dependent protein kinase 1) expression.

According to an embodiment of the present invention, the pharmaceutical composition may be used for cancer or solid tumor cancer.

According to an embodiment of the present invention, the solid tumor cancer may be glioma, glioblastoma, medulloblastoma, meningioma, pituitary adenoma, optic glioma, spinal cord tumor, schwannoma, brain metastases, primary CNS lymphoma, non-small cell lung cancer, small cell lung cancer, Lung adenocarcinoma, Lung squamous cell carcinoma, Large cell carcinoma, Malignant mesothelioma, Tracheal cancer, Laryngeal cancer, Pharyngeal cancer, Nasal cavity cancer, Oral cancer, Tongue cancer, Salivary gland cancer, Laryngeal cancer, Pharyngeal cancer, Nasal cavity cancer, Paranasal sinus cancer, Thyroid cancer, Parathyroid cancer, Olfactory neuroblastoma, Invasive ductal carcinoma, Invasive lobular carcinoma, Ductal carcinoma in situ, Inflammatory breast cancer, Triple-negative breast cancer, Papillary carcinoma, Mucinous carcinoma, Tubular carcinoma, Medullary carcinoma, Metastatic breast cancer, Esophageal cancer, Gastric cancer, Small intestine cancer, Colon cancer, Rectal cancer, Hepatocellular carcinoma, Cholangiocarcinoma, Gallbladder cancer, Pancreatic cancer, Anal cancer, Renal cell carcinoma, Urothelial carcinoma, Bladder cancer, Ureteral cancer, Urethral cancer, Prostate cancer, Testicular cancer, Penile cancer, Adrenocortical carcinoma, Pheochromocytoma, Endometrial cancer, Cervical cancer, Uterine sarcoma, Ovarian cancer, Fallopian tube cancer, Vaginal cancer, Vulvar cancer, Hydatidiform mole, Choriocarcinoma, Germinoma, Testicular cancer, Penile cancer, Prostate cancer, Epididymal cancer, Seminal vesicle cancer, Urethral cancer, Germinoma, Choriocarcinoma, Teratoma, Leydig cell tumor, Osteosarcoma, Chondrosarcoma, Ewing sarcoma, Fibrosarcoma, Synovial sarcoma, Liposarcoma, Rhabdomyosarcoma, Leiomyosarcoma, Desmoid tumor, Giant cell tumor of bone, Skin cancer, Melanoma, Basal cell carcinoma, Squamous cell carcinoma, Kaposi's sarcoma, Neuroendocrine tumor, Gastrointestinal stromal tumor (GIST), Retinoblastoma, Wilms tumor, or Neuroblastoma.

According to the present invention, the pharmaceutical composition may be used for the treatment of blood cancer.

According to an embodiment of the present invention, the blood cancer may be acute lymphoblastic leukemia (ALL), acute myeloid leukemia (AML), chronic lymphocytic leukemia (CLL), chronic myeloid leukemia (CML), non-Hodgkin lymphoma (NHL), Hodgkin lymphoma (HL), peripheral T-cell lymphoma (PTCL), anaplastic large cell lymphoma, cutaneous T-cell lymphoma (CTCL), Plasmablastic lymphoma, Burkitt lymphoma, Multiple myeloma, Myelodysplastic syndromes (MDS), or Myeloproliferative neoplasms (MPN).

According to an embodiment of the present invention, the autoimmune diseases may include systemic lupus erythematosus (SLE), rheumatoid arthritis (RA), Sjogren's syndrome, multiple sclerosis, inflammatory bowel disease (Crohn's disease or ulcerative colitis), psoriatic arthritis, autoimmune hemolytic anemia, immune thrombocytopenia (ITP). Glomerulonephritis, systemic lupus erythematosus (SLE), glomerulonephritis, ankylosing spondylitis, myasthenia gravis, rheumatoid arthritis (RA), multiple sclerosis (MS), MS), systemic sclerosis, pernicious anemia, autoimmune anemia, inflammatory bowel disease (IBD), insulin-dependent diabetes mellitus (IDDM), type 1 diabetes, Graves' disease, Graves hyperthyroidism, Kikuchi disease, hemophagocytic lymphohistiocytosis, adult-onset Still's disease, Behcet's disease, immune thrombocytopenia (ITP), alopecia areata, IgG4-related disease, psoriasis, asthma, or transplant rejection. ITP), alopecia areata, IgG4-related disease, psoriasis, asthma, or transplant rejection.

According to the present invention, the pharmaceutical composition may be used for the purpose of preventing or treating Alzheimer's disease.

According to the present invention, the prevention or treatment may involve inhibiting the activity of PDK1 (phosphoinositide-dependent kinase-1).

According to the present invention, the pharmaceutical composition may inhibit pathological vascular neogenesis in the eye by inhibiting the activity of PDK1.

According to the present invention, the pathological vascular neogenesis may be induced by macular degeneration, diabetic retinopathy, retinal vein occlusion, or proliferative retinal diseases.

According to the present invention, a pharmaceutical composition comprising the compound described above or its pharmaceutically acceptable salt and pharmaceutically acceptable excipients is provided.

Another aspect of the present invention provides a pharmaceutical composition for the treatment or prevention of cancer or autoimmune diseases, comprising the compound represented by chemical formula 1 or its pharmaceutically acceptable salts.

In the pharmaceutical compositions according to the present invention, unless otherwise specifically stated, the terms used herein shall have the same meanings as those described above.

The term “treatment” as used herein means all of the following: improvement, alleviation, reversal, or cure of cancer or autoimmune diseases by administration of the composition according to the present invention.

The term “prevention” as used herein means all of the following: inhibition, delay, or prevention of the occurrence or recurrence of cancer or autoimmune diseases by administration of the composition according to the present invention.

The pharmaceutical composition of the present invention comprises the compound, its isomers, solvates, hydrates, crystalline forms, or salts thereof in an effective amount, and may be administered to a subject in need of prevention or treatment of cancer or autoimmune diseases.

The term “administration” as used herein means the physical introduction of the composition into the subject using any of the various methods and delivery systems known to those skilled in the art.

Such administration may be performed, for example, by oral administration, or by intravenous, intramuscular, subcutaneous, intraperitoneal, spinal, or other non-oral administration, such as injection or infusion, but is not limited thereto.

The frequency of administration may be, for example, single, multiple, or over one or more extended periods.

The pharmaceutical compositions of the present invention may be formulated as oral or non-oral dosage forms according to the administration routes described above.

The term “subject” as used herein includes humans and any non-human animals, wherein the non-human animals may include vertebrates, such as primates, dogs, cattle, horses, pigs, rodents, such as mice, rats, guinea pigs, etc.

In this specification, the term “subject” is used interchangeably with ‘individual’ and “patient.”

In particular, the subject to which the compound or composition according to the present invention is administered may be a cancer patient, or a cancer patient suffering from an autoimmune disease or having a high risk of developing an autoimmune disease.

Additionally, the subject to which the compound or composition according to the present invention is administered may be an autoimmune disease patient, or an autoimmune disease patient who has cancer or is at high risk of developing cancer.

Furthermore, the effective amount may be a “therapeutic effective amount” or a “preventive effective amount.”

The term “therapeutic effective dose” refers to any amount of a drug or therapeutic agent, used alone or in combination with other therapeutic agents, that can reduce the severity of disease symptoms, increase the frequency and duration of symptom-free periods, or prevent damage or disability caused by disease-related pain.

The term “preventive effective dose” refers to any amount of a drug or therapeutic agent that suppresses the onset or recurrence of a disease in the subject.

The level of the effective dose may be determined based on factors such as the severity of the target organism, age, gender, drug activity, drug sensitivity, administration time, administration route, and excretion rate, treatment duration, concomitant medications, and other factors well-known in the medical field.

Additionally, the dosage of the pharmaceutical composition may vary depending on the subject's age, gender, weight, route of administration, severity of the disease, etc., and specifically, the composition of the present invention may be administered at a dose of 0.1 to 100 mg/kg per day, once or multiple times, or at intervals of several days to several months, depending on the subject's symptoms.

Furthermore, the term “pharmaceutically acceptable salt” used in the present invention refers to a salt that is acceptable for administration to the subject.

Such salts with counterions are understood to have acceptable mammalian safety for a given administration regimen.

Such salts may also be derived from pharmaceutically acceptable inorganic or organic bases and pharmaceutically acceptable inorganic or organic acids, and may contain organic and inorganic counterions.

The neutral form of the compounds described in the present invention may be converted to the corresponding salt form by contacting the compound with a base or acid and isolating the resulting salt.

Additionally, the terms “pharmaceutically acceptable excipient,” “pharmaceutically acceptable diluent,” “pharmaceutically acceptable carrier,” and “pharmaceutically acceptable adjuvant” are used interchangeably and generally refer to excipients, diluents, carriers, or adjuvants that are safe, non-toxic, and not biologically or otherwise undesirable, and are useful for manufacturing pharmaceutical compositions, including excipients, diluents, carriers, and adjuvants that are acceptable for veterinary use and human pharmaceutical use.

The term ‘pharmaceutically acceptable excipient’ includes one or more excipients, diluents, carriers, and/or adjuvants.

Additionally, the term ‘pharmaceutical composition’ means a composition suitable for administration to a subject, such as a mammal, particularly a human.

Generally, a “pharmaceutical composition” is sterile and preferably free from contaminants that could cause undesirable reactions in the target organism (i.e., the compounds in the pharmaceutical composition are of pharmaceutical grade).

Pharmaceutical compositions may be processed for administration to target organisms or patients requiring them via a variety of different routes, including oral, buccal, rectal, non-oral, intra-abdominal, intradermal, intratracheal, intramuscular, subcutaneous, and others.

Additionally, the pharmaceutical composition may further include appropriate carriers, excipients, and diluents commonly used in the manufacture of pharmaceutical compositions.

Examples of carriers, excipients, and diluents that may be included in the composition include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil, but are not limited to these.

Additionally, the pharmaceutical composition may be administered in combination with other therapeutic agents.

In such cases, the pharmaceutical composition of the present invention and the other therapeutic agents may be administered simultaneously, sequentially, or individually.

The other therapeutic agents may be drugs such as compounds or proteins having preventive, therapeutic, or improving effects on cancer or autoimmune diseases, but are not limited thereto.

Furthermore, the pharmaceutical composition may be formulated to be administered simultaneously, sequentially, or individually with other therapeutic agents.

For example, the compounds, their isomers, solvates, hydrates, crystalline forms, or salts, and other therapeutic agents may be administered simultaneously as a single formulation, or as separate formulations simultaneously, sequentially, or individually.

For administration simultaneously, sequentially, or individually, the compounds, their isomers, solvates, hydrates, crystalline forms, or salts of the compounds, and other therapeutic agents included in the pharmaceutical composition of the present invention may be formulated separately in different containers or formulated together in the same container.

Additionally, the therapeutic agents other than the compounds, their isomers, solvates, hydrates, crystalline forms, or salts of the compounds included in the pharmaceutical composition of the present invention may have the same or different therapeutic effective amounts, administration times, administration intervals, administration routes, treatment periods, etc.

Another aspect of the present invention provides a method for treating or preventing cancer or autoimmune diseases, comprising the step of administering the compound represented by chemical formula 1 or its salts to a subject.

In the method for treating or preventing cancer or autoimmune diseases according to the present invention, unless otherwise specifically stated, the terms used herein shall have the same meanings as those defined above.

Additionally, in the method for treating or preventing cancer or autoimmune diseases according to the present invention, the compound or its salt may be administered to the subject simultaneously, sequentially, or individually with other formulations.

The term “simultaneous” administration means administering the compound or its salt and another therapeutic agent as a single formulation at the same time, or administering the compound or its salt and another therapeutic agent as separate formulations at the same time, in which case the routes of administration may be different.

The term “sequential” administration refers to administering the compound or its salt and other therapeutic agents in a relatively continuous manner, allowing the minimum possible time between administrations.

The term “separate” administration refers to administering the compound or its salt and other therapeutic agents at intervals of a certain time.

### Advantageous Effects

According to the present invention, the compound of formula 1 or its salt, which has PDK1 inhibitory, regulatory, or antagonistic activity, exhibits excellent efficacy in killing cancer cells or inhibiting tumor growth and proliferation, and has excellent safety for the human body.

Therefore, they can be usefully employed for the prevention, treatment, or improvement of cancer.

Additionally, the compounds of formula 1 or their salts according to the present invention, which exhibit excellent PDK1 inhibitory activity, not only inhibit T cell proliferation but also induce B cell apoptosis, thereby exhibiting potent immune-suppressive functions. Thus, they can be usefully employed for the treatment or prevention of autoimmune diseases that form autoantibodies, in addition to transplant rejection reactions.

Furthermore, by selectively inhibiting PDK1, the survival and proliferation of fibroblasts can be suppressed, and fibrosis progression can be blocked, making it useful for the treatment or prevention of fibrotic diseases such as idiopathic pulmonary fibrosis (IPF) or non-alcoholic steatohepatitis (NASH).

Furthermore, PDK1 inhibition can be useful for anti-aging or rejuvenation by reducing the activity of the AKT and mTOR pathways.

Additionally, by inhibiting PDK1 activity, it can mitigate neurotoxicity caused by Aβ and enhance the activity of TACE to promote the non-amyloid pathway, making it useful for the treatment or prevention of Alzheimer's disease.

Furthermore, the present invention can effectively block the metastasis process of tumors by inhibiting EMT, reducing mobility and invasiveness, inhibiting angiogenesis, and inhibiting Akt/mTOR signal transduction in cancer cells, thereby exhibiting metastasis inhibition and therapeutic effects in various solid tumors such as breast cancer, lung cancer, and pancreatic cancer.

### DESCRIPTION OF DRAWINGS

FIG. 1 is a conceptual diagram of the PDK1 domain map, showing the PKD, PHD, and PIF domains.

FIG. 2 is a conceptual diagram showing PDK1 and its downstream signaling pathways in T cells extracted from mouse spleens.

FIG. 3 is a graph showing the results of flow cytometry analysis according to the present invention,

FIG. 4 is a graph showing the IC50 value for PDK1 kinase activity inhibition in enzyme kinetics analysis of the compound according to the present invention,

FIG. 5 is a graph showing the results of measuring the reaction rate according to substrate concentration changes for the compound according to the present invention,

FIG. 6 is a graph showing the results of analyzing stained cells of the compound according to the present invention using flow cytometry,

FIG. 7 is a photograph of mice implanted with human-derived B-cell lymphoma cell line SU-DHL-8 (ATCC, Cat. #CRL-2961) subcutaneously to test the anticancer efficacy of the compound according to the present invention,

FIG. 8 shows photographs of the mice from FIG. 7 after administration of the compound according to the present invention, along with the elapsed days and measured sizes,

FIG. 9 is a graph showing that the body weight of mice administered the compound according to the present invention remains constant over time (days),

FIG. 10 is a schedule showing the psoriasis treatment schedule for mice administered the compound according to the present invention,

FIG. 11 is a photograph of the skin of mice administered the compound according to the present invention at 8 days to assess the extent of psoriasis treatment,

FIG. 12 is a photograph of the skin thickness of mice administered the compound according to the present invention at the 8-day time point to determine the psoriasis treatment efficacy,

FIG. 13 is a graph showing the results of measuring the PASI (Psoriasis Area and Severity Index) score to evaluate the treatment efficacy of the compound according to the present invention in psoriasis-induced mice,

FIG. 14 is a graph showing the results of analyzing the concentration of psoriasis-related cytokines (IL-17A) in the heart serum of mice administered the compound according to the present invention,

FIG. 15 is a graph showing the results of measuring the body weight of mice with lupus administered the compound according to the present invention,

FIG. 16 is a graph showing the results of analyzing the protein concentration in the urine of mice with lupus administered the compound according to the present invention using ‘Quick Start™ Bradford 1× Dye Reagent (Bio-Rad, Cat. #5000205)’,

FIG. 17 is a graph showing the results of measuring the concentration of anti-nuclear antibodies (Anti-dsDNA Ab) in serum isolated from mice administered the compound according to the present invention using the ‘Mouse Anti-dsDNA IgG ELISA Kit (Alpha Diagnostic International, Cat. #5120)’,

FIG. 18 is a graph showing the results of measuring the concentration of total IgG in serum isolated from mice administered the compound according to the present invention using the ‘IgG (Total) Mouse Uncoated ELISA Kit (Invitrogen, Cat. #88-50400)’.

FIG. 19 is a graph showing the results of staining mouse spleen cells administered with the compound according to the present invention and analyzing them by flow cytometry,

FIG. 20 is a graph showing the results of gene expression analysis of cytokines in the kidneys of lupus mice administered with the compound according to the present invention to confirm the effect on systemic immune responses,

FIG. 21 is a figure showing the results of observing kidney tissue from lupus mice administered the compound according to the present invention, which were sectioned, stained, and observed to confirm the effects on kidney tissue.

FIG. 22 is a photograph obtained by performing immunofluorescence staining and observing under a fluorescence microscope to confirm the degree of IgG deposition in the kidney tissue of lupus mice administered the compound according to the present invention,

FIGS. 23 and 24 are photographs showing the alleviated alopecia symptoms on the face of lupus mice administered the compound according to the present invention,

FIG. 23 shows photographs after treating with G015F at 30 mg/kg for 2 months (60 days), and FIG. 24 shows photographs after removing the skin and examining the tissue after treating with GO15F, G048, and G088 for 14 days,

FIG. 25 shows photographs taken using an optical microscope after performing Ki-67 immunohistochemistry (Ki-67 IHC) to confirm the cell proliferation activity of hair follicles in the skin tissue of lupus mice administered the compound according to the present invention.

FIG. 26 is a photograph of the paw of a rheumatoid mouse administered the compound according to the present invention,

FIG. 27 is a photograph of joint tissue extracted and observed to confirm the severity of rheumatoid arthritis in mice administered the compound according to the present invention,

FIG. 28 is a photograph of joint tissue extracted and observed to confirm the severity of rheumatoid arthritis in mice administered the compound according to the present invention,

FIG. 29 is a graph showing the results of analyzing the serum of rheumatoid mice administered the compound according to the present invention using an IL-17A, TNF-α Mouse ELISA kit,

FIG. 30 is a photograph of HCT-116 colon cancer cells stained and photographed under a microscope to evaluate the metastasis inhibitory efficacy of the compound according to the present invention,

FIG. 31 is a photograph of mice with human-derived colon cancer cells implanted subcutaneously, taken at different time points,

FIG. 32 is a figure showing the results of evaluating the tumor growth inhibitory efficacy of the compound according to the present invention in mice with human-derived colon cancer cells implanted subcutaneously,

FIG. 33 is a graph showing the results of measuring the total IgG concentration in the serum of normal mice and mice induced with a sensitized animal model, and expressing the results as relative concentration values,

FIG. 34 is a graph showing the results of analyzing changes in B cell subtypes within spleen cells of mice to confirm the effect of administering the compound according to the present invention on changes in the ratio of immune cells in the body of sensitized mice,

FIG. 35 is a graph showing the results of analyzing changes in T cell subtypes within spleen cells of mice to confirm the effect of administering the compound according to the present invention on changes in the ratio of immune cells in sensitized mice.

FIG. 36 is a graph showing the results of measuring the total IgG concentration in the serum of normal mice and mice induced with a sensitized animal model, and expressing these values as relative concentrations.

FIG. 37 is a graph showing the results of analyzing the ratio of CD3- and B220-positive cells in each cell using flow cytometry after administering the compound according to the present invention to mice with skin grafts and isolating immune cells from the spleen.

FIG. 38 is a photograph of mice with skin grafts administered with the compound according to the present invention.

FIG. 39 is a figure showing the results of analyzing P-galactosidase activity and Ki-67 expression after treating NHDF (Normal Human Dermal Fibroblast, Promocell) cells with the compound according to the present invention,

FIG. 40 is a figure showing the results of observing fluorescent expression of Ki-67 by applying FACS analysis to NHDF (Normal Human Dermal Fibroblast, Promocell) cells treated with the compound according to the present invention,

FIG. 41 is a photograph of a mouse model with induced pulmonary fibrosis,

FIG. 42 is a photograph of a mouse with induced pulmonary fibrosis administered with the compound according to the present invention, followed by extraction of the lung, staining, pathological analysis, and photography,

FIG. 43 is a photograph of a mouse with induced pulmonary fibrosis administered with the compound according to the present invention, followed by extraction of the lung, immunohistochemical (IHC) staining, analysis, and photography,

FIG. 44 is a diagram showing the results of Western blot analysis performed after treating human-derived lung fibroblast MRC-5 cells with the compound according to the present invention, extracting proteins, and transferring them to a PVDF membrane,

FIG. 45 is a diagram showing the experimental method and schedule for administering the compound according to the present invention to atopic-induced mice,

FIG. 46 is a photograph showing the extent of skin lesions on the back of atopic-induced mice administered the compound according to the present invention,

FIG. 47 is a result of pathological tissue analysis of the back region of atopic-induced mice administered the compound according to the present invention, analyzed through H&E and Toluidine Blue staining,

FIG. 48 is a graph obtained through a comparative analysis of the binding affinity between PDK1 and G088 using Microscale Thermophoresis (MST) according to the invention.

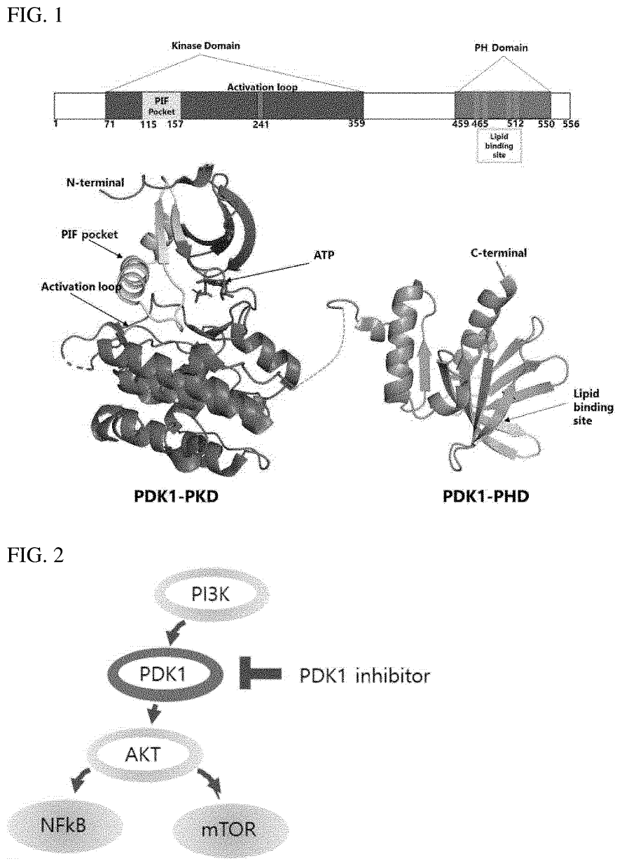

### MODE FOR INVENTION

The present invention will now be described in more detail with reference to the embodiments.

However, these embodiments are intended to illustrate the present invention and are not intended to limit the scope of the present invention.

Furthermore, those skilled in the art will appreciate that various modifications and changes may be made to the present invention without departing from the scope of the present invention.

Unless otherwise defined in this specification, terms used herein shall have the meanings commonly understood in the technical field to which the present invention pertains.

Furthermore, unless otherwise defined in the context, singular terms shall include plural terms, and plural terms shall include singular terms.

### Manufacturing Example

The compounds corresponding to the examples and comparative examples of the present invention were prepared according to the reaction formula 1 below.

Compounds with different substituents were also prepared through similar steps, but not all are explicitly stated in this specification.

Those skilled in the art can easily prepare compounds with different substituents by referring to the following representative examples.

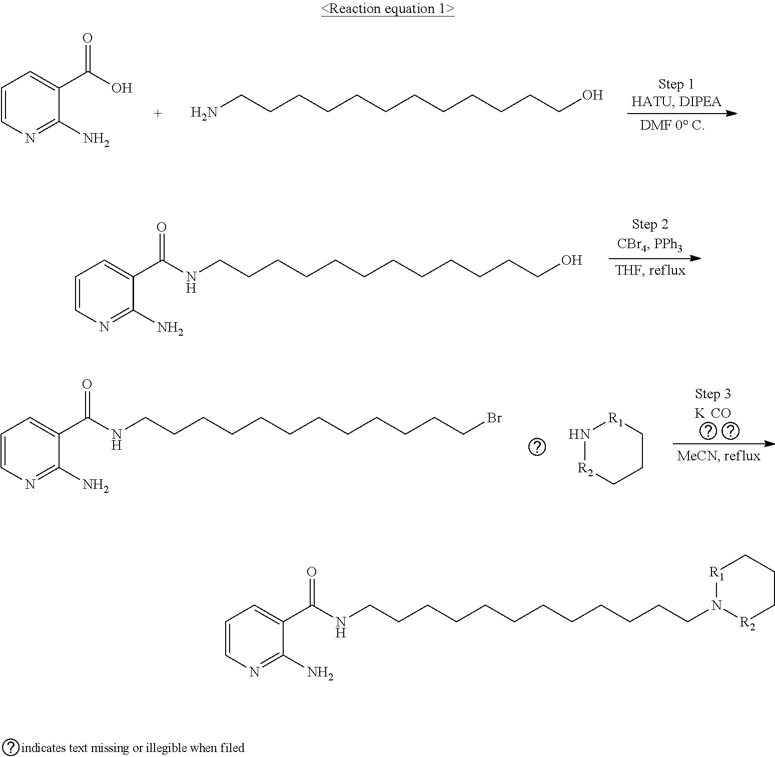

Reaction equation 1, when examined in detail, involves adding carboxylic acid derivatives (1.0 equiv.), 12-aminododecan-1-ol (1.5 equiv.), HATU (2.0 equiv.), and DIPEA (2.0 equiv.) to a round-bottom flask, dissolving them in DMF, stirring for 3 hours, and after the reaction is complete, Step 1 is completed by reverse-phase purification using MPLC under conditions of H₂O (0.1% formic acid) and acetonitrile. In a round-bottom flask, PPh₃ (2.8 equiv.) is dissolved in CH₂Cl₂, CBr₄ (1.4 equiv.) is added, and after 10 minutes, the product from Step 1 is added and stirred for 1 hour. After the reaction is complete, purify by silica gel column chromatography (hexane/ethyl acetate) to complete Step 2. Add the product from Step 2, a secondary amine derivative (3.0 equiv.), and K₂CO₃ (10.0 equiv.) to a round-bottom flask, dissolve in acetonitrile, and stir for 24 hours. After the reaction is complete, purify using amine column chromatography (CH₂Cl₂/MeOH) to obtain the product of Step 3.

Therefore, by substituting the bromine (product after Step 2) at the terminal position of the reaction equation 1 with a terminal group, the following compounds can be easily synthesized.

This is because the third step (Step 3) of reaction 1 can be performed using similar reaction 2, 3, and 4.

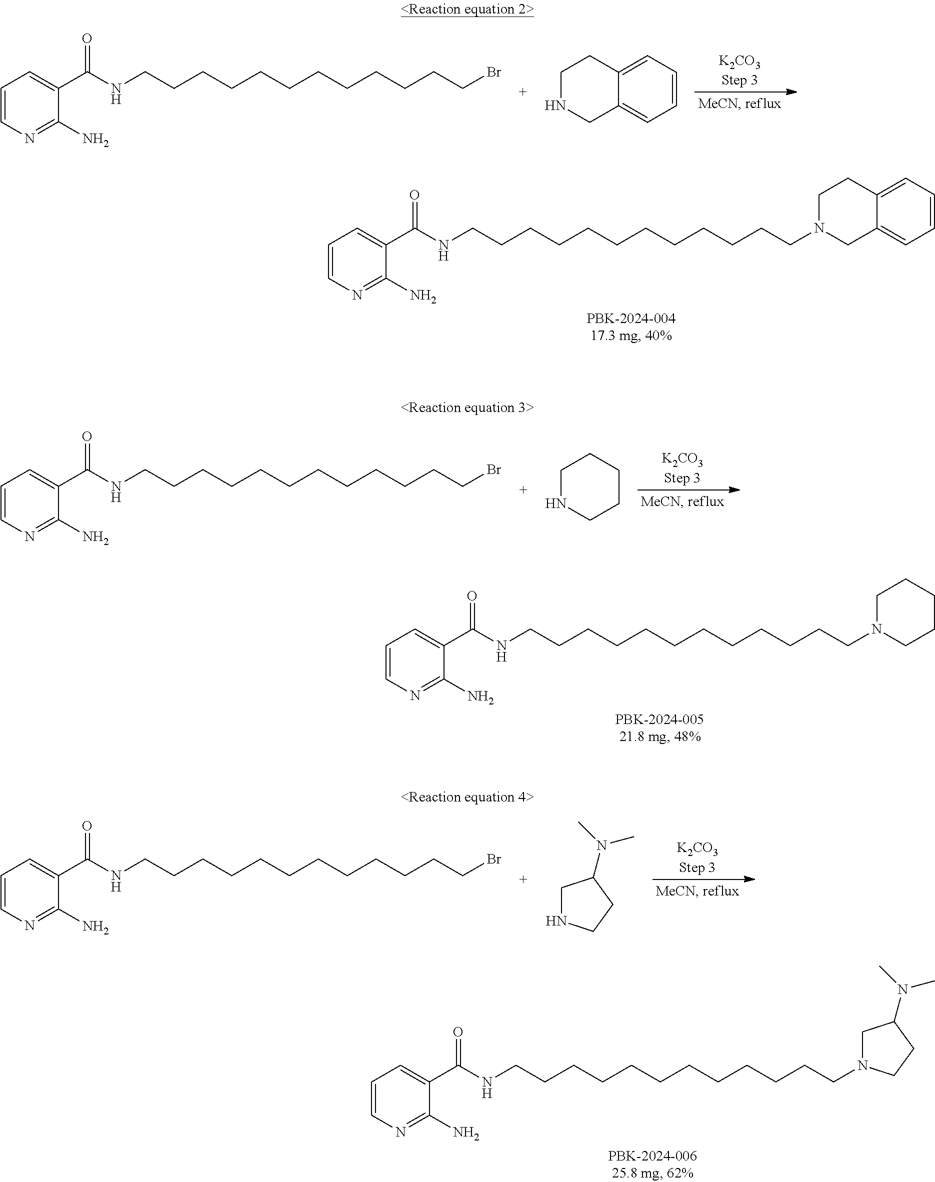

Compounds such as PBK-2024-007, which can be easily synthesized using the above reactions, can be observed.

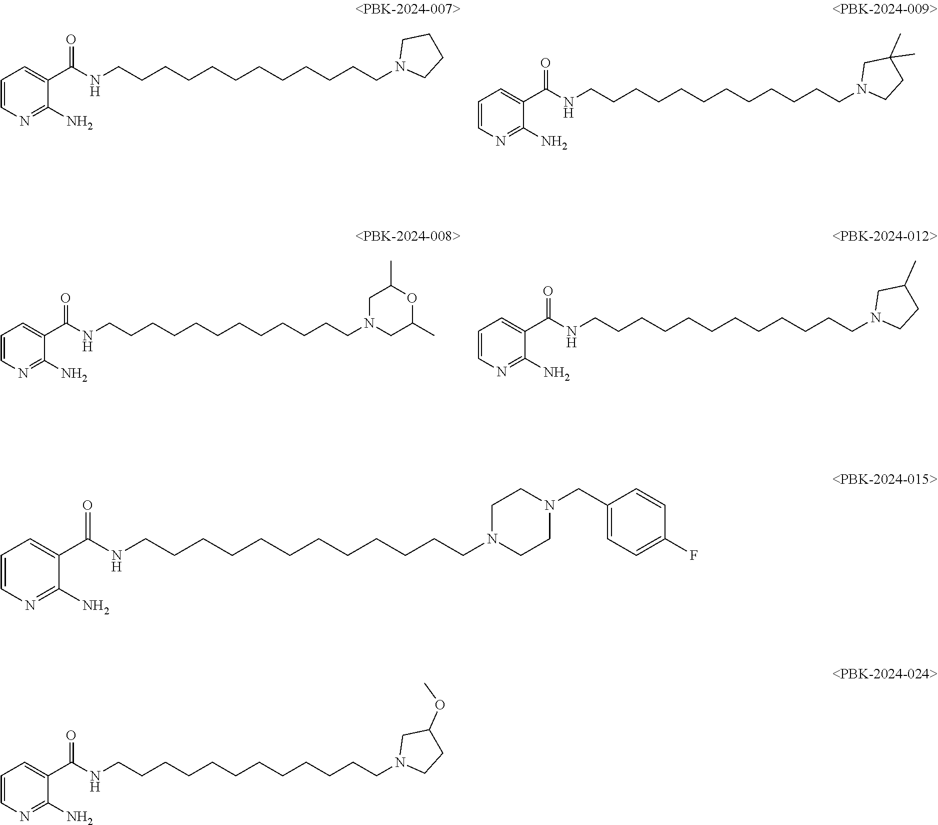 [SMILES: Nc1ncccc1C(=O)NCCCCCCCCCCCCN1CCCC1 ; CC1(C)CCN(CCCCCCCCCCCCNC(=O)c2cccnc2N)C1 ; CC1CN(CCCCCCCCCCCCNC(=O)c2cccnc2N)CC(C)O1 ; CC1CCN(CCCCCCCCCCCCNC(=O)c2cccnc2N)C1 ; Nc1ncccc1C(=O)NCCCCCCCCCCCCN1CCN(Cc2ccc(F)cc2)CC1 ; COC1CCN(CCCCCCCCCCCCNC(=O)c2cccnc2N)C1]

Additionally, even when the pyridine ring is replaced with a benzene ring in the above reaction, a similar reaction 5 can be utilized.

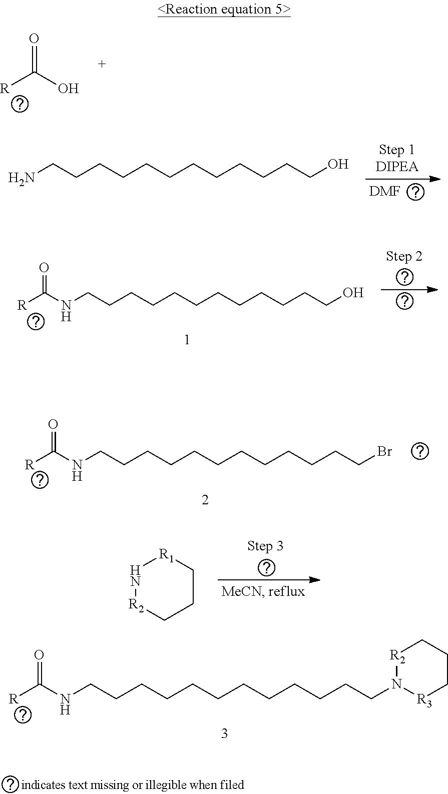

Add carboxylic acid derivatives (1.0 equiv.), 12-aminododecan-1-ol (1.5 equiv.), HATU (2.0 equiv.), and DIPEA (2.0 equiv.) to a round-bottom flask, dissolve in DMF, and stir for 3 hours.

After the reaction is complete, use MPLC to perform reverse-phase purification under conditions of H₂O (0.1% formic acid) and acetonitrile to complete step 1 and obtain product 1.

Dissolve PPh₃ (2.8 equiv.) in CH₂Cl₂ in a round-bottom flask, add CBr₄ (1.4 equiv.), and after 10 minutes, add product 1 and stir for 1 hour.

After the reaction is complete, purify by silica gel column chromatography (hexane/ethyl acetate) to obtain product 2.

In a round-bottom flask, add product 2, a secondary amine derivative (3.0 equiv.), and K₂CO₃ (10.0 equiv.), dissolve in acetonitrile, and stir for 24 hours.

After the reaction is complete, purify by amine column chromatography (CH₂Cl₂/MeOH) to obtain product 3.

The chemical structures, physical properties, identification data, molecular weights, etc., of the compounds prepared based on the reaction equations described above are as follows.

### Various Compound Examples

The chemical structures, physical properties, identification data, molecular weights, etc., of the compounds prepared as described above are representative examples as follows.

The chemical structures of the compounds are listed first, followed by their identification information.

### Chemical Structures of Compounds

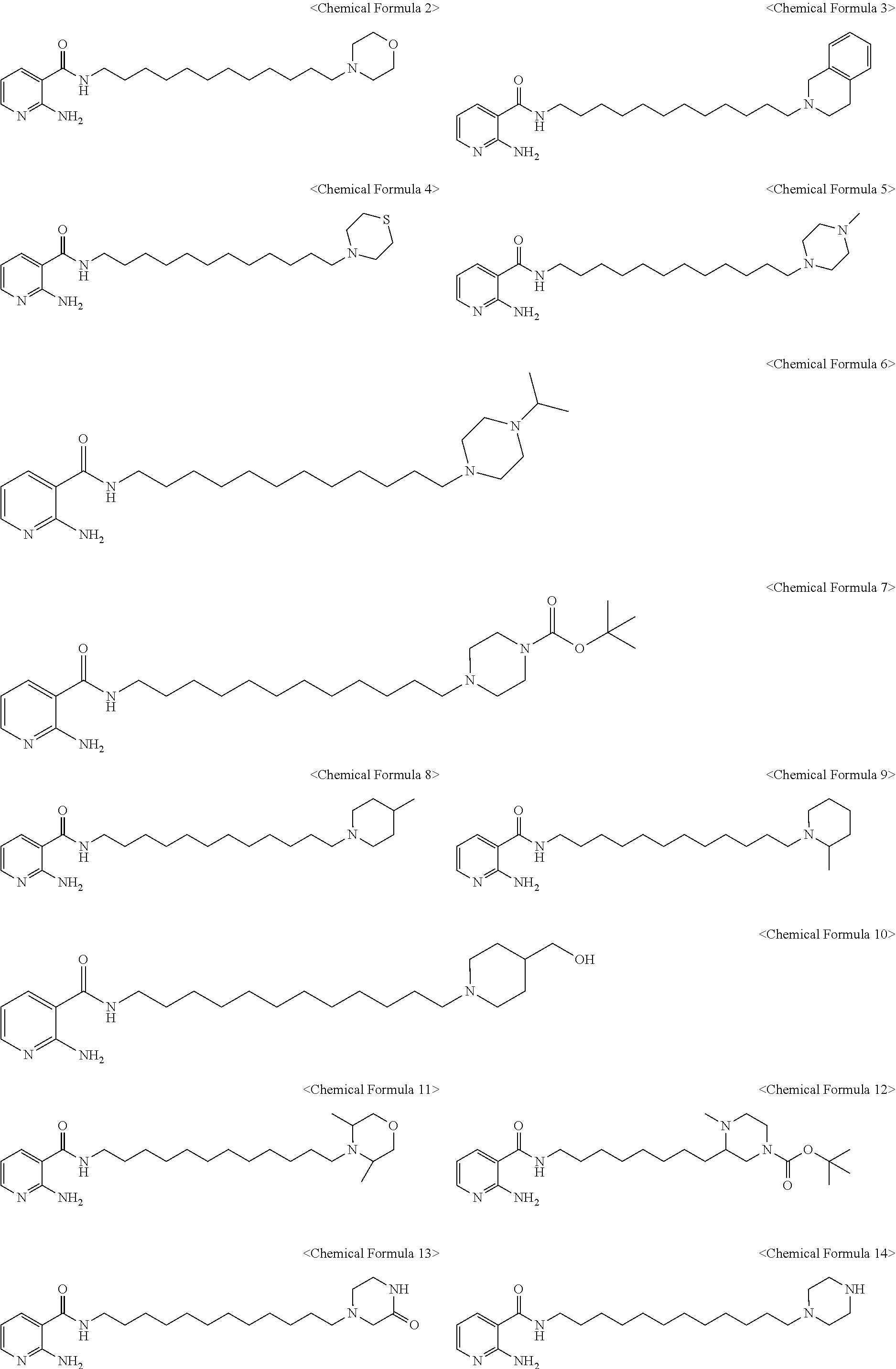

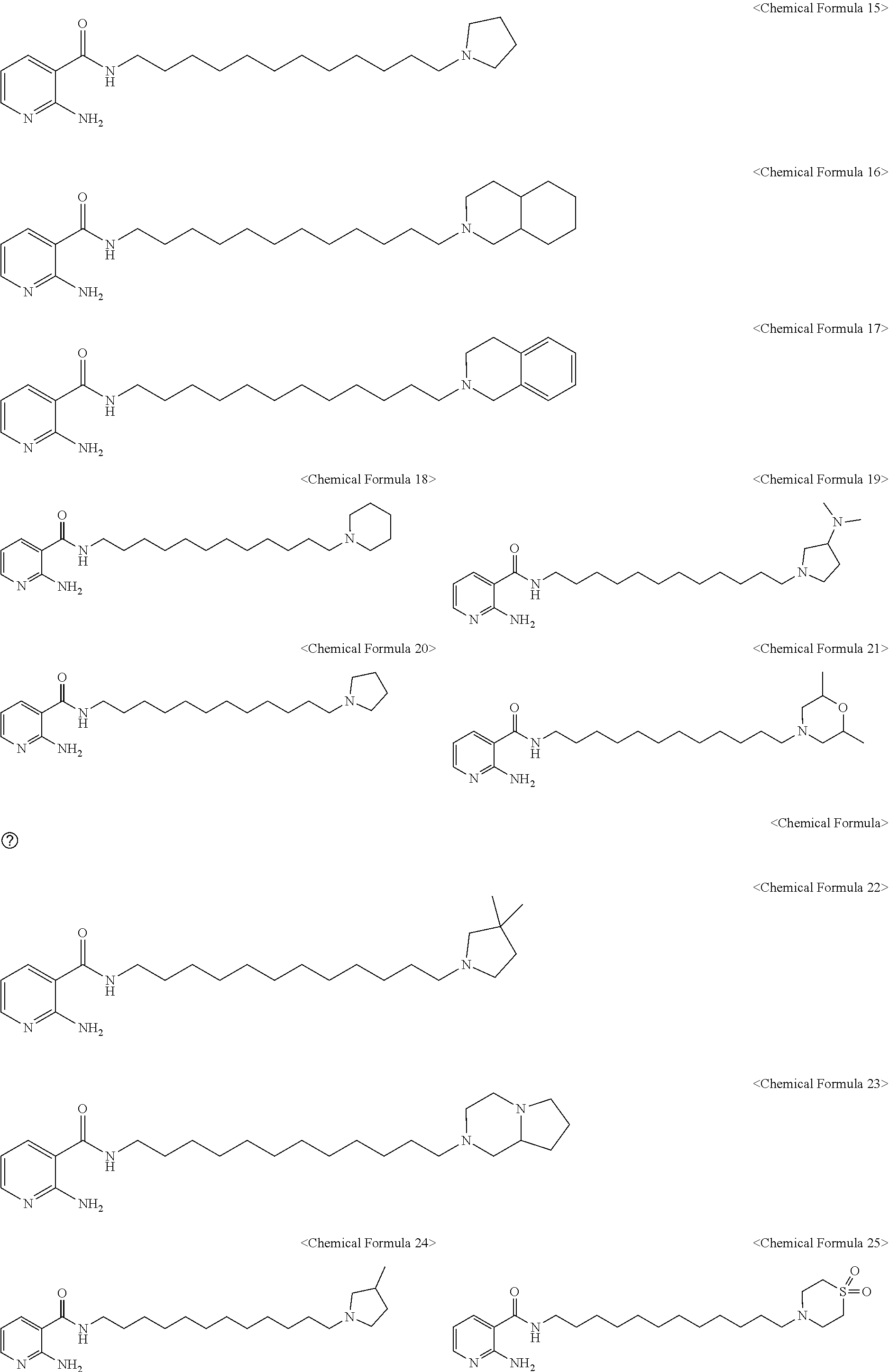

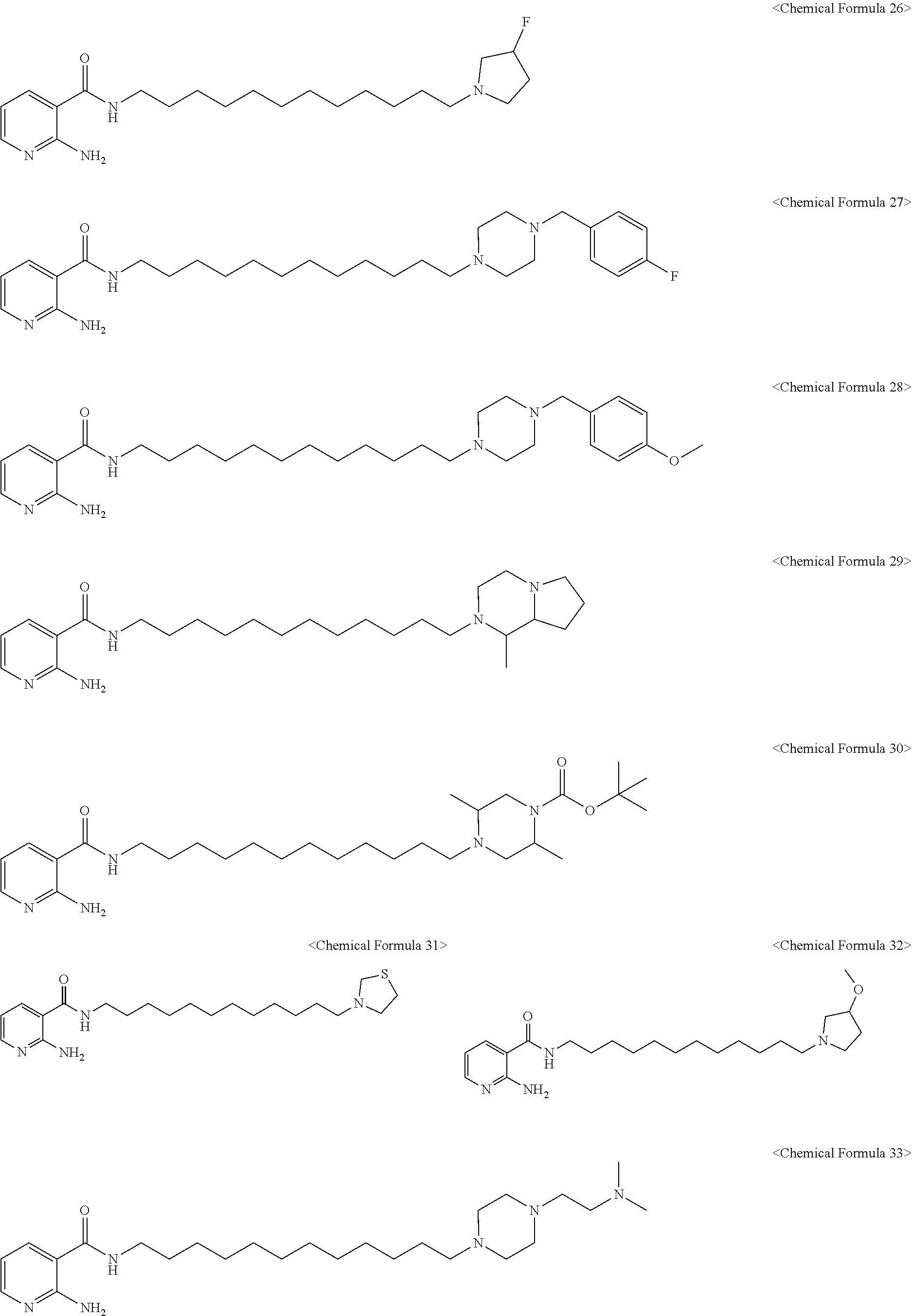

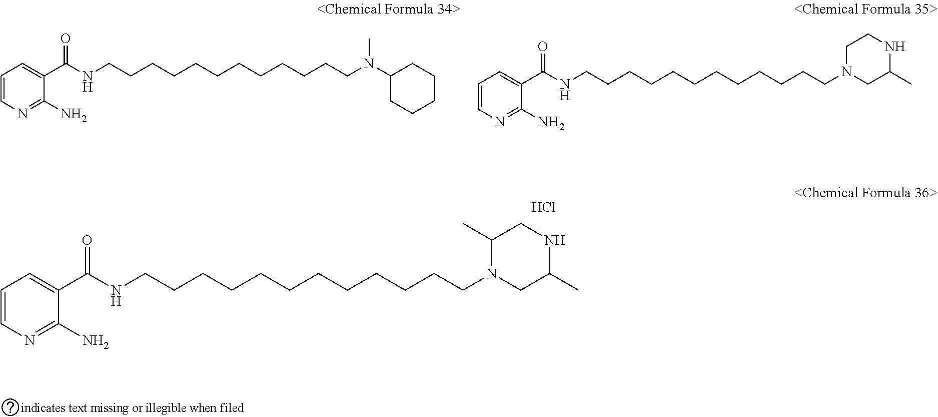 [SMILES: CN(CCCCCCCCCCCCNC(=O)c1cccnc1N)C1CCCCC1 ; CC1CN(CCCCCCCCCCCCNC(=O)c2cccnc2N)CCN1 ; Cl ; CC1CN(CCCCCCCCCCCCNC(=O)c2cccnc2N)C(C)CN1]

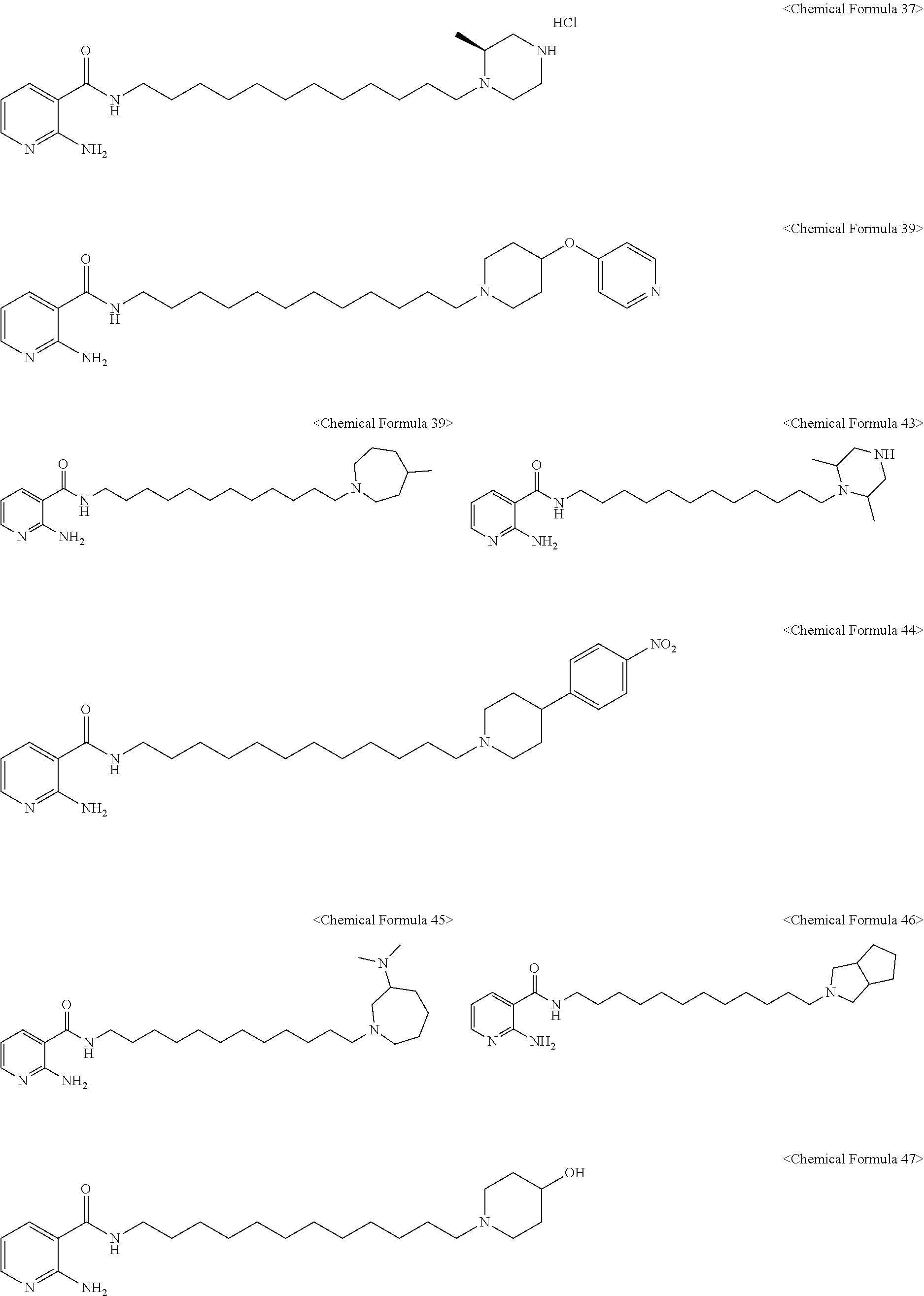

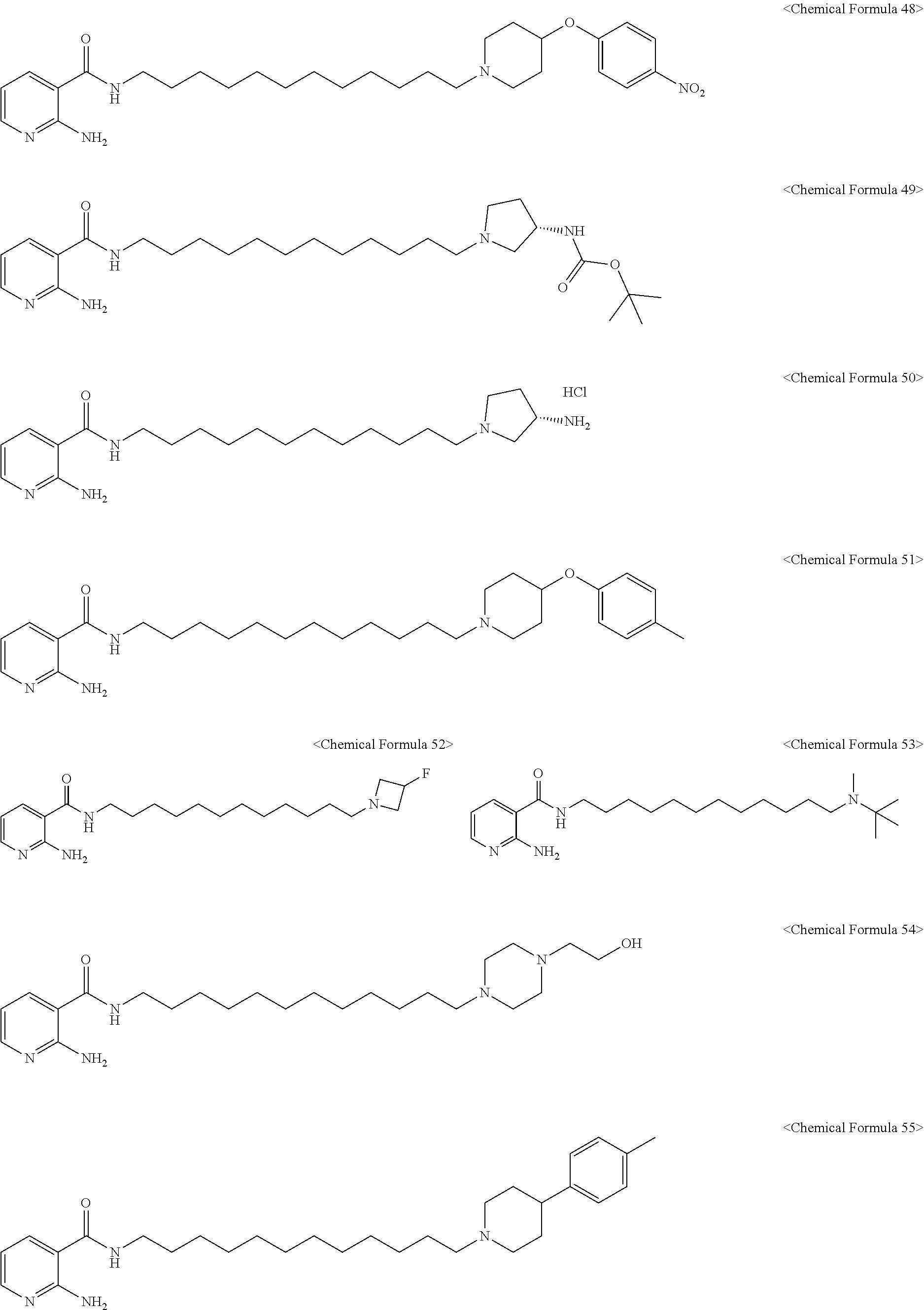

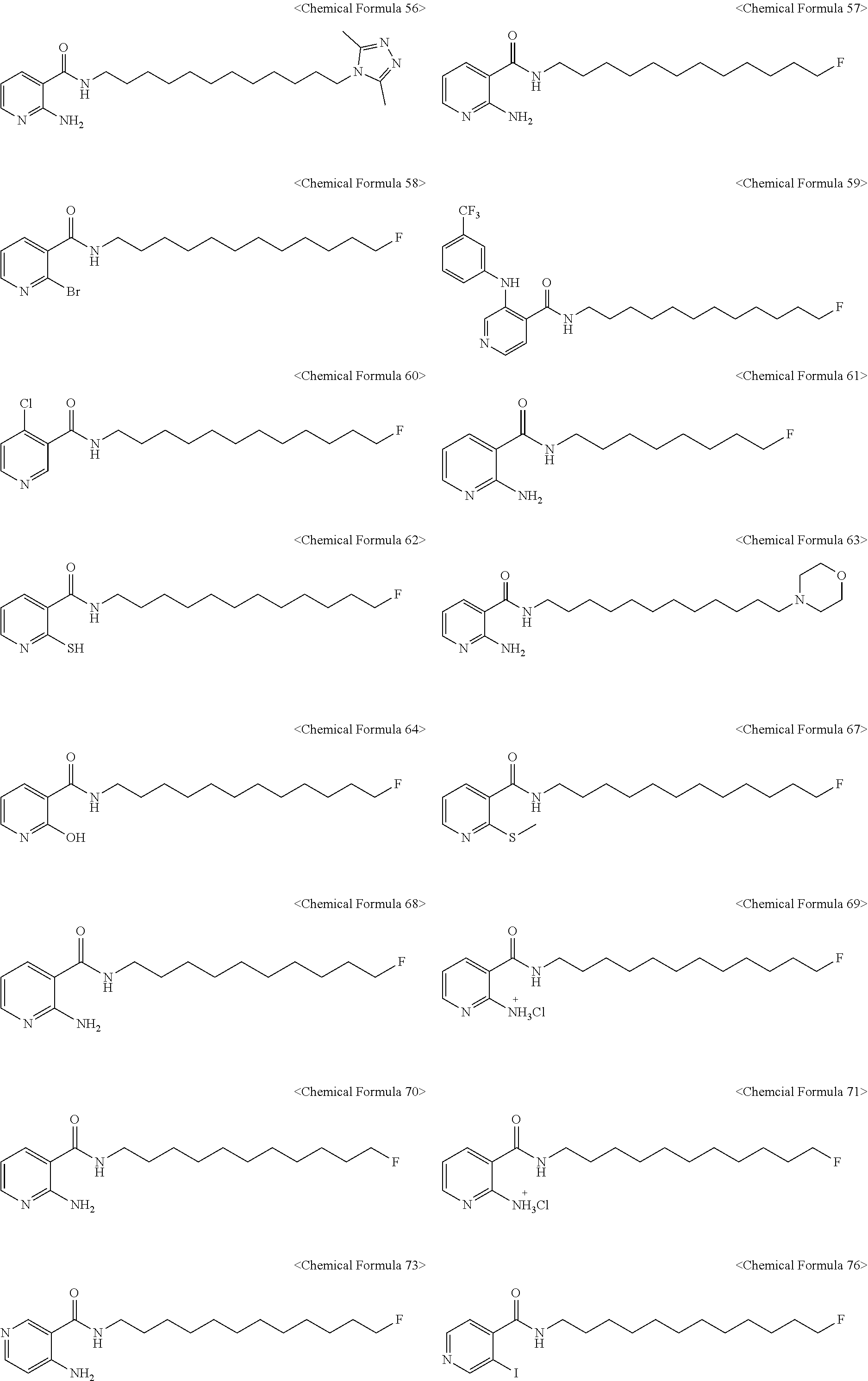

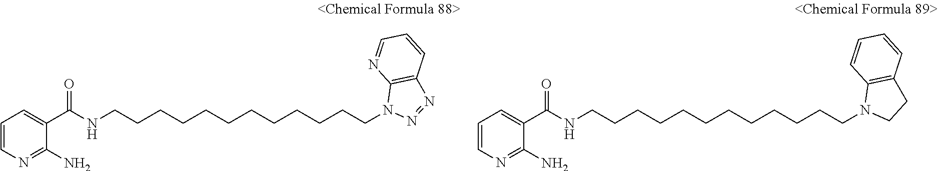 [SMILES: Nc1ncccc1C(=O)NCCCCCCCCCCCCn1nnc2cccnc21 ; Nc1ncccc1C(=O)NCCCCCCCCCCCCN1CCc2ccccc21]

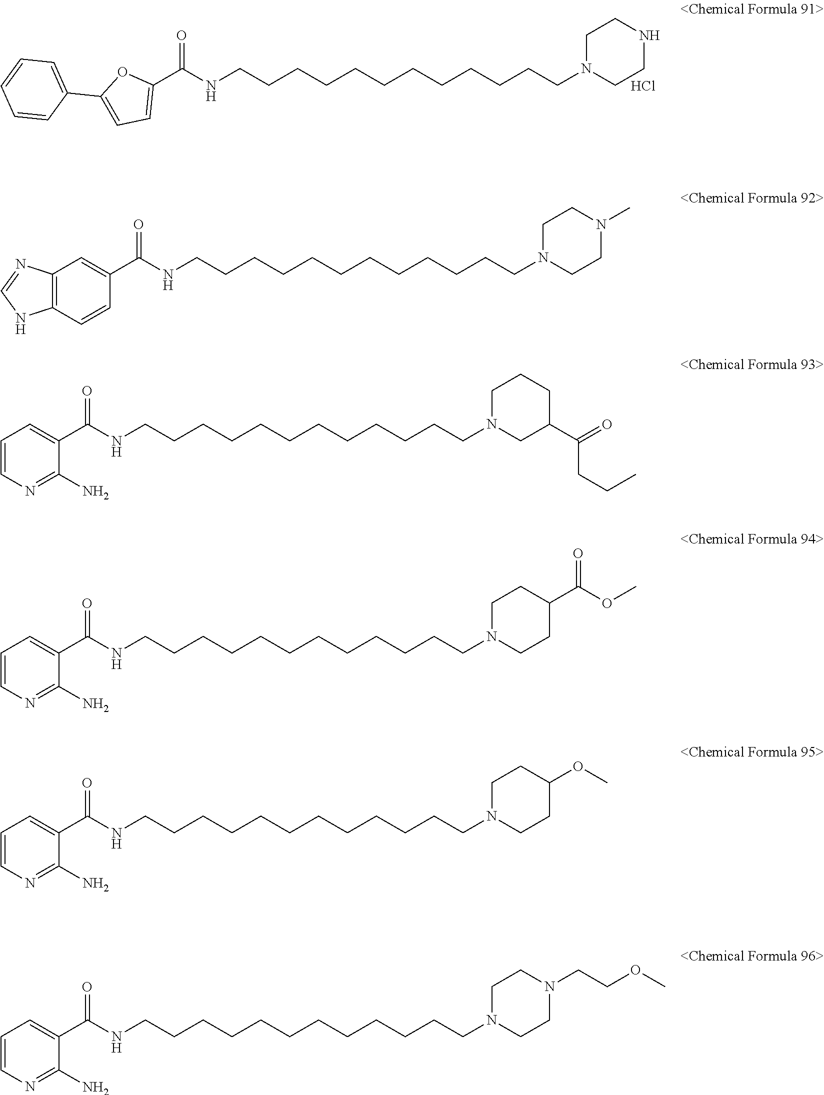

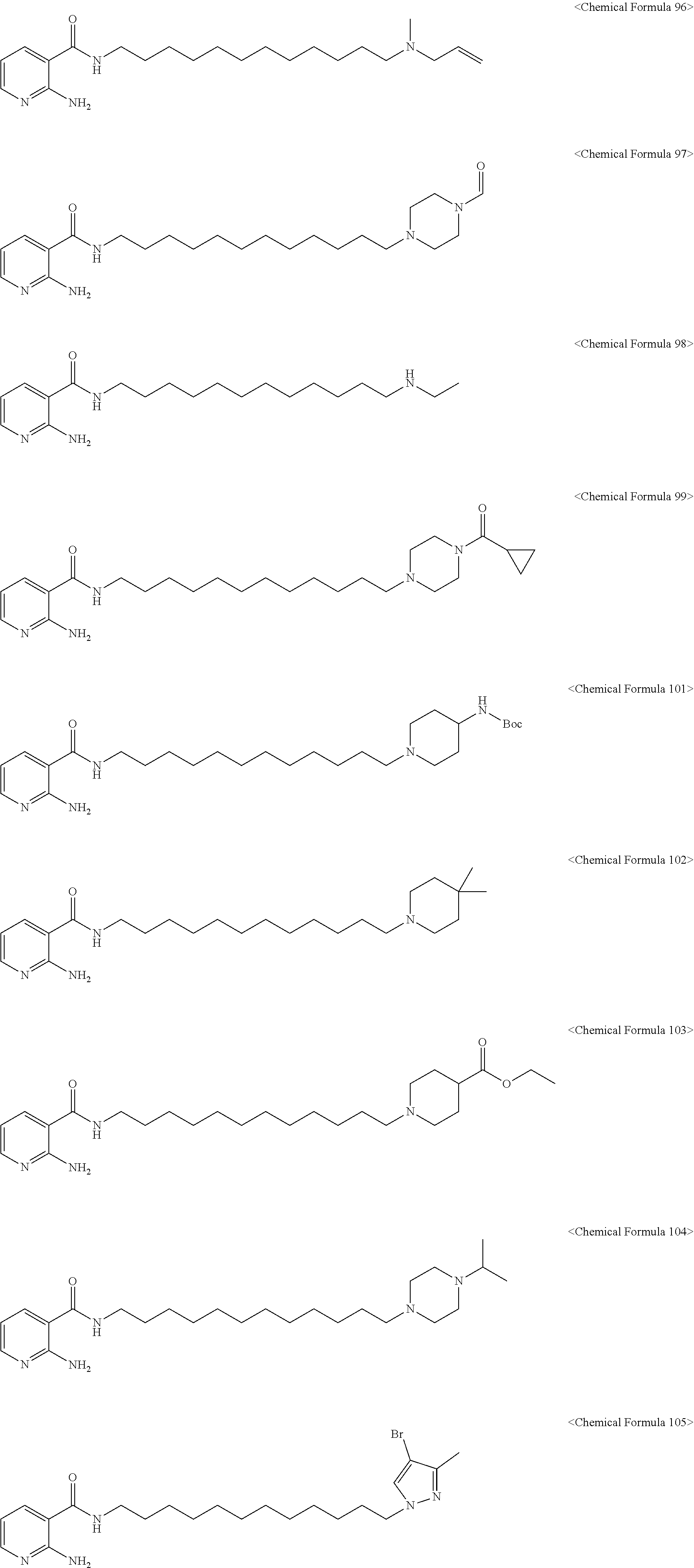

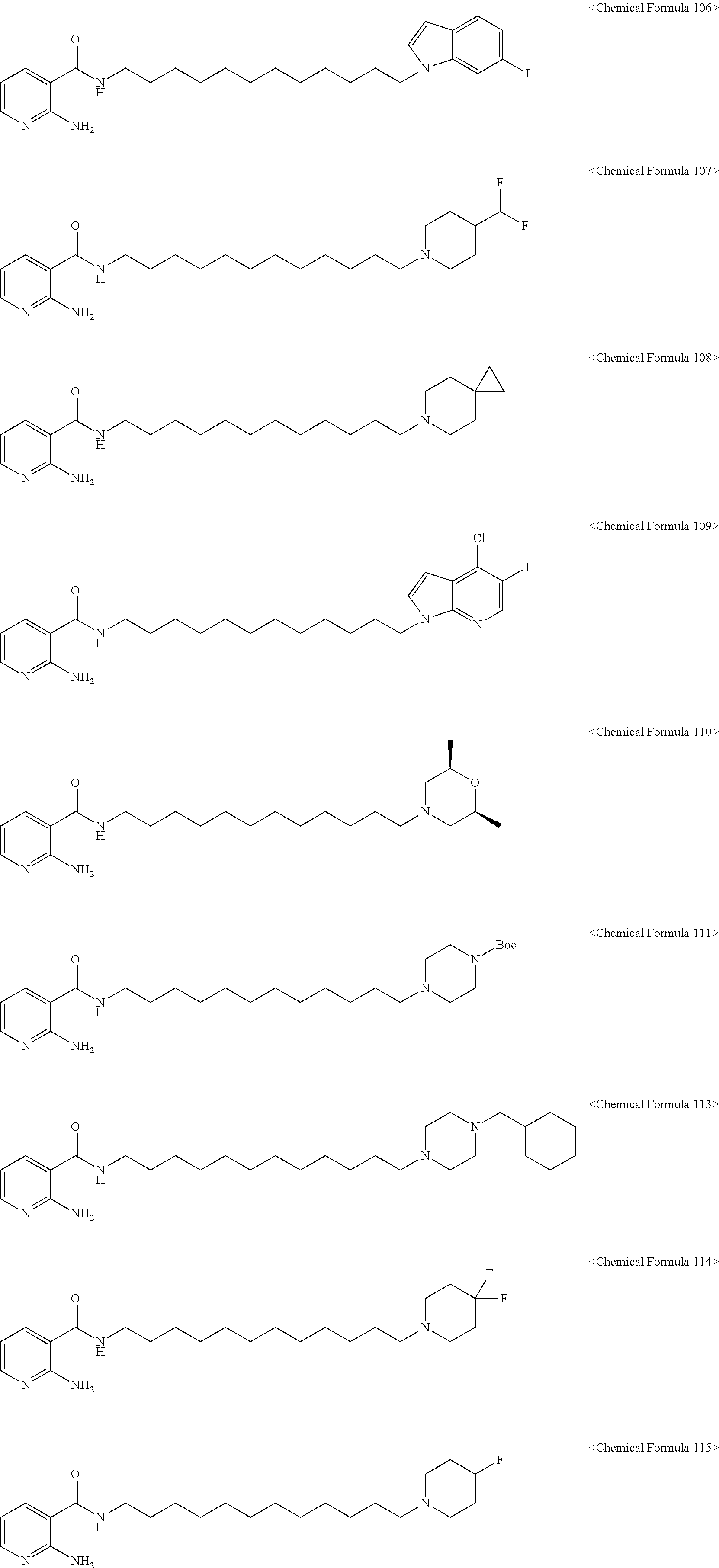

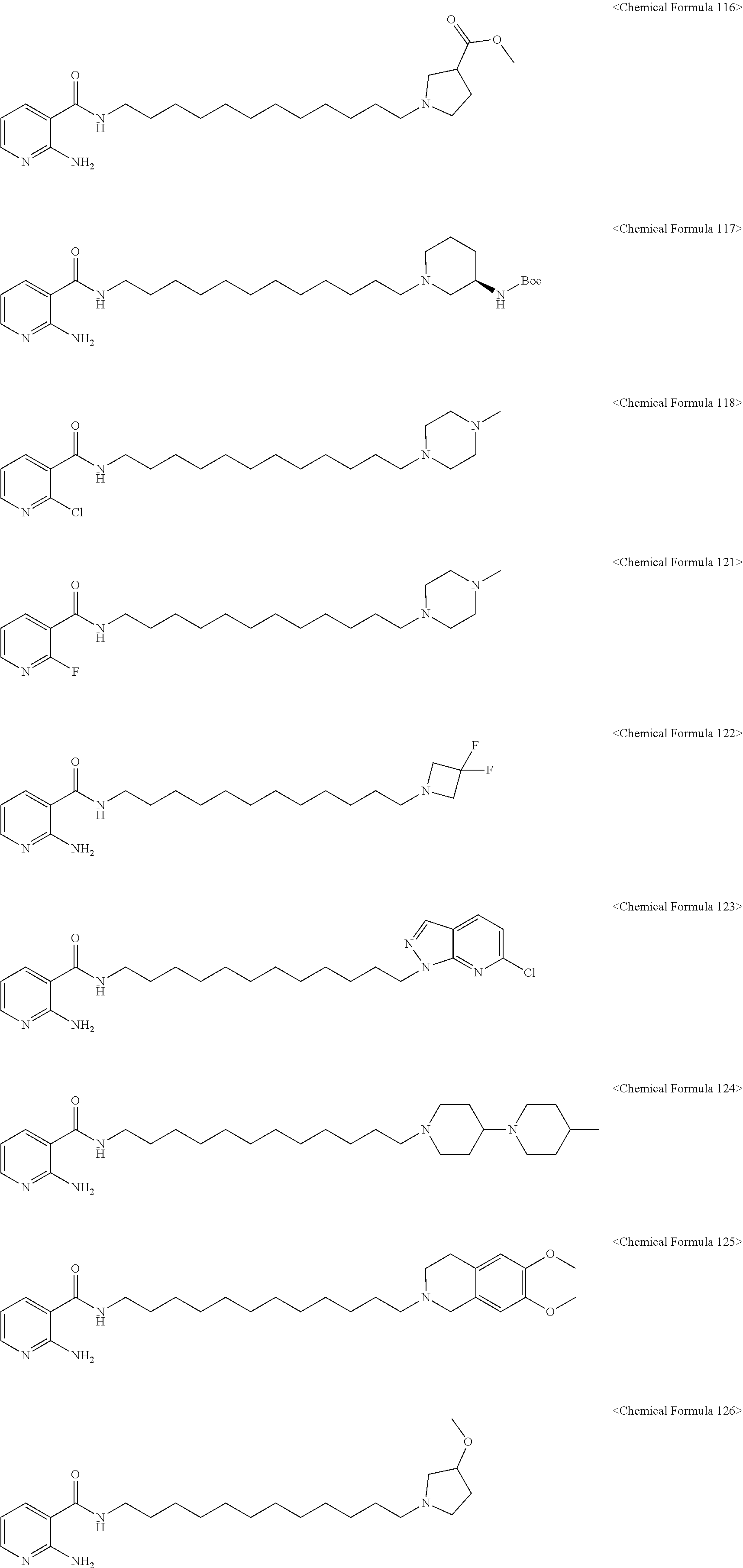

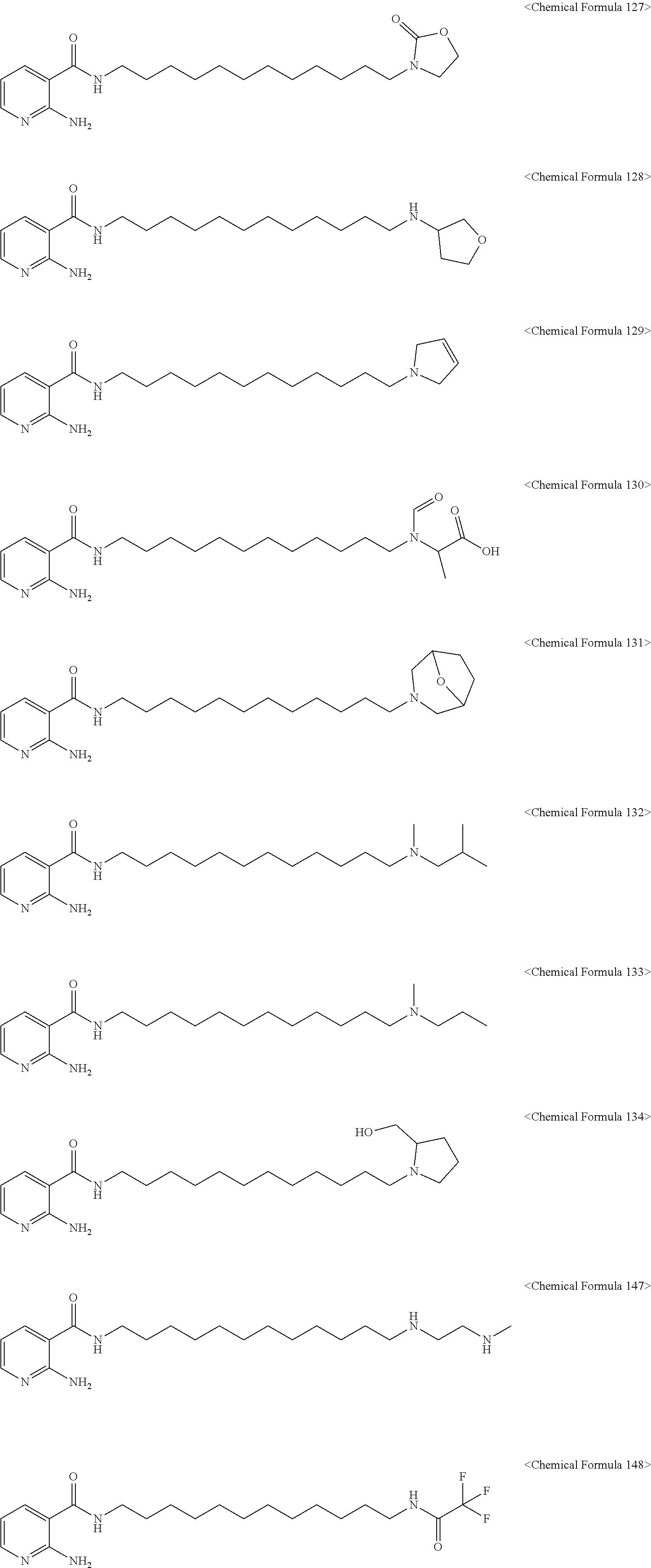

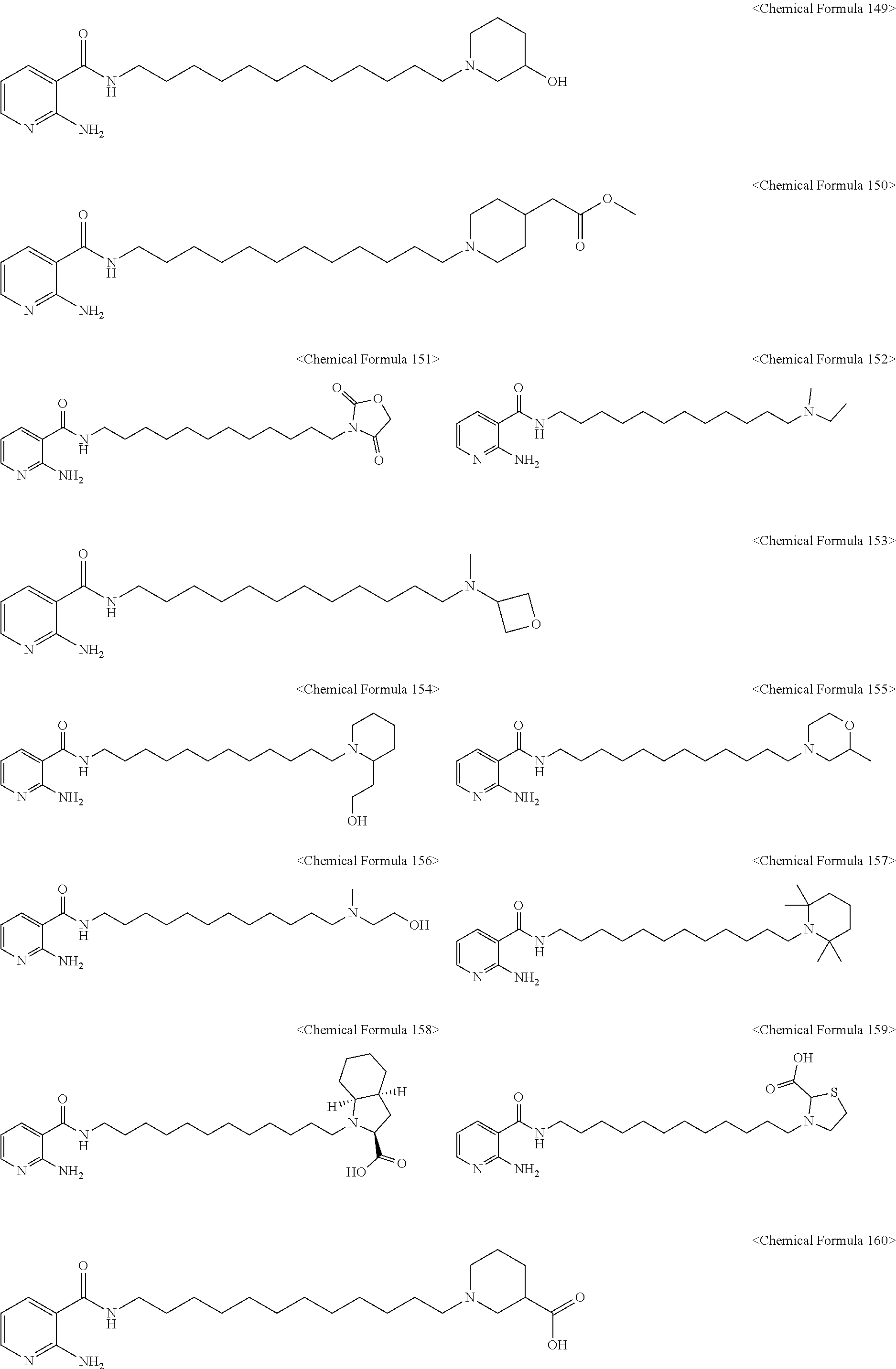

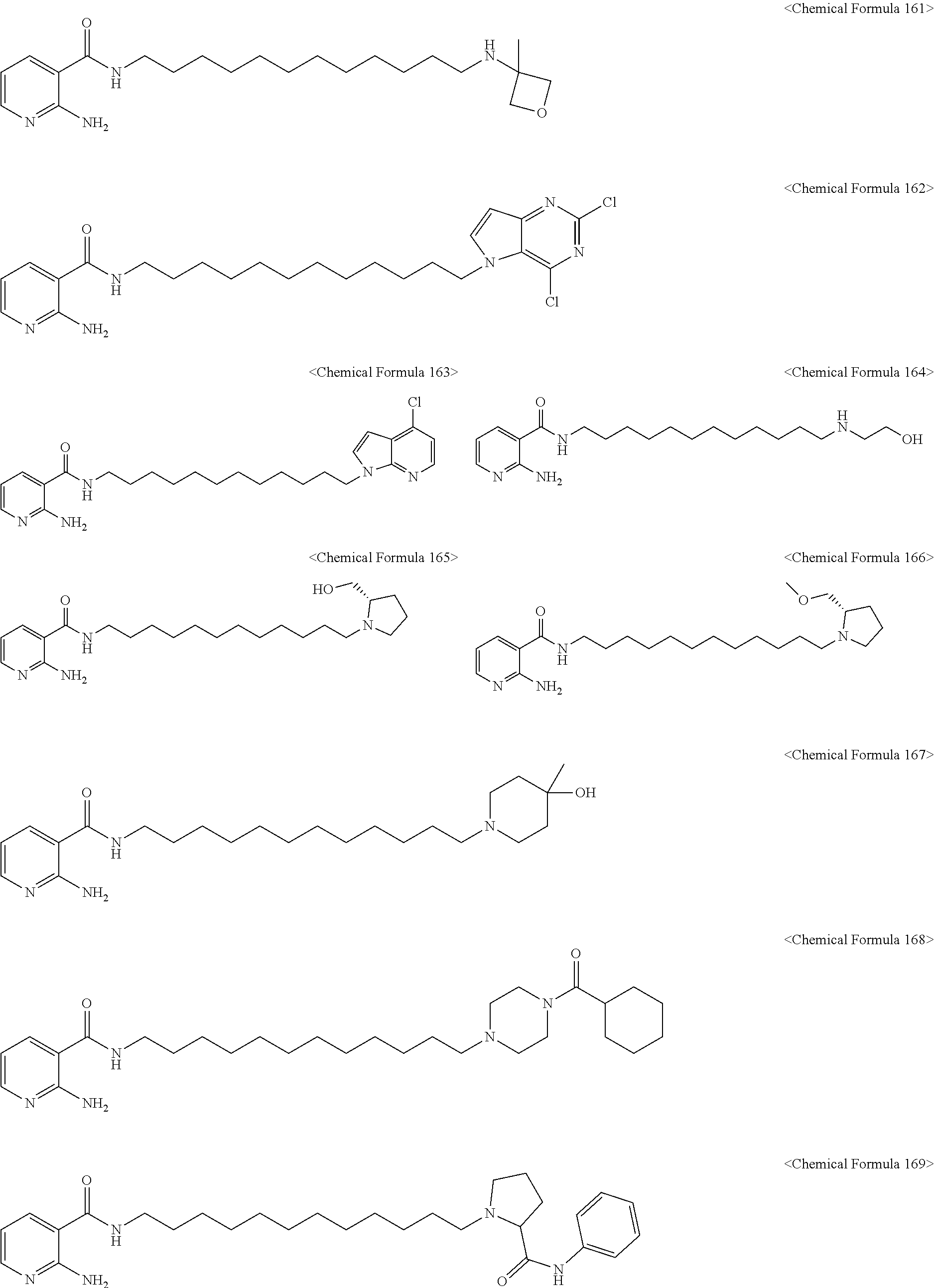

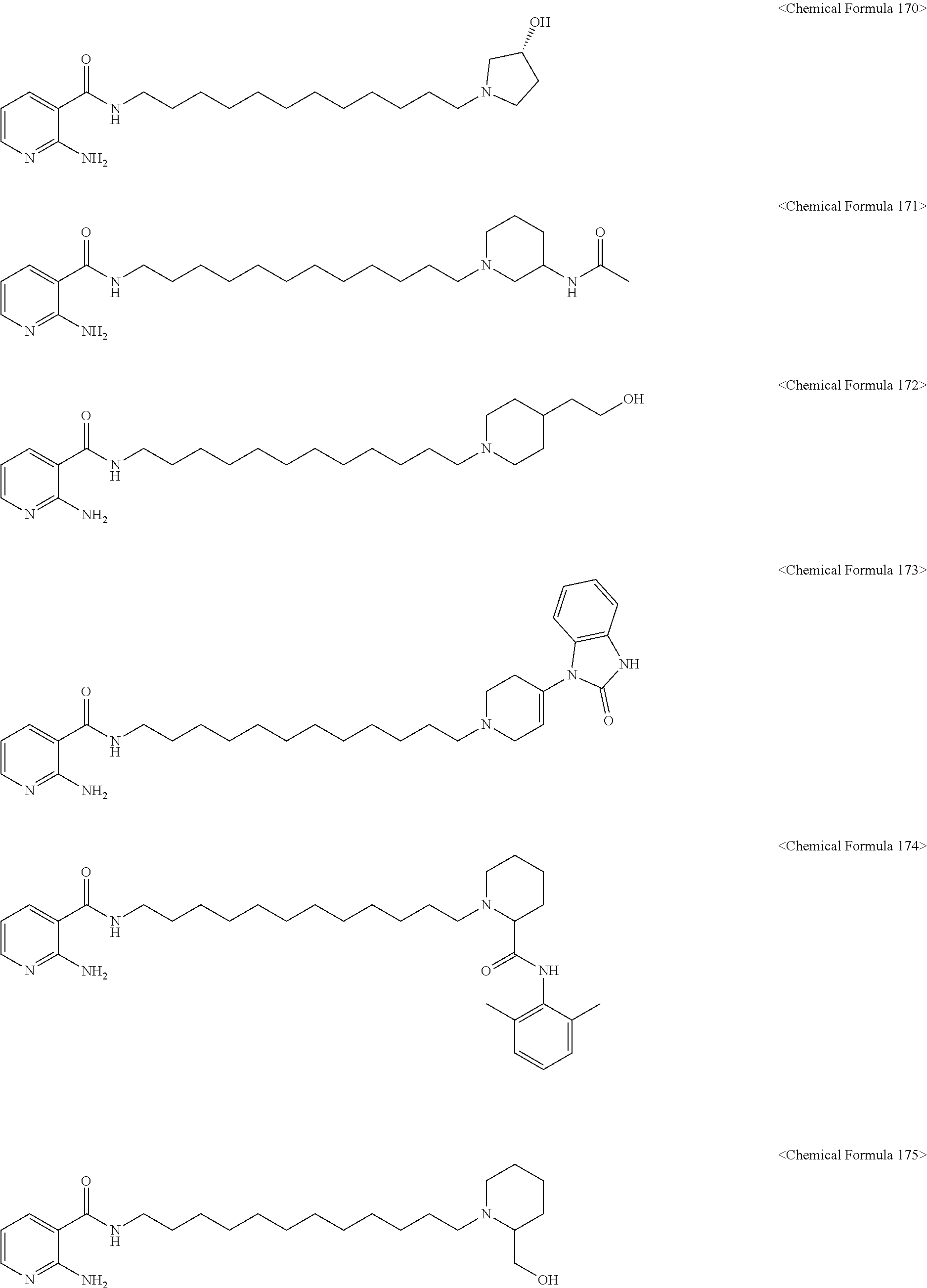 [SMILES: Nc1ncccc1C(=O)NCCCCCCCCCCCCN1CC[C@@H](O)C1 ; CC(=O)NC1CCCN(CCCCCCCCCCCCNC(=O)c2cccnc2N)C1 ; Nc1ncccc1C(=O)NCCCCCCCCCCCCN1CCC(CCO)CC1 ; Nc1ncccc1C(=O)NCCCCCCCCCCCCN1CC=C(n2c(=O)[nH]c3ccccc32)CC1 ; Cc1cccc(C)c1NC(=O)C1CCCCN1CCCCCCCCCCCCNC(=O)c1cccnc1N ; Nc1ncccc1C(=O)NCCCCCCCCCCCCN1CCCCC1CO]

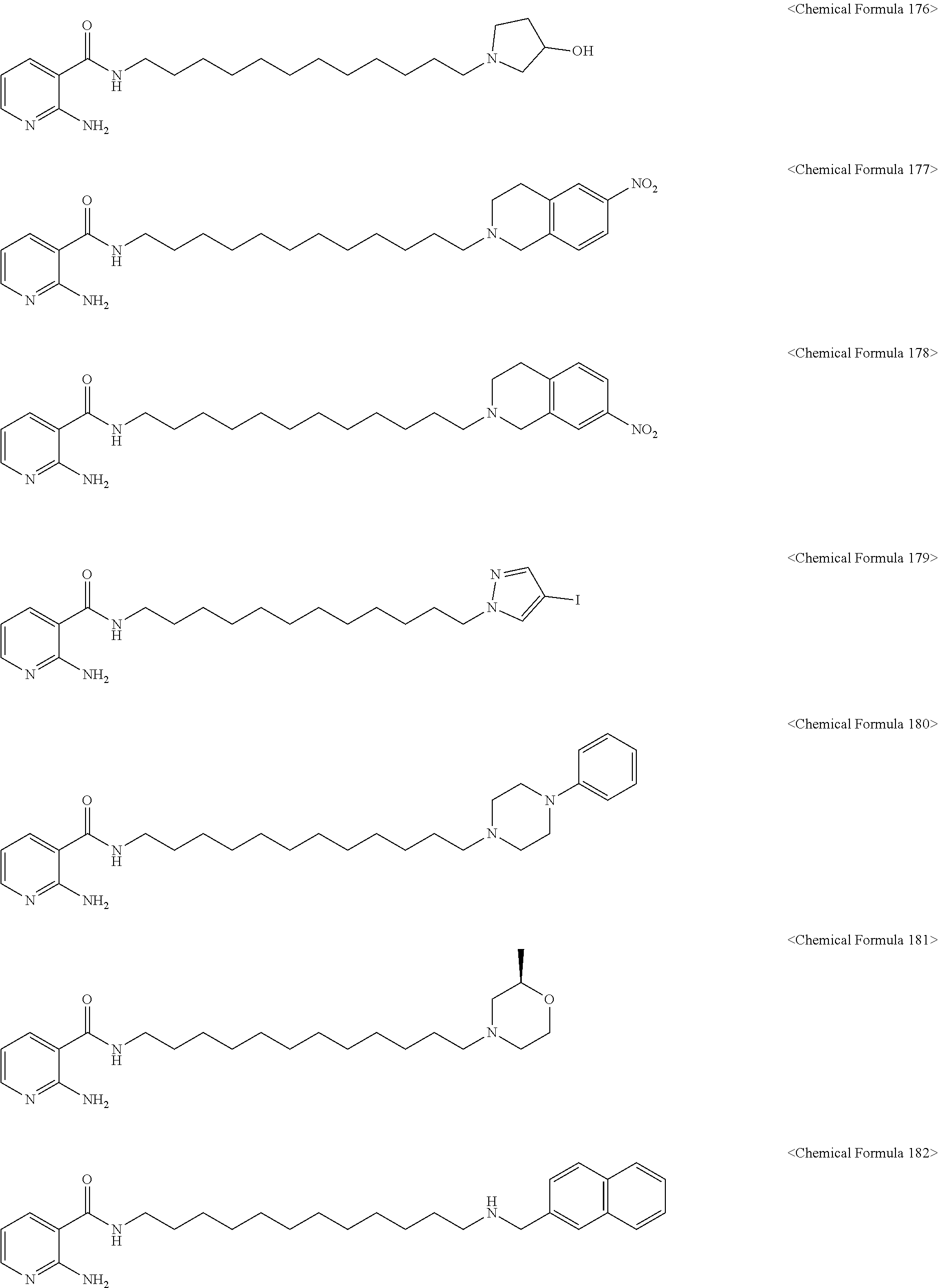

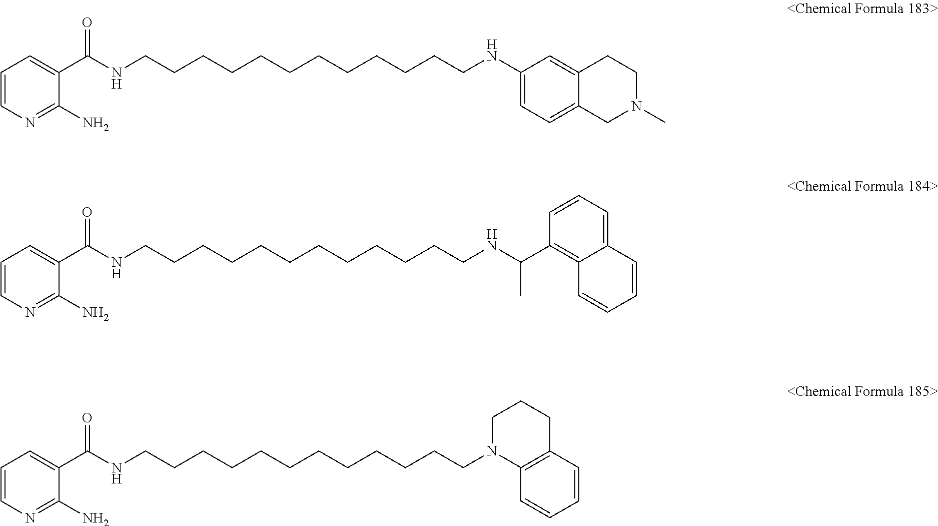 [SMILES: CN1CCc2cc(NCCCCCCCCCCCCNC(=O)c3cccnc3N)ccc2C1 ; CC(NCCCCCCCCCCCCNC(=O)c1cccnc1N)c1cccc2ccccc12 ; Nc1ncccc1C(=O)NCCCCCCCCCCCCN1CCCc2ccccc21]

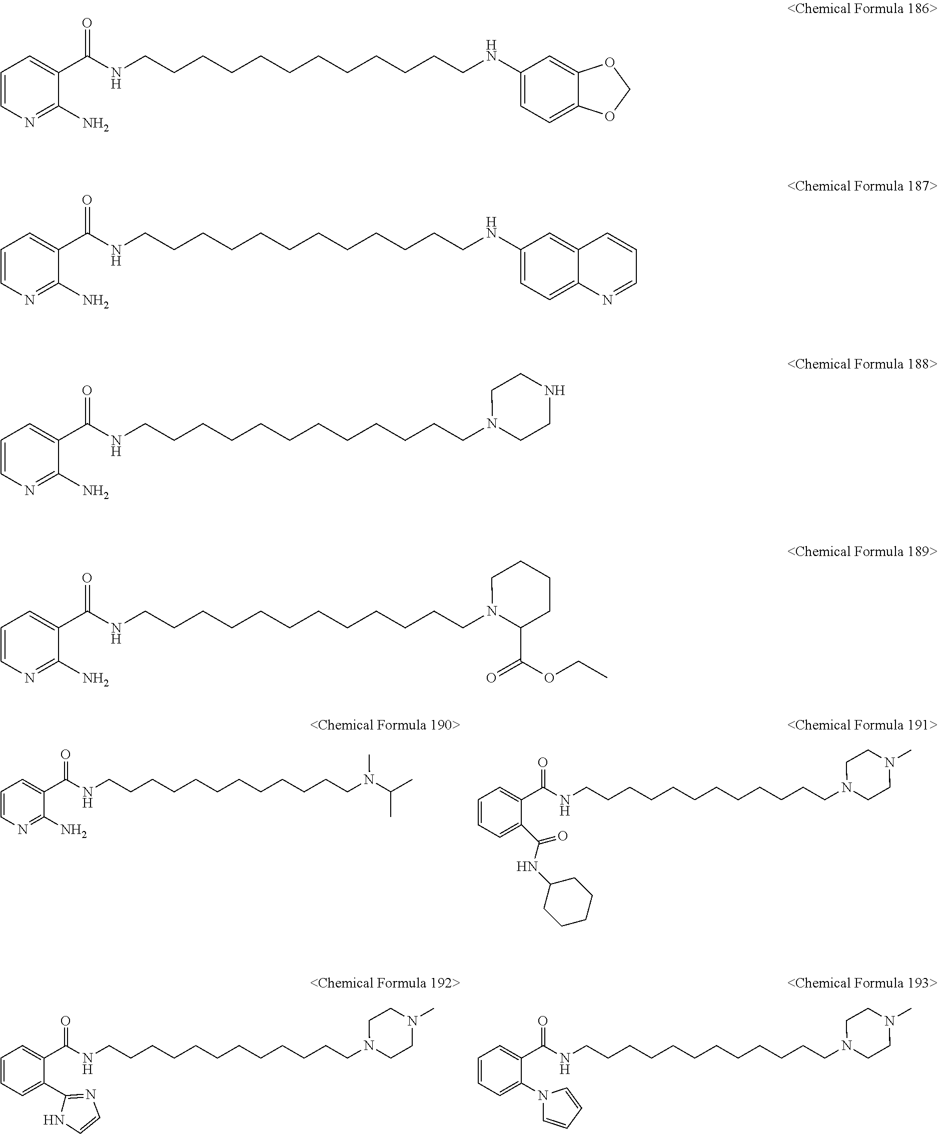

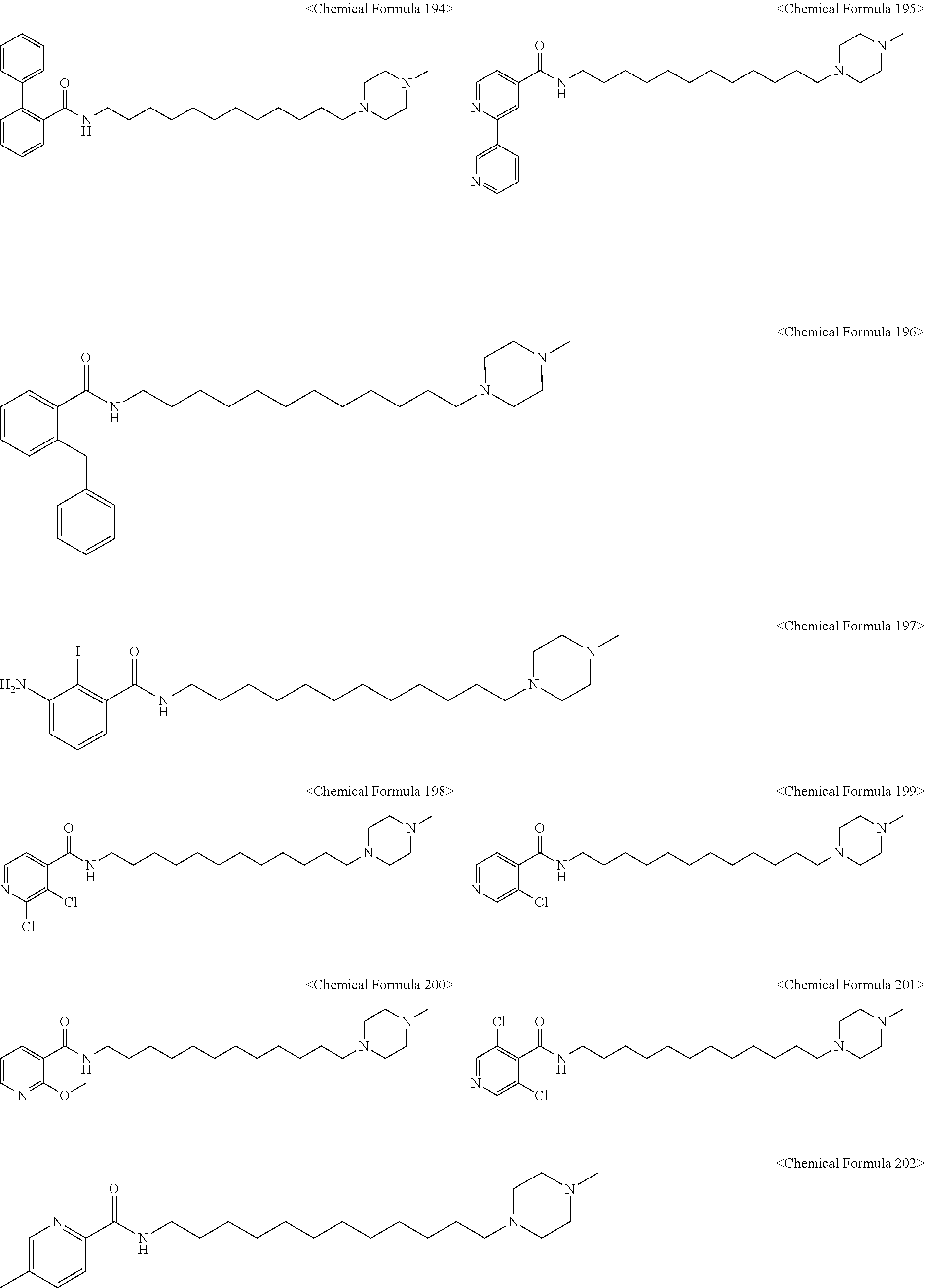

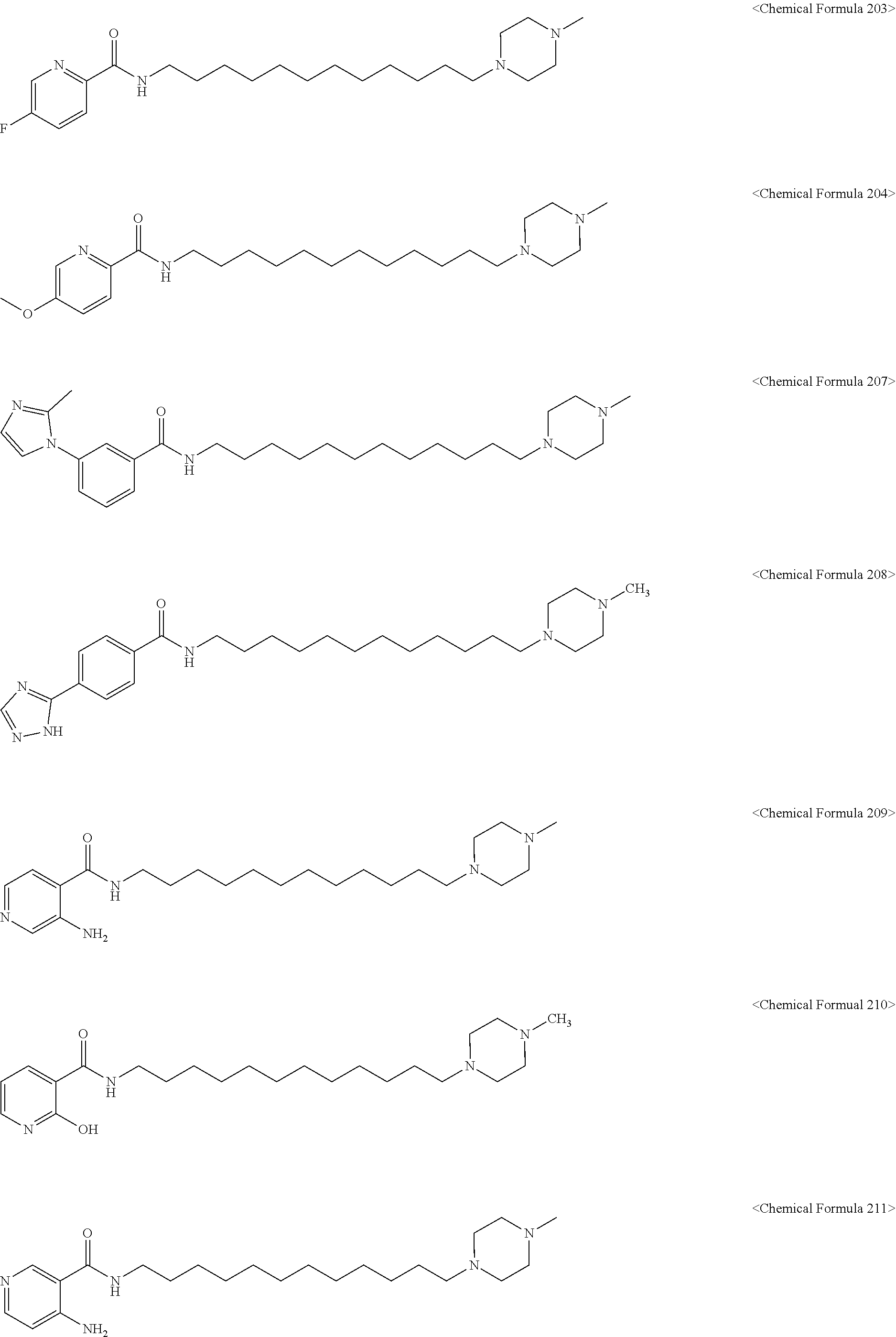

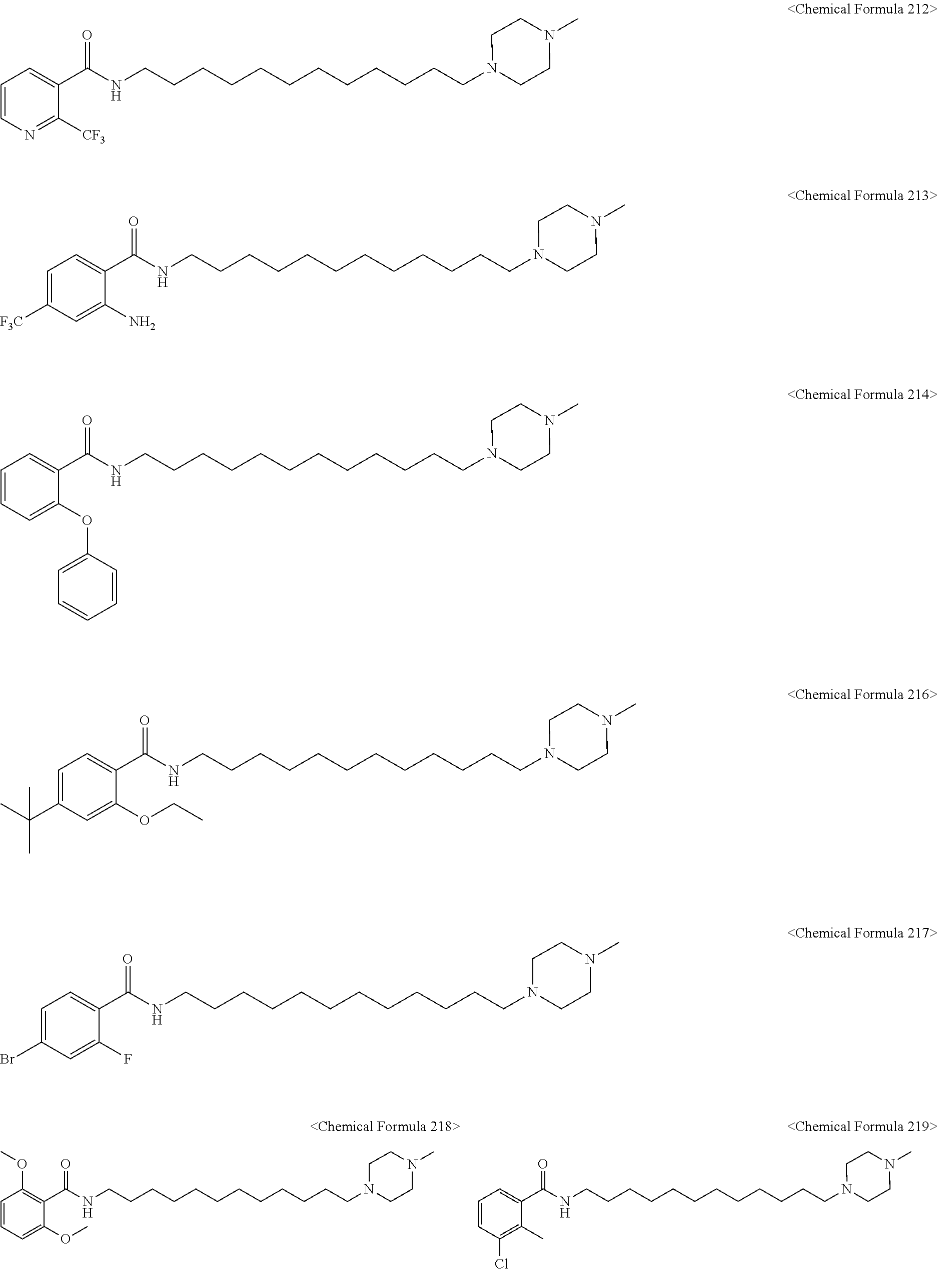

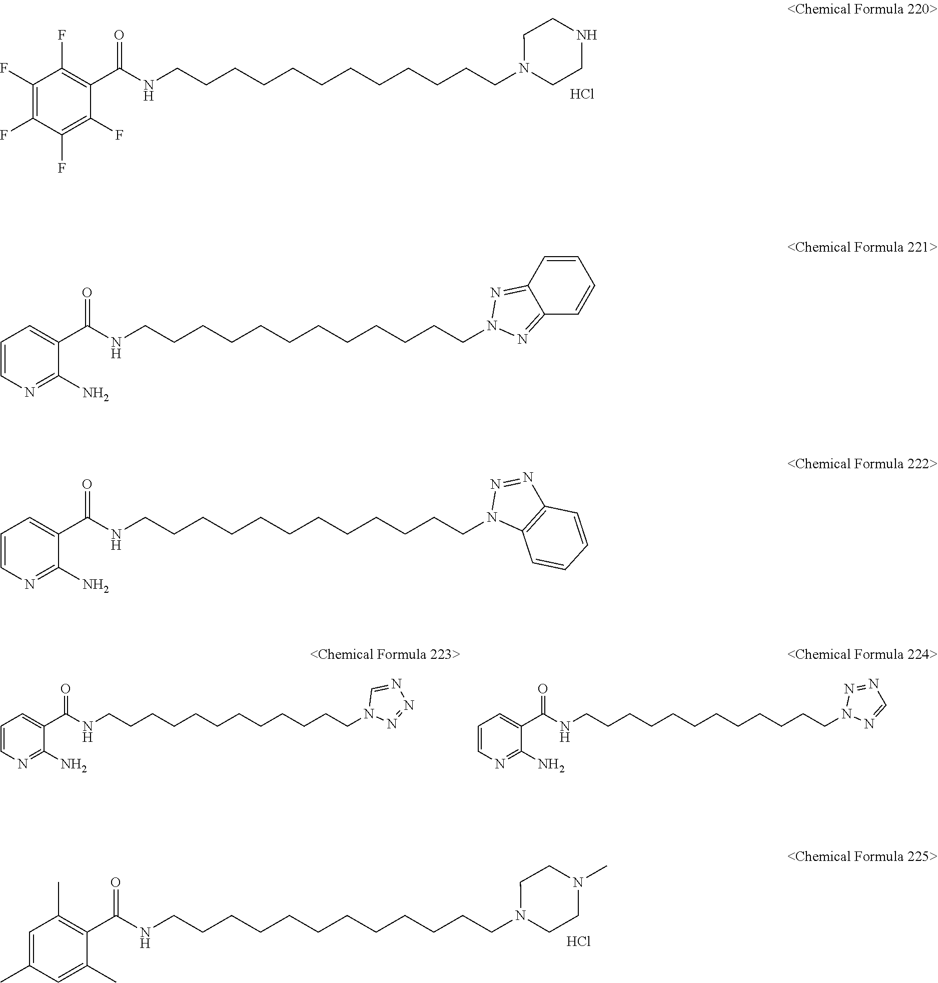

### Compound Identification Information

2. Compound 2: 2-Amino-N-(12-morpholin-4-yl-dodecyl)-nicotinamide (used in conjunction with GO48)

### <Chemical Formula 2>

(Such chemical structures can be referred to as “chemical formulas,” and the 15 numbers can be used to denote the same compound numbers. For example, since it is Compound 2, it can be referred to or denoted as “Chemical Formula 2,” and the same applies to subsequent cases.)

White solid, 1H NMR (400 MHz, CDCl₃) δ 8.13 (dd, J=4.79, 1.75 Hz, 1H), 7.55 (dd, J=7.69, 1.76 Hz, 1H), 6.57 (dd, J=7.64, 4.86 Hz, 1H), 6.28 (s, 2H), 5.99 (s, 1H), 3.70 (t, J=4.67 Hz, 4H), 3.41-3.36 (m, 2H), 2.41 (t, J=4.25 Hz, 4H), 2.29 (dd, J=8.77, 6.75 Hz, 2H), 1.58 (t, J=7.29, 2H), 1.46 (t, J=7.29 Hz, 2H), 1.37-1.25 (m, 16H) Ms caled for C22H38N4O2: 390.57.

### 3. Compound 3: 2-Amino-N-[12-(3,4-Dihydro-1H-Isoquinolin-2-Yl)-Dodecyl]-Nicotinamide (G051 Alias)

### <Chemical Formula 3>

White, 1H NMR (400 MHz, cdcl3) δ 8.13 (dd, J=4.73, 1.76 Hz, 1H), 7.55 (dd, J=7.69, 1.77 Hz, 1H), 7.09 (dq, J=6.07, 2.99 Hz, 3H), 6.99 (dd, J=5.04, 2.83 Hz, 1H), 6.57 (dd, J=7.72, 4.97 Hz, 1H), 6.27 (d, J=0.56 Hz, 2H), 5.97 (t, J=1.40 Hz, 1H), 3.60 (s, 2H), 3.39 (td, J=7.15, 5.84 Hz, 2H) 2.89 (t, J=5.90, 2H), 2.70 (t, J=5.96 Hz, 2H), 2.47 (dd, J=8.65, 6.77 Hz, 2H) 1.62-1.54 (m, 2H), 1.37-1.24 (m, 18H) Ms caled for C₂₇H40N4O: 436.64

### 4. Compound 4: 2-Amino-N-(12-Thio-Morpholin-4-Yl-Dodecyl)-Nicotinamide (Also Known as G077)

### <Chemical Formula 4>

White-yellow solid, 1H NMR (400 MHz, CDCl₃) δ 8.12 (dd, J=4.86, 1.75 Hz, 1H), 7.55 (dd, J=7.68, 1.75 Hz, 1H), 6.57 (dd, J=7.68, 4.86 Hz, 1H), 6.28 (s, 2H), 6.02 (s, 1H), 3.41-3.36 (m, 2H), 2.66 (d, J=1.72 Hz, 8H), 2.31 (d, J=15.47 Hz, 2H), 1.57 (t, J=7.19 Hz, 2H), 1.44-1.25 (m, 18H), Ms caled for C22H38N40S: 406.63.

### 5. Compound 5: 2-Amino-N-(12-(Piperazin-1-Yl)Dodecyl)-Nicotinamide (G078, Also Known as)

### <Chemical Formula 5>

White solid, 1H NMR (400 MHz, CDCl₃) δ 8.12 (dd, J=4.87, 1.74 Hz, 1H), 7.55 (dd, J=7.69, 1.72 Hz, 1H), 6.57 (dd, J=7.67, 4.86 Hz, 1H), 6.38 (d, J=0.36 Hz, 2H), 6.02 (s, 1H), 3.41-3.36 (m, 2H), 2.45-2.28 (m, 1OH), 1.85 (s, 3H), 1.57 (t, J=7.18 Hz, 2H), 1.47-1.25 (m, 18H), Ms caled for C23H41N5O: 403.62.

### 6. Compound 6: 12-[4-(2-Methylpropyl)Piperazin-1-Yl]Dodecyl-N-(6-Aminopurine-2-Yl)Carboxamide (Also Known as G079)

### <Chemical Formula 6>

White solid, 1H NMR (400 MHz, CDCl₃) δ 8.13 (dd, J=4.85, 1.77 Hz, 1H), 7.55 (dd, J=7.69, 1.76 Hz, 1H), 6.58 (d, J=4.84 Hz, 1H), 6.28 (s, 2H), 6.03 (s, 1H), 3.38 (td, J=7.15, 5.80 Hz, 2H), 2.48-2.38 (m, 6H), 1.78-1.74 (m, 4H), 1.60-1.47 (m, 4H), 1.37-1.22 (m, 16), Ms caled for C25H45N5O: 431.67.

### 7. Compound 7: 12-[4-(2-Methoxy-2-Oxoethyl)Piperazin-1-Yl]Dodecyl-N-(6-Aminopurine-2-Yl)Carboxamide (Also Known as G080)

### <Chemical Formula 7>

White solid, 1H NMR (400 MHz, CDCl₃) δ 8.13 (t, J=2.46 Hz, 1H), 7.54 (s, 1H), 6.57 (dd, J=7.63, 4.83 Hz, 1H), 6.28 (s, 2H), 6.02 (s, 1H), 3.43-3.38 (m, 6H), 2.32 (dt, J=20.10, 6.32 Hz, 6H), 1.58 (t, J=7.12 Hz, 2H), 1.44-1.25 (m, 2H), 1.26 (d, J=28.73 Hz, 18H), 1.03 (d, 27H), Ms caled for C27H47N5O3: 489.71.

8. Compound 8: 12-(Piperazin-1-Yl)Dodecyl-N-(6-Aminopurine-2-yl)Carboxamide (Also Known as G081)

### <Chemical Formula 8>

White solid, 1H NMR (400 MHz, CDCl₃) δ 8.13 (dd, J=4.90, 1.75 Hz, 1H), 7.55 (dd, J=7.70, 1.72 Hz, 1H), 6.57 (dd, J=7.66, 4.88 Hz, 1H), 6.27 (s, 2H), 5.99 (s, 1H), 3.41-3.36 (m, 2H), 2.86 (d, J=11.65, 2H), 2.25 (dd, J=9.16, 6.66 Hz, 2H), 1.85 (td, J=11.56, 1.64 Hz, 3H), 1.58 (td, J=13.93, 6.70 Hz, 4H), 1.37-1.21 (m, 20H), 0.89 (d, J=6.28 Hz, 3H), Ms caled for C24H42N4O: 402.63.

9. Compound 9: 12-(4-Hydroxypiperidin-1-Yl)Dodecyl-N-(6-Aminopurine-2-yl)Carboxamide (Also Known as G083)<Chemical formula 9>

White Solid, 1H NMR (400 MHz, CDCl₃) δ 8.12 (Dd, J=4.81, 1.64 Hz, 1H), 7.55 (dd, J=7.65, 1.57 Hz, 1H), 6.56 (dd, J=7.64, 4.86 Hz, 1H), 6.28 (s, 2H), 6.01 (s, 1H), 3.38 (q, J=6.55 Hz, 2H), 2.83 (d, J=11.23 Hz, 1H), 2.61 (ddd, J=12.79, 10.32, 5.81 Hz, 1H), 2.30 (ddd, J=12.82, 10.18, 5.58 Hz, 1H), 2.23 (t, J=5.90 Hz, 1H), 2.11 (td, J=11.09, 2.94 Hz, 1H), 1.59-1.25 (m, 26H), 1.03 (d, J=6.23 Hz, 3H), Ms caled for C24H42N4O: 402.63.

### 10. Compound 10: 12-(4-Hydroxypiperazin-1-Yl)Dodecyl-N-(6-Aminopurazin-2-Yl)Carboxamide (Also Known as GO85)

### <Chemical Formula 10>

White-yellow solid, 1H NMR (400 MHz, CDCl₃) δ 8.13 (dd, J=4.83, 1.62 Hz, 1H), 7.56 (dd, J=7.64, 1.54 Hz, 1H), 6.57 (dd, J=7.63, 4.86 Hz, 1H), 6.28 (s, 2H), 6.04 (s, 1H), 3.48 (d, J=6.36 Hz, 2H), 3.38 (q, J=6.55 Hz, 2H), 3.03 (d, J=10.74 Hz, 2H), 2.37 (t, J=7.82 Hz, 2H), 2.03-1.94 (m, 2H), 1.76-1.74 (m, 7H), 1.42-1.20 (m, 18H), Ms caled for C24H42N4O2: 418.63.

### 11. Compound 11: 12-[4-(2,2-Dimethylmorpholin-4-Yl)]Dodecyl-N-(6-Aminopurine-2-Yl)Carboxamide (GO86 Alias)

### <Chemical Formula 11>

White solid, 1H NMR (400 MHz, CDCl₃) δ 8.12 (dd, J=4.76, 1.70 Hz, 1H), 7.55 (dd, J=7.70, 1.74 Hz, 1H), 6.57 (dd, J=7.77, 5.03 Hz, 1H), 6.28 (s, 2H), 6.02 (s, 1H), 3.68-3.64 (m, 2H), 3.41-3.36 (m, 2H), 2.74-2.71 (m, 2H), 2.28-2.25 (m, 2H), 1.78 (s, 2H), 1.65 (t, J=10.86 Hz, 2H), 1.57 (t, J=7.15 Hz, 2H), 1.45 (s, 2H), 1.33-1.25 (m, 14H), 1.13 (d, J=6.31 Hz, 6H), Ms caled for C24H42N4O2: 418.63.

### 12. Compound 12: Tert-Butyl 3-(8-(2-Aminonicotinamido)Octyl)-4-Methylpiperazine-1-Carboxylate (Also Known as KMC-003P)

### <Chemical Formula 12>

Off-white solid, ¹H NMR (400 MHz, CDCl₃): δ=9.68 (s, 1H), 8.85 (t, J=5.3 Hz, 1H), 8.30 (d, J=7.7 Hz, 1H), 8.10 (dd, J=1.5, 5.9 Hz, 2H), 6.87 (dd, J=6.0, 7.5 Hz, 1H), 3.20 (dd, J=6.7, 13.1 Hz, 4H), 3.08-3.00 (m, 1H), 2.78 (s, 3H), 1.88-1.78 (m, 1H), 1.62-1.48 (m, 4H), 1.42 (s, 9H), 1.28 (s, 12H), Ms caled for C24H41N₅O3: 447.62.

### 13. Compound 13: 12-(4-Hydroxypiperidin-1-Yl)Dodecyl-N-(6-Aminopurine-2-Yl)Carboxamide (Also Known as GO87)

### <Chemical Formula 13>

White-yellow solid, 1H NMR (400 MHz, CDCl₃) δ 8.13 (dd, J=4.83, 1.53 Hz, 1H), 7.56 (dd, J=7.64, 1.65 Hz, 1H), 6.57 (dd, J=7.67, 4.93 Hz, 1H), 6.29 (s, 2H), 6.08 (t, J=1.07 Hz, 1H), 6.02-5.94 (m, 1H), 3.39-3.34 (m, 4H), 3.10 (s, 2H), 2.62 (t, J=5.39 Hz, 2H), 2.38 (t, J=7.47 Hz, 2H), 1.57 (d, J=7.26 Hz, 2H), 1.46 (s, 2H), 1.32-1.25 (m, 16H), Ms caled for C₂₂H₃₇N5O2: 403.57.

### 14. Compound 14: 2-Amino-N-(12-(Piperazin-1-Yl)Dodecyl)Nicotinamide (Hereinafter Referred to as G088)

### <Chemical Formula 14>

White-yellow solid, 1H NMR (400 MHz, cdcl3) δ 8.12 (dd, J=4.79, 1.61 Hz, 1H), 7.55 (dd, J=7.68, 1.71 Hz, 1H), 6.56 (dd, J=7.73, 4.98 Hz, 1H), 6.28 (s, 2H), 6.05 (s, 1H), 3.37 (q, J=6.57 Hz, 2H), 2.88 (t, J=4.91 Hz, 4H), 2.40 (s, 4H), 2.31-2.27 (m, 2H), 1.57 (t, J=7.16 Hz, 2H), 1.48-1.43 (m, 2H), 1.26 (d, J=27.82 Hz, 17), Ms caled for C₂₂H₃₉N5O: 389.59.

### 15. Compound 15: 12-[4-(2-Hydroxyethyl)Piperazin-1-Yl]Dodecyl-N-(6-Aminopurine-2-Yl)Carboxamide (Also Known as G089)

### <Chemical Formula 15>

White solid, 1H NMR (400 MHz, CDCl₃) δ 8.13 (dd, J=4.85, 1.77 Hz, 1H), 7.55 (dd, J=7.69, 1.76 Hz, 1H), 6.58 (d, J=4.84 Hz, 1H), 6.28 (s, 2H), 6.03 (s, 1H), 3.38 (td, J=7.15, 5.80 Hz, 2H), 2.48-2.38 (m, 6H), 1.78-1.74 (m, 4H), 1.60-1.47 (m, 4H), 1.37-1.22 (m, 16), Ms caled for C22H38N4O: 347.57.

### 16. Compound 16: 2-Amino-N-(12-(Octahydroisoquinolin-2(1H)-Yl)Dodecyl) Nicotinamide (Hereinafter Referred to as PBK-2024-003)

### <Chemical Formula 16>

White-yellow solid, ¹H NMR (400 MHz, DMSO): δ=8.38 (t, J=5.5 Hz, 1H), 8.24 (d, J=1.5 Hz, 1H), 8.05 (dd, J=1.8, 4.8 Hz, 1H), 7.85 (dd, J=1.8, 7.7 Hz, 1H), 7.03 (s, 2H), 6.56 (dd, J=4.8, 7.7 Hz, 1H), 3.95 (s, 9H), 3.19 (q, J=6.6 Hz, 2H), 3.10-2.97 (m, 1H), 2.91-2.82 (m, 1H), 2.07 (s, 1H), 1.65 (s, 3H), 1.59-1.43 (m, 6H), 1.31-1.21 (m, 18H), 0.92-0.87 ppm (m, 3H), Ms caled for C27H₄₆N₄O: 442.69.

17. Compound 17: 2-Amino-N-(12-(3,4-dihydroisoquinolin-2(1H)-yl)dodecyl) nicotinamide (2-amino-N-[12-(3,4-dihydro-1H-isoquinolin-2-yl)-dodecyl]-nicotinamide) (also known as PBK-2024-004)

### <Chemical Formula 17>

White solid, ¹H NMR (400 MHz, CDCl₃): δ=8.15 (s, 1H), 7.60 (d, J=7.7 Hz, 1H), 7.18-7.11 (m, 3H), 7.06-7.02 (m, 1H), 6.60 (dd, J=4.8, 7.5 Hz, 1H), 6.33 (t, J=18.1 Hz, 2H), 6.09 (s, 1H), 3.80 (s, 2H), 3.44-3.36 (m, 2H), 3.00-2.89 (m, 6H), 2.68-2.61 (m, 3H), 1.39-1.24 ppm (m, 15H), Ms caled for C27H40N4O: 436.64.

18. Compound 18: 2-amino-N-(12-(piperidin-1-yl)dodecyl)nicotinamide (also known as PBK-2024-005)

### <Chemical Formula 18>

White solid, ¹H NMR (400 MHz, CDCl₃): δ=8.15 (dd, J=1.6, 4.8 Hz, 1H), 7.59 (dd, J=1.6, 7.7 Hz, 1H), 6.60 (dd, J=4.9, 7.7 Hz, 1H), 6.32 (s, 2H), 6.06 (s, 1H), 3.44-3.37 (m, 2H), 2.54-2.34 (m, 6H), 1.28 ppm (s, 18H), Ms caled for C23H40N40: 388.60.

19. Compound 19: 2-Amino-N-(12-(3-(dimethylamino)pyrrolidin-1-yl) dodecyl) nicotinamide (also known as PBK-2024-006)

### <Chemical Formula 19>

Brown solid, ¹H NMR (400 MHz, CDCl₃): δ=8.15 (dd, J=1.8, 4.9 Hz, 1H), 7.58 (dd, J=1.8, 7.7 Hz, 1H), 6.60 (dd, J=4.9, 7.7 Hz, 1H), 6.29 (s, 2H), 6.06-6.02 (m, 1H), 3.44-3.37 (m, 2H), 3.02-2.77 (m, 4H), 2.24 (s, 12H), 2.09-1.97 (m, 3H), 1.80-1.70 ppm (m, 1H), Ms caled for C24H43N5O: 417.64.

20. Compound 20: 2-Amino-N-(12-(pyrrolidin-1-yl)dodecyl)nicotinamide (also known as PBK-2024-007)
<Chemical formula 20>

White solid, ¹H NMR (400 MHz, DMSO): δ=8.38 (t, J=5.3 Hz, 1H), 8.06-8.03 (m, 1H), 7.84 (d, J=7.7 Hz, 1H), 7.02 (s, 2H), 6.56 (dd, J=4.8, 7.7 Hz, 1H), 2.40-2.29 (m, 6H), 1.65 (t, J=3.2 Hz, 4H), 1.53-1.37 (m, 6H), 1.24 ppm (s, 18H), Ms caled for C22H38N4O: 374.57.

21. Compound 21: 2-Amino-N-(12-(2,6-dimethylmorpholino) dodecyl) nicotinamide (also known as PBK-2024-008)
<Chemical formula 21>

White solid, ¹H NMR (400 MHz, CDCl₃): δ=8.15 (dd, J=1.8, 4.8 Hz, 1H), 7.58 (dd, J=1.8, 7.7 Hz, 1H), 6.60 (dd, J=4.9, 7.7 Hz, 1H), 6.29 (s, 2H), 6.04 (s, 1H), 3.86-3.80 (m, 2H), 3.44-3.37 (m, 2H), 2.86-2.84 (m, 2H), 2.39 (s, 2H), 1.64-1.55 (m, 6H), 1.32-1.24 (m, 18H), 1.17 ppm (d, J=6.4 Hz, 6H), Ms caled for C24H42N4O2: 418.63.

22. Compound 22: 2-Amino-N-(12-(3,3-dimethylpyrrolidin-1-yl)dodecyl) nicotinamide (also known as PBK-2024-009)
<Chemical formula 22>

Brown solid, ¹H NMR (400 MHz, CDCl₃): δ=8.15 (dd, J=1.8, 4.8 Hz, 1H), 7.58 (dd, J=1.8, 7.7 Hz, 1H), 6.60 (dd, J=4.9, 7.7 Hz, 1H), 6.29 (s, 2H), 6.04 (s, 1H), 3.86-3.80 (m, 2H), 3.44-3.37 (m, 2H), 2.86-2.84 (m, 2H), 2.39 (s, 2H), 1.64-1.55 (m, 6H), 1.32-1.24 (m, 18H), 1.17 ppm (d, J=6.4 Hz, 6H), Ms caled for C24H42N₄O: 402.64.

23. Compound 23: N-(12-(hexahydropyrrolo[1,2-a]pyrazin-2(1H)-yl)dodecyl) nicotinamide (also known as PBK-2024-010)
<Chemical formula 23>

White solid, ¹H NMR (400 MHz, DMSO): δ=8.37 (s, 1H), 8.05 (dd, J=1.8, 4.7 Hz, 1H), 7.85 (dd, J=1.8, 7.7 Hz, 1H), 7.03 (s, 2H), 6.60-6.54 (m, 1H), 2.95-2.82 (m, 3H), 2.71 (d, J=10.4 Hz, 1H), 2.30-2.22 (m, 3H), 2.10-1.85 (m, 6H), 1.70-1.59 (m, 6H), 1.27-1.21 ppm (m, 24H), Ms caled for C25H43N4O: 429.63.

24. Compound 24: 2-Amino-N-(12-(3-methylpyrrolidin-1-yl)dodecyl) nicotinamide (also known as PBK-2024-012)
<Chemical formula 24>

White-yellow solid, 1H NMR (400 MHz, CDCl₃) δ 8.15 (dd, J=1.7, 4.9 Hz, 1H), 7.59 (dd, J=1.7, 7.7 Hz, 1H), 6.60 (dd, J=4.9, 7.6 Hz, 1H), 6.31 (s, 2H), 6.06 (s, 1H), 3.40 (q, J=6.7 Hz, 2H), 2.95-2.90 (m, 1H), 2.81-2.81 (m, 1H), 2.54-2.42 (m, 3H), 2.35-2.26 (m, 1H), 2.10-2.01 (m, 2H), 1.62-1.53 (m, 5H), 1.34-1.24 (m, 16H), 1.04 (d, J=6.8 Hz, 3H), Ms caled for C23H40N4O: 388.60.

25. Compound 25: 2-Amino-N-(12-(1,1-dioxidothiomorpholino)dodecyl) nicotinamide (also known as PBK-2024-013)
<Chemical formula 25>

White solid, 1H NMR (400 MHz, CDCl₃) δ 8.05 (dd, J=1.5, 5.2 Hz, 1H), 7.75-7.72 (m, 1H), 6.98 (s, 2H), 6.66 (dd, J=5.3, 7.6 Hz, 1H), 6.27 (s, 1H), 3.41 (q, J=6.8 Hz, 2H), 3.11-3.05 (m, 4H), 3.03-2.98 (m, 4H), 2.51 (t, J=7.5 Hz, 2H), 1.65-1.57 (m, 2H), 1.48-1.46 (m, 2H), 1.31-1.25 (m, 16H), Ms caled for C22H38N4O3S: 438.63.

26. Compound 26: 2-Amino-N-(12-(3-fluoropyrrolidin-1-yl) dodecyl) nicotinamide (also known as PBK-2024-014)
<Chemical formula 26>

White-yellow solid, 1H NMR (400 MHz, CDCl₃) δ 8.15 (dd, J=1.7, 4.9 Hz, 1H), 7.58 (dd, J=1.7, 7.7 Hz, 1H), 6.60 (dd, J=4.9, 7.7 Hz, 1H), 6.31 (s, 2H), 6.03 (s, 1H), 5.27-5.13 (m, 1H), 3.44-3.37 (m, 2H), 2.97-2.84 (m, 2H), 2.56-2.56 (m, 2H), 2.24-2.03 (m, 2H), 1.63-1.55 (m, 6H), 1.36-1.24 (m, 16H), Ms caled for C22H37FN40: 392.56.

27. Compound 27: 2-Amino-N-(12-(4-(4-fluorobenzyl) piperazin-1-yl) dodecyl) nicotinamide (also known as PBK-2024-015)
<Chemical formula 27>

White solid, ¹H NMR (400 MHz, CDCl₃): δ=8.15 (dd, J=1.8, 4.9 Hz, 1H), 7.58 (dd, J=1.6, 7.7 Hz, 1H), 7.02-6.97 (m, 2H), 6.60 (dd, J=4.8, 7.7 Hz, 1H), 6.31 (s, 2H), 6.06 (s, 1H), 3.50-3.37 (m, 6H), 2.55-2.41 ppm (m, 12H), Ms caled for C29H44FN5O: 497.70.

28. Compound 28: 2-Amino-N-(12-(4-(4-methoxybenzyl)piperazin-1-yl) dodecyl) nicotinamide (also known as PBK-2024-016)
<Chemical formula 28>

White solid, ¹H NMR (400 MHz, CDCl₃): δ=8.15 (dd, J=1.8, 4.9 Hz, 1H), 7.58 (dd, J=1.8, 7.7 Hz, 1H), 6.85 (d, J=8.7 Hz, 2H), 6.60 (dd, J=4.8, 7.7 Hz, 1H), 6.31 (s, 2H), 6.05-6.01 (m, 1H), 3.80 (s, 3H), 3.49-3.37 (m, 4H), 2.77-2.37 (m, 9H), 1.27 ppm (s, 18H), Ms caled for C30H47N5O2: 509.74.

29. Compound 29: 2-Amino-N-(12-(1-methylhexahydropyrrolo[1,2-a]pyrazin-2(1H)-yl) dodecyl)nicotinamide (hereinafter referred to as PBK-2024-018)
<Chemical formula 29>

White solid, 1H NMR (400 MHz, CDCl₃) δ 8.14 (1H, dd, J=1.8, 4.9 Hz), 7.64 (1H, dd, J=1.7, 7.7 Hz), 6.60 (1H, dd, J=4.9, 7.7 Hz), 6.36-6.24 (3H, m), 3.44-3.36 (2H, m), 3.19-3.05 (3H, m), 2.92-2.90 (1H, m), 2.81-2.69 (3H, m), 2.59-2.53 (1H, m), 2.36-2.28 (2H, m), 1.98-1.77 (4H, m), 1.66-1.51 (4H, m), 1.41-1.22 (19H, m). 443.68, Ms caled for C26H45N5O: 443.67.

30. Compound 30: tert-Butyl 4-(12-(2-aminonicotinamido) dodecyl)-2,5-dimethylpiperazine-1-carboxylate (hereinafter referred to as PBK-2024-020)
<Chemical formula 30>

White solid, 1H NMR (400 MHz, DMSO-d6) δ 8.39 (t, J=5.3 Hz, 1H), 8.05 (dd, J=1.8, 4.8 Hz, 1H), 7.85 (dd, J=1.8, 7.7 Hz, 1H), 7.03 (s, 2H), 6.56 (dd, J=4.8, 7.7 Hz, 1H), 4.09-4.05 (m, 2H), 3.50 (d, J=12.9 Hz, 1H), 3.19 (q, J=6.6 Hz, 2H), 3.11 (dd, J=3.3, 13.0 Hz, 1H), 2.86-2.84 (m, 1H), 2.33-2.26 (m, 1H), 2.26-2.19 (m, 2H), 1.51-1.47 (m, 2H), 1.38 (s, 9H), 1.26-1.22 (m, 16H), 1.13 (d, J=6.7 Hz, 3H), 0.80 (d, J=6.5 Hz, 3H), Ms caled for C29H₅₁N5O3: 517.76.

31. Compound 31: 2-Amino-N-(12-(thiazolidin-3-yl)dodecyl)nicotinamide) (hereinafter referred to as PBK-2024-021)
<Chemical formula 31>

White solid, 1H NMR (400 MHz, CDCl₃) δ 8.01 (dd, J=1.7, 5.1 Hz, 1H), 7.65 (dd, J=1.7, 7.7 Hz, 1H), 6.61-6.52 (m, 3H), 6.36-6.34 (m, 1H), 4.02 (s, 2H), 3.36-3.29 (m, 2H), 3.05 (t, J=6.3 Hz, 2H), 2.84 (t, J=6.3 Hz, 2H), 2.36 (t, J=7.6 Hz, 2H), 1.56-1.50 (m, 2H), 1.50-1.43 (m, 2H), 1.26-1.17 (m, 16H), Ms caled for C21H36N405S: 392.61.

32. Compound 32: 2-Amino-N-(12-(3-methoxypyrrolidin-1-yl) dodecyl) nicotinamide (hereinafter referred to as PBK-2024-024)
<Chemical formula 32>

White solid, ¹H NMR (400 MHz, CDCl₃): δ=8.15 (dd, J=1.7, 4.8 Hz, 1H), 7.59 (dd, J=1.6, 7.7 Hz, 2H), 6.60 (dd, J=4.9, 7.7 Hz, 1H), 6.31 (s, 2H), 6.10 (s, 1H), 4.00-3.94 (m, 1H), 3.40 (dd, J=7.1, 13.1 Hz, 2H), 3.29 (s, 3H), 3.12-3.02 (m, 1H), 2.79-2.59 (m, 6H), 2.15-2.02 (m, 2H), 1.91 (dd, J=6.1, 7.0 Hz, 2H), 1.34-1.24 ppm (m, 21H), Ms caled for C23H40N4O2: 404.60.

33. Compound 33: 2-Amino-N-(12-(4-(2-(dimethylamino)ethyl)piperazin-1-yl) dodecyl) nicotinamide (also known as PBK-2024-025)
<Chemical formula 33>

White solid, ¹H NMR (400 MHz, CDCl₃): δ=8.15 (dd, J=1.7, 4.8 Hz, 1H), 7.59 (dd, J=1.6, 7.7 Hz, 1H), 6.60 (dd, J=4.9, 7.7 Hz, 1H), 6.32 (s, 2H), 6.05 (s, 1H), 3.43-3.37 (m, 2H), 2.59-2.52 (m, 12H), 2.39 (t, J=7.8 Hz, 2H), 2.32 (s, 6H), 2.15 (d, J=6.9 Hz, 2H), 2.07-2.00 (m, 3H), 1.27 ppm (s, 18H), Ms caled for C26H48N6O: 460.71.

34. Compound 34: 2-Amino-N-(12-(cyclohexyl(methyl)amino) dodecyl) nicotinamide (also known as PBK-2024-026)

<Chemical formula 34>

White solid, ¹H NMR (400 MHz, CDCl₃): δ=8.15 (dd, J=1.8, 4.9 Hz, 1H), 7.59 (dd, J=1.6, 7.7 Hz, 1H), 6.60 (dd, J=4.9, 7.7 Hz, 1H), 6.31 (s, 2H), 6.07 (s, 1H), 3.40 (dd, J=7.2, 13.1 Hz, 2H), 2.51 (s, 4H), 2.33 (s, 3H), 2.03 (d, J=7.2 Hz, 1H), 1.87 (s, 3H), 1.68-1.53 (m, 6H), 1.27 (s, 24H), 1.15-1.05 ppm (m, 2H)), Ms caled for C25H48N6O: 416.65.

35. Compound 35: 2-Amino-N-(12-(2-methylpiperazin-1-yl) dodecyl) nicotinamide (also known as PBK-2024-029)
<Chemical formula 35>

White solid, ¹H NMR (400 MHz, CDCl₃): δ=8.15 (dd, J=1.6, 4.9 Hz, 1H), 7.59 (dd, J=1.6, 7.7 Hz, 1H), 6.60 (dd, J=4.9, 7.7 Hz, 1H), 6.32 (s, 2H), 6.09 (s, 1H), 4.23 (s, 1H), 3.82 (d, J=13.4 Hz, 1H), 3.44-3.37 (m, 2H), 3.22-3.13 (m, 1H), 2.86-2.68 (m, 2H), 2.41-2.14 (m, 3H), 2.10-1.73 (m, 2H), 1.64-1.55 (m, 2H), 0.96-0.71 ppm (m, 1H)), Ms caled for C23H₄₁N₅O: 403.62.

36. Compound 36: 2-Amino-N-(12-(2,5-dimethylpiperazin-1-yl) dodecyl) nicotinamide hydrochloride (also known as PBK-2024-030)
<Chemical formula 36>

White solid, ¹H NMR (400 MHz, DMSO): δ=10.25 (d, J=4.8 Hz, 1H), 10.00-9.91 (m, 1H), 8.99 (s, 1H), 8.42 (d, J=7.2 Hz, 3H), 8.18 (d, J=5.5 Hz, 1H), 6.97 (t, J=6.8 Hz, 1H), 4.03 (q, J=7.1 Hz, 1H), 3.77-3.56 (m, 6H), 3.29-3.17 (m, 9H), 3.00 (d, J=12.0 Hz, 2H), 1.99 (s, 1H), 1.67-1.65 (m, 2H), 1.53 (s, 3H), 1.43-1.23 (m, 24H), 1.17 (t, J=7.1 Hz, 2H) ppm. Ms caled for C24H44C1N50: 454.10.

37. Compound 37: (S)-2-Amino-N-(12-(2-methylpiperazin-1-yl) dodecyl) nicotinamide hydrochloride (also known as PBK-2024-032)
<Chemical formula 37>

White solid, ¹H NMR (400 MHz, CDCl₃): δ=9.03 (1H, t, J=5.3 Hz), 8.44 (3H, dd, J=1.3, 7.6 Hz), 8.18 (1H, dd, J=1.5, 6.1 Hz), 6.96 (1H, dd, J=6.1, 7.5 Hz), 4.03 (1H, ddd, J=7.1, 7.1, 7.1 Hz), 3.30-3.19 (12H, m), 3.10-2.98 (6H, m), 1.99 (1H, s), 1.65 (3H, s), 1.55-1.44 (3H, m), 1.38 (4H, d, J=6.5 Hz), Ms caled for C23H42ClN50: 440.07.

38. Compound 38: 2-Amino-N-(12-(4-(pyridin-4-yloxy)piperidin-1-yl)dodecyl) nicotinamide (hereinafter referred to as PBK-2024-033)
<Chemical formula 38>

White solid, ¹H NMR (400 MHz, CDCl₃): δ=8.44 (dd, J=1.5, 4.9 Hz, 3H), 8.15 (dd, J=1.7, 4.8 Hz, 1H), 7.62 (dd, J=1.7, 7.7 Hz, 1H), 6.80 (dd, J=1.5, 4.9 Hz, 3H), 6.60 (dd, J=4.9, 7.7 Hz, 1H), 6.31 (s, 1H), 6.21 (s, 1H), 4.71-4.52 (m, 1H), 3.44-3.37 (m, 3H), 2.92-2.77 (m, 4H), 2.63-2.57 (m, 3H), 2.39-2.29 (m, 3H), 2.09-2.00 (m, 3H), 1.38-1.24 ppm (m, 21H), Ms caled for C28H43N5O2: 481.69.

39. Compound 39: 2-Amino-N-(12-(4-methylazepan-1-yl) dodecyl) nicotinamide (hereinafter referred to as PBK-2024-034)
<Chemical formula 39>

White solid, ¹H NMR (400 MHz, CDCl₃): δ=8.15 (dd, J=1.6, 4.9 Hz, 1H), 7.63 (dd, J=1.6, 7.7 Hz, 1H), 6.60 (dd, J=4.9, 7.7 Hz, 1H), 6.31 (s, 1H), 6.21 (s, 1H), 3.40 (dd, J=7.1, 13.1 Hz, 2H), 3.03-2.86 (m, 9H), 2.66 (t, J=8.0 Hz, 2H), 2.52 (s, 3H), 2.05 (t, J=12.8 Hz, 2H), 1.28 ppm (s, 18H), Ms caled for C25H44N4O: 416.65.

43. Compound 43: N-(12-((3,5-dimethylimidazolidin-2-yl)dodecyl)pyrido[2,3-d]pyrimidin-7-amine (hereinafter referred to as G092)
<Chemical formula 43>

White solid, ¹H NMR (400 MHz, CDCl₃) δ 8.13 (dd, J=4.85, 1.74 Hz, 1H), 7.55 (dd, J=7.67, 1.71 Hz, 1H), 6.57 (dd, J=7.64, 4.85, 1H), 6.27 (s, 2H), 5.99 (d, J=0.45 Hz, 1H), 3.41-3.36 (m, 2H), 2.91 (ddd, J=9.79, 6.63, 2.91 Hz, 2H), 2.78 (dd, J=11.01, 1.68 Hz, 2H), 2.27 (dd, J=8.90, 6.78 Hz, 2H), 1.60-1.45 (m, 6H), 1.37-1.25 (m, 16H), 1.02 (d, J=6.36 Hz, 6H), Ms caled for C24H43N5O: 417.63.

44. Compound 44: 2-Amino-N-(12-(4-(4-nitrophenyl)piperidin-1-yl)dodecyl)nicotinamide (hereinafter referred to as PBK-2025-001)
<Chemical formula 44>

White solid, ¹H NMR (400 MHz, CDCl₃): δ=8.18 (d, J=8.6 Hz, 2H), 8.14 (dd, J=1.7, 5.1 Hz, 1H), 7.63 (dd, J=1.9, 8.0 Hz, 1H), 7.44 (d, J=9.2 Hz, 2H), 6.60 (dd, J=5.2, 7.9 Hz, 1H), 6.31 (s, 2H), 6.20 (s, 1H), 3.50 (s, 2H), 3.40 (q, J=7.0 Hz, 3H), 2.79 (s, 4H), 2.54 (s, 4H), 1.98 (d, J=15.8 Hz, 2H), 1.81 (s, 2H), 1.64-1.57 (m, 2H), 1.31 ppm (d, J=32.8 Hz, 21H), Ms caled for C29H43N5O3: 509.68.

45. Compound 45: 2-Amino-N-(12-(3-(dimethylamino)azepan-1-yl)dodecyl)nicotinamide (hereinafter referred to as PBK-2025-002)
<Chemical formula 45>

Brown semi-liquid, 1H NMR (400 MHz, CDCl₃): δ=8.07 (dd, J=1.6, 4.9 Hz, 1H), 7.55 (dd, J=1.6, 7.7 Hz, 1H), 6.52 (dd, J=4.9, 7.7 Hz, 1H), 6.26 (s, 2H), 6.17 (s, 1H), 3.36-3.29 (m, 2H), 2.78-2.65 (m, 2H), 2.61-2.39 (m, 6H), 2.25 (s, 6H), 1.76 (dd, J=4.7, 8.2 Hz, 1H), 1.20 ppm (s, 15H), Ms caled for C26H47N5O: 445.70.

46. Compound 46: 2-Amino-N-(12-(hexahydrocyclopenta[c]pyrrol-2(1H)-yl)dodecyl)nicotinamide (hereinafter referred to as PBK-2025-004)
<Chemical formula 46>

White solid, ¹H NMR (400 MHz, CDCl₃): δ=8.15 (dd, J=1.8, 4.9 Hz, 1H), 7.63 (dd, J=1.7, 7.7 Hz, 1H), 6.60 (dd, J=4.9, 7.7 Hz, 1H), 6.30 (s), 6.21 (s), 3.43-3.23 (m, 4H), 2.79 (s), 2.63-2.23 (m, 4H), 1.73-1.49 (m, 1OH), 1.40-1.23 ppm (m, 18H), Ms caled for C25H42N4O: 414.64.

47. Compound 47: 2-Amino-N-(12-(4-hydroxypiperidin-1-yl)dodecyl)nicotinamide (hereinafter referred to as PBK-2025-011)
<Chemical formula 47>

White solid, ¹H NMR (400 MHz, CDCl₃): δ=8.14 (dd, J=1.7, 4.8 Hz, 1H), 7.63 (dd, J=1.5, 7.7 Hz, 1H), 6.60 (dd, J=4.9, 7.7 Hz, 1H), 6.32 (s, 2H), 6.24 (s, 1H), 3.87 (s, 1H), 3.44-3.36 (m, 2H), 2.95 (s, 2H), 2.53-2.10 (m, 9H), 1.74-1.68 (m, 2H), 1.68-1.57 (m, 4H), 1.40-1.23 ppm (m, 18H), Ms caled for C23H40N4O2: 404.60.

48. Compound 48: 2-Amino-N-(12-(4-hydroxypiperidin-1-yl) dodecyl) nicotinamide (hereinafter referred to as PBK-2025-012)
<Chemical formula 48>

White solid, ¹H NMR (400 MHz, CDCl₃): δ=8.23-8.14 (m, 3H), 7.62 (dd, J=1.6, 7.8 Hz, 1H), 6.99-6.93 (m, 2H), 6.60 (dd, J=4.8, 7.7 Hz, 1H), 6.31-6.16 (m, 3H), 4.66 (s, 1H), 3.44-2.91 (m, 6H), 2.70 (s, 2H), 2.47 (d, J=7.4 Hz, 2H), 2.07-2.01 (m, 2H), 1.65-1.55 (m, 2H), 1.39-1.23 ppm (m, 18H), Ms caled for C29H43N5O4: 525.69.

49. Compound 49: tert-Butyl(S)-(1-(12-(2-aminonicotinamido) dodecyl) pyrrolidin-3-yl)carbamate (hereinafter referred to as PBK-2025-013)
<Chemical formula 49>

White solid, ¹H NMR (400 MHz, CDCl₃): δ=8.15 (dd, J=1.8, 4.8 Hz, 1H), 7.62 (dd, J=1.7, 7.7 Hz, 1H), 6.60 (dd, J=4.9, 7.7 Hz, 1H), 6.33 (s, 2H), 6.18 (s, 1H), 4.43-4.30 (m, 1H), 3.44-3.37 (m, 3H), 3.23-3.08 (m, 1H), 2.96-2.90 (m, 1H), 2.79 (s, 3H), 2.46-2.31 (m, 1H), 2.07-1.97 (m, 1H), 1.72 (s, 2H), 1.38-1.26 ppm (m, 18H), Ms caled for C27H47N5O3: 489.69.

50. Compound 50: (S)-2-Amino-N-(12-(3-aminopyrrolidin-1-yl)dodecyl) nicotinamide hydrochloride (hereinafter referred to as PBK-2025-014)
<Chemical formula 50>

White solid, ¹H NMR (400 MHz, DMSO): δ=9.06 (t, J=4.8 Hz, 1H), 8.77-8.43 (m, 5H), 8.19 (dd, J=1.4, 6.1 Hz, 1H), 6.97 (dd, J=6.1, 7.5 Hz, 1H), 4.05-3.65 (m, 3H), 3.27-3.18 (m, 5H), 2.34-2.12 (m, 2H), 1.67 (s, 2H), 1.51 (t, J=6.5 Hz, 2H), 1.27 ppm (s, 15), Ms caled for C22H40C1N50: 426.05.

51. Compound 51: 2-Amino-N-(12-(4-(p-tolyloxy)piperidin-1-yl)dodecyl) nicotinamide (also known as PBK-2025-015)
<Chemical formula 51>

White solid, ¹H NMR (400 MHz, CDCl₃): δ=8.15 (dd, J=1.8, 4.9 Hz, 1H), 7.62 (dd, J=1.6, 7.7 Hz, 1H), 7.09 (d, J=8.4 Hz, 2H), 6.82-6.77 (m, 2H), 6.60 (dd, J=4.9, 7.7 Hz, 1H), 6.32 (s, 1H), 6.20 (s, 1H), 4.47 (s, 1H), 3.40 (q, J=6.8 Hz, 2H), 2.98 (s, 3H), 2.68 (s, 2H), 2.31 (d, J=23.0 Hz, 3H), 2.01 (s, 2H), 1.76 (s, 2H), 1.65-1.56 (m, 2H), 1.29 ppm (d, J=16.1 Hz, 20H), Ms caled for C30H46N4O2: 494.71.

52. Compound 52: 2-Amino-N-(12-(3-fluoroazetidin-1-yl) dodecyl)nicotinamide (hereinafter referred to as PBK-2025-016)
<Chemical formula 52>

White solid, 1H NMR (400 MHz, CDCl₃) δ 8.16 (dd, J=1.7, 4.9 Hz, 1H), 7.57 (dd, J=1.7, 7.7 Hz, 1H), 6.60 (dd, J=4.9, 7.7 Hz, 1H), 6.30 (s, 2H), 6.01 (s, 1H), 5.11 (dq, J=26.0, 22.7 Hz, 1H), 3.71-3.62 (m, 2H), 3.40 (td, J=5.0, 10.1 Hz, 2H), 3.13-3.02 (m, 2H), 2.46 (t, J=7.1 Hz, 2H), 1.64-1.55 (m, 2H), 1.27 (s, 18H), Ms caled for C21H35FN40: 378.53.

53. Compound 53: 2-Amino-N-(12-(tert-butyl(methyl)amino) dodecyl) nicotinamide (hereinafter referred to as PBK-2025-017)
<Chemical formula 53>

White solid, 1H NMR (400 MHz, CDCl₃) δ 8.15 (dd, J=1.8, 4.9 Hz, 1H), 7.57 (dd, J=1.7, 7.7 Hz, 1H), 6.60 (dd, J=4.9, 7.7 Hz, 1H), 6.30 (s, 2H), 6.02 (s, 1H), 3.40 (td, J=5.0, 10.1 Hz, 2H), 2.30 (t, J=7.6 Hz, 2H), 2.20 (s, 3H), 1.64-1.55 (m, 2H), 1.44-1.43 (m, 2H), 1.28 (s, 16H), 1.05 (s, 9H), Ms caled for C23H42N4O: 390.62.

54. Compound 54: 2-Amino-N-(12-(4-(2-hydroxyethyl)piperazin-1-yl)dodecyl) nicotinamide (hereinafter referred to as PBK-2025-018)
<Chemical formula 54>

White solid, ¹H NMR (400 MHz, CDCl₃): δ=8.15 (dd, J=1.7, 4.8 Hz, 1H), 7.62 (dd, J=1.7, 7.7 Hz, 1H), 6.60 (dd, J=4.9, 7.7 Hz, 1H), 6.33-6.16 (m, 3H), 3.71-3.66 (m, 2H), 3.44-3.36 (m, 2H), 2.78-2.63 (m, 12H), 2.56-2.48 (m, 3H), 1.63-1.57 (m, 4H), 1.41-1.23 ppm (m, 18H), Ms caled for C24H43N5O2: 433.64.

55. Compound 55: 2-Amino-N-(12-(4-(p-tolyl)piperidin-1-yl)dodecyl) nicotinamide (hereinafter referred to as PBK-2025-023)
<Chemical formula 55>

White solid, ¹H NMR (400 MHz, CDCl₃): δ=8.14 (dd, J=1.7, 4.8 Hz, 1H), 7.63 (dd, J=1.5, 7.7 Hz, 1H), 7.17-7.11 (m, 5H), 6.59 (dd, J=4.9, 7.7 Hz, 1H), 6.34 (s, 2H), 6.22 (s, 1H), 3.44-3.34 (m, 6H), 2.66-2.59 (m, 4H), 2.32 (s, 9H), 1.91 (d, J=13.1 Hz, 2H), 1.65-1.55 (m, 3H), 1.32-1.24 ppm (m, 21H), Ms caled for C30H₄₆N₄O: 478.73.

56. Compound 56: 2-Amino-N-(12-(3,5-dimethyl-4H-1,2,4-triazol-4-yl) dodecyl)nicotinamide (hereinafter referred to as PBK-2025-048)
<Chemical formula 56>

Clear liquid, ¹H NMR (400 MHz, CDCl₃): δ=8.03 (d, J=5.2 Hz, 1H), 7.59 (d, J=7.1 Hz, 1H), 6.57-6.50 (m, 3H), 3.88 (t, J=7.3 Hz, 2H), 3.37-3.31 (m, 2H), 2.32 (s, 3H), 2.25 (s, 3H), 1.77-1.68 (m, 2H), 1.56-1.49 ppm (m, 2H), Ms caled for C22H36N6O: 400.57.

57. Compound 57: 2-Amino-N-(12-fluoro-dodecyl)-nicotinamide (hereinafter referred to as GO15F)
<Chemical formula 57>

White solid, 1H NMR (400 MHz, CDCl₃) δ 8.14 (dd, J=4.73, 1.66 Hz, 1H), 7.55 (dd, J=7.67, 1.55 Hz, 1H), 6.58 (dd, J=7.59, 4.85 Hz, 1H), 6.28 (d, J=0.71 Hz, 2H), 5.97 (d, J=1.38 Hz, 1H), 4.48 (t, J=6.23 Hz, 1H), 4.36 (t, J=6.18 Hz, 1H), 3.39 (q, J=6.77 Hz, 2H), 1.74-1.55 (m, 4H), 1.41-1.27 (m, 16H) MS caled for C18H30FN302: 323.24.

58. Compound 58: 2-Bromo-N-(12-fluoro-dodecyl)-nicotinamide (hereinafter referred to as CS06-005-F)
<Chemical formula 58>

Dark yellow solid, 1H NMR (400 MHz, Chloroform-d) δ 8.35 (dd, J=4.6, 2.0 Hz, 1H), 7.85 (dd, J=7.6, 2.0 Hz, 1H), 7.28 (dd, J=7.6, 4.7 Hz, 1H), 6.16 (s, 1H), 4.43 (t, J=6.2 Hz, 1H), 4.31 (t, J=6.2 Hz, 1H), 3.40 (td, J=7.1, 5.8 Hz, 2H), 1.69-1.48 (m, 4H), 1.35-1.04 (m, 16H) Ms caled for C18H28BrFN2O: 387.34.

59. Compound 59: N-(12-fluoro-dodecyl)-3-(3-trifluoromethyl-phenylamino)-isonicotinamide (hereinafter referred to as PBK-069-F)
<Chemical formula 59>

Off-white solid, 1H NMR (400 MHz, Chloroform-d) δ 7.44-7.29 (m, 5H), 7.27 (s, 1H), 7.20 (d, J=7.5 Hz, 1H), 6.93-6.81 (m, 1H), 4.49 (t, J=6.2 Hz, 1H), 4.37 (t, J=6.2 Hz, 1H), 3.42 (q, J=6.7 Hz, 2H), 1.75-1.55 (m, 4H), 1.48-1.12 (m, 16H), Ms calculated for C25H35F4N30: 467.53.

60. Compound 60: 4-Chloro-N-(12-fluoro-dodecyl)-nicotinamide (hereinafter referred to as PBK-048-F)
<Chemical formula 60>

Brown solid, 1H NMR (400 MHz, methanol-d4) δ 8.57-8.48 (m, 2H), 7.58 (d, J=5.4 Hz, 1H), 4.46 (t, J=6.1 Hz, 1H), 4.34 (t, J=6.1 Hz, 1H), 3.38 (t, J=7.0 Hz, 2H), 1.65 (dtd, J=21.9, 9.0, 8.6, 6.5 Hz, 4H), 1.32 (s, 17H), Ms caled for C18H28BrFN2O: 342.88.

61. Compound 61: 2-Amino-N-(8-fluoro-octyl)-nicotinamide (hereinafter referred to as G015-F-8)
<Chemical formula 61>

White solid, 1H NMR (400 MHz, CDCl₃) δ 8.13 (dd, J=4.80, 1.72 Hz, 1H), 7.55 (dd, J=7.69, 1.75 Hz, 1H), 6.57 (dd, J=7.71, 4.95 Hz, 1H), 6.29 (s, 2H), 6.00 (s, 1H), 4.48 (t, J=6.15 Hz, 1H), 4.36 (t, J=6.15 Hz, 1H), 3.42-3.37 (m, 2H), 1.72-1.55 (m, 4H), 1.40-1.24 (m, 8H), MS caled for C14H22FN30: 267.35.

62. Compound 62: N-(12-fluoro-dodecyl)-2-mercapto-nicotinamide (hereinafter referred to as G059)
<Chemical formula 62>

Yellow solid, 1H NMR (400 MHz, CDCl₃) δ 10.62 (d, J=4.4 Hz, 1H), 8.84 (dd, J=7.49, 1.85 Hz, 1H), 7.67 (dd, J=6.04, 1.82 Hz, 1H), 6.95 (dd, J=7.61, 6.10 Hz, 1H), 4.47 (t, J=6.20 Hz, 1H), 4.36 (t, J=6.23 Hz, 1H), 3.513.46 (m, 2H), 1.72-1.61 (m, 4H), 1.46-1.23 (m, 16H), Ms caled for C18H29FN2OS: 340.50.

63. Compound 63: 2-Amino-N-(10-morpholin-4-yl-decyl)-nicotinamide (hereinafter referred to as G050)
<Chemical formula 63>

White solid, 1H NMR (400 MHz, CDCl₃) δ 8.14 (dd, J=4.94, 1.78 Hz, 1H), 7.55 (dd, J=7.64, 1.74 Hz, 1H), 6.58 (dd, J=7.63, 4.86 Hz, 1H), 6.27 (s, 2H), 3.70 (t, J=4.68 Hz, 4H), 3.42-3.37 (m, 2H), 3.41-3.37 (m, 2H), 2.41 (t, J=4.30 Hz, 4H), 2.30 (dd, J=8.77, 6.73 Hz, 2H), 1.57 (s, 2H), 1.46 (d, J=1.22 Hz, 2H), 1.36-1.28 (m, 12H), Ms caled for C20H34N4O2: 362.52.

64. Compound 64: 3-Hydroxy-pyridine-2-carboxylic acid (12-fluoro-dodecyl)-amide (hereinafter referred to as G062)
<Chemical formula 64>

White solid, 1H NMR (400 MHz, CDCl₃) δ12.67 (s, 1H), 9.14 (s, 1H), 8.10 (dd, J=4.48, 1.33 Hz, 1H), 7.47 (dd, J=8.47, 4.36 Hz, 1H), 7.35 (dd, J=8.47, 1.37 Hz, 1H), 4.43 (t, J=6.14 Hz, 1H), 4.31 (t, J=6.13 Hz, 1H), 3.24 (t, J=6.90 Hz, 2H), 1.64-1.49 (m, 4H), 1.29-1.13 (m, 16H), Ms caled for C18H29FN202: 324.44.

67. Compound 67: N-(12-fluoro-dodecyl)-2-methylsulfonyl-nicotinamide (hereinafter referred to as PBK-058-F)
<Chemical formula 67>

Off-white solid, 1H NMR (400 MHz, methanol-d4) δ 9.27 (dd, J=4.9, 1.7 Hz, 1H), 8.44 (dd, J=7.6, 1.8 Hz, 1H), 7.90 (dd, J=7.6, 4.9 Hz, 1H), 5.18 (dt, J=47.7, 6.1 Hz, 2H), 4.09 (d, J=1.6 Hz, 3H), 3.30 (s, 3H), 2.44 (ddt, J=32.8, 14.4, 6.8 Hz, 4H), 2.11 (s, 15H), Ms calcd for C19H31FN202S: 354.53.

68. Compound 68: 2-Amino-N-(10-fluoro-decyl)-nicotinamide (hereinafter referred to as G044)
<Chemical formula 68>

Pale-yellow solid, 1H NMR (400 MHz, CDCl₃) δ 8.13 (dd, J=4.81, 1.72 Hz, 1H), 7.55 (dd, J=7.70, 1.75 Hz, 1H), 6.57 (dd, J=7.70, 4.93 Hz, 1H), 6.29 (s, 2H), 6.01 (s, 1H), 4.47 (t, J=6.19 Hz, 1H), 4.35 (t, J=6.18 Hz, 1H), 3.41-3.36 (m, 2H), 1.73-1.55 (m, 4H), 1.40-1.24 (m, 12H), Ms caled for C16H26FN30: 295.

69. Compound 69: 3-(12-fluoro-dodecylcarbamoyl)-pyridin-2-yl-ammonium chloride (hereinafter referred to as GO15-F-salt)
<Chemical formula 69>

White solid, 1H NMR (400 MHz, CDCl₃) δ 8.48 (s, 2H), 8.19 (d, J=7.43 Hz, 1H), 7.69 (d, J=5.89 Hz, 1H), 7.35 (s, 1H), 6.79 (t, J=6.85 Hz, 1H), 4.48 (t, J=6.18 Hz, 1H), 4.36 (t, J=6.19 Hz, 1H), 3.44-3.39 (m, 2H), 1.72-1.59 (m, 4H), 1.39-1.27 (m, 16H), Ms caled for C18H31ClFN30: 359.91.

70. Compound 70: 2-Amino-N-(11-fluoro-undecyl)-nicotinamide (hereinafter referred to as G045)
<Chemical formula 70>

White solid, 1H NMR (400 MHz, CDCl₃) δ 7.55 (dd, J=7.64, 1.73 Hz, 1H), 4.58 (dd, J=7.63, 4.79 Hz, 1H), 6.58 (dd, J=7.63, 4.79 Hz, 1H), 6.27 (s, 2H), 5.98 (s, 1H), 4.48 (t, J=6.20 Hz, 1H), 4.36 (t, J=6.18 Hz, 1H), 3.42-3.37 (m, 2H), 1.74-1.55 (m, 4H), 1.41-1.28 (m, 14H), Ms caled for C17H28FN30: 309.43.

71. Compound 71: 3-(11-fluoro-undecylcarbamoyl)-pyridin-2-yl-ammonium chloride (hereinafter referred to as G045-salt)
<Chemical formula 71>

White solid, 1H NMR (400 MHz, CDCl₃) δ 8.50 (s, 3H), 8.39 (d, J=7.23 Hz, 1H), 7.86 (s, 1H), 7.68 (d, J=5.92 Hz, 1H), 6.80 (t, J=6.72 Hz, 1H), 4.47 (t, J=6.18 Hz, 1H), 4.35 (t, J=6.17 Hz, 1H), 3.39 (q, J=6.67 Hz, 2H), 1.73-1.60 (m, 4H), 1.40-1.23 (m, 14H), Ms caled for C17H29ClFN40: 345.89.

73. Compound 73: 4-Amino-N-(12-fluoro-dodecyl)-nicotinamide (hereinafter referred to as G060)
<Chemical formula 73>

White solid, 1H NMR (400 MHz, CDCl₃) δ 8.48 (s, 1H), 8.10 (d, J=5.81 Hz, 1H), 6.49 (d, J=5.84 Hz, 2H), 6.38 (s, 1H), 6.18 (s, 2H), 4.47 (t, J=6.20 Hz, 1H), 4.35 (t, J=6.20 Hz, 1H), 3.39 (q, J=6.57 Hz, 2H), 1.73-1.57 (m, 4H) 1.36-1.26 (m, 16H), Ms caled for C18H30FN3O: 323.46.

76. Compound 76: N-(12-fluoro-dodecyl)-3-iodo-isonicotinamide (hereinafter referred to as G073)
<Chemical formula 76>

White solid, 1H NMR (400 MHz, CDCl₃) δ 8.95 (s, 1H), 8.55 (d, J=4.76 Hz, 1H), 7.30-7.29 (m, 1H), 5.86 (s, 1H), 4.47 (t, J=6.20 Hz, 1H), 4.35 (t, J=6.19 Hz, 1H), 3.46-3.41 (m, 2H), 1.73-1.59 (m, 4H), 1.43-1.22 (m, 16H), Ms caled for C18H28FIN20: 434.34.

77. Compound 77 (C1): 2-Bromo-N-(10-cyano-decyl)benzamide (hereinafter referred to as PBK-CNB-1)
<Chemical formula 77>

Yellow oil, 1H NMR (400 MHz, Chloroform-d) δ 7.59-7.52 (m, 2H), 7.35 (td, J=7.5, 1.2 Hz, 2H), 5.99 (s, 1H), 3.47-3.42 (m, 2H), 2.34 (t, J=7.1 Hz, 3H), 1.67-1.62 (m, 4H), 1.32 (d, J=10.3 Hz, 12H), MS caled for C18H25BrN2O: 365.32.

78. Compound 78 (C2): 2-amino-N-(3-morpholin-4-yl-propyl)nicotinamide (hereinafter referred to as G035)
<Chemical formula 78>

Yellow solid, 1H NMR (500 MHz, CDCl₃) δ 8.16-8.09 (m, 2H), 7.64 (dd, J=7.64, 1.50 Hz, 1H), 6.59 (dd, J=7.63, 4.86 Hz, 1H), 6.37 (s, 2H), 3.68 (t, J=4.48 Hz, 4H), 3.51 (q, J=5.49 Hz, 2H), 2.57-2.50 (m, 6H), 1.77 (dt, J=11.73, 5.88 Hz, 2H), MS caled for C13H₂₀N₄O₂: 264.16.

79. Compound 79 (C3): 2-amino-N-(10-cyano-decyl)-nicotinamide (hereinafter referred to as CNA)
<Chemical formula 79>

Yellow solid, 1H NMR (400 MHz, Chloroform-d) δ 8.05 (dd, J=4.9, 1.8 Hz, 1H), 7.56 (dd, J=7.7, 1.8 Hz, 1H), 6.54 (dd, J=7.6, 4.9 Hz, 1H), 6.39 (s, 2H), 6.12 (t, J=5.8 Hz, 1H), 3.33 (td, J=7.4, 5.8 Hz, 2H), 2.27 (t, J=7.1 Hz, 2H), 1.56 (d, J=35.6 Hz, 4H), 1.39-1.21 (m, 12H), MS caled for C17H26N4O: 302.42.

80. Compound 80 (C4): 12-(2-bromo-benzoylamino)-dodecanoic acid ethyl ester (hereinafter referred to as CS06-010-2)
<Chemical formula 80>
81. Compound 81 (C5): 12-(2-bromo-benzoylamino)-dodecanoic acid (hereinafter referred to as CS06-010-3)
<Chemical formula 81>
82. Compound 82 (C6): 12-[(3-bromo-pyridin-4-carbonyl)-amino]-dodecanoic acid ethyl ester (hereinafter referred to as CS06-010-4)
<Chemical formula 82>
83. Compound 83 (C7): 12-[(3-bromo-pyridin-4-carbonyl)-amino]-dodecanoic acid (hereinafter referred to as CS06-010-5)
<Chemical formula 83>
88. Compound 88: N-(12-(3H-[1,2,3]triazolo[4,5-b]pyridin-3-yl)dodecyl)-2-aminonicotinamide (hereinafter referred to as PBK-2025-069)
<Chemical formula 88>

White-yellow solid, ¹H NMR (400 MHz, CDCl₃): δ=8.68-8.55 (m, 1H), 8.26-8.10 (m, 1H), 7.97-7.94 (m, 1H), 7.61-7.57 (m, 1H), 7.24 (q, J=4.2 Hz, 1H), 6.60 (d, J=8.1 Hz, 2H), 6.49 (dt, J=0.9, 6.4 Hz, 1H), 6.22 (d, J=23.5 Hz, 1H), 4.63 (q, J=7.4 Hz, 2H), 3.31-3.24 (m, 2H), 2.05-1.91 (m, 2H), 1.52-1.42 (m, 2H), 1.19 ppm (d, J=41.6 Hz, 18H), Ms caled for C23H33N7O: 423.57.

89. Compound 89: 2-Amino-N-(12-(indolin-1-yl)dodecyl)nicotinamide (hereinafter referred to as PBK-2025-070)
<Chemical formula 89>

Yellow oil, ¹H NMR (400 MHz, CDCl₃): δ=8.29 (s, 1H), 8.00 (dd, J=1.7, 5.1 Hz, 1H), 7.60 (dd, J=1.7, 7.7 Hz, 1H), 7.26 (t, J=2.9 Hz, 2H), 7.24 (s, 2H), 7.22-7.17 (m, 3H), 6.75 (s, 2H), 6.51 (dd, J=5.0, 7.7 Hz, 1H), 6.28 (s, 1H), 4.41 (s, 4H), 3.36-3.29 (m, 2H), 3.07-3.01 (m, 2H), 1.79-1.71 (m, 2H), 1.57-1.49 (m, 2H), 1.31-1.15 ppm (m, 18H), Ms caled for C26H38N4O: 422.62.

90. Compound 90: 5-Phenyl-N-(12-(piperazin-1-yl)dodecyl)furan-2-carboxamide hydrochloride (hereinafter referred to as PBK-2025-085)
<Chemical formula 90>

White-yellow solid, ¹H NMR (400 MHz, DMSO): δ=11.66 (s, 1H), 9.64 (s, 2H), 8.25-8.20 (m, 1H), 7.76-7.67 (m, 1H), 7.30 (t, J=10.5 Hz, 2H), 3.64 (s, 5H), 3.29-3.08 (m, 7H), 2.95-2.89 (m, 2H), 1.68 (s, 2H), 1.42-1.38 (m, 2H), 1.27-1.14 ppm (m, 21H), Ms caled for C27H42C1N302: 476.10.

91. Compound 91: N-(12-(4-methylpiperazin-1-yl)dodecyl)-1H-benzo[d]imidazole-5-carboxamide (hereinafter referred to as KP-16)
<Chemical formula 91>

Yellow solid, 1H NMR (400 MHz, CD3OD) δ 8.28 (s, 1H), 8.13 (bs, 1H), 7.77 (d, J=8.6 Hz, 1H), 7.66 (d, J=8.6 Hz, 1H), 3.40 (t, J=7.2 Hz, 2H), 2.52 (bs, 8H), 2.38-2.34 (m, 2H), 2.29 (s, 3H), 1.65 (p, J=7.2 Hz, 2H), 1.53-1.48 (m, 2H), 1.43-1.31 (m, 16H), Ms caled for C25H₄₁N₅O: 427.63.

92. Compound 92: ethyl 1-(12-(2-aminonicotinamido)dodecyl)piperidine-3-carboxylate (hereinafter referred to as K-PB-1)
<Chemical formula 92>

Oil, Yield: 29%, 1H NMR (400 MHz, CDCl₃) δ8.17 (dd, J=4.8, 1.4 Hz, 1H), 7.59 (dd, J=7.7, 1.5 Hz, 1H), 6.62 (dd, J=7.6, 4.8 Hz, 1H), 6.32 (s, 2H), 6.04 (s, 1H), 4.15 (q, J=7.1 Hz, 2H), 3.43 (dd, J=13.2, 7.0 Hz, 2H), 3.05 (d, J=7.1 Hz, 1H), 2.83 (s, 1H), 2.61 (s, 1H), 2.37 (s, 2H), 2.16 (d, J=10.2 Hz, 1H), 2.02 (dd, J=28.1, 8.5 Hz, 2H), 1.75 (dd, J=9.7, 3.6 Hz, 2H), 1.62 (dt, J=14.5, 7.2 Hz, 3H), 1.51 (d, J=8.8 Hz, 2H), 1.44-1.25 (m, 19H). MS caled for C26H44N4O3(M+H)+: 460, found 461

93. Compound 93: methyl 1-(12-(2-aminonicotinamido)dodecyl)piperidine-4-carboxylate (hereinafter referred to as K-PB-2)
<Chemical formula 93>

Oil, Yield: 32%, 1H NMR (400 MHz, CDCl₃) δ8.16 (dd, J=4.8, 1.5 Hz, 1H), 7.66-7.56 (m, 1H), 6.61 (dd, J=7.6, 4.9 Hz, 1H), 6.32 (s, 2H), 6.11 (s, 1H), 3.70 (s, 3H), 3.42 (dd, J=13.3, 6.8 Hz, 2H), 3.01-2.88 (m, 2H), 2.40 (d, J=7.0 Hz, 3H), 2.26-2.08 (m, 2H), 2.02 (dd, J=21.2, 9.4 Hz, 2H), 1.94-1.78 (m, 2H), 1.70-1.59 (m, 2H), 1.56 (d, J=12.0 Hz, 2H), 1.32 (d, J=28.8 Hz, 16H). MS caled for C25H42N4O3(M+H)+:446, found 447

94. Compound 94: 2-amino-N-(12-(4-methoxypiperidin-1-yl)dodecyl) nicotinamide (hereinafter referred to as K-PB-3)
<Chemical formula 94>

Oil, Yield: 29%, 1H NMR (400 MHz, CDCl₃) δ8.16 (dd, J=4.7, 1.5 Hz, 1H), 7.65-7.54 (m, 1H), 6.61 (dd, J=7.6, 4.9 Hz, 1H), 6.33 (s, 2H), 6.20 (s, 1H), 3.42 (dd, J=13.1, 7.0 Hz, 1H), 3.35 (s, 3H), 2.95 (s, 2H), 2.36-2.19 (m, 2H), 2.03 (t, J=12.7 Hz, 2H), 1.90 (s, 2H), 1.76 (s, 2H), 1.61 (dd, J=14.2, 7.0 Hz, 2H), 1.47-1.25 (m, 16H). MS caled for C24H42N4O2(M+H)+: 418, found 419

95. Compound 95: 2-amino-N-(12-(4-(2-methoxyethyl)piperazin-1-yl)dodecyl)nicotinamide (hereinafter referred to as K-PB-4)
<Chemical formula 95>

Oil, Yield: 28%, 1H NMR (400 MHz, CDCl₃) δ8.17 (dd, J=4.9, 1.7 Hz, 1H), 7.60 (dd, J=7.7, 1.7 Hz, 1H), 6.61 (dd, J=7.7, 4.9 Hz, 1H), 6.32 (s, 2H), 6.06 (s, 1H), 3.53 (t, J=5.6 Hz, 2H), 3.42 (dd, J=13.0, 7.1 Hz, 2H), 3.37 (s, 3H), 2.62 (dd, J=7.2, 3.9 Hz, 8H), 2.42 (dd, J=9.1, 6.7 Hz, 2H), 2.25 (dd, J=16.5, 9.0 Hz, 2H), 1.62 (dt, J=14.7, 7.4 Hz, 2H), 1.53 (s, 2H), 1.32 (d, J=29.2 Hz, 16H). MS caled for C25H45N5O2(M+H)+: 447, found 448

96. Compound 96: N-(12-(allyl(methyl)amino)dodecyl)-2-aminonicotinamide (hereinafter referred to as K-PB-5)
<Chemical formula 96>

Oil, Yield: 36%, 1H NMR (400 MHz, CDCl₃) δ8.08 (d, J=4.8 Hz, 2H), 7.50 (d, J=7.6 Hz, 2H), 6.53 (dd, J=7.6, 4.9 Hz, 1H), 6.23 (s, 4H), 5.93 (s, 2H), 5.79 (ddt, J=16.7, 10.2, 6.5 Hz, 2H), 5.13-5.00 (m, 4H), 3.33 (dd, J=13.2, 6.8 Hz, 4H), 2.91 (d, J=6.5 Hz, 4H), 2.28-2.21 (m, 4H), 2.13 (s, 6H), 1.52 (dd, J=14.4, 7.2 Hz, 5H), 1.38 (d, J=6.6 Hz, 5H), 1.23 (d, J=27.8 Hz, 16H). MS caled for C22H38N4O(M+H)+:374, found 375

97. Compound 97: 2-amino-N-(12-(4-formylpiperazin-1-yl)dodecyl) nicotinamide (hereinafter referred to as K-PB-6)
<Chemical formula 97>

White solid, Yield: 46%, 1H NMR (400 MHz, CDCl₃) δ8.08 (dd, J=4.8, 1.7 Hz, 1H), 7.95 (s, 1H), 7.50 (dd, J=7.7, 1.7 Hz, 1H), 6.52 (dd, J=7.7, 4.9 Hz, 1H), 6.23 (s, 2H), 5.97 (s, 1H), 3.53-3.45 (m, 2H), 3.40-3.26 (m, 4H), 2.42-2.30 (m, 4H), 2.30-2.24 (m, 2H), 1.53 (dt, J=14.7, 7.3 Hz, 2H), 1.47-1.36 (m, 2H), 1.23 (d, J=27.1 Hz, 16H). MS caled for C23H39N5O2(M+H)+: 417, found 418

98. Compound 98: 2-amino-N-(12-(ethylamino)dodecyl)nicotinamide (hereinafter referred to as K-PB-7)
<Chemical formula 98>

Oil, Yield: 25%, 1H NMR (400 MHz, CDCl₃) δ8.08 (dd, J=4.8, 1.8 Hz, 1H), 7.50 (dd, J=7.7, 1.7 Hz, 1H), 6.52 (dd, J=7.7, 4.9 Hz, 1H), 6.22 (s, 2H), 5.94 (s, 1H), 3.39-3.25 (m, 2H), 2.62-2.47 (m, 4H), 1.53 (dt, J=14.8, 7.3 Hz, 2H), 1.45-1.37 (m, 2H), 1.23 (d, J=27.9 Hz, 16H), 1.03 (t, J=7.1 Hz, 3H). MS caled for C20H₃₆N₄O(M+H)+: 348, found 349

99. Compound 99: 2-amino-N-(12-(4-(cyclopropanecarbonyl)piperazin-1-yl) dodecyl) nicotinamide (hereinafter referred to as K-PB-8)
<Chemical formula 99>

Oil, Yield: 35%, 1H NMR (400 MHz, CDCl₃) δ8.17 (dd, J=4.8, 1.6 Hz, 1H), 7.60 (dd, J=7.7, 1.6 Hz, 1H), 6.61 (dd, J=7.6, 4.9 Hz, 1H), 6.33 (s, 2H), 6.09 (s, 1H), 3.69 (d, J=18.8 Hz, 4H), 3.42 (dd, J=13.1, 7.0 Hz, 2H), 2.45 (d, J=25.0 Hz, 4H), 2.36 (dd, J=13.0, 5.2 Hz, 2H), 1.75 (ddd, J=12.7, 8.1, 4.7 Hz, 1H), 1.62 (dt, J=14.7, 7.3 Hz, 2H), 1.51 (s, 2H), 1.32 (d, J=26.6 Hz, 16H), 1.02-0.97 (m, 2H), 0.80-0.73 (m, 2H). MS caled for C26H43N5O2(M+H)+: 457, found 458

101. Compound 101: tert-Butyl (1-(12-(2-aminonicotinamido) dodecyl) piperidin-4-yl) carbamate (hereinafter referred to as K-PB-10)
<Chemical formula 101>

yellow oil, Yield: 44%, 1H NMR (400 MHz, CDCl₃) δ10.36 (s, 2H), 8.01 (s, 1H), 8.00 (d, J=5.7 Hz, 1H), 7.33 (s, 1H), 6.75 (d, J=15.2 Hz, 1H), 6.71 (t, J=9.0 Hz, 1H), 3.72 (m, 4H), 3.61 (d, J=13.8 Hz, 2H), 3.43-3.38 (m, 2H), 3.14 (m, 4H), 2.93 (dd, J=10.3, 6.0 Hz, 2H), 2.74 (d, J=10.5 Hz, 1H), 1.47 (d, J=6.7 Hz, 18H), 1.43 (s, 9H). MS caled for C28H49N5O3 (M+H)+: 503, found 504

102. Compound 102: 2-Amino-N-(12-(4,4-dimethylpiperidin-1-yl)dodecyl)nicotinamide (hereinafter referred to as K-PB-11)
<Chemical formula 102>

White solid, Yield: 17%, 1H NMR (400 MHz, CDCl₃) δ8.15 (s, 1H), 7.63 (d, J=7.5 Hz, 1H), 6.65-6.56 (m, 1H), 6.32 (s, 2H), 6.20 (s, 1H), 3.52-3.33 (m, 4H), 2.97-2.88 (m, 2H), 2.86-2.69 (m, 2H), 2.10-1.99 (m, 4H), 1.88-1.83 (m, 2H), 1.38-1.27 (m, 18H), 1.05 (s, 6H). MS caled for C25H44N4O (M+H)+: 416, found 417

103. Compound 103: Ethyl 1-(12-(2-aminonicotinamido)dodecyl)piperidine-4-carboxylate (hereinafter referred to as K-PB-12)
<Chemical formula 103>

White solid, Yield: 47.6%, 1H NMR (400 MHz, CDCl₃) δ8.15 (dd, J=4.9, 1.8 Hz, 1H), 7.60 (dd, J=7.7, 1.7 Hz, 1H), 6.59 (dd, J=7.7, 4.9 Hz, 1H), 6.30 (s, 2H), 6.12 (s, 1H), 4.14 (q, J=7.1 Hz, 2H), 3.40 (td, J=7.2, 5.9 Hz, 2H), 2.98 (s, 2H), 2.49 (s, 2H), 2.40 (s, 2H), 2.04 (s, 2H), 1.93 (s, 2H), 1.65-1.55 (m, 4H), 1.34 (s, 2H), 1.34-1.22 (m, 18H), MS caled for C26H44N4O3 (M+H)+: 460, found 461

104. Compound 104: 2-Amino-N-(12-(4-isopropylpiperazin-1-yl)dodecyl) nicotinamide (hereinafter referred to as K-PB-13)
<Chemical formula 104>

White solid, Yield: 42%, 1H NMR (400 MHz, CDCl₃) δ8.15 (dd, J=4.8, 1.7 Hz, 1H), 7.59 (dd, J=7.7, 1.7 Hz, 1H), 6.59 (dd, J=7.6, 4.9 Hz, 1H), 6.30 (s, 2H), 6.08 (s, 1H), 3.40 (dd, J=13.1, 7.1 Hz, 2H), 2.83 (s, 1H), 2.73 (s, 8H), 2.45-2.39 (m, 2H), 1.63-1.57 (m, 2H), 1.30 (d, J=29.4 Hz, 18H), 1.14 (s, 3H), 1.12 (s, 3H), MS caled for C25H45N5O (M+H)+: 431, found 432

105. Compound 105: 2-Amino-N-(12-(4-bromo-3-methyl-1H-pyrazol-1-yl)dodecyl)nicotinamide (hereinafter referred to as K-PB-14)
<Chemical formula 105>

Yellow solid, Yield: 23%, 1H NMR (400 MHz, CDCl₃) δ8.15 (d, J=4.8 Hz, 1H), 7.56 (d, J=7.6 Hz, 1H), 7.26 (d, J=0.9 Hz, 1H), 6.59 (dd, J=7.2, 5.3 Hz, 1H), 6.29 (s, 2H), 6.00 (s, 1H), 4.01 (dt, J=12.1, 7.2 Hz, 2H), 3.40 (dd, J=13.3, 6.6 Hz, 2H), 2.22 (s, 3H), 1.85-1.73 (m, 2H), 1.61-1.56 (m, 2H), 1.36-1.21 (m, 16H). MS caled for C₂₂H₃₄BrN5O (M+H)+: 464, found 465

106. Compound 106: 2-Amino-N-(12-(6-iodo-1H-indol-1-yl)dodecyl)nicotinamide (hereinafter referred to as K-PB-15)
<Chemical formula 106>

Brown oil, Yield: 39%, 1H NMR (400 MHz, CDCl₃) δ8.14 (d, J=4.7 Hz, 1H), 7.68 (s, 1H), 7.56 (d, J=7.6 Hz, 1H), 7.36 (d, J=1.3 Hz, 1H), 7.01 (d, J=3.1 Hz, 1H), 6.59 (dd, J=7.6, 4.9 Hz, 1H), 6.44 (dd, J=3.1, 0.6 Hz, 1H), 6.33 (s, 2H), 6.00 (s, 1H), 4.05 (t, J=7.2 Hz, 2H), 3.40 (dd, J=13.0, 7.0 Hz, 2H), 1.83-1.78 (m, 2H), 1.60 (dd, J=14.1, 7.3 Hz, 2H), 1.38-1.22 (m, 18H)., MS caled for C26H35IN40 (M+H)+: 546, found 547

107. Compound 107: 2-Amino-N-(12-(4-(difluoromethyl)piperidin-1-yl)dodecyl)nicotinamide (hereinafter referred to as K-PB-16)
<Chemical formula 107>

Yellow solid, Yield: 72%, 1H NMR (400 MHz, CDCl₃) δ8.15 (dd, J=4.9, 1.7 Hz, 1H), 7.57 (dd, J=7.7, 1.7 Hz, 1H), 6.60 (dd, J=7.7, 4.8 Hz, 1H), 6.29 (s, 2H), 6.00 (s, 1H), 3.40 (dd, J=13.0, 7.1 Hz, 2H), 3.03 (s, 2H), 2.36 (s, 2H), 1.94 (s, 2H), 1.80 (d, J=7.1 Hz, 2H), 1.75 (d, J=7.1 Hz, 1H), 1.61 (d, J=7.1 Hz, 4H), 1.37-1.35 (m, 1H), 1.35-1.23 (m, 18H)., MS caled for C24H40F2N40 (M+H)+: 438, found 439

108. Compound 108: N-(12-(6-Azaspiro[2.5]octan-6-yl)dodecyl)-2-aminonicotinamide (hereinafter referred to as K-PB-17)
<Chemical formula 108>

White solid, Yield: 39%, 1H NMR (400 MHz, CDCl₃) δ 8.12 (d, J=3.5 Hz, 1H), 7.74 (d, J=7.6 Hz, 1H), 6.61 (dd, J=7.6, 4.9 Hz, 1H), 6.44 (s, 2H), 6.39 (s, 1H), 3.60-3.49 (m, 2H), 3.41 (dd, J=13.2, 7.0 Hz, 2H), 3.01-2.94 (m, 2H), 2.81-2.68 (m, 3H), 1.93 (s, 4H), 1.35-1.25 (m, 18H), 0.54 (s, 2H), 0.36 (s, 2H)., MS caled for C25H42N4O (M+H)+: 414, found 415

109. Compound 109: 2-Amino-N-(12-(4-chloro-5-iodo-1H-pyrrolo[2,3-b]pyridin-1-yl)dodecyl) nicotinamide (hereinafter referred to as K-PB-18)
<Chemical formula 109>

Yellow solid, Yield: 36%, 1H NMR (400 MHz, CDCl₃) δ8.54 (s, 1H), 8.14 (d, J=4.8 Hz, 1H), 7.56 (d, J=7.7 Hz, 1H), 7.19 (d, J=3.4 Hz, 1H), 6.61-6.55 (m, 1H), 6.52 (dd, J=3.5, 0.9 Hz, 1H), 6.31 (s, 2H), 6.03 (s, 1H), 4.24 (t, J=7.1 Hz, 2H), 3.42-3.37 (m, 2H), 1.61-1.57 (m, 2H), 1.33-1.22 (m, 18H). MS caled for C25H33C1IN50 (M+H)+: 581, found 582

110. Compound 110: 2-amino-N-(12-((2S,6R)-2,6-dimethylmorpholino)dodecyl)nicotinamide (hereinafter referred to as K-PB-19)
<Chemical formula 110>

Yellow solid, Yield: 80%, 1H NMR (400 MHz, CDCl₃) δ8.15 (dd, J=4.9, 1.8 Hz, 1H), 7.57 (dd, J=7.7, 1.7 Hz, 1H), 6.59 (dd, J=7.7, 4.9 Hz, 1H), 6.29 (s, 2H), 5.99 (s, 1H), 3.77-3.63 (m, 2H), 3.40 (td, J=7.2, 5.9 Hz, 2H), 2.76 (d, J=11.1 Hz, 2H), 2.36-2.25 (m, 2H), 1.76-1.43 (m, 12H), 1.40-1.27 (m, 1OH), 1.17 (s, 3H), 1.15 (s, 3H)., MS caled for C24H42N4O2 (M+H)+: 418, found 419

111. Compound 111: tert-butyl-4-(12-(2-aminonicotinamido)dodecyl) piperazine-1-carboxylate (hereinafter referred to as K-PB-20)
<Chemical formula 111>

Yellow solid, Yield: 55%, 1H NMR (400 MHz, CDCl₃) δ8.15 (dd, J=4.9, 1.8 Hz, 1H), 7.57 (dd, J=7.7, 1.7 Hz, 1H), 6.59 (dd, J=7.7, 4.9 Hz, 1H), 6.29 (s, 2H), 6.01 (s, 1H), 3.51-3.33 (m, 6H), 2.43-2.34 (m, 4H), 2.34-2.28 (m, 2H), 1.69-1.53 (m, 6H), 1.52-1.42 (m, 11H), 1.40-1.27 (m, 12H)., MS caled for C27H47N5O3 (M+H)+: 489, found 490

113. Compound 113: 2-amino-N-(12-(4-(cyclohexylmethyl)piperazin-1-yl)dodecyl)nicotinamide (hereinafter referred to as K-PB-22)
<Chemical formula 113>

Yellow solid, Yield: 87%, 1H NMR (400 MHz, CDCl₃) δ8.15 (dd, J=4.9, 1.8 Hz, 1H), 7.59 (dd, J=7.7, 1.7 Hz, 1H), 6.60 (dd, J=7.7, 4.9 Hz, 1H), 6.30 (s, 2H), 6.06 (s, 1H), 3.46-3.34 (m, 2H), 3.00-2.41 (m, J=38.6 Hz, 1OH), 2.21 (s, 2H), 1.80-1.42 (m, 14H), 1.42-1.26 (m, 12H), 1.16 (ddd, J=12.5, 8.0, 2.4 Hz, 3H), 0.87 (dd, J=22.4, 10.7 Hz, 2H)., MS caled for C29H51N5O (M+H)+: 485, found 486

114. Compound 114: 2-amino-N-(12-(4,4-difluoropiperidin-1-yl)dodecyl)nicotinamide (hereinafter referred to as K-PB-23)
<Chemical formula 114>

Yellow solid, Yield: 33%, 1H NMR (400 MHz, CDCl₃) δ8.18 (dd, J=4.8, 1.7 Hz, 1H), 7.59 (dd, J=7.6, 1.3 Hz, 1H), 6.62 (dd, J=7.6, 4.9 Hz, 1H), 6.31 (s, 2H), 6.01 (s, 1H), 3.43 (dd, J=13.4, 6.7 Hz, 2H), 2.61-2.48 (m, 4H), 2.42-2.34 (m, 2H), 2.08-1.95 (m, 4H), 1.67-1.45 (m, 1OH), 1.44-1.30 (m, 1OH). MS caled for C23H38F2N40 (M+H)+: 424, found 425

115. Compound 115: 2-amino-N-(12-(4-fluoropiperidin-1-yl)dodecyl)nicotinamide (hereinafter referred to as K-PB-24)
<Chemical formula 115>

Yellow solid, Yield: 64%, 1H NMR (400 MHz, CDCl₃) δ8.15 (dd, J=4.9, 1.8 Hz, 1H), 7.59 (dd, J=7.7, 1.7 Hz, 1H), 6.60 (dd, J=7.7, 4.9 Hz, 1H), 6.30 (s, 2H), 6.05 (s, 1H), 4.84 (d, J=48.9 Hz, 2H), 3.45-3.34 (m, 2H), 2.92-2.57 (m, J=72.2 Hz, 5H), 2.31 (s, 2H), 2.04 (s, 2H), 1.83-1.28 (m, J=113.7, 70.4, 15.8 Hz, 20H)., MS caled for C23H39FN40 (M+H)+: 406, found 407

116. Compound 116: Methyl-1-(12-(2-aminonicotinamido)dodecyl)pyrrolidine-3-carboxylate (hereinafter referred to as K-PB-25)
<Chemical formula 116>

Yellow solid, Yield: 29%, 1H NMR (400 MHz, CDCl₃) δ8.18 (dd, J=4.9, 1.8 Hz, 1H), 7.60 (dd, J=7.7, 1.7 Hz, 1H), 6.62 (dd, J=7.7, 4.9 Hz, 1H), 6.32 (s, 2H), 6.05 (s, 1H), 3.73 (s, 3H), 3.49-3.36 (m, 2H), 3.17 (s, 2H), 2.79 (s, 1H), 2.62 (s, 2H), 2.29-2.11 (m, J=28.5 Hz, 2H), 1.78-1.44 (m, 12H), 1.42-1.29 (m, 10H)., MS caled for C24H40N4O3 (M+H)+: 432, found 433

117. Compound 117: tert-butyl-(R)-(1-(12-(2-aminonicotinamido)dodecyl)piperidin-3-yl)carbamate (hereinafter referred to as K-PB-26)
<Chemical formula 117>

Yellow solid, Yield: 32%, 1H NMR (400 MHz, CDCl₃) δ8.17 (dd, J=4.9, 1.8 Hz, 1H), 7.60 (dd, J=7.7, 1.7 Hz, 1H), 6.62 (dd, J=7.7, 4.9 Hz, 1H), 6.32 (s, 2H), 6.05 (s, 1H), 5.03 (s, 1H), 3.77 (s, 1H), 3.43 (dd, J=13.0, 7.2 Hz, 2H), 2.57-2.23 (m, 6H), 1.80-1.53 (m, 12H), 1.47 (s, 12H), 1.42-1.29 (m, J=12.8, 5.9 Hz, 9H)., MS caled for C28H49N5O3 (M+H)+: 503, found 504

118. Compound 118: 2-chloro-N-(12-(4-methylpiperazin-1-yl)dodecyl)nicotinamide (hereinafter referred to as K-PB-31)
<Chemical formula 118>

White solid, Yield: 32%, 1H NMR (400 MHz, CDCl₃) δ8.46 (dd, J=4.8, 2.0 Hz, 1H), 8.11 (dd, J=7.6, 2.0 Hz, 1H), 7.35 (dd, J=7.6, 4.8 Hz, 1H), 6.47 (s, 1H), 3.58 (s, 8H), 3.51-3.45 (m, 2H), 3.04-2.98 (m, 2H), 1.77-1.68 (m, 2H), 1.64 (dt, J=14.6, 7.2 Hz, 2H), 1.45-1.20 (m, 16H)., MS caled for C23H39C1N40 (M+H)+: 422, found 423

121. Compound 121: 2-fluoro-N-(12-(4-methylpiperazin-1-yl)dodecyl) nicotinamide (hereinafter referred to as K-PB-40)
<Chemical formula 121>

White solid, Yield: 35% 1H NMR (400 MHz, CDCl₃) δ8.58 (ddd, J=9.8, 7.5, 2.1 Hz, 1H), 8.36-8.28 (m, 1H), 7.36 (ddd, J=7.4, 4.8, 2.4 Hz, 1H), 6.84 (s, 1H), 3.58 (s, 8H), 3.48 (td, J=7.1, 1.2 Hz, 2H), 3.05-2.98 (m, 2H), 2.84 (s, 3H), 1.79-1.56 (m, 4H), 1.43-1.18 (m, 16H)., MS caled for C23H39FN40 (M+H)+: 406, found 407

122. Compound 122: 2-Amino-N-(12-(3,3-difluoroazetidin-1-yl)dodecyl) nicotinamide (hereinafter referred to as K-PB-44)
<Chemical formula 122>

Yellow solid, Yield: 35%, 1H NMR (400 MHz, CDCl₃) δ8.15 (dd, J=4.8, 1.6 Hz, 1H), 7.57 (dd, J=7.6, 1.5 Hz, 1H), 6.59 (dd, J=7.6, 4.9 Hz, 1H), 6.30 (s, 2H), 6.02 (s, 1H), 3.53 (t, J=12.1 Hz, 4H), 3.40 (dd, J=13.2, 6.9 Hz, 2H), 2.52 (t, J=7.1 Hz, 2H), 1.60 (dt, J=14.6, 7.2 Hz, 2H), 1.37-1.25 (m, 18H)., MS caled for C21H34F2N40 (M+H)+: 396, found 397

123. Compound 123: 2-Amino-N-(12-(6-chloro-1H-pyrazolo[3,4-b]pyridin-1-yl) dodecyl)nicotinamide (hereinafter referred to as K-PB-45)
<Chemical formula 123>

White solid, Yield: 19%, 1H NMR (400 MHz, CDCl₃) δ8.14 (s, 1H), 7.96 (d, J=8.5 Hz, 1H), 7.90 (s, 1H), 7.61 (d, J=7.5 Hz, 1H), 7.04 (d, J=8.6 Hz, 1H), 6.60 (dd, J=6.3, 5.2 Hz, 1H), 6.41 (s, 2H), 6.10 (s, 1H), 4.40 (t, J=7.0 Hz, 2H), 3.43-3.38 (m, 2H), 2.10-1.93 (m, 2H), 1.66-1.53 (m, 2H), 1.36-1.19 (m, 16H)., MS caled for C24H33C1N60 (M+H)+: 457, found 458

124. Compound 124: 2-Amino-N-(12-(4-(4-methylpiperazin-1-yl)piperidin-1-yl)dodecyl) nicotinamide (hereinafter referred to as K-PB-46)
<Chemical formula 124>

White solid, Yield: 82%, 1H NMR (400 MHz, CDCl₃) δ8.15 (d, J=4.8 Hz, 1H), 7.57 (d, J=7.6 Hz, 1H), 6.62-6.55 (m, 1H), 6.30 (s, 2H), 6.02 (s, 1H), 3.40 (dd, J=13.3, 6.5 Hz, 2H), 2.99 (d, J=11.4 Hz, 2H), 2.60 (s, 4H), 2.46 (s, 4H), 2.28 (s, 3H), 2.27-2.22 (m, 1H), 1.90 (m, 4H), 1.79 (d, J=12.1 Hz, 2H), 1.60 (t, J=7.4 Hz, 4H), 1.46 (m, 2H), 1.30 (m, 16H)., MS caled for C28H₅₀N₆O (M+H)+: 486, found 487

125. Compound 125: 2-Amino-N-(12-(6,7-dimethoxy-3,4-dihydroisoquinolin-2(1H)-yl)dodecyl)nicotinamide (hereinafter referred to as K-PB-47)
<Chemical formula 125>

White solid, Yield: 40%, 1H NMR (400 MHz, CDCl₃) δ8.13 (s, 1H), 7.57 (d, J=25.3 Hz, 1H), 6.59 (s, 1H), 6.55 (s, 1H), 6.51 (s, 1H), 6.31 (s, 2H), 6.16 (s, 1H), 3.87 (s, 3H), 3.83 (s, 3H), 3.39 (m, 2H), 2.97 (m, 2H), 2.97 (m, 1H), 2.73 (m, 1H), 1.74 (s, 3H), 1.59 (m, 4H), 1.26 (m, 18H)., MS caled for C29H44N4O3 (M+H)+: 496, found 497

126. Compound 126: 2-amino-N-(12-(3-methoxypyrrolidin-1-yl)dodecyl) nicotinamide (hereinafter referred to as K-PB-48)
<Chemical formula 126>

Oil, Yield: 13%, 1H NMR (400 MHz, CDCl₃) δ8.16 (d, J=4.1 Hz, 1H), 7.61 (t, J=22.1 Hz, 1H), 6.62 (dd, J=7.1, 5.1 Hz, 1H), 6.28 (d, J=40.1 Hz, 2H), 6.20 (s, 1H), 4.10 (s, 1H), 3.87-3.60 (m, 2H), 3.42 (dd, J=13.3, 6.6 Hz, 2H), 3.34 (d, J=0.8 Hz, 3H), 3.15-2.83 (m, 4H), 2.23 (dd, J=12.0, 5.2 Hz, 2H), 1.84 (dd, J=14.8, 7.4 Hz, 2H), 1.62 (dd, J=14.3, 7.1 Hz, 2H), 1.47-1.23 (m, 16H). MS caled for C23H40N4O2(M+H)+:404, found 405

127. Compound 127: 2-amino-N-(12-(2-oxooxazolidin-3-yl)dodecyl) nicotinamide (hereinafter referred to as K-PB-49)
<Chemical formula 127>

Oil, Yield: 15%, 1H NMR (400 MHz, CDCl₃) δ8.17 (d, J=4.7 Hz, 1H), 7.61 (d, J=7.6 Hz, 1H), 6.62 (dd, J=7.6, 4.9 Hz, 1H), 6.32 (s, 2H), 6.11 (s, 1H), 4.34 (t, J=8.0 Hz, 2H), 3.57 (t, J=8.0 Hz, 2H), 3.43 (dd, J=13.4, 6.7 Hz, 2H), 3.27 (t, J=7.3 Hz, 2H), 1.58 (dd, J=15.3, 7.4 Hz, 4H), 1.31 (d, J=14.0 Hz, 16H). MS caled for C21H34N4O3(M+H)+:390, found 391

128. Compound 128: 2-amino-N-(12-((tetrahydrofuran-3-yl)amino)dodecyl) nicotinamide (hereinafter referred to as K-PB-50)
<Chemical formula 128>

Oil, Yield: 22%, 1H NMR (400 MHz, CDCl₃) δ8.17 (dd, J=4.8, 1.4 Hz, 1H), 7.60 (dd, J=7.7, 1.7 Hz, 1H), 6.62 (dd, J=7.6, 4.9 Hz, 1H), 6.33 (s, 2H), 6.07 (s, 1H), 3.96 (dd, J=14.7, 8.1 Hz, 1H), 3.88-3.73 (m, 2H), 3.68 (dd, J=9.2, 3.9 Hz, 1H), 3.52-3.46 (m, 1H), 3.43 (dd, J=13.4, 6.6 Hz, 2H), 2.72-2.58 (m, 2H), 2.18-2.03 (m, 4H), 1.82 (ddd, J=12.5, 7.7, 4.2 Hz, 1H), 1.68-1.50 (m, 4H), 1.43-1.26 (m, 16H). MS caled for C22H38N4O2(M+H)+:390, found 391

129. Compound 129: 2-amino-N-(12-(2,5-dihydro-1H-pyrrol-1-yl)dodecyl) nicotinamide (hereinafter referred to as K-PB-51)
<Chemical formula 129>

Oil, Yield: 30%, 1H NMR (400 MHz, CDCl₃) δ8.17 (dd, J=4.8, 1.7 Hz, 1H), 7.69-7.55 (m, 1H), 6.62 (dd, J=7.7, 4.9 Hz, 1H), 6.32 (s, 2H), 6.14 (dd, J=13.8, 11.7 Hz, 1H), 5.82 (s, 2H), 3.73 (d, J=30.7 Hz, 4H), 3.43 (dd, J=13.0, 7.1 Hz, 2H), 2.87-2.69 (m, 2H), 1.62 (dt, J=14.6, 7.2 Hz, 4H), 1.31 (d, J=16.1 Hz, 16H). MS caled for C₂₂H₃₆N₄O(M+H)+: 372, found 373

130. Compound 130: N-(12-(2-aminonicotinamido)dodecyl)-N-formylalanine (hereinafter referred to as K-PB-52)
<Chemical formula 130>

Oil, Yield: 18%, 1H NMR (400 MHz, CDCl₃) δ8.21 (s, 1H), 8.16 (dd, J=14.7, 11.5 Hz, 1H), 7.63-7.54 (m, 1H), 6.62 (dd, J=7.6, 4.9 Hz, 1H), 6.29 (d, J=30.3 Hz, 2H), 6.07 (s, 1H), 4.76-4.63 (m, 1H), 4.18 (t, J=6.7 Hz, 2H), 3.43 (dd, J=13.1, 7.0 Hz, 2H), 1.75-1.60 (m, 4H), 1.48 (t, J=8.4 Hz, 3H), 1.30 (s, 16H). MS caled for C22H36N4O4 (M+H)+: 420, found 421

131. Compound 131: 1-(2-aminopyridin-3-yl)-14-(8-oxa-3-azabicyclo[3.2.1]octan-3-yl)tetradecan-1-one (hereinafter referred to as K-PB-53)
<Chemical formula 131>

White solid, Yield: 35%, 1H NMR (400 MHz, CDCl₃) δ8.17 (dd, J=4.8, 1.7 Hz, 1H), 7.59 (dd, J=7.7, 1.7 Hz, 1H), 6.61 (dd, J=7.7, 4.9 Hz, 1H), 6.32 (s, 2H), 6.04 (s, 1H), 4.28 (dd, J=4.3, 2.1 Hz, 2H), 3.46-3.31 (m, 2H), 2.58 (dd, J=10.0, 0.9 Hz, 2H), 2.26 (dd, J=12.5, 4.6 Hz, 4H), 1.95 (dt, J=7.8, 3.6 Hz, 2H), 1.84 (dd, J=7.3, 4.3 Hz, 2H), 1.62 (dt, J=14.7, 7.3 Hz, 2H), 1.50-1.27 (m, 18H). MS caled for C24H40N4O2 (M+H)+:416, found 417

132. Compound 132: 1-(2-aminopyridin-3-yl)-14-(isobutyl(methyl)amino)tetradecan-1-one (hereinafter referred to as K-PB-54)
<Chemical formula 132>

Oil, Yield: 32%, 1H NMR (400 MHz, CDCl₃) δ8.07 (d, J=3.7 Hz, 1H), 7.53 (dt, J=15.4, 7.7 Hz, 1H), 6.53 (dd, J=7.6, 4.9 Hz, 1H), 6.23 (s, 2H), 6.06 (s, 1H), 3.33 (dd, J=13.1, 7.0 Hz, 2H), 2.70-2.57 (m, 2H), 2.48 (s, 3H), 2.44 (d, J=7.1 Hz, 2H), 1.88 (ddd, J=20.3, 13.7, 6.9 Hz, 1H), 1.63-1.48 (m, 4H), 1.35-1.17 (m, 16H), 0.94 (d, J=6.6 Hz, 6H). MS caled for C23H42N4O (M+H)+: 390, found 391

133. Compound 133: 1-(2-aminopyridin-3-yl)-14-(methyl(propyl)amino)tetradecan-1-one (hereinafter referred to as K-PB-55)
<Chemical formula 133>

Oil, Yield: 26%, 1H NMR (400 MHz, CDCl₃) δ8.08 (d, J=3.5 Hz, 1H), 7.53 (dd, J=7.7, 1.7 Hz, 1H), 6.53 (dd, J=7.7, 4.9 Hz, 1H), 6.23 (s, 2H), 6.02 (s, 1H), 3.33 (dd, J=13.0, 7.1 Hz, 2H), 2.54 (dd, J=16.0, 10.0 Hz, 4H), 2.39 (s, 3H), 1.63-1.47 (m, 6H), 1.34-1.19 (m, 16H), 0.88 (t, J=7.4 Hz, 3H). MS caled for C₂₂H40N4O (M+H)+: 376, found 377

134. Compound 134: 1-(2-aminopyridin-3-yl)-14-(2-(hydroxymethyl)pyrrolidin-1-yl)tetradecan-1-one (hereinafter referred to as K-PB-56)
<Chemical formula 134>

Oil, Yield: 20%, 1H NMR (400 MHz, CDCl₃) δ8.17 (dd, J=4.8, 1.5 Hz, 1H), 7.62 (dt, J=9.1, 4.6 Hz, 1H), 6.62 (dd, J=7.6, 4.9 Hz, 1H), 6.33 (s, 2H), 6.15 (s, 1H), 3.81 (dd, J=12.1, 3.3 Hz, 1H), 3.71 (dd, J=12.1, 5.1 Hz, 1H), 3.54 (s, 1H), 3.42 (dt, J=12.6, 6.3 Hz, 2H), 3.12-2.96 (m, 2H), 2.68-2.55 (m, 2H), 2.08-1.96 (m, 2H), 1.96-1.85 (m, 2H), 1.79 (s, 1H), 1.62 (dt, J=14.6, 7.4 Hz, 4H), 1.29 (s, 16H). MS caled for C23H40N4O2 (M+H)+:404, found 405

147. Compound 147: 1-(2-aminopyridin-3-yl)-14-((2-(methylamino)ethyl)amino)tetradecan-1-one (hereinafter referred to as K-PB-57)
<Chemical formula 147>

Oil, Yield: 27%, 1H NMR (400 MHz, CDCl₃) δ8.17 (dd, J=4.9, 1.8 Hz, 1H), 7.59 (dt, J=18.1, 9.0 Hz, 1H), 6.61 (dd, J=7.7, 4.9 Hz, 1H), 6.32 (s, 2H), 6.12 (s, 1H), 3.48-3.35 (m, 2H), 2.78 (t, J=6.2 Hz, 2H), 2.51-2.38 (m, 2H), 2.34 (dd, J=21.7, 14.1 Hz, 2H), 2.29-2.18 (m, 3H), 1.61 (dd, J=14.6, 7.4 Hz, 2H), 1.46 (dd, J=16.9, 10.2 Hz, 2H), 1.32 (d, J=27.7 Hz, 18H). MS caled for C21H₃₉N₅O (M+H)+: 377, found 378

148. Compound 148: N-(14-(2-aminopyridin-3-yl)-14-oxotetradecyl)-2,2,2-trifluoroacetamide (hereinafter referred to as K-PB-58)
<Chemical formula 148>

Oil, Yield: 19%, 1H NMR (400 MHz, CDCl₃) δ8.17 (dd, J=4.9, 1.7 Hz, 1H), 7.60 (dd, J=7.7, 1.7 Hz, 1H), 6.62 (dd, J=7.7, 4.9 Hz, 1H), 6.36 (s, 1H), 6.31 (s, 2H), 6.05 (s, 1H), 3.40 (ddd, J=19.3, 13.3, 6.9 Hz, 4H), 1.61 (dd, J=15.1, 7.1 Hz, 4H), 1.31 (d, J=14.4 Hz, 16H). MS caled for C20H31F3N402 (M+H)+:416, found 417

149. Compound 149: 1-(2-aminopyridin-3-yl)-14-(3-hydroxypiperidin-1-yl)tetradecan-1-one (hereinafter referred to as K-PB-59)
<Chemical formula 149>

Oil, Yield: 17%, 1H NMR (400 MHz, CDCl₃) δ8.17 (dd, J=4.9, 1.8 Hz, 1H), 7.61 (dd, J=7.7, 1.7 Hz, 1H), 6.62 (dd, J=7.7, 4.9 Hz, 1H), 6.33 (s, 2H), 6.09 (s, 1H), 3.96 (s, 1H), 3.46-3.37 (m, 2H), 2.64 (s, 2H), 2.52-2.44 (m, 2H), 2.03 (dd, J=12.5, 6.8 Hz, 2H), 1.93 (s, 2H), 1.73-1.54 (m, 6H), 1.33 (d, J=29.0 Hz, 16H). MS caled for C23H40N4O2 (M+H)+:404, found 405

150. Compound 150: methyl 2-(1-(12-(2-aminonicotinamido)dodecyl)piperidin-4-yl)acetate (hereinafter referred to as K-PB-60)
<Chemical formula 150>

Oil, Yield: 16%, 1H NMR (400 MHz, CDCl₃) δ8.17 (d, J=4.8 Hz, 1H), 7.63 (d, J=7.6 Hz, 1H), 6.62 (dd, J=7.6, 4.9 Hz, 1H), 6.32 (s, 2H), 6.16 (s, 1H), 3.70 (s, 3H), 3.43 (dd, J=13.3, 6.8 Hz, 2H), 3.36 (s, 2H), 2.74 (s, 2H), 2.47 (s, 2H), 2.34 (d, J=6.6 Hz, 2H), 2.01 (dd, J=17.9, 11.8 Hz, 3H), 1.88 (s, 2H), 1.77 (s, 2H), 1.62 (dd, J=14.1, 7.0 Hz, 2H), 1.31 (d, J=17.5 Hz, 16H). MS caled for C26H44N4O3 (M+H)+: 460, found 461

151. Compound 151: 3-(14-(2-aminopyridin-3-yl)-14-oxotetradecyl) oxazolidine-2,4-dione (hereinafter referred to as K-PB-61)
<Chemical formula 151>

Oil, Yield: 44%, 1H NMR (400 MHz, CDCl₃) δ8.17 (dd, J=4.8, 1.6 Hz, 1H), 7.59 (dd, J=7.6, 1.5 Hz, 1H), 6.61 (dd, J=7.6, 4.9 Hz, 1H), 6.32 (s, 2H), 6.07 (s, 1H), 4.70 (s, 2H), 3.55 (dd, J=19.2, 11.8 Hz, 2H), 3.42 (dd, J=13.0, 7.0 Hz, 2H), 1.64 (ddd, J=28.3, 14.1, 6.9 Hz, 4H), 1.30 (d, J=16.4 Hz, 16H). MS caled for C21H32N4O4 (M+H)+:404, found 405

152. Compound 152: 1-(2-aminopyridin-3-yl)-14-(ethyl(methyl)amino)tetradecan-1-one (hereinafter referred to as K-PB-62)
<Chemical formula 152>

Oil, Yield: 13%, 1H NMR (400 MHz, CDCl₃) δ8.26-8.14 (m, 1H), 7.65 (dd, J=7.7, 1.5 Hz, 1H), 6.62 (dd, J=7.6, 4.9 Hz, 1H), 6.34 (s, 2H), 6.21 (s, 1H), 3.43 (dd, J=13.2, 7.0 Hz, 2H), 3.09 (d, J=7.3 Hz, 2H), 2.98-2.88 (m, 2H), 2.73 (s, 3H), 2.03 (dd, J=15.1, 9.2 Hz, 2H), 1.84 (s, 2H), 1.44 (t, J=7.3 Hz, 3H), 1.32 (d, J=24.8 Hz, 16H). MS caled for C21H38N4O (M+H)+:362, found 363

153. Compound 153: 1-(2-aminopyridin-3-yl)-14-(methyl(oxetan-3-yl)amino)tetradecan-1-one (hereinafter referred to as K-PB-63)
<Chemical formula 153>

Oil, Yield: 42%, 1H NMR (400 MHz, CDCl₃) δ8.14 (dd, J=4.9, 1.7 Hz, 1H), 7.59 (dd, J=7.7, 1.7 Hz, 1H), 6.59 (dd, J=7.7, 4.9 Hz, 1H), 6.39 (d, J=43.8 Hz, 2H), 6.18 (s, 1H), 4.62 (p, J=6.4 Hz, 4H), 3.53 (p, J=6.7 Hz, 1H), 3.44-3.36 (m, 2H), 2.21-2.14 (m, 2H), 2.11 (s, 3H), 1.66-1.55 (m, 2H), 1.42 (dd, J=13.8, 7.2 Hz, 2H), 1.31 (d, J=26.1 Hz, 16H). MS caled for C22H38N4O2 (M+H)+:390, found 391

154. Compound 154: 1-(2-aminopyridin-3-yl)-14-(2-(2-hydroxyethyl) piperidin-1-yl)tetradecan-1-one (hereinafter referred to as K-PB-64)
<Chemical formula 154>

Oil, Yield: 36%, 1H NMR (400 MHz, CDCl₃) δ8.06 (dd, J=4.8, 1.5 Hz, 1H), 7.94 (s, 1H), 7.58 (dd, J=7.6, 1.4 Hz, 1H), 6.53 (dd, J=7.6, 4.9 Hz, 1H), 6.26 (s, 2H), 3.88 (s, 1H), 3.77-3.61 (m, 1H), 3.41 (s, 2H), 3.33 (dd, J=13.0, 7.0 Hz, 2H), 2.89 (s, 4H), 2.81 (s, 4H), 1.93 (d, J=17.7 Hz, 2H), 1.80 (d, J=8.2 Hz, 4H), 1.58-1.50 (m, 2H), 1.35-1.14 (m, 16H), MS caled for C25H44N4O2 (M+H)+:432, found 433

155. Compound 155: 1-(2-aminopyridin-3-yl)-14-(2-methylmorpholino)tetradecan-1-one (hereinafter referred to as K-PB-65)
<Chemical formula 155>

Oil, Yield: 56%, 1H NMR (400 MHz, CDCl₃) δ8.15 (dd, J=4.9, 1.7 Hz, 1H), 7.59 (dd, J=7.7, 1.7 Hz, 1H), 6.59 (dd, J=7.7, 4.9 Hz, 1H), 6.33 (s, 2H), 6.12 (s, 1H), 3.85 (ddd, J=11.3, 3.3, 1.4 Hz, 1H), 3.73-3.59 (m, 2H), 3.45-3.35 (m, 2H), 2.74 (ddd, J=20.6, 11.3, 1.7 Hz, 2H), 2.35-2.28 (m, 2H), 2.08 (td, J=11.5, 3.4 Hz, 1H), 1.77 (dd, J=11.0, 10.3 Hz, 1H), 1.60 (dt, J=14.7, 7.4 Hz, 2H), 1.51-1.43 (m, 2H), 1.31 (d, J=26.3 Hz, 16H), 1.15 (d, J=6.3 Hz, 3H). MS caled for C23H40N4O2 (M+H)+: 404, found 405

156. Compound 156: 1-(2-aminopyridin-3-yl)-14-((2-hydroxyethyl)(methyl)amino)tetradecan-1-one (hereinafter referred to as K-PB-66)
<Chemical formula 156>

Oil, Yield: 18%, 1H NMR (400 MHz, CDCl₃) δ8.17 (d, J=4.8 Hz, 1H), 7.61 (d, J=7.7 Hz, 1H), 6.62 (dd, J=7.6, 4.9 Hz, 1H), 6.33 (s, 2H), 6.11 (s, 1H), 3.83-3.71 (m, 2H), 3.43 (dd, J=13.2, 6.8 Hz, 2H), 2.85-2.77 (m, 2H), 2.72-2.62 (m, 2H), 2.51 (s, 3H), 2.09-1.98 (m, 1H), 1.62 (dt, J=14.6, 7.3 Hz, 4H), 1.31 (d, J=11.2 Hz, 16H). MS caled for C21H38N4O2 (M+H)+:378, found 377

157. Compound 157: 2-amino-N-(12-(2,2,6,6-tetramethylpiperidin-1-yl) dodecyl) nicotinamide (hereinafter referred to as K-PB-67)
<Chemical formula 157>

Light yellow solid, Yield: 43% 1H NMR (400 MHz, CDCl₃) δ8.14 (d, J=4.8 Hz, 1H), 7.72-7.49 (m, 1H), 6.59 (dd, J=7.6, 4.9 Hz, 1H), 6.30 (s, 2H), 3.40 (dd, J=13.4, 6.8 Hz, 2H), 2.99-2.67 (m, 2H), 1.79-1.61 (m, 18H), 1.43-1.20 (m, 20H). MS caled for C27H48N4O (M+H)+: 444, found 445

158. Compound 158: (2S,3aS,7aS)-1-(12-(2-aminonicotinamido)dodecyl) octahydro-1H-indole-2-carboxylic acid (hereinafter referred to as K-PB-68)
<Chemical formula 158>

Colorless oil, Yield: 27% 1H NMR (400 MHz, MeOD) δ8.03 (dd, J=5.0, 1.8 Hz, 1H), 7.85 (dd, J=7.7, 1.8 Hz, 1H), 6.65 (dd, J=7.7, 5.0 Hz, 1H), 3.98 (dd, J=9.5, 6.3 Hz, 1H), 3.57 (dd, J=12.5, 6.0 Hz, 1H), 3.30-3.23 (m, 2H), 3.17-3.05 (m, 1H), 2.56-2.38 (m, 2H), 2.15-2.05 (m, 1H), 1.97-1.81 (m, 1H), 1.78-1.68 (m, 1H), 1.68-1.50 (m, 8H), 1.43-1.28 (m, 18H)., MS caled for C27H44N4O3 (M+H)+: 472, found 473

159. Compound 159: 3-(12-(2-aminonicotinamido)dodecyl)thiazolidine-2-carboxylic acid (hereinafter referred to as K-PB-69)
<Chemical formula 159>

White solid, Yield: 20% 1H NMR (400 MHz, CDCl₃) δ8.17 (dd, J=4.8, 1.5 Hz, 1H), 7.59 (dd, J=7.7, 1.4 Hz, 1H), 6.62 (dd, J=7.6, 4.9 Hz, 1H), 6.32 (s, 2H), 6.03 (s, 1H), 4.94 (s, 1H), 4.27-4.11 (m, 2H), 3.74-3.62 (m, 1H), 3.43 (dd, J=13.2, 6.9 Hz, 2H), 3.15-2.98 (m, 2H), 2.94-2.78 (m, 1H), 1.70-1.58 (m, 4H), 1.44-1.15 (m, 16H)., MS caled for C22H36N403S (M+H)+: 436, found 437

160. Compound 160: 1-(12-(2-aminonicotinamido)dodecyl)piperidine-3-carboxylic acid (hereinafter referred to as K-PB-70)
<Chemical formula 160>

Colorless oil, Yield: 16% 1H NMR (400 MHz, CDCl₃) δ8.14 (dd, J=4.9, 1.8 Hz, 1H), 7.60 (dd, J=7.7, 1.7 Hz, 1H), 6.59 (dd, J=7.7, 4.9 Hz, 1H), 6.32 (s, 2H), 6.17 (s, 1H), 3.40 (dd, J=13.0, 7.1 Hz, 2H), 3.26-3.16 (m, 1H), 3.13-3.00 (m, 1H), 2.74-2.69 (m, 1H), 2.55 (t, J=7.6 Hz, 2H), 2.43-2.33 (m, 1H), 2.32-2.15 (m, 1H), 2.12-1.96 (m, 1H), 1.96-1.81 (m, 1H), 1.75-1.66 (m, 2H), 1.65-1.54 (m, 4H), 1.41-1.20 (m, 16H)., MS caled for C24H40N4O3 (M+H)+: 432, found 433

161. Compound 161: 2-amino-N-(12-((3-methyloxetan-3-yl)amino)dodecyl) nicotinamide (hereinafter referred to as K-PB-71)
<Chemical formula 161>

White solid, Yield: 61% 1H NMR (400 MHz, MeOD) δ8.03 (dd, J=5.0, 1.8 Hz, 1H), 7.84 (dd, J=7.7, 1.8 Hz, 1H), 6.65 (dd, J=7.7, 5.0 Hz, 1H), 4.62 (d, J=6.4 Hz, 2H), 4.40 (d, J=6.8 Hz, 2H), 3.35-3.31 (m, 2H), 2.73-2.59 (m, 2H), 1.64-1.53 (m, 4H), 1.51 (s, 3H), 1.40-1.29 (m, 16H). MS caled for C22H38N4O₂ (M+H)+: 390, found 391

162. Compound 162: 2-Amino-N-(12-(2,4-dichloro-5H-pyrrolo[3,2-d]pyrimidin-5-yl)dodecyl)nicotinamide (hereinafter referred to as K-PB-72)
<Chemical formula 162>

Yellow oil, Yield: 13%, 1H NMR (400 MHz, CDCl₃) δ 8.17 (dd, J=5.0, 1.7 Hz, 1H), 7.62 (dd, J=7.6, 1.6 Hz, 1H), 7.11 (s, 1H), 6.68 (s, 2H), 6.60 (dd, J=7.6, 5.0 Hz, 1H), 6.37 (d, J=2.9 Hz, 1H), 6.12 (s, 1H), 4.37 (t, J=7.2 Hz, 2H), 3.45-3.40 (m, 2H), 1.59 (dt, J=13.9, 7.0 Hz, 4H), 1.27 (m, 16H)., MS caled for C24H32C1-2N602 (M+H)+: 491, found 492

163. Compound 163: 2-Amino-N-(12-(4-chloro-1H-pyrrolo[2,3-b]pyridin-1-yl) dodecyl) nicotinamide (hereinafter referred to as K-PB-73)
<Chemical formula 163>

Yellow oil, Yield: 30%, 1H NMR (400 MHz, CDCl₃) δ 9.31 (s, 2H), 8.23 (d, J=5.3 Hz, 1H), 7.96 (dd, J=7.5, 1.6 Hz, 1H), 7.78 (dd, J=6.2, 1.6 Hz, 1H), 7.27 (s, 1H), 7.11 (d, J=5.3 Hz, 1H), 6.73 (dd, J=7.5, 6.2 Hz, 1H), 6.58 (d, J=3.5 Hz, 1H), 6.53 (t, J=5.3 Hz, 1H), 4.31-4.25 (m, 2H), 3.41 (dd, J=13.1, 7.1 Hz, 2H), 1.91-1.80 (m, 2H), 1.64-1.56 (m, 2H), 1.33-1.22 (m, 16H). MS caled for C25H34NClN5O3 (M+H)+: 456, found 457

164. Compound 164: 2-Amino-N-[2-(2-hydroxyethylamino) dodecyl]nicotinamide) (hereinafter referred to as K-PB-74)
<Chemical formula 164>

White solid, Yield: 71%, 1H NMR (400 MHz, MeOD) δ8.31 (dd, J=7.6, 1.5 Hz, 1H), 8.02 (dd, J=6.2, 1.5 Hz, 1H), 6.95 (dd, J=7.5, 6.3 Hz, 1H), 3.87-3.70 (m, 2H), 3.36 (t, J=7.3 Hz, 2H), 3.14-3.09 (m, 2H), 3.05-2.98 (m, 2H), 1.68 (d, J=7.7 Hz, 2H), 1.65-1.56 (m, 2H), 1.45-1.28 (m, 16H). MS caled for C20H₃₆N₄O₂ (M+H)+: 364.28, found 365.25

165. Compound 165: 2-Amino-N-[12-(3-hydroxypyrrolidin-1-yl)dodecyl]nicotinamide (hereinafter referred to as K-PB-75)
<Chemical formula 165>

White solid, Yield: 63%, 1H NMR (400 MHz, MeOD) δ8.32 (dd, J=7.6, 1.6 Hz, 1H), 8.02 (dd, J=6.2, 1.6 Hz, 1H), 6.96 (dd, J=7.5, 6.3 Hz, 1H), 3.88 (dd, J=12.2, 3.7 Hz, 1H), 3.75-3.61 (m, 2H), 3.57 (dd, J=6.1, 3.6 Hz, 1H), 3.37 (dd, J=12.7, 5.5 Hz, 3H), 3.15 (dt, J=11.3, 8.1 Hz, 1H), 3.10-2.99 (m, 1H), 2.28-2.15 (m, 1H), 2.11 (ddd, J=10.7, 7.7, 3.7 Hz, 1H), 2.06-1.95 (m, 1H), 1.94-1.84 (m, 1H), 1.73 (dd, J=15.2, 7.4 Hz, 2H), 1.61 (dd, J=14.1, 6.9 Hz, 2H), 1.45-1.28 (m, 16H). MS caled for C23H40N4O2 (M+H)+: 404.31, found 405.30

166. Compound 166: 2-Amino-N-[12-(4-methoxypyrrolidin-1-yl)dodecyl]nicotinamide (hereinafter referred to as K-PB-76)
<Chemical formula 166>

White solid, Yield: 74%, 1H NMR (400 MHz, MeOD) δ8.33 (dd, J=7.6, 1.6 Hz, 1H), 8.02 (dd, J=6.2, 1.6 Hz, 1H), 6.96 (dd, J=7.5, 6.3 Hz, 1H), 3.74-3.60 (m, 3H), 3.56 (dd, J=11.7, 7.7 Hz, 1H), 3.43 (s, 3H), 3.40-3.33 (m, 3H), 3.16 (dt, J=11.3, 8.1 Hz, 1H), 3.10-2.98 (m, 1H), 2.29-2.18 (m, 1H), 2.18-2.06 (m, 1H), 1.99 (tt, J=15.2, 7.8 Hz, 1H), 1.87 (dt, J=14.4, 7.2 Hz, 1H), 1.73 (d, J=6.9 Hz, 2H), 1.67-1.56 (m, 2H), 1.35 (d, J=16.0 Hz, 16H). MS caled for C24H42N4O2 (M+H)+: 418.33, found 419.35

167. Compound 167: 2-Amino-N-[12-(4-hydroxypiperidin-1-yl)dodecyl]nicotinamide (hereinafter referred to as K-PB-77)
<Chemical formula 167>

White solid, Yield: 35%, 1H NMR (400 MHz, MeOD) δ8.33 (dd, J=7.6, 1.5 Hz, 2H), 8.02 (dd, J=6.2, 1.5 Hz, 2H), 6.96 (dd, J=7.5, 6.3 Hz, 2H), 3.37 (dd, J=17.2, 9.9 Hz, 4H), 3.21 (td, J=11.6, 6.3 Hz, 2H), 3.13-3.05 (m, 2H), 1.82 (dd, J=9.6, 4.0 Hz, 4H), 1.74 (d, J=8.0 Hz, 2H), 1.67-1.57 (m, 2H), 1.35 (d, J=20.6 Hz, 16H), 1.29 (s, 3H). MS caled for C24H42N4O2 (M+H)+: 418.33, found 419.35

168. Compound 168: 2-Amino-N-[12-{4-(cyclohexanecarbonyl)piperazin-1-yl}dodecyl]nicotinamide (hereinafter referred to as K-PB-78)
<Chemical formula 168>

White solid, Yield: 69%, 1H NMR (400 MHz, MeOD) δ8.36 (dd, J=7.6, 1.6 Hz, 1H), 8.02 (dd, J=6.3, 1.6 Hz, 1H), 6.98 (dd, J=7.5, 6.4 Hz, 1H), 4.22 (s, 1H), 3.72-3.44 (m, 3H), 3.40-3.33 (m, 3H), 3.19-3.11 (m, 3H), 2.99 (d, J=14.1 Hz, 1H), 2.71-2.60 (m, 1H), 1.77 (dd, J=21.2, 12.7 Hz, 7H), 1.61 (dd, J=14.2, 7.0 Hz, 2H), 1.53-1.14 (m, 22H).MS caled for C29H49N5O2 (M+H)+: 499.38, found 500.40

169. Compound 169: 2-Amino-N-[12-{1-benzoyl-2,5-dihydro-1H-pyrrol-2-one-3-yl}dodecyl]nicotinamide (hereinafter referred to as K-PB-79)
<Chemical formula 169>

White solid, Yield: 59%, 1H NMR (400 MHz, MeOD) δ8.33 (dd, J=7.5, 1.2 Hz, 1H), 8.02 (dd, J=6.2, 1.5 Hz, 1H), 7.60 (d, J=7.9 Hz, 2H), 7.36 (t, J=8.0 Hz, 2H), 7.16 (t, J=7.4 Hz, 1H), 6.97 (dd, J=7.4, 6.4 Hz, 1H), 4.34-4.22 (m, 1H), 3.88-3.70 (m, 1H), 3.39-3.33 (m, 3H), 3.22 (ddd, J=16.2, 14.8, 7.6 Hz, 2H), 2.70-2.58 (m, 1H), 2.29-2.03 (m, 3H), 1.79-1.68 (m, 2H), 1.60 (dd, J=14.2, 7.0 Hz, 2H), 1.32 (d, J=19.2 Hz, 16H). MS caled for C29H43N5O2 (M+H)+: 493.34, found 494.35

170. Compound 170: 2-Amino-N-[12-[(3S)-3-hydroxypyrrolidin-1-yl]dodecyl]nicotinamide) (hereinafter referred to as K-PB-80)
<Chemical formula 170>

White solid, Yield: 64%, 1H NMR (400 MHz, MeOD) δ8.32 (dd, J=7.6, 1.5 Hz, 1H), 8.04 (dd, J=6.2, 1.5 Hz, 1H), 6.97 (dd, J=7.5, 6.3 Hz, 1H), 4.57 (s, 1H), 3.76 (s, 2H), 3.50 (s, 1H), 3.41-3.35 (m, 2H), 3.19 (m, 3H), 2.14 (m, J=67.7 Hz, 2H), 1.74 (s, 2H), 1.63 (dd, J=14.1, 7.0 Hz, 2H), 1.37 (d, J=16.5 Hz, 16H). MS caled for C22H38N4O2 (M+H)+: 390.29, found 391.30

171. Compound 171: 2-Amino-N-[12-{4-(acetamido)piperidin-1-yl}dodecyl]nicotinamide) (hereinafter referred to as K-PB-81)
<Chemical formula 171>

White solid, Yield: 46%, 1H NMR (400 MHz, MeOD) δ8.35 (dd, J=7.6, 1.5 Hz, 1H), 8.02 (dd, J=6.3, 1.6 Hz, 1H), 6.98 (dd, J=7.5, 6.3 Hz, 1H), 4.02 (s, 1H), 3.58 (dd, J=28.7, 10.0 Hz, 2H), 3.36 (t, J=7.3 Hz, 3H), 3.20-3.03 (m, 2H), 2.85 (t, J=12.5 Hz, 1H), 2.62 (t, J=11.3 Hz, 1H), 2.18-1.99 (m, 2H), 1.96 (s, 3H), 1.88-1.46 (m, 5H), 1.44-1.24 (m, 16H). MS caled for C25H43N5O2 (M+H)+: 445.34, found 446.30

172. Compound 172: 2-Amino-N-[12-(4-(2-hydroxyethyl)piperidin-1-yl) dodecyl]nicotinamide (hereinafter referred to as K-PB-82)
<Chemical formula 172>

White solid, Yield: 23%, 1H NMR (400 MHz, MeOD) δ8.34 (dd, J=7.6, 1.5 Hz, 1H), 8.02 (dd, J=6.3, 1.5 Hz, 1H), 6.97 (dd, J=7.5, 6.4 Hz, 1H), 3.63 (t, J=6.4 Hz, 2H), 3.56 (d, J=12.4 Hz, 2H), 3.36 (t, J=7.2 Hz, 2H), 3.11-3.00 (m, 2H), 3.00-2.85 (m, 2H), 2.00 (d, J=14.3 Hz, 2H), 1.82-1.66 (m, 3H), 1.61 (dd, J=14.0, 6.9 Hz, 2H), 1.52 (dd, J=13.1, 6.5 Hz, 2H), 1.45 (d, J=14.4 Hz, 2H), 1.42-1.27 (m, 16H). MS caled for C25H44N4O2 (M+H)+: 432.34, found 433.35

173. Compound 173: 2-Amino-N-[12-{4-[1-(benzimidazol-1-yl)acetyl]piperazin-1-yl]dodecyl]nicotinamide (hereinafter referred to as K-PB-83)
<Chemical formula 173>

White solid, Yield: 47%, 1H NMR (400 MHz, MeOD) δ8.29 (dd, J=7.6, 1.5 Hz, 1H), 8.03 (dd, J=6.2, 1.5 Hz, 1H), 7.22-7.06 (m, 4H), 6.95 (dd, J=7.5, 6.2 Hz, 1H), 6.05 (s, 1H), 4.06 (s, 2H), 3.64 (s, 2H), 3.40-3.33 (m, 4H), 2.94 (s, 2H), 2.69-2.54 (m, 1H), 1.83 (s, 2H), 1.70-1.54 (m, 2H), 1.39 (d, J=33.3 Hz, 16H). MS caled for C30H42N6O₂ (M+H)+: 518.33, found 519.35

174. Compound 174: 2-Amino-N-[12-{4-[(3,5-dimethylbenzoyl)amino]piperidin-1-yl}dodecyl]nicotinamide (hereinafter referred to as K-PB-84)
<Chemical formula 174>

White solid, Yield: 46%, 1H NMR (400 MHz, MeOD) δ8.29 (dd, J=7.6, 1.4 Hz, 1H), 8.02 (dd, J=6.1, 1.4 Hz, 1H), 7.23-7.07 (m, 3H), 6.94 (dd, J=7.5, 6.2 Hz, 1H), 4.06 (d, J=10.9 Hz, 1H), 3.70 (d, J=10.7 Hz, 1H), 3.39-3.33 (t, 2H), 3.15 (dd, J=20.6, 11.8 Hz, 3H), 2.40 (d, J=14.9 Hz, 1H), 2.22 (s, 6H), 2.01 (dd, J=19.7, 14.4 Hz, 3H), 1.92-1.67 (m, 4H), 1.60 (dd, J=14.1, 6.9 Hz, 2H), 1.47-1.23 (m, 16H). MS caled for C32H49N5O2 (M+H)+: 535.38, found 536.40

175. Compound 175: 2-Amino-N-[12-(4-(2-hydroxyethyl)piperidin-1-yl) dodecyl]nicotinamide (hereinafter referred to as K-PB-85)
<Chemical formula 175>

White solid, Yield: 61%, 1H NMR (400 MHz, MeOD) δ8.32 (dd, J=7.6, 1.5 Hz, 1H), 8.03 (dd, J=6.2, 1.5 Hz, 1H), 6.96 (dd, J=7.5, 6.3 Hz, 1H), 3.98 (dd, J=12.5, 3.4 Hz, 1H), 3.58 (dd, J=12.5, 1.9 Hz, 1H), 3.48 (d, J=12.2 Hz, 1H), 3.39-3.33 (m, 2H), 3.24-3.11 (m, 2H), 3.11-2.99 (m, 1H), 1.97-1.53 (m, 11H), 1.48-1.20 (m, 16H). MS caled for C24H42N4O2 (M+H)+: 418.33, found 419.30

176. Compound 176: 2-Amino-N-[12-(3-hydroxypyrrolidin-1-yl)dodecyl]nicotinamide (hereinafter referred to as K-PB-86)
<Chemical formula 176>

White solid, Yield: 63%, 1H NMR (400 MHz, MeOD) δ8.32 (dd, J=7.6, 1.5 Hz, 1H), 8.04 (dd, J=6.2, 1.5 Hz, 1H), 6.97 (dd, J=7.5, 6.3 Hz, 1H), 4.57 (s, 1H), 3.76 (s, 2H), 3.50 (s, 1H), 3.41-3.35 (m, 2H), 3.19 (m, 3H), 2.14 (m, J=67.7 Hz, 2H), 1.74 (s, 2H), 1.63 (dd, J=14.1, 7.0 Hz, 2H), 1.37 (d, J=16.5 Hz, 16H). MS caled for C22H38N4O2 (M+H)+: 390.29, found 391.30

177. Compound 177: 2-amino-N-(12-(6-nitro-3,4-dihydroisoquinolin-2(1H)-yl) dodecyl)nicotinamide (hereinafter referred to as K-PB-87)
<Chemical formula 177>

Yellow solid, Yield: 49%, 1H NMR (400 MHz, CDCl₃) δ9.43 (s, 2H), 8.18-8.11 (m, 2H), 8.01 (dd, J=7.6, 1.5 Hz, 1H), 7.86 (dd, J=6.2, 1.6 Hz, 1H), 7.34 (d, J=8.5 Hz, 1H), 6.74 (dd, J=7.5, 6.2 Hz, 1H), 6.56-6.51 (m, 1H), 4.81 (s, 2H), 4.17 (s, 2H), 3.84 (s, 2H), 3.45 (dd, J=13.2, 6.9 Hz, 4H), 3.30-3.06 (m, 4H), 1.91-1.80 (m, 2H), 1.68-1.56 (m, 2H), 1.46-1.25 (m, 12H)., MS caled for C27H39N5O3 (M+H)+: 481, found 482

178. Compound 178: 2-amino-N-(12-(7-nitro-3,4-dihydroisoquinolin-2(1H)-yl) dodecyl)nicotinamide (hereinafter referred to as K-PB-88)
<Chemical formula 178>

Yellow solid, Yield: 50%, 1H NMR (400 MHz, CDCl₃) δ8.18 (dd, J=4.8, 1.8 Hz, 1H), 8.00 (dd, J=8.4, 2.4 Hz, 1H), 7.94 (d, J=2.2 Hz, 1H), 7.59 (dd, J=7.7, 1.7 Hz, 1H), 7.26 (d, J=8.4 Hz, 1H), 6.62 (dd, J=7.7, 4.9 Hz, 1H), 6.31 (s, 2H), 6.01 (s, 1H), 3.71 (s, 2H), 3.48-3.37 (m, 2H), 3.01 (t, J=5.9 Hz, 2H), 2.78 (t, J=5.9 Hz, 2H), 2.59-2.51 (m, 2H), 1.66-1.57 (m, 8H), 1.43-1.31 (m, J=12.0 Hz, 12H)., MS caled for C27H39N5O3 (M+H)+: 481, found 482

179. Compound 179: 2-amino-N-(12-(4-iodo-1H-pyrazol-1-yl)dodecyl) nicotinamide (hereinafter referred to as K-PB-89)
<Chemical formula 179>

Yellow solid, Yield: 20%, 1H NMR (400 MHz, CDCl₃) δ9.31 (s, 2H), 7.93 (dd, J=7.5, 1.6 Hz, 1H), 7.87 (dd, J=6.1, 1.6 Hz, 1H), 7.52 (s, 1H), 7.44 (s, 1H), 6.75 (dd, J=7.5, 6.2 Hz, 1H), 6.23 (s, 1H), 4.13 (t, J=7.2 Hz, 2H), 3.45 (dd, J=13.1, 7.1 Hz, 2H), 1.90-1.80 (m, 2H), 1.63 (dt, J=14.9, 7.6 Hz, 2H), 1.44-1.18 (m, 16H)., MS caled for C21H32IN50 (M+H)+: 497, found 498

180. Compound 180: 2-amino-N-(12-(4-phenylpiperazin-1-yl)dodecyl) nicotinamide (hereinafter referred to as K-PB-90)
<Chemical formula 180>

Yellow solid, Yield: 11%, 1H NMR (400 MHz, CDCl₃) δ8.15 (dd, J=4.8, 1.7 Hz, 1H), 7.57 (dd, J=7.7, 1.7 Hz, 1H), 7.29-7.27 (m, J=2.0 Hz, 1H), 7.25-7.23 (m, J=2.3 Hz, 1H), 6.93 (d, J=7.9 Hz, 2H), 6.86 (t, J=7.0 Hz, 1H), 6.59 (dd, J=7.7, 4.9 Hz, 1H), 6.29 (s, 2H), 6.00 (s, 1H), 3.41 (dd, J=13.0, 7.1 Hz, 2H), 3.24 (s, 4H), 2.64 (s, 4H), 2.41 (s, 2H), 1.64-1.55 (m, 8H), 1.39-1.26 (m, 12H). MS caled for C28H43N5O (M+H)+: 465, found 466

181. Compound 181: (R)-2-amino-N-(12-(2-methylmorpholino)dodecyl) nicotinamide (hereinafter referred to as K-PB-91)
<Chemical formula 181>

Yellow Oil, Yield: 28%, 1H NMR (400 MHz, CDCl₃) δ8.18 (dd, J=4.9, 1.8 Hz, 1H), 7.59 (dd, J=7.7, 1.7 Hz, 1H), 6.62 (dd, J=7.6, 4.9 Hz, 1H), 6.31 (s, 2H), 6.01 (s, 1H), 3.87 (ddd, J=11.3, 3.2, 1.4 Hz, 1H), 3.72 (dd, J=11.4, 2.4 Hz, 1H), 3.69-3.63 (m, 1H), 3.43 (td, J=7.2, 5.9 Hz, 2H), 2.76 (dd, J=21.1, 11.4 Hz, 2H), 2.37-2.29 (m, 2H), 2.14-2.05 (m, 1H), 1.83-1.74 (m, 1H), 1.67-1.58 (m, 8H), 1.53-1.47 (m, 2H), 1.44-1.30 (m, 10H), 1.17 (d, J=6.3 Hz, 3H)., MS caled for C23H40N4O2 (M+H)+: 404, found 405

182. Compound 182: 2-amino-N-(12-((naphthalen-2-ylmethyl)amino)dodecyl) nicotinamide (hereinafter referred to as K-PB-92)
<Chemical formula 182>

White solid, Yield: 35%, 1H NMR (400 MHz, CDCl₃) δ9.50 (s, 2H), 9.19 (s, 1H), 8.05 (d, J=6.3 Hz, 1H), 7.89 (d, J=8.2 Hz, 1H), 7.82-7.76 (m, 2H), 7.70 (d, J=4.8 Hz, 1H), 7.56 (dd, J=11.1, 4.1 Hz, 1H), 7.53-7.49 (m, 1H), 7.46 (d, J=6.2 Hz, 1H), 7.43-7.38 (m, 1H), 7.06-6.97 (m, 1H), 6.67-6.60 (m, 1H), 4.26 (s, 2H), 3.38 (dd, J=13.1, 6.8 Hz, 2H), 2.85 (s, 2H), 1.58 (td, J=14.0, 6.8 Hz, 4H), 1.37-1.16 (m, 15H), 0.91-0.80 (m, 2H)., MS caled for C29H40N4O (M+H)+: 460, found 461

183. Compound 183: 2-amino-N-(12-((2-methyl-1,2,3,4-tetrahydroisoquinolin-6-yl)amino) dodecyl)nicotinamide (hereinafter referred to as K-PB-93)
<Chemical formula 183>

Yellow solid, Yield: 57%, 1H NMR (400 MHz, MeOD) δ8.35 (dd, J=7.6, 1.6 Hz, 1H), 8.04 (dd, J=6.3, 1.6 Hz, 1H), 7.03-6.96 (m, 2H), 6.79 (dd, J=8.2, 2.3 Hz, 1H), 6.76 (d, J=2.0 Hz, 1H), 4.47 (q, J=14.8 Hz, 2H), 3.73-3.64 (m, 2H), 3.38 (dd, J=12.1, 4.8 Hz, 4H), 3.16 (dd, J=5.7, 4.0 Hz, 2H), 3.12 (s, 3H), 1.88 (dt, J=12.5, 7.0 Hz, 2H), 1.66-1.61 (m, 2H), 1.41-1.30 (m, 16H). MS caled for C28H43N5O (M+H)+: 465, found 466

184. Compound 184: 2-amino-N-(12-((1-(naphthalen-1-yl)ethyl)amino)dodecyl) nicotinamide (hereinafter referred to as K-PB-94)
<Chemical formula 184>

White solid, Yield: 47%, 1H NMR (400 MHz, CDCl₃) δ10.00 (s, 1H), 9.27 (s, 2H), 8.09 (d, J=6.6 Hz, 1H), 8.03 (d, J=8.5 Hz, 1H), 7.98-7.91 (m, 2H), 7.82 (d, J=5.6 Hz, 1H), 7.68-7.56 (m, 3H), 6.96 (t, J=5.3 Hz, 1H), 6.78-6.70 (m, 1H), 5.25 (s, 1H), 3.42 (dd, J=13.1, 6.8 Hz, 2H), 2.89-2.79 (m, 1H), 2.73-2.63 (m, J=17.9, 9.3 Hz, 1H), 1.81 (d, J=6.7 Hz, 3H), 1.72-1.55 (m, 4H), 1.39-1.13 (m, 15H), 0.93-0.81 (m, 2H)., MS caled for C30H42N4O (M+H)+: 474, found 475

185. Compound 185: 2-amino-N-(12-(3,4-dihydroquinolin-1(2H)-yl)dodecyl) nicotinamide (hereinafter referred to as K-PB-95)
<Chemical formula 185>

Green oil, Yield: 19%, 1H NMR (400 MHz, CDCl₃) δ9.42 (s, 2H), 7.99 (dd, J=7.6, 1.4 Hz, 1H), 7.84 (dd, J=6.2, 1.5 Hz, 1H), 7.14 (t, J=7.8 Hz, 1H), 7.06 (d, J=7.4 Hz, 1H), 6.91 (d, J=8.2 Hz, 1H), 6.82 (t, J=7.2 Hz, 1H), 6.74 (dd, J=7.4, 6.2 Hz, 1H), 6.46 (s, 1H), 3.45 (dd, J=13.1, 7.1 Hz, 2H), 3.41-3.35 (m, 2H), 3.32-3.23 (m, 2H), 2.84 (t, J=6.5 Hz, 2H), 2.10-1.98 (m, 2H), 1.73-1.55 (m, 4H), 1.45-1.01 (m, 16H)., MS caled for C27H40N4O (M+H)+: 436, found 437

186. Compound 186: 2-amino-N-(12-(benzo[d][1,3]dioxol-5-ylamino)dodecyl) nicotinamide (hereinafter referred to as K-PB-96)
<Chemical formula 186>

Yellow solid, Yield: 43%, 1H NMR (400 MHz, CDCl₃) δ9.28 (s, 2H), 8.10 (dd, J=7.6, 1.6 Hz, 1H), 7.81 (dd, J=6.2, 1.6 Hz, 1H), 6.97 (t, J=5.5 Hz, 1H), 6.87 (td, J=4.5, 2.2 Hz, 2H), 6.80-6.72 (m, 2H), 6.03 (s, 2H), 3.43 (dd, J=13.1, 7.0 Hz, 2H), 3.22-3.12 (m, 2H), 1.66 (ddd, J=21.5, 18.6, 11.2 Hz, 4H), 1.41-1.20 (m, 15H), 0.94-0.79 (m, J=12.7, 8.3, 5.5 Hz, 2H)., MS caled for C25H₃₆N₄O₃ (M+H)+: 440, found 441

187. Compound 187: 2-amino-N-(12-(quinolin-6-ylamino)dodecyl)nicotinamide (hereinafter referred to as K-PB-97)
<Chemical formula 187>

Yellow solid, Yield: 48%, 1H NMR (400 MHz, MeOD) δ9.67 (s, 1H), 8.55 (d, J=6.9 Hz, 1H), 8.48 (dd, J=7.0, 1.5 Hz, 1H), 8.27 (dd, J=7.6, 1.6 Hz, 1H), 8.04 (dd, J=6.0, 1.6 Hz, 1H), 7.79 (t, J=8.0 Hz, 1H), 7.64 (d, J=8.1 Hz, 1H), 7.38 (dd, J=7.8, 0.8 Hz, 1H), 6.94 (dd, J=7.6, 6.1 Hz, 1H), 4.72-4.65 (m, 2H), 3.39-3.34 (m, 4H), 2.11 (dt, J=14.4, 7.2 Hz, 2H), 1.62 (dt, J=14.7, 7.4 Hz, 2H), 1.47-1.29 (m, 14H)., MS caled for C27H₃₇N₅O (M+H)+: 447, found 448

189. Compound 189: ethyl 1-(14-(2-aminopyridin-3-yl)-14-oxotetradecyl) piperidine-2-carboxylate (hereinafter referred to as K-PB-99)
<Chemical formula 189>

Oil, Yield: 59%, 1H NMR (400 MHz, CDCl₃) δ8.16 (dd, J=4.9, 1.8 Hz, 1H), 7.59 (dd, J=7.7, 1.7 Hz, 1H), 6.60 (dd, J=7.7, 4.9 Hz, 1H), 6.32 (s, 2H), 6.09 (s, 1H), 4.28-4.14 (m, 2H), 3.49-3.35 (m, 2H), 3.17-2.96 (m, 2H), 2.51 (ddd, J=12.4, 9.5, 6.6 Hz, 1H), 2.26 (ddd, J=12.3, 9.5, 6.1 Hz, 1H), 2.18-2.07 (m, 1H), 1.88-1.69 (m, 3H), 1.69-1.55 (m, 4H), 1.49 (t, J=10.3 Hz, 2H), 1.42-1.32 (m, 4H), 1.32-1.22 (m, 16H). MS caled for C26C44N403 (M+H)+: 460, found 461

190. Compound 190: 2-amino-N-(12-(isopropyl(methyl)amino)dodecyl) nicotinamide (hereinafter referred to as K-PB-100)
<Chemical formula 190>

Oil, Yield: 14%, 1H NMR (400 MHz, CDCl₃) δ8.07 (dd, J=4.8, 1.7 Hz, 1H), 7.55 (dd, J=7.7, 1.7 Hz, 1H), 6.53 (dd, J=7.7, 4.9 Hz, 1H), 6.24 (s, 2H), 6.10 (s, 1H), 3.38-3.28 (m, 2H), 2.78 (s, 2H), 2.53 (s, 3H), 1.96 (d, J=6.9 Hz, 1H), 1.77 (s, 2H), 1.53 (dd, J=14.4, 7.1 Hz, 5H), 1.28 (d, J=6.6 Hz, 6H), 1.20 (s, 16H). MS caled for C22C40N40 (M+H)+: 376, found 377

191. Compound 191: N1-Cyclohexyl-N2-(12-(4-methylpiperazin-1-yl)dodecyl)phthalamide (KP-01, also known as)
<Chemical formula 191>

Yellow, 1H NMR (400 MHz, CDCl₃) δ 7.63-7.54 (m, 2H), 7.50-7.42 (m, 2H), 6.76 (s, NH), 6.55 (s, NH), 3.91 (d, J=9.9 Hz, 1H), 3.38 (dd, J=12.5, 5.9 Hz, 2H), 2.59 (bs, 8H), 2.46-2.37 (m, 2H), 2.35 (s, 3H), 1.97 (d, J=10.8 Hz, 2H), 1.74 (d, J=12.8 Hz, 2H), 1.65-1.48 (m, 5H), 1.45-1.20 (m, 21H) Ms caled for C31H₅₂N₄O₂: 512.78

192. Compound 192: 2-(1H-imidazol-2-yl)-N-(12-(4-methylpiperazin-1-yl)dodecyl)benzamide (KP-06, also known as)
<Chemical formula 192>

White, 1H NMR (400 MHz, CD3OD) δ 7.77 (d, J=7.8 Hz, 1H), 7.57-7.46 (m, 3H), 7.14 (s, 2H), 3.35-3.28 (m, 2H, overlapped with CD₃OD peak), 2.56 (bs, 8H), 2.43-2.39 (m, 2H), 2.32 (s, 3H), 1.56-1.50 (m, 4H), 1.33-1.30 (m, 16H). Ms caled for C27H43N5O: 453.67

193. Compound 193: N-(12-(4-methylpiperazin-1-yl)dodecyl)-2-(1H-pyrrol-1-yl)benzamide (KP-07 alias)
<Chemical formula 193>

Brown, 1H NMR (400 MHz, CD3OD) δ 7.53 (td, J=7.7, 1.7 Hz, 1H), 7.49 (dd, J=7.7, 1.6 Hz, 1H), 7.43-7.38 (m, 2H), 6.90 (t, J=2.1 Hz, 2H), 6.23 (t, J=2.1 Hz, 2H), 3.16 (t, J=7.1 Hz, 2H), 2.81 (bs, 8H), 2.65-2.61 (m, 2H), 2.49 (s, 3H), 1.62-1.55 (m, 2H), 1.43-1.19 (m, 18H) Ms calculated for C28H44N40: 452.69

194. Compound 194: N-(12-(4-methylpiperazin-1-yl)dodecyl)-[1,1′-biphenyl]-2-carboxamide (KP-08, also known as)
<Chemical formula 194>

White, 1H NMR (400 MHz, CD3OD) δ 7.52-7.25 (m, 9H), 3.12 (t, J=7.0 Hz, 2H), 2.87 (bs, 8H), 2.71-2.67 (m, 2H), 2.53 (s, 3H), 1.63-1.55 (m, 2H), 1.35-1.20 (m, 16H), 1.12-1.04 (m, 2H). Ms caled for C30H45N3O: 463.71

195. Compound 195: N-(12-(4-methylpiperazin-1-yl)dodecyl)-[2,2′-bipyridine]-4-carboxamide (KP-10, also known as)
<Chemical formula 195>

White, 1H NMR (400 MHz, CD3OD) δ 8.78 (dd, J=5.1, 0.9 Hz, 1H), 8.70-8.68 (m, 2H), 8.38 (dt, J=8.0, 0.9 Hz, 1H), 7.96 (td, J=7.8, 1.7 Hz, 1H), 7.75 (dd, J=5.1, 1.7 Hz, 1H), 7.46 (ddd, J=7.5, 4.8, 1.1 Hz, 1H), 3.42 (t, J=7.2 Hz, 2H), 2.60 (bs, 8H), 2.46-2.42 (m, 2H), 2.35 (s, 3H), 1.66 (p, J=7.0 Hz, 2H), 1.56-1.49 (m, 2H), 1.44-1.29 (m, 16H) Ms calculated for C28H43N5O: 465.69

196. Compound 196: 2-Benzyl-N-(12-(4-methylpiperazin-1-yl)dodecyl)benzamide (KP-21, also known as)

### <Compound 196>

White, 1H NMR (400 MHz, CD3OD) δ 7.36-7.32 (m, 2H), 7.27-7.20 (m, 4H), 7.15-7.11 (m, 3H), 4.14 (s, 2H), 3.25 (t, J=7.0 Hz, 2H), 2.50 (bs, 8H), 2.37-2.33 (m, 2H), 2.28 (s, 3H), 1.52-1.46 (m, 4H), 1.32-1.30 (m, 16H) Ms calculated for C31H47N3O: 477.74

197. Compound 197: 3-Amino-2-iodo-N-(12-(4-methylpiperazin-1-yl)dodecyl)benzamide (KP-23, also known as)
<Chemical formula 197>

White, 1H NMR (400 MHz, CD3OD) δ 7.12 (dd, J=8.0, 7.3 Hz, 1H), 6.81 (dd, J=8.1, 1.5, 1H), 6.57 (dd, 7.3, 1.5 Hz, 1H), 3.33 (m, 2H, overlapped with CD₃OD peak), 2.54 (bs, 8H), 2.41-2.37 (m, 2H), 2.31 (s, 3H), 1.62 (p, J=7.2 Hz, 2H), 1.56-1.50 (m, 2H), 1.45-1.29 (m, 16H) Ms calculated for C24H41IN40: 528.52

198. Compound 198: 2,3-Dichloro-N-(12-(4-methylpiperazin-1-yl)dodecyl)isonicotinamide (KP-25, alternative name)
<Chemical formula 198>

White, 1H NMR (400 MHz, CD3OD) δ 8.37 (dd, J=4.8, 0.8 Hz, 1H), 7.38 (dd, J=4.9, 0.8 Hz, 1H), 3.37 (t, J=7.0 Hz, 2H), 2.68 (bs, 8H), 2.53-2.49 (m, 2H), 2.40 (s, 3H), 1.65-1.51 (m, 4H), 1.44-1.29 (m, 16H) Ms calculated for C23H38Cl2N4O: 457.48

199. Compound 199: 3-Chloro-N-(12-(4-methylpiperazin-1-yl)dodecyl)isonicotinamide (KP-26, also known as)
<Chemical formula 199>

White, 1H NMR (400 MHz, CD3OD) δ 8.67 (s, 1H), 8.57 (d, J=4.9 Hz, 1H), 7.46 (d, J=4.9 Hz, 1H), 3.39 (t, J=7.0 Hz, 2H), 2.54 (bs, 8H), 2.41-2.37 (m, 2H), 2.31 (s, 3H), 1.64 (p, J=7.1 Hz, 2H), 1.57-1.50 (m, 2H), 1.46-1.31 (m, 16H) Ms caled for C23H39C1N40: 423.04

200. Compound 200: 2-Methoxy-N-(12-(4-methylpiperazin-1-yl)dodecyl)nicotinamide (KP-27 alias)
<Chemical formula 200>

White, 1H NMR (400 MHz, CD3OD) δ 8.30 (dd, J=4.9, 2.0 Hz, 1H), 8.27 (dd, J=7.6, 2.0 Hz, 1H), 7.12 (dd, 7.5, 4.9 Hz, 1H), 4.09 (s, 3H), 3.42 (t, J=7.1 Hz, 2H), 2.53 (bs, 8H), 2.40-2.36 (m, 2H), 2.31 (s, 3H), 1.64 (p, J=7.1 Hz, 2H), 1.56-1.48 (m, 2H), 1.44-1.31 (m, 16H) Ms caled for C24H42N4O2: 418.63

201. Compound 201: 3,5-Dichloro-N-(12-(4-methylpiperazin-1-yl)dodecyl)isonicotinamide (KP-28, also known as)
<Chemical formula 201>

White, 1H NMR (400 MHz, CD3OD) δ 8.49 (s, 2H), 3.29 (t, J=6.9 Hz, 2H), 2.41 (bs, 8H), 2.28-2.24 (m, 2H), 2.19 (s, 3H), 1.53 (p, J=7.2 Hz, 2H), 1.45-1.36 (m, 2H), 1.36-1.19 (m, 16H) Ms caled for C23H38C12N40: 457.48

202. Compound 202: 5-Methyl-N-(12-(4-methylpiperazin-1-yl)dodecyl)picolinamide (KP-29 alias)
<Chemical formula 202>

White, 1H NMR (400 MHz, CD3OD) δ 8.47 (s, 1H), 7.99 (d, J=8.0 Hz, 1H), 7.77 (d, J=8.1 Hz, 1H), 3.42 (t, J=7.2 Hz, 2H), 2.52 (bs, 8H), 2.43 (s, 3H), 2.43-2.35 (m, 2H), 2.30 (s, 3H), 1.64 (p, J=7.2 Hz, 2H), 1.56-1.48 (m, 2H), 1.42-1.32 (m, 16H) Ms calculated for C24H42N40: 402.63

203. Compound 203: 5-Fluoro-N-(12-(4-methylpiperazin-1-yl)dodecyl)picolinamide (KP-30, also known as)
<Chemical formula 203>

White, 1H NMR (400 MHz, CD3OD) δ 8.54 (d, J=2.8 Hz, 1H), 8.17 (dd, J=8.7, 4.5 Hz, 1H), 7.75 (td, 8.6, 2.9 Hz, 1H), 3.42 (t, J=7.1 Hz, 2H), 2.53 (bs, 8H), 2.39-2.35 (m, 2H), 2.30 (s, 3H), 1.64 (p, J=7.3 Hz, 2H), 1.56-1.50 (m, 2H), 1.42-1.32 (m, 16H) Ms calculated for C23H39FN4O: 406.59

204. Compound 204: 5-methoxy-N-(12-(4-methylpiperazin-1-yl)dodecyl)picolinamide (KP-31, also known as)
<Chemical formula 204>

White, 1H NMR (400 MHz, CD3OD) δ 8.18 (d, J=2.8 Hz, 1H), 7.94 (d, J=8.7 Hz, 1H), 7.36 (dd, J=8.7, 2.9 Hz, 1H), 3.83 (s, 3H), 3.29 (t, J=7.0 Hz, 2H), 2.41 (bs, 8H), 2.27-2.23 (m, 2H), 2.18 (s, 3H), 1.52 (p, J=7.2 Hz, 2H), 1.44-1.36 (m, 2H), 1.29-1.20 (m, 16H) Ms calculated for C24H42N4O2: 418.63

207. Compound 207: 3-(2-methyl-1H-imidazol-1-yl)-N-(12-(4-methylpiperazin-1-yl)dodecyl)benzamide (KP-53, also known as)
<Chemical formula 207>

White, 1H NMR (400 MHz, CD3OD) δ 7.93 (d, J=7.8 Hz, 1H), 7.83 (s, 1H), 7.65 (t, J=7.9 Hz, 1H), 7.59 (dq, J=7.9, 1.0 Hz, 1H), 7.23 (d, J=1.2 Hz, 1H), 7.00 (d, J=1.2 Hz, 1H), 3.39 (t, J=7.2 Hz, 2H), 2.51 (bs, 8H), 2.38-2.34 (m, 5H), 2.29 (s, 3H), 1.63 (p, J=7.2 Hz, 2H), 1.54-1.46 (m, 2H), 1.41-1.29 (m, 16H) Ms calcd for C28H45N5O: 467.70

208. Compound 208: N-(12-(4-methylpiperazin-1-yl)dodecyl)-4-(1H-1,2,4-triazol-5-yl)benzamide (KP-54 alias)
<Chemical formula 208>

White, 1H NMR (400 MHz, CD3OD) δ 7.72 (d, J=8.6 Hz, 2H), 7.43 (d, J=8.6 Hz, 2H), 7.05 (s, 1H), 3.37 (t, J=7.1 Hz, 2H), 2.49 (bs, 8H), 2.35-2.32 (m, 2H), 2.27 (s, 3H), 1.62 (p, J=7.1 Hz, 2H), 1.53-1.47 (m, 2H), 1.39-1.30 (m, 16H) Ms caled for C26H42N6O: 454.66

209. Compound 209: 3-Amino-N-(12-(4-methylpiperazin-1-yl)dodecyl)isonicotinamide (KP-61 alias)
<Chemical formula 209>

White, 1H NMR (400 MHz, CD3OD) δ 8.09 (s, 1H), 7.75 (d, J=5.3 Hz, 1H), 7.33 (d, J=5.3 Hz, 1H), 3.33 (t, J=7.2 Hz, 2H, overlapped with CD3OD peak), 2.50 (bs, 8H), 2.37-2.33 (m, 2H), 2.28 (s, 3H), 1.61 (p, J=7.2 Hz, 2H), 1.54-1.46 (m, 2H), 1.40-1.31 (m, 16H) Ms calculated for C23H₄₁N5O: 403.62

210. Compound 210: 2-Hydroxy-N-(12-(4-methylpiperazin-1-yl)dodecyl)nicotinamide (KP-62, also known as)
<Chemical formula 210>

White, 1H NMR (400 MHz, CD3OD) δ 8.45 (dd, J=7.2, 2.2 Hz, 1H), 7.66 (dd, J=6.3, 2.2 Hz, 1H), 6.56 (dd, J=7.2, 6.2 Hz, 1H), 3.40 (t, J=7.0 Hz, 2H), 2.50 (bs, 8H), 2.37-2.33 (m, 2H), 2.28 (s, 3H), 1.61 (p, J=7.2 Hz, 2H), 1.54-1.47 (m, 2H), 1.42-1.31 (m, 16H). Ms caled for C23H40N4O2: 404.60

211. Compound 211: 4-Amino-N-(12-(4-methylpiperazin-1-yl)dodecyl)nicotinamide (KP-63, also known as)
<Chemical formula 211>

White, 1H NMR (400 MHz, CD3OD) δ 8.28 (s, 1H), 7.87 (d, J=5.9 Hz, 1H), 6.55 (d, J=6.0 Hz, 1H), 3.25-3.23 (m, 2H, overlapped with CD₃OD peak), 2.40 (bs, 8H), 2.27-2.23 (m, 2H), 2.18 (s, 3H), 1.50 (p, J=7.0 Hz, 2H), 1.44-1.37 (m, 2H), 1.29-1.19 (m, 16H) Ms calculated for C23H41N5O: 403.62

212. Compound 212: 4-Amino-N-(12-(4-methylpiperazin-1-yl)dodecyl)nicotinamide (KP-64, also known as)
<Chemical formula 212>

White, 1H NMR (400 MHz, CD3OD) δ 8.75 (d, J=4.6 Hz, 1H), 7.95 (d, J=7.9 Hz, 1H), 7.70 (dd, J=7.8, 4.8 Hz, 1H), 3.35 (t, 7.1 Hz, 2H), 2.69 (bs, 8H), 2.55-2.51 (m, 2H), 2.41 (s, 3H), 1.62-1.53 (m, 4H), 1.42-1.33 (m, 16H). Ms caled for C24H39F3N40: 456.60

213. Compound 213: 2-Amino-N-(12-(4-methylpiperazin-1-yl)dodecyl)-4-(trifluoromethyl)benzamide (KP-65 alias)
<Chemical formula 213>

White, 1H NMR (400 MHz, CD3OD) δ 7.53 (d, J=8.2 Hz, 1H), 7.01 (s, 1H), 6.81 (d, J=8.2 Hz, 1H), 3.33 (t, J=7.1 Hz, 2H, overlapped with CD3OD peak), 2.49 (bs, 8H), 2.36-2.32 (m, 2H), 2.28 (s, 3H), 1.61 (p, J=7.0 Hz, 2H), 1.54-1.46 (m, 2H), 1.40-1.31 (m, 16H) Ms calculated for C25H41F3N4O: 470.63

214. Compound 214: N-(12-(4-methylpiperazin-1-yl)dodecyl)-2-phenoxybenzamide (KP-66, also known as)
<Chemical formula 214>

White, 1H NMR (400 MHz, CD3OD) δ 7.81 (dd, J=7.8, 1.8 Hz, 1H), 7.44 (ddd, J=8.3, 7.3, 1.7 Hz, 1H), 7.40-7.35 (m, 2H), 7.23 (td, J=7.7, 1.1 Hz, 1H), 7.15 (tt, J=7.4, 1.1 Hz, 1H), 7.03-7.00 (m, 2H), 6.92 (dd, J=8.4, 1.0 Hz, 1H), 3.33 (t, J=7.0 Hz, 2H, overlapped with CD3OD peak), 2.50 (bs, 8H), 2.37-2.33 (m, 2H), 2.28 (s, 3H), 1.53-1.45 (m, 4H), 1.32-1.22 (m, 16H) Ms calculated for C30H45N3O₂: 479.71

216. Compound 216: 4-(tert-butyl)-2-ethoxy-N-(12-(4-methylpiperazin-1-yl)dodecyl)benzamide (KP-68, also known as)
<Chemical formula 216>

White, 1H NMR (400 MHz, CDCl₃) δ 7.71 (dd, J=7.6, 1.3 Hz, 1H), 7.47 (td, J=7.5, 1.5 Hz, 1H), 7.44-7.33 (m, 7H), 5.17 (s, NH), 3.13 (dd, J=12.9, 6.9 Hz, 2H), 2.75 (bs, 8H), 2.58-2.50 (m, 2H), 2.44 (s, 3H), 1.62-1.49 (m, 2H), 1.31-1.09 (m, 16H), 1.01-0.95 (m, 2H) Ms calculated for C30H53N3O2: 487.77

217. Compound 217: 4-Bromo-2-fluoro-N-(12-(4-methylpiperazin-1-yl)dodecyl)benzamide (KP-69, also known as)
<Chemical formula 217>

Pale yellow solid, 1H NMR (400 MHz, CD3OD) δ 7.52-7.47 (m, 1H), 7.41-7.33 (m, 2H), 3.27 (t, J=7.1 Hz, 2H), 2.40 (bs, 8H), 2.27-2.23 (m, 2H), 2.18 (s, 3H), 1.51 (p, J=7.1 Hz, 2H), 1.43-1.36 (m, 2H), 1.30-1.17 (m, 16H) Ms calculated for C24H39BrFN30: 484.50

218. Compound 218: 2,6-Dimethoxy-N-(12-(4-methylpiperazin-1-yl) dodecyl)benzamide (hereinafter referred to as PBK-2025-052)
<Chemical formula 218>

white solid, ¹H NMR (400 MHz, CDCl₃): δ=8.06 (d, J=8.7 Hz, 1H), 7.63 (s, 1H), 7.14 (s, 1H), 6.47 (dd, J=2.4, 8.8 Hz, 1H), 6.35 (d, J=2.3 Hz, 1H), 3.81 (s, 3H), 3.72 (s, 3H), 3.34-3.27 (m, 2H), 2.65-2.38 (m, 6H), 2.27 (t, J=7.8 Hz, 3H), 2.21 (s, 4H), 1.50-1.43 (m, 2H), 1.15 ppm (s, 18H), Chemical Formula: C26H45N3O₃, M.W.: 447.66

219. Compound 219: 3-Chloro-2-methyl-N-(12-(4-methylpiperazin-1-yl)dodecyl)benzamide (also known as PBK-2025-083)
<Chemical formula 219>

white solid, ¹H NMR (400 MHz, CDCl₃): δ=7.40 (d, J=11.0 Hz, 1H), 7.24-7.13 (m, 2H), 5.78 (s, 1H), 3.46-3.38 (m, 2H), 2.56 (s, 9H), 2.46-2.32 (m, 9H), 1.65-1.48 (m, 4H), 1.34-1.23 ppm (m, 18H). Chemical Formula: C25H42C1N₃O, M.W.: 436.08

220. Compound 220: 2,3,4,5,6-Pentafluoro-N-(12-(piperazin-1-yl)dodecyl)benzamide hydrochloride (hereinafter referred to as PBK-2025-115)
<Chemical formula 220>

white solid, ¹H NMR (400 MHz, DMSO): δ=11.80 (s, 1H), 9.77 (s, 2H), 8.97 (s, 1H), 3.65 (s, 2H), 3.53-3.45 (m, 4H), 3.30-3.20 (m, 5H), 3.08 (s, 3H), 1.69 (s, 2H), 1.52-1.46 (m, 2H), 1.29 ppm (s, 20H). Chemical Formula: C23H35ClF5N3O, M.W.: 500.00

221. Compound 221: N-(12-(2H-benzo[d][1,2,3]triazol-2-yl)dodecyl)-2-aminonicotinamide (hereinafter referred to as PBK-2025-136)

### <Chemical Formula 221>

white solid, ¹H NMR (400 MHz, CDCl₃): δ=8.09 (dd, J=1.5, 5.0 Hz, 1H), 7.86 (dd, J=3.1, 6.5 Hz, 2H), 7.66 (dd, J=1.6, 7.7 Hz, 1H), 7.38 (dd, J=3.1, 6.6 Hz, 2H), 6.62 (dd, J=5.0, 7.7 Hz, 3H), 6.26 (s, 1H), 4.72 (t, J=7.2 Hz, 2H), 3.43-3.36 (m, 2H), 2.15-2.07 (m, 2H), 1.64-1.56 (m, 2H), 1.38-1.22 ppm (m, 21H). Chemical Formula: C24H34N6O, M.W.: 422.58

222. Compound 222: N-(12-(1H-benzo[d][1,2,3]triazol-1-yl)dodecyl)-2-aminonicotinamide (hereinafter referred to as PBK-2025-137)

### <Chemical Formula 222>

white solid, ¹H NMR (400 MHz, CDCl₃): δ=8.10 (dd, J=1.7, 4.9 Hz, 1H), 8.04 (d, J=8.4 Hz, 1H), 7.69 (dd, J=1.6, 7.7 Hz, 1H), 7.56-7.47 (m, 2H), 7.37 (t, J=7.4 Hz, 1H), 6.59 (q, J=4.1 Hz, 1H), 6.50 (s, 2H), 6.44 (s, 1H), 4.64 (t, J=7.1 Hz, 2H), 3.40 (dd, J=7.0, 13.1 Hz, 2H), 2.00 (dq, J=7.1, 7.1 Hz, 2H), 1.64-1.55 (m, 2H), 1.37-1.21 ppm (m, 18H).Chemical Formula: C24H34N6O, M.W.: 422.58

223. Compound 223: N-(12-(1H-tetrazol-1-yl)dodecyl)-2-aminonicotinamide (hereinafter referred to as PBK-2025-138)

### <Chemical Formula 223>

white solid, ¹H NMR (400 MHz, CDCl₃): δ=8.50 (s, 1H), 8.01 (d, J=4.1 Hz, 1H), 7.81 (d, J=6.3 Hz, 1H), 7.06 (s, 2H), 6.67 (q, J=4.5 Hz, 1H), 6.54 (s, 1H), 4.65 (t, J=6.9 Hz, 2H), 3.41 (q, J=6.7 Hz, 2H), 2.06-1.98 (m, 2H), 1.65-1.56 (m, 2H), 1.36-1.23 ppm (m, 24H). Chemical Formula: C19H31N7O, M.W.: 373.51

224. Compound 224: N-(12-(2H-tetrazol-2-yl)dodecyl)-2-amino nicotinamide (hereinafter referred to as PBK-2025-139)

### <Chemical Formula 224>

white solid, ¹H NMR (400 MHz, CDCl₃): δ=8.50 (s, 1H), 8.01 (d, J=4.1 Hz, 1H), 7.81 (d, J=6.3 Hz, 1H), 7.06 (s, 2H), 6.67 (q, J=4.5 Hz, 1H), 6.54 (s, 1H), 4.65 (t, J=6.9 Hz, 2H), 3.41 (q, J=6.7 Hz, 2H), 2.06-1.98 (m, 2H), 1.65-1.56 (m, 2H), 1.36-1.23 ppm (m, 24H). Chemical Formula: C19H31N7O, M.W.: 373.51

225. Compound 225: 2,4,6-Trimethyl-N-(12-(4-methylpiperazin-1-yl)dodecyl)benzamide hydrochloride (hereinafter referred to as PBK-2025-141)

### <Chemical Formula 225>

white solid, ¹H NMR (400 MHz, MeO D): δ=6.86 (s, 2H), 3.81 (d, J=27.7 Hz, 2H), 3.57-3.55 (m, 2H), 3.48 (s, 1H), 3.01 (s, 3H), 2.26-2.24 (m, 9H), 1.83-1.75 (m, 2H), 1.64-1.56 (m, 2H), 1.40-1.31 ppm (m, 18H). Chemical Formula: C2H48ClN3O, M.W.: 466.15

### Experiment Example 1: PDK1 Inhibitory Efficacy Experiment

To evaluate the PDK1 inhibitory efficacy of a series of compounds according to the present invention, the relative enzyme activity (%) was analyzed at a single concentration (30 μM).

All experiments were performed using the ‘PDK1 Kinase Enzyme System and ADP-Glo™ Kinase assay (Promega, Cat #V9681)’.

All materials used in the experiments were diluted using a 1× reaction solution (6 mM Tris-HCl, 6 mM MgCl2, 6 mM NaCl, 1 mM DTT, pH 7.5) and used as such.

PDK1 was diluted 0.12-fold with the 1× reaction buffer and added at 5 μL per well.

For samples containing both ATP and substrate (PDKtide), ATP and substrate were diluted 0.0625-fold and 0.25-fold, respectively, with the 1× reaction buffer, and 10 μL of the mixture was added per well.

For samples with ATP but no substrate, ATP was diluted 0.0625-fold with 1× reaction solution, and 10 μL of the mixture was added to each well.

The mixture was added to a 96-well white plate (LUMITRAC™ 600 μClear™ Bottom High-Binding 96-well Microplate, Greiner Bio-One, Cat. #655094) according to the experimental conditions as shown in Table 2, and the reaction was allowed to proceed at room temperature for 60 minutes.

TABLE 2

| | | | PDK1 + |
|---|---|---|---|
| | PDK1 + DMSO + | PDK1 + | compound + |
| | substrate (PDKtide) | DMSO | substrate(PDKtide) |
| 30 ng PDK1(μL) | 10.00 | 10.00 | 10.00 |
| 100 μM ATP + 2 μg | 10.00 | 0.00 | 10.00 |
| PDKtide (μL) | | | |
| 100 μM ATP (μL) | 0.00 | 10.00 | 0.00 |
| 30 μM compound | 0.00 | 0.00 | 5.00 |
| (μL) | | | |
| 5% DMSO (μL) | 5.00 | 5.00 | 0.00 |
| Total volume (μL) | 25.00 | 25.00 | 25.00 |

Confirm relative enzyme activity (00 activity)

Add 25 μL of ADP-Glo™ Reagent to each well, incubate at room temperature for 40 minutes to remove any remaining ATP, then add 50 μL of Kinase Detection Reagent and incubate at room temperature for 60 minutes. Ensure that the plate is not exposed to light during the entire process.

Then, the luminescence of each well was measured using a luminometer (Plate-reading luminometer, SpectraMax M2e, Molecular Devices).

For each well, the average value of the wells containing PDK1, substrate, and DMS0 was set to 10000, and the average value of the wells containing only PDK1 and DMS0 was set to 000. All values were normalized and summarized in Table 3.

As a result, it was confirmed that the compounds according to the present invention effectively inhibit PDK1 at a single concentration, thereby enabling regulation.

Here, “regulation” refers to inhibition but may also be interpreted as antagonism.

That is, the compounds of the present invention may be understood to block other agonists (e.g., RSK) from activating PDK1.

Alternatively, the compounds of the present invention may act as antagonists by binding to the RSK binding site at the allosteric site and inhibiting PDK1 activity.

TABLE 3
PDK1 inhibitory efficacy (% activity)
at a single concentration (30 μM)

| Compound No. | Compound name | % activity |
|---|---|---|
| 2 | GO48 | 18.82 |
| 3 | GO51 | 14.29 |
| 4 | GO77 | 2.41 |
| 5 | GO78 | 0.41 |
| 6 | GO79 | 2.21 |
| 7 | GO80 | 0.31 |
| 8 | GO81 | 3.99 |
| 9 | GO83 | 3.11 |
| 10 | GO85 | 0.73 |
| 11 | GO86 | 2.53 |
| 12 | KMC-003P | 5.35 |
| 13 | GO87 | 2.10 |
| 14 | GO88 | 2.01 |
| 15 | GO89 | 10.41 |
| 16 | PBK-2024-003 | 40.75 |
| 17 | PBK-2024-004 | 15.02 |
| 18 | PBK-2024-005 | 19.51 |
| 19 | PBK-2024-006 | 18.01 |
| 20 | PBK-2024-007 | 13.13 |
| 21 | PBK-2024-008 | 18.20 |
| 22 | PBK-2024-009 | 35.45 |
| 23 | PBK-2024-010 | 12.77 |
| 24 | PBK-2024-012 | 16.63 |
| 25 | PBK-2024-013 | 16.11 |
| 26 | PBK-2024-014 | 11.35 |
| 27 | PBK-2024-015 | 19.89 |
| 28 | PBK-2024-016 | 19.51 |
| 29 | PBK-2024-018 | 11.02 |
| 30 | PBK-2024-020 | 6.95 |
| 31 | PBK-2024-021 | 10.34 |
| 32 | PBK-2024-024 | 39.00 |
| 33 | PBK-2024-025 | 16.00 |
| 34 | PBK-2024-026 | 30.70 |
| 35 | PBK-2024-029 | 32.34 |
| 36 | PBK-2024-030 | 24.07 |
| 37 | PBK-2024-032 | 20.37 |
| 38 | PBK-2024-033 | 14.03 |
| 39 | PBK-2024-034 | 42.00 |
| 43 | GO92 | 1.04 |
| 44 | PBK-2025-001 | 3.14 |
| 45 | PBK-2025-002 | 4.45 |
| 46 | PBK-2025-004 | 2.99 |
| 47 | PBK-2025-011 | 2.02 |
| 48 | PBK-2025-012 | 3.24 |
| 49 | PBK-2025-013 | 3.35 |
| 50 | PBK-2025-014 | 3.21 |
| 51 | PBK-2025-015 | 3.18 |
| 52 | PBK-2025-016 | 7.25 |
| 53 | PBK-2025-017 | 2.56 |
| 54 | PBK-2025-018 | 14.65 |
| 55 | PBK-2025-023 | 16.35 |
| 56 | PBK-2025-048 | 7.82 |
| 57 | GO15-F | 10.72 |
| 58 | CS06-005F | 19.33 |
| 59 | PBK-069-F | 22.92 |
| 60 | PBK-048-F | 28.52 |
| 61 | GO15-F-8 | 24.15 |
| 62 | GO59 | 35.83 |
| 63 | GO50 | 10.60 |
| 64 | GO62 | 41.77 |
| 67 | PBK-058-F | 28.51 |
| 68 | GO44 | 28.20 |
| 69 | GO15-F-salt | 10.27 |
| 70 | GO45 | 30.58 |
| 71 | GO45-salt | 21.14 |
| 73 | GO60 | 10.60 |
| 76 | GO73 | 34.52 |
| 77 | PBK-CNB-1 | 61.70 |
| 78 | GO35 | 73.05 |
| 79 | CNA | 51.64 |
| 88 | PBK-2025-069 | 18.28 |
| 89 | PBK-2025-070 | 3.29 |
| 90 | PBK-2025-085 | 19.72 |
| 91 | KP-16 | 3.45 |
| 92 | K-PB-1 | 3.94 |
| 93 | K-PB-2 | 3.88 |
| 94 | K-PB-3 | 3.90 |
| 95 | K-PB-4 | 3.79 |
| 96 | K-PB-5 | 3.95 |
| 97 | K-PB-6 | 6.86 |
| 98 | K-PB-7 | 15.89 |
| 99 | K-PB-8 | 9.56 |
| 101 | K-PB-10 | 16.03 |
| 102 | K-PB-11 | 4.06 |
| 103 | K-PB-12 | 3.99 |
| 104 | K-PB-13 | 2.93 |
| 105 | K-PB-14 | 26.93 |
| 106 | K-PB-15 | 29.67 |
| 107 | K-PB-16 | 12.70 |
| 108 | K-PB-17 | 27.10 |
| 109 | K-PB-18 | 30.88 |
| 110 | K-PB-19 | 18.00 |
| 111 | K-PB-20 | 3.95 |
| 113 | K-PB-22 | 4.23 |
| 114 | K-PB-23 | 4.96 |
| 115 | K-PB-24 | 4.73 |
| 116 | K-PB-25 | 15.84 |
| 117 | K-PB-26 | 7.57 |
| 118 | K-PB-31 | 32.76 |
| 121 | K-PB-40 | 28.10 |
| 122 | K-PB-44 | 18.11 |
| 123 | K-PB-45 | 19.75 |
| 124 | K-PB-46 | 19.56 |
| 125 | K-PB-47 | 14.81 |
| 126 | K-PB-48 | 13.50 |
| 127 | K-PB-49 | 22.12 |
| 128 | K-PB-50 | 7.65 |
| 129 | K-PB-51 | 6.96 |
| 130 | K-PB-52 | 24.19 |
| 131 | K-PB-53 | 6.63 |
| 132 | K-PB-54 | 10.21 |
| 133 | K-PB-55 | 13.79 |
| 134 | K-PB-56 | 26.56 |
| 147 | K-PB-57 | 11.33 |
| 148 | K-PB-58 | 7.35 |
| 149 | K-PB-59 | 4.01 |
| 150 | K-PB-60 | 11.17 |
| 151 | K-PB-61 | 21.94 |
| 152 | K-PB-62 | 13.49 |
| 153 | K-PB-63 | 15.93 |
| 154 | K-PB-64 | 8.38 |
| 155 | K-PB-65 | 4.39 |
| 156 | K-PB-66 | 19.17 |
| 157 | K-PB-67 | 10.94 |
| 158 | K-PB-68 | 22.71 |
| 159 | K-PB-69 | 15.16 |
| 160 | K-PB-70 | 9.03 |
| 161 | K-PB-71 | 11.77 |
| 162 | K-PB-72 | 21.96 |
| 163 | K-PB-73 | 23.80 |
| 164 | K-PB-74 | 5.98 |
| 165 | K-PB-75 | 19.63 |
| 166 | K-PB-76 | 18.24 |
| 167 | K-PB-77 | 10.19 |
| 168 | K-PB-78 | 13.28 |
| 169 | K-PB-79 | 7.40 |
| 170 | K-PB-80 | 7.92 |
| 171 | K-PB-81 | 8.84 |
| 172 | K-PB-82 | 10.06 |
| 173 | K-PB-83 | 19.34 |
| 174 | K-PB-84 | 18.45 |
| 175 | K-PB-85 | 15.51 |
| 176 | K-PB-86 | 10.77 |
| 177 | K-PB-87 | 32.94 |
| 178 | K-PB-88 | 30.61 |
| 179 | K-PB-89 | 23.57 |
| 180 | K-PB-90 | 16.39 |
| 181 | K-PB-91 | 15.73 |
| 182 | K-PB-92 | 24.51 |
| 183 | K-PB-93 | 23.98 |
| 184 | K-PB-94 | 34.75 |
| 185 | K-PB-95 | 27.28 |
| 186 | K-PB-96 | 30.69 |
| 187 | K-PB-97 | 38.50 |
| 188 | K-PB-98 | |
| 189 | K-PB-99 | 13.23 |
| 190 | K-PB-100 | 27.5 |
| 191 | KP-01 | 11.90 |
| 192 | KP-06 | 14.75 |
| 193 | KP-07 | 12.78 |
| 194 | KP-08 | 15.44 |
| 195 | KP-10 | 13.08 |
| 196 | KP-21 | 19.51 |
| 197 | KP-23 | 21.94 |
| 198 | KP-25 | 20.55 |
| 199 | KP-26 | 28.42 |
| 200 | KP-27 | 21.66 |
| 201 | KP-28 | 26.14 |
| 202 | KP-29 | 19.90 |
| 203 | KP-30 | 20.75 |
| 204 | KP-31 | 24.18 |
| 207 | KP-53 | 19.53 |
| 208 | KP-54 | 18.99 |
| 209 | KP-61 | 20.68 |
| 210 | KP-62 | 25.70 |
| 211 | KP-63 | 21.44 |
| 212 | KP-64 | 31.87 |
| 213 | KP-65 | 23.72 |
| 214 | KP-66 | 20.60 |
| 216 | KP-68 | 18.95 |
| 217 | KP-69 | 25.20 |
| 218 | PBK-2025-052 | 11.90 |
| 219 | PBK-2025-083 | 23.98 |
| 220 | PBK-2025-115 | 21.68 |
| 221 | PBK-2025-136 | 22.70 |
| 222 | PBK-2025-137 | 19.25 |
| 223 | PBK-2025-138 | 21.34 |
| 224 | PBK-2025-139 | 23.22 |
| 225 | PBK-2025-141 | 25.43 |

### Quantitative Evaluation of PDK1 Kinase Activity Inhibition

As shown in the table below, this invention establishes an in vitro kinase assay system to quantitatively evaluate PDK1 kinase activity inhibition, and utilizes this system to compare and analyze the activity of new PDK1 inhibitor candidate compounds.

Specifically, after reacting ATP (100 μM), substrate (PDKtide, 0.5 μg/L), and PDK1 enzyme (30 ng) at a specific concentration, the difference in ATP consumption between the group treated with each candidate compound and the DMSO control group was measured using luciferase-based luminescence signals (RLU).

Based on the correlation between the luminescence signal and the ATP-to-ADP conversion rate, the candidate compounds of the present invention induced a significant reduction in ATP consumption compared to DMSO, indicating that they effectively inhibit PDK1 substrate phosphorylation.

This suggests that the compounds of the present invention possess a mechanism of action that inhibits PDK1 enzyme activity and exhibit superior inhibitory activity compared to existing compounds.

### Phosphorylation Inhibition Confirmation

Additionally, to confirm whether the compounds of the present invention inhibit the phosphorylation of PDK1 (phosphoinositide-dependent kinase 1) in cancer cells, flow cytometry was performed using human B-cell lymphoma cells (SU-DHL-8, ATCC CRL-2961).

SU-DHL-8 cells were cultured in RPMI-1640 medium (containing 10% FBS, 100 U/mL penicillin, and 100 μg/mL streptomycin), and the cell concentration was adjusted to 1×106 cells/mL and seeded into a 12-well plate. After stabilizing the cells, the compound of the present invention was added, and the cells were cultured for 24 hours.

After drug treatment, the cells were harvested, and the Fc receptor blocking process was performed. The cells were then fixed and permeabilized using the Intracellular Staining Kit (Cat. No. BD554715) from BD Biosciences. Subsequently, the intracellular pPDK1 was stained using the pPDK1-PE fluorescently labeled antibody (BD Biosciences).

Subsequently, the stained cells were analyzed using a flow cytometer, and the results showed that the compound G015F of the present invention exhibited an inhibitory effect on PDK1 phosphorylation compared to the control group, while G048, G086, and G088 demonstrated stronger PDK1 inhibition.

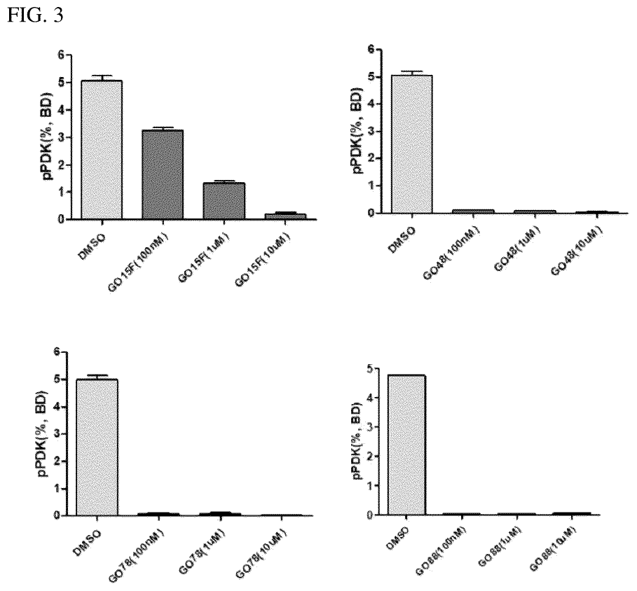

These FACS results are presented in FIG. 3 in a bar graph format.

Referring to the figure, the vertical axis represents the phosphorylation level of PDK1 (phospho-PDK1 expression level), and the horizontal axis denotes the concentration-dependent treatment conditions of each compound.

When the compounds of the present invention were treated at various concentrations, the phospho-PDK1 expression level decreased with increasing concentration compared to the control group. In particular, G048, G086, and G088 exhibited high inhibitory effects even at low concentrations, demonstrating excellent activity. This indicates that these compounds possess superior efficacy in regulating PDK1 phosphorylation.

These results suggest that the compounds of the present invention are associated with the inhibitory effect on PDK1 kinase activity and that the inhibition of PDK1 phosphorylation leads to the suppression of kinase activity.

Since PDK1 is involved in signal transduction pathways, the compounds of the present invention are considered to have the potential to contribute to the treatment of various diseases by regulating these pathways, thereby serving as a foundation for new therapeutic strategies targeting PDK1.

Additionally, enzyme kinetics analysis was performed to quantitatively analyze the effect of the compounds of the present invention on the enzymatic activity of PDK1 (3-Phosphoinositide-dependent protein kinase-1) and to confirm the inhibition pattern.

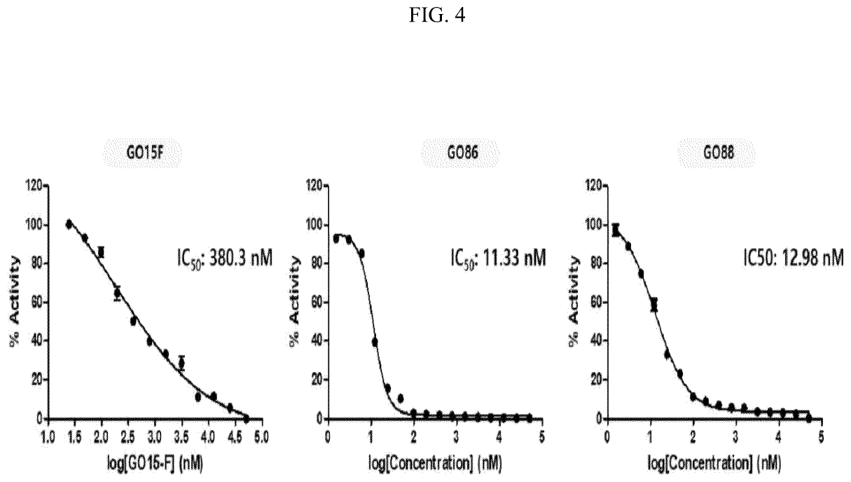

Prior to the analysis, the half-maximal inhibitory concentration (IC50) values of the compounds G015F, G086, and G088 of the present invention for inhibiting PDK1 kinase activity were determined, and the results are shown in FIG. 4.

The results showed that the IC50 values of the compounds G015F, GO86, and GO88 were 380.3 nM, 11.33 nM, and 12.98 nM, respectively.

This confirmed that the compounds of the present invention exhibit excellent inhibitory activity against PDK1.

### Allosteric Site Inhibition Confirmation

The ADP-Glo™ Kinase Assay kit from Promega was used to measure changes in enzyme activity as a function of the concentrations of the reaction substrates (ATP and peptide substrate) and the compounds of the present invention.

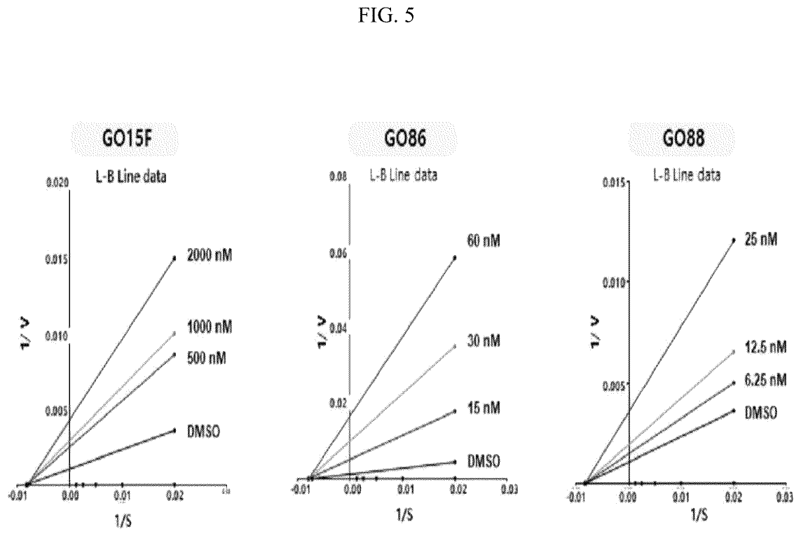

The compounds of the present invention were treated at concentrations of G015F (500, 1000, 2000 nM), G086 (15, 30, 60 nM), and G088 (6.25, 12.5, 25 nM), and the reaction rates were measured as a function of substrate concentration at each concentration, as shown in FIG. 5.

Based on the measured reaction rates, a Lineweaver-Burk plot was constructed to analyze changes in Km and Vmax.

The Lineweaver-Burk plot plots the inverse of the reaction rate (1/V) on the vertical axis and the inverse of the substrate concentration (1/[S]) on the horizontal axis, enabling the analysis of Km and Vmax of the enzyme reaction in a linear form.

As a result, Vmax decreased with increasing concentrations of the active compound, but there was no significant change in Km.

This clearly indicates that the compound of the present invention acts as a non-competitive inhibitor of PDK1.

These results suggest that the compound of the present invention inhibits PDK1 activity without competing with the substrate, thereby effectively contributing to the regulation of overactive PDK1-related signaling pathways.

### Experimental Example 2: Verification of T Cell Proliferation Inhibitory Effect

To verify the T cell proliferation inhibitory effect of the compounds according to the present invention, the following experiment was performed based on flow cytometry analysis using carboxyfluorescein diacetate succinimidyl ester (CFSE), a fluorescent dye that binds to intracellular molecules via covalent bonds.

Spleens were extracted from 7-week-old C57BL/6 mice, ground, and then separated into single cells using a strainer (40 m pore size, SPL). Red blood cells were removed using ACK (Ammonium-Chloride-Potassium) lysis buffer to isolate white blood cells.

CD90.2 microbeads (130-121-278, Miltenyi Biotech.) were added, and the mixture was incubated at 4° C. for 20 minutes. Finally, using a MACS Magnetic Stand and LS column, T cells were isolated from the spleen.

The T cells isolated from the spleen were resuspended in 1 mL of free medium (RPMI-1640+200 U/mL penicillin+200 μg/mL streptomycin), and 0.3 μL of CFSE (10 mM) was added. The mixture was incubated at 37° C. for 5 minutes.

Free medium (10 mL) was added to stop the reaction, and the cells were centrifuged to obtain the cell pellet.

RPMI 1640 medium containing 10% FBS, 100 U/mL penicillin, 100 μg/mL streptomycin, and resuspended the cells. The cells were then seeded into a 96-well plate at a density of 2×10⁵ cells per well and activated with CD3 and CD28 antibodies (0.5 g/mL each) to activate T cells.

The compound diluted in DMSO was added at concentrations of 10 nM, 100 nM, 1 μM, 10 M, 30 μM, and 50 M, with three wells per concentration, and cultured at 37° C. and 5% CO₂ for three days. The cells were then analyzed using a FACS Canto instrument.

The results obtained were the T cell proliferation inhibition rate (%), which is shown in Table 4.

The compounds belonging to the chemical formula 1 of the present invention exhibit excellent efficacy in inhibiting T cell proliferation, whereas the compounds corresponding to the control group show no or very low efficacy in inhibiting T cell proliferation.

The proliferation inhibition rate (%) was calculated using the following formula:

$$Proliferation inhibition rate ( \% ) = 100 - ( ( fluorescence intensity of the sample treated with the compound + CD 3 + CD 28 ) \div ( fluorescence intensity of the control sample ( DMSO + CD 3 + CD 28 ) ) \times 100$$

As shown in Table 4 below, after treating T cells isolated from mouse spleens with the compound of the present invention, stimulating them with anti-CD3 and anti-CD28 antibodies, and performing CFSE analysis, it was confirmed that T cell proliferation was significantly inhibited in the group treated with the compound of the present invention dissolved in DMSO compared to the control group treated with DMSO alone.

This suggests that the compounds of the present invention have a mechanism of action and function that regulates the proliferation of immune cells by interfering with signaling pathways related to T cell activation or proliferation, thereby exhibiting excellent effects in regulating immune responses or suppressing excessive immune activation.

TABLE 4
Proliferation inhibition rate (%)

| | concentration | | | | | |
|---|---|---|---|---|---|---|
| name | 50 μM | 30 μM | 10 μM | 1 μM | 100 nM | 10 nM |
| PBK-069-F | 99.82 | 78.36 | −8.32 | 0.00 | 0.00 | 0.00 |
| CS06-005-F | 95.79 | 78.89 | 20.03 | 1.92 | 1.78 | −4.38 |
| PBK-048-F | 100.00 | 100.00 | 63.55 | 6.85 | 8.77 | −1.92 |
| PBK-058-F | 100.00 | 73.81 | −3.83 | 0.00 | 0.00 | 0.00 |
| GO15-F | 100.00 | 100.00 | 100.00 | 16.20 | −5.28 | −9.32 |
| GO35 | 8.76 | 4.51 | 0.68 | −3.73 | −4.19 | −7.14 |
| GO15-F-salt | 99.20 | 89.94 | 65.36 | 7.14 | −4.66 | −4.04 |
| GO15-F-8 | 93.16 | 85.42 | 34.85 | −5.75 | −6.37 | −10.25 |
| GO48 | 100.00 | 100.00 | 100.00 | 0.00 | 0.00 | −1.47 |
| GO50 | 100.00 | 100.00 | 100.00 | 11.16 | 4.85 | −9.84 |
| GO51 | 100.00 | 100.00 | 88.33 | 0.00 | 0.00 | 4.99 |
| GO58 | 89.48 | 18.95 | 16.18 | 6.44 | −0.27 | −6.03 |
| GO59 | 100.00 | 100.00 | −18.49 | 0.00 | 0.00 | 0.00 |
| GO60 | 100.00 | 100.00 | 84.76 | 7.40 | 5.75 | −6.44 |
| GO62 | 97.00 | 80.43 | 8.94 | 6.99 | 8.63 | −4.11 |
| GO73 | 97.78 | 23.66 | −4.95 | 0.00 | 0.00 | 0.00 |
| GO58 | 89.48 | 18.95 | 16.18 | 0.00 | 0.00 | 0.00 |
| PBK-CNB-1 | 32.20 | 3.80 | 2.09 | 15.34 | −3.97 | 1.51 |
| CNA | 34.69 | 27.88 | 10.99 | 7.12 | 1.23 | −2.19 |
| GO77 | 100.00 | 100.00 | 100.00 | 100.00 | 100.00 | 2.70 |
| GO78 | 100.00 | 100.00 | 100.00 | 100.00 | 100.00 | −27.49 |
| GO79 | 100.00 | 100.00 | 100.00 | 100.00 | 100.00 | −18.46 |
| GO80 | 100.00 | 100.00 | 100.00 | 100.00 | 100.00 | 5.14 |
| GO81 | 100.00 | 100.00 | 100.00 | 100.00 | 32.45 | 4.55 |
| GO83 | 100.00 | 100.00 | 100.00 | 100.00 | 100.00 | 38.33 |
| GO85 | 100.00 | 100.00 | 100.00 | 100.00 | 100.00 | 100.00 |
| GO86 | 100.00 | 100.00 | 100.00 | 100.00 | 100.00 | −25.47 |
| GO87 | 100.00 | 100.00 | 81.15 | 100.00 | 1.71 | 9.13 |
| GO88 | 100.00 | 98.95 | 85.09 | 100.00 | 100.00 | 17.83 |
| GO89 | 100.00 | 100.00 | 100.00 | 100.00 | 100.00 | −0.40 |
| PBK-2024-003 | 100.00 | 100.00 | 100.00 | 100.00 | −2.93 | 4.13 |
| PBK-2024-004 | 100.00 | 100.00 | 100.00 | 100.00 | −14.00 | 0.00 |
| PBK-2024-005 | 100.00 | 100.00 | 100.00 | 100.00 | 76.13 | 1.87 |
| PBK-2024-006 | 100.00 | 100.00 | 100.00 | 100.00 | 100.00 | −14.40 |
| PBK-2024-007 | 100.00 | 100.00 | 100.00 | 100.00 | 100.00 | 3.46 |
| PBK-2024-008 | 100.00 | 100.00 | 100.00 | 100.00 | 100.00 | −13.87 |
| PBK-2024-009 | 100.00 | 100.00 | 100.00 | 100.00 | 100.00 | −0.80 |
| PBK-2024-010 | 100.00 | 100.00 | 100.00 | 100.00 | 100.00 | −12.80 |
| PBK-2024-012 | 100.00 | 100.00 | 100.00 | 100.00 | 100.00 | −7.33 |
| PBK-2024-013 | 100.00 | 100.00 | 100.00 | 100.00 | 89.43 | −2.67 |
| PBK-2024-014 | 100.00 | 100.00 | 100.00 | 100.00 | 100.00 | 2.13 |
| PBK-2024-015 | 100.00 | 100.00 | 100.00 | 100.00 | 83.33 | 2.27 |
| PBK-2024-016 | 100.00 | 100.00 | 100.00 | 100.00 | 100.00 | −0.27 |
| PBK-2024-018 | 100.00 | 100.00 | 100.00 | 100.00 | 100.00 | 4.15 |
| PBK-2024-020 | 100.00 | 100.00 | 100.00 | 100.00 | 100.00 | 1.34 |
| PBK-2024-021 | 100.00 | 100.00 | 100.00 | 100.00 | 77.15 | 8.01 |
| PBK-2024-024 | 100.00 | 100.00 | 100.00 | 100.00 | 100.00 | 0.29 |
| PBK-2024-025 | 100.00 | 100.00 | 100.00 | 100.00 | 100.00 | −36.05 |
| PBK-2024-026 | 100.00 | 100.00 | 100.00 | 100.00 | 100.00 | 2.08 |
| PBK-2024-029 | 100.00 | 100.00 | 100.00 | 100.00 | 100.00 | 8.33 |
| PBK-2024-030 | 100.00 | 100.00 | 100.00 | 100.00 | 100.00 | −38.89 |
| PBK-2024-032 | 100.00 | 100.00 | 100.00 | 100.00 | 33.33 | −4.14 |
| PBK-2024-033 | 100.00 | 100.00 | 100.00 | 100.00 | 100.00 | −33.51 |
| PBK-2024-034 | 100.00 | 100.00 | 100.00 | 100.00 | 100.00 | −58.92 |
| GO92 | 100.00 | 100.00 | 100.00 | 100.00 | 100.00 | −10.80 |
| PBK-2025-001 | 100.00 | 100.00 | 100.00 | 100.00 | 100.00 | −6.21 |
| PBK-2025-002 | 100.00 | 100.00 | 100.00 | 100.00 | 100.00 | 0.16 |
| PBK-2025-004 | 100.00 | 100.00 | 100.00 | 100.00 | 100.00 | −10.52 |
| PBK-2025-011 | 100.00 | 100.00 | 100.00 | 100.00 | 100.00 | −28.82 |
| PBK-2025-012 | 100.00 | 100.00 | 100.00 | 100.00 | 100.00 | −18.31 |
| PBK-2025-013 | 100.00 | 100.00 | 100.00 | 100.00 | 100.00 | −22.61 |
| PBK-2025-014 | 100.00 | 100.00 | 100.00 | 100.00 | 100.00 | −7.48 |
| PBK-2025-015 | 100.00 | 100.00 | 100.00 | 100.00 | 16.10 | 0.77 |
| PBK-2025-016 | 100.00 | 100.00 | 100.00 | 100.00 | 13.77 | 11.34 |
| PBK-2025-017 | 100.00 | 100.00 | 100.00 | 100.00 | 100.00 | −26.59 |
| PBK-2025-018 | 100.00 | 100.00 | 100.00 | 100.00 | 100.00 | −43.03 |
| PBK-2025-023 | 100.00 | 100.00 | 100.00 | 100.00 | 100.00 | 89.67 |
| PBK-2025-048 | 100.00 | 100.00 | 100.00 | 81.11 | 2.91 | −2.51 |
| PBK-2025-069 | 100.00 | 100.00 | 100.00 | 56.26 | 4.29 | −3.22 |
| PBK-2025-070 | 100.00 | 100.00 | 100.00 | 100.00 | 100.00 | −3.34 |
| PBK-2025-085 | 100.00 | 100.00 | 100.00 | 66.18 | −2.03 | −8.27 |
| KP-16 | 100.00 | 100.00 | 100.00 | 100.00 | 100.00 | −14.62 |
| K-PB-01 | 100.00 | 100.00 | 100.00 | 100.00 | 100.00 | −9.65 |
| K-PB-02 | 100.00 | 100.00 | 100.00 | 100.00 | 100.00 | −12.87 |
| K-PB-03 | 100.00 | 100.00 | 100.00 | 100.00 | 100.00 | −25.44 |
| K-PB-04 | 100.00 | 100.00 | 100.00 | 100.00 | 100.00 | 94.37 |
| K-PB-05 | 100.00 | 100.00 | 100.00 | 100.00 | 100.00 | −0.58 |
| K-PB-06 | 100.00 | 100.00 | 100.00 | 100.00 | 40.64 | 3.51 |
| K-PB-07 | 100.00 | 100.00 | 100.00 | 100.00 | −3.95 | −4.82 |
| K-PB-08 | 100.00 | 100.00 | 100.00 | 100.00 | 100.00 | −12.13 |
| K-PB-09 | 100.00 | 100.00 | 100.00 | 100.00 | 7.60 | 2.92 |
| K-PB-10 | 100.00 | 100.00 | 100.00 | 100.00 | 100.00 | −0.15 |
| K-PB-11 | 100.00 | 100.00 | 100.00 | 100.00 | 100.00 | −4.53 |
| K-PB-12 | 100.00 | 100.00 | 100.00 | 100.00 | 100.00 | −6.43 |
| K-PB-13 | 100.00 | 100.00 | 100.00 | 100.00 | 100.00 | 100.00 |
| K-PB-14 | 100.00 | 100.00 | 100.00 | 38.89 | 8.33 | 6.73 |
| K-PB-15 | 100.00 | 100.00 | 100.00 | 0.44 | −1.90 | 0.44 |
| K-PB-16 | 100.00 | 100.00 | 100.00 | 100.00 | 100.00 | −35.23 |
| K-PB-17 | 100.00 | 100.00 | 100.00 | 100.00 | 28.51 | 0.88 |
| K-PB-18 | 100.00 | 100.00 | 100.00 | 20.91 | 8.48 | 0.15 |
| K-PB-19 | 100.00 | 100.00 | 100.00 | 100.00 | 100.00 | −36.70 |
| K-PB-20 | 100.00 | 100.00 | 100.00 | 100.00 | 100.00 | 0.00 |
| K-PB-22 | 100.00 | 100.00 | 100.00 | 100.00 | 100.00 | 11.11 |
| K-PB-23 | 100.00 | 100.00 | 100.00 | 100.00 | 26.17 | −18.27 |
| K-PB-24 | 100.00 | 100.00 | 100.00 | 100.00 | 100.00 | −11.40 |
| K-PB-25 | 100.00 | 100.00 | 100.00 | 100.00 | 26.17 | −1.90 |
| K-PB-26 | 100.00 | 100.00 | 100.00 | 100.00 | 100.00 | −15.79 |
| K-PB-31 | 100.00 | 88.60 | 34.50 | 12.57 | 4.82 | 4.24 |
| K-PB-40 | 100.00 | 100.00 | 30.00 | 0.00 | 0.00 | 0.00 |
| K-PB-44 | 100.00 | 100.00 | 100.00 | 80.00 | 0.00 | 0.00 |
| K-PB-45 | 100.00 | 100.00 | 100.00 | 48.36 | 4.78 | −2.54 |
| K-PB-46 | 100.00 | 100.00 | 100.00 | 30.00 | 20.00 | 0.00 |
| K-PB-47 | 100.00 | 100.00 | 100.00 | 100.00 | 100.00 | −4.48 |
| K-PB-48 | 100.00 | 100.00 | 100.00 | 100.00 | 100.00 | 0.00 |
| K-PB-49 | 100.00 | 100.00 | 100.00 | 15.07 | 10.30 | −2.24 |
| K-PB-50 | 100.00 | 100.00 | 100.00 | 100.00 | 100.00 | −7.16 |
| K-PB-51 | 100.00 | 100.00 | 100.00 | 100.00 | 90.00 | 0.00 |
| K-PB-52 | 100.00 | 100.00 | 85.43 | 13.58 | −5.22 | −4.33 |
| K-PB-53 | 100.00 | 100.00 | 100.00 | 100.00 | 100.00 | −6.12 |
| KP-01 | 100.00 | 100.00 | 93.26 | 7.39 | 0.00 | 0.00 |
| KP-06 | 100.00 | 100.00 | 99.81 | 0.96 | −0.84 | −3.25 |
| KP-07 | 100.00 | 100.00 | 100.00 | 11.96 | 4.87 | 5.02 |
| KP-08 | 100.00 | 100.00 | 100.00 | −1.68 | −2.40 | −4.09 |
| KP-10 | 100.00 | 100.00 | 100.00 | −7.83 | 2.81 | 1.77 |
| KP-21 | 100.00 | 100.00 | 100.00 | 12.30 | 4.05 | 5.21 |
| KP-23 | 100.00 | 100.00 | 97.99 | 16.35 | 10.27 | 7.81 |
| KP-25 | 100.00 | 100.00 | 100.00 | 20.12 | 9.41 | 5.35 |
| KP-26 | 100.00 | 100.00 | 40.00 | 0.00 | 0.00 | 0.00 |
| KP-27 | 100.00 | 100.00 | 80.00 | 0.00 | 0.00 | 0.00 |
| KP-28 | 100.00 | 100.00 | 78.44 | 8.54 | 6.66 | 7.24 |
| KP-29 | 100.00 | 100.00 | 100.00 | 18.96 | 13.17 | 4.05 |
| KP-30 | 100.00 | 100.00 | 100.00 | 6.80 | 1.16 | −4.49 |
| KP-31 | 100.00 | 100.00 | 95.01 | 12.30 | −0.14 | −2.60 |
| KP-40 | 97.99 | 89.32 | 9.84 | 4.34 | 3.62 | 11.14 |
| KP-53 | 100.00 | 100.00 | 100.00 | −3.04 | −3.91 | −5.35 |
| KP-54 | 100.00 | 100.00 | 100.00 | 10.56 | −0.58 | 2.46 |
| KP-61 | 100.00 | 100.00 | 84.52 | 18.81 | 1.59 | −1.45 |
| KP-62 | 100.00 | 100.00 | 42.84 | 2.75 | −3.18 | −4.34 |
| KP-63 | 100.00 | 100.00 | 90.01 | 9.70 | −0.87 | −2.17 |
| KP-64 | 100.00 | 71.78 | 20.55 | −4.34 | −3.91 | −2.89 |
| KP-65 | 100.00 | 100.00 | 100.00 | 8.54 | 6.37 | −0.14 |
| KP-66 | 100.00 | 100.00 | 100.00 | 17.37 | 12.16 | 2.32 |
| KP-68 | 100.00 | 100.00 | 100.00 | 13.02 | 2.32 | 2.03 |
| PBK-2025-052 | 100.00 | 100.00 | 100.00 | 12.40 | 2.74 | 1.31 |
| PBK-2025-083 | 100.00 | 100.00 | 66.47 | 3.48 | 2.18 | −5.37 |

### Experiment Example 3: Evaluation of Anticancer Efficacy against Cancer Cell Lines

To evaluate the anticancer efficacy of the compounds according to the present invention against cancer cell lines, the following experiments were performed.

Human-derived B lymphoma cell line SU-DHL-8 (Cat. #CRL-2961) purchased from ATCC was cultured in RPMI-1640 medium containing 1000 FBS, 100 U/mL penicillin, and 100 μg/mL streptomycin.

The cell culture medium was then harvested, centrifuged at 1,300 rpm for 5 minutes, and the supernatant was removed. The cells were resuspended in the same medium and dispensed into a 96-well plate (Costar 96-well cell culture plate, Corning) at 90 μL per well, resulting in the conditions specified in Table 5.

The compounds according to the present invention or the control group (DMSO) were added at concentrations of 0.05, 0.1, 0.5, 1, 10, 30, and 50 PM, 10 μL each, to the plate, and the plate was incubated at 37° C. in a 5% CO₂ incubator for 48 hours.

Then, 10 μL of CCK-8 was added to each well, and the plates were incubated again at 37° C. in a 5% CO₂ incubator for 4 hours. The absorbance was measured at 450 nm, and the relative cell survival rate compared to untreated cells was presented in Table 6.

Human-derived breast cancer cell line MCF-7, human-derived lung cancer cell line A549, human-derived colorectal cancer cell line HCT-116, human pancreatic cancer cell line AsPC-1, glioblastoma cell line A172, and melanoma cell line Malme-3 μM were obtained from the Korean Cell Bank. gastric cancer cell line SNU-719, and bladder cancer cell line T24 were cultured in RPMI-1640 medium containing 10% FBS.

Similarly, human-derived cervical cancer cell line HeLa and renal cancer cell line ACHN, obtained from the Korean Cell Bank, were cultured in DMEM medium containing 10% FBS, 100 U/mL penicillin, and 100 μg/mL streptomycin.

Human-derived head and neck cancer cell line Fadu and osteosarcoma cell line Saos-2 were cultured in MEM medium containing 10% FBS, 100 U/mL penicillin, and 100 μg/mL streptomycin.

Following stabilization, the cells were harvested using Trypsin-EDTA treatment, centrifuged at 1,200 rpm for 3 minutes, the supernatant was removed, and the cells were resuspended in the same medium. They were then seeded into a 96-well plate (Costar 96-well cell culture plate, Coming) at 100 μL per well, and the conditions were adjusted as indicated in Table 5.

The cells were cultured in a 5% CO₂ incubator at 37° C. for 24 hours, and after stabilization of the cells, each plate was washed with 200 μL of PBS per well and treated with 50 μL of culture medium per well.

The compounds according to the present invention or the control group (DMS0) were added at concentrations of 0, 10, 30, and 50 μM in 50 μL aliquots to the plates, and the plates were cultured at 37° C. in a 500 CO₂ incubator for 48 hours.

After that, 10 μL of 2CCK-8 was added to each well, and the plate was incubated again at 37° C. in a 5% CO₂ incubator for 2 hours. The absorbance was measured at 450 nm, and the relative cell survival rate compared to untreated cells was presented in Tables 7 to 19.

TABLE 5

| Cell line | Culture conditions | Final cell count |
|---|---|---|
| SU-DHL-8 | RPMI with 10% FBS, | 2 × 10⁴cells/well |
| (Human B lymphocyte) | 100 U/mL penicillin, | |
| MCF-7 | 100 μg/mL | 2 × 10⁴cells/well |
| (Human Breast Cancer) | streptomycin | |
| HCT-116 | | 3.5 × 10⁴cells/well |
| (Human Colorectal | | |
| Carcinoma) | | |
| Malme-3M | | 5 × 10³cells/well |
| (Human melanoma) | | |
| SNU-719 | | 5 × 10³cells/well |
| (Human gastric cancer) | | |
| T24 | | 5 × 10³cells/well |
| (Human bladder cancer) | | |
| AsPC-1 | | 5 × 10³cells/well |
| (Human pancreatic | | |
| cancer) | | |
| A172 | | 5 × 10³cells/well |
| (Human glioblastoma) | | |
| KMS12PE | | 5 × 10⁴cells/well |
| (Human multiple | | |
| myeloma) | | |
| KMS26 | | 5 × 10⁴cells/well |
| (Human multiple | | |
| myeloma) | | |
| KMS34 | | 5 × 10⁴cells/well |
| (Human multiple | | |
| myeloma) | | |
| HeLa | DMEM with 10% FBS, | 1.5 × 10⁴cells/well |
| (Human Cervical | 100 U/mL penicillin, | |
| Carcinoma) | 100 μg/mL | |
| ACHN | streptomycin | 5 × 10³cells/well |
| (Human kidney cnacer) | | |
| Saos-2 | MEM with 10% FBS, | 5 × 10³cells/well |
| (Human Osteosarcoma) | 100 U/mL penicillin, | |
| Fadu | 100 μg/mL | 5 × 10³cells/well |
| (Human Head and Neck | streptomycin | |
| cancer) | | |

In other words, when the survival rate of untreated cells (DMSO) is set at 100%, the relative cell survival rate in each group treated with the drug at different concentrations is indicated.

Based on these results, it was verified that the compound according to the present invention has anticancer activity.

To evaluate the anticancer activity of the compounds according to the present invention at concentrations resulting in less than 5000 cell survival at 1 μM, the concentration-dependent cell survival rates were measured using various human-derived cancer cell lines (SU-D,L-8 and 12 others). The results showed a significant decrease in cell survival rate in the groups treated with the compounds according to the present invention compared to the DMSO control group.

This suggests that the compounds of the present invention exhibit anticancer activity by inhibiting cell survival and proliferation-related signaling pathways or through cytotoxic mechanisms such as apoptosis induction, and are considered to possess broad-spectrum and excellent cytotoxic efficacy against various types of cancer.

TABLE 6
Relative cell viability (%)
Human B Lymphocyte cell line, SU-DHL-8.

| concentration | | | | |
|---|---|---|---|---|
| name | 50 μM | 30 μM | 10 μM | 1 μM |
| GO48 | 24.67 | 25.29 | 26.01 | 31.02 |
| GO50 | 27.92 | 29.14 | 29.82 | 32.96 |
| GO51 | 23.47 | 24.66 | 25.67 | 29.13 |
| GO59 | 19.31 | 22.91 | 24.90 | 32.48 |
| GO60 | 0.91 | 0.98 | 5.03 | 11.92 |
| GO62 | 6.33 | 6.51 | 14.08 | 23.28 |
| GO63 | 7.38 | 9.63 | 15.20 | 17.92 |
| Kmedi-87-F | 1.34 | 4.10 | 4.93 | 15.42 |
| GO15 salt | 11.94 | 15.51 | 18.75 | 30.47 |
| GO77 | 4.45 | 4.68 | 5.49 | 7.58 |
| GO78 | 3.67 | 4.63 | 5.87 | 8.80 |
| GO79 | 3.94 | 4.66 | 6.39 | 7.83 |
| GO80 | 3.17 | 3.48 | 4.16 | 5.17 |
| GO81 | 4.49 | 5.07 | 6.49 | 10.82 |
| GO83 | 3.14 | 3.36 | 4.19 | 5.32 |
| GO85 | 4.86 | 5.52 | 6.21 | 7.37 |
| GO86 | 3.29 | 4.82 | 6.09 | 7.63 |
| KMC-003P | 1.13 | 1.64 | 3.79 | 7.60 |
| GO88 | 0.32 | 1.16 | 3.31 | 9.28 |
| PBK-2024-003 | 4.94 | 5.98 | 6.56 | 7.33 |
| PBK-2024-004 | 5.21 | 5.69 | 6.11 | 10.58 |
| PBK-2024-005 | 3.44 | 4.09 | 4.50 | 5.28 |
| PBK-2024-006 | 5.08 | 5.98 | 7.51 | 8.51 |
| PBK-2024-007 | 3.68 | 4.89 | 5.34 | 7.74 |
| PBK-2024-008 | −0.22 | 3.70 | 7.68 | 9.64 |
| PBK-2024-009 | 1.67 | 2.45 | 2.85 | 6.46 |
| PBK-2024-010 | 1.97 | 9.17 | 15.71 | 16.11 |
| PBK-2024-011 | 0.52 | 1.57 | 3.28 | 9.85 |
| PBK-2024-012 | −0.24 | 0.62 | 1.65 | 8.38 |
| PBK-2024-013 | 0.95 | 0.11 | 1.26 | 9.73 |
| PBK-2024-014 | −0.99 | 0.05 | 1.04 | 1.70 |
| PBK-2024-015 | 0.56 | 2.03 | 4.97 | 6.24 |
| PBK-2024-016 | −1.55 | 0.33 | 2.82 | 5.46 |
| PBK-2024-017 | −1.01 | −0.18 | 0.23 | 0.94 |
| PBK-2024-018 | 1.03 | 1.25 | 1.86 | 3.85 |
| PBK-2024-019 | 7.80 | 9.46 | 16.84 | 18.14 |
| PBK-2024-020 | 1.06 | 1.86 | 2.60 | 7.90 |
| PBK-2024-021 | 0.23 | 1.00 | 4.36 | 5.57 |
| PBK-2024-024 | 1.45 | 1.97 | 3.01 | 3.48 |
| PBK-2024-025 | 2.66 | 3.35 | 4.32 | 10.08 |
| PBK-2024-026 | 1.37 | 2.45 | 3.11 | 3.63 |
| PBK-2024-029 | −0.64 | 0.42 | 0.64 | 1.18 |
| PBK-2024-030 | 4.25 | 5.98 | 13.93 | 17.42 |
| PBK-2024-032 | 2.32 | 3.17 | 4.45 | 6.38 |
| PBK-2024-033 | 1.42 | 1.79 | 3.30 | 4.16 |
| PBK-2024-034 | 4.27 | 6.24 | 8.41 | 11.48 |
| PBK-2025-001 | 1.41 | 2.54 | 4.91 | 6.14 |
| PBK-2025-002 | −1.31 | 0.53 | 1.27 | 1.91 |
| PBK-2025-004 | −0.92 | 2.08 | 2.44 | 4.80 |
| PBK-2025-011 | 8.33 | 9.40 | 10.03 | 11.70 |
| PBK-2025-012 | 8.63 | 9.22 | 10.42 | 11.96 |
| PBK-2025-013 | 4.09 | 7.48 | 8.02 | 8.93 |
| PBK-2025-014 | 4.31 | 6.18 | 8.18 | 9.20 |
| PBK-2025-015 | 0.48 | 0.89 | 1.98 | 3.48 |
| PBK-2025-016 | 9.15 | 10.14 | 11.59 | 14.24 |
| PBK-2025-017 | 2.07 | 2.78 | 4.02 | 5.59 |
| PBK-2025-018 | 2.23 | 3.26 | 4.05 | 6.42 |
| PBK-2025-023 | 0.97 | 1.10 | 4.92 | 9.36 |
| PBK-2025-048 | 0.00 | 11.20 | 19.60 | 32.40 |
| PBK-2025-069 | 2.23 | 5.06 | 5.98 | 12.58 |
| PBK-2025-070 | −0.86 | 0.48 | 0.82 | 1.33 |
| PBK-2025-085 | 1.94 | 2.48 | 5.94 | 7.19 |
| GO15-F | 11.94 | 15.63 | 18.75 | 19.00 |
| GO15-F-8 | 21.01 | 42.03 | 36.41 | 48.82 |
| GO15-F-salt | 0.77 | 1.05 | 15.16 | 27.80 |
| GO44 | −5.64 | −2.90 | 15.27 | 16.05 |
| GO45 | −0.57 | 1.45 | 4.94 | 5.01 |
| GO45-salt | 0.75 | 2.04 | 4.05 | 5.57 |
| GO92 | 1.58 | 2.34 | 11.93 | 22.61 |
| KP-16 | 1.59 | 1.84 | 3.12 | 3.93 |
| K-PB-40 | 31.11 | 29.04 | 28.99 | 34.89 |
| K-PB-44 | 35.70 | 32.93 | 31.91 | 31.84 |
| K-PB-45 | 48.83 | 44.59 | 42.17 | 42.24 |
| K-PB-46 | 31.72 | 32.10 | 30.70 | 30.43 |
| K-PB-47 | 42.71 | 42.04 | 42.57 | 41.97 |
| K-PB-48 | 30.44 | 32.32 | 31.98 | 33.03 |
| K-PB-51 | 34.27 | 35.05 | 33.99 | 34.26 |
| K-PB-53 | 42.46 | 42.71 | 42.13 | 44.80 |

TABLE 7
Relative cell viability (%)
Human Cervical Carcinoma cell line, HeLa

| Concentration | | | | |
|---|---|---|---|---|
| Name | 50 μM | 30 μM | 10 μM | 1 μM |
| GO51 | 0.73 | 1.07 | 3.26 | 16.44 |

TABLE 8
Relative cell viability (%)
Human Breast Cancer cell line, MCF-7

| Concentration | | | | |
|---|---|---|---|---|
| Name | 50 μM | 30 μM | 10 μM | 1 μM |
| GO51 | 1.95 | 4.02 | 9.10 | 35.36 |

TABLE 9
Relative cell viability (%)
Human Colorectal Carcinoma cell line, HCT-116

| Concentration | | | | |
|---|---|---|---|---|
| Name | 50 μM | 30 μM | 10 μM | 1 μM |
| GO85 | 8.90 | 10.07 | 12.04 | 13.03 |
| GO86 | 25.26 | 24.78 | 24.91 | 21.36 |
| GO88 | 7.61 | 9.38 | 12.31 | 14.20 |
| GO89 | 9.08 | 10.00 | 11.46 | 12.07 |
| PBK-2024-010 | 12.96 | 14.00 | 14.63 | 13.08 |
| PBK-2024-021 | 11.99 | 12.31 | 13.20 | 11.61 |
| PBK-2024-026 | 7.09 | 10.44 | 12.46 | 13.78 |
| PBK-2025-016 | 11.84 | 12.71 | 13.31 | 33.57 |
| PBK-2025-017 | 7.91 | 9.30 | 12.33 | 10.94 |

TABLE 10
Relative cell viability (%)
Human Kidney Cancer cell line, ACHN

| Concentration | | | | |
|---|---|---|---|---|
| Name | 50 μM | 30 μM | 10 μM | 1 μM |
| GO78 | 28.30 | 29.23 | 30.02 | 29.50 |
| GO89 | 30.30 | 30.56 | 30.00 | 31.67 |
| PBK-2024-004 | 33.05 | 35.10 | 33.60 | 35.03 |
| PBK-2025-017 | 26.79 | 33.16 | 32.98 | 36.73 |

TABLE 11
Relative cell viability (%)
Human Pancreas Adenocarcinoma cell line, AsPC-1

| Concentration | | | | |
|---|---|---|---|---|
| Name | 50 μM | 30 μM | 10 μM | 1 μM |
| GO85 | 35.83 | 40.21 | 41.10 | 40.40 |
| GO88 | 30.49 | 36.76 | 45.39 | 43.06 |
| GO89 | 41.48 | 44.15 | 42.80 | 40.88 |
| PBK-2024-004 | 33.19 | 35.65 | 38.13 | 43.44 |
| PBK-2024-010 | 42.00 | 42.68 | 44.55 | 42.38 |
| PBK-2024-021 | 36.64 | 39.43 | 41.04 | 40.83 |
| PBK-2024-026 | 35.89 | 39.02 | 40.27 | 39.48 |
| PBK-2025-017 | 43.14 | 43.45 | 43.59 | 44.59 |
| PBK-2025-070 | 17.80 | 17.81 | 19.18 | 30.32 |

TABLE 12
Relative cell viability (%)
Human Melanoma cancer cell, Malme-3M

| Concentration | | | | |
|---|---|---|---|---|
| Name | 50 μM | 30 μM | 10 μM | 1 μM |
| GO85 | 5.97 | 6.03 | 6.19 | 6.00 |
| GO86 | 38.87 | 36.80 | 37.40 | 44.06 |
| GO88 | 15.86 | 16.27 | 31.23 | 43.71 |
| PBK-2024-026 | 9.89 | 11.18 | 26.77 | 47.03 |
| PBK-2025-017 | 12.03 | 19.39 | 31.35 | 41.11 |

TABLE 13
Relative cell viability (%)
Human Gastric cancer cell, SNU-719

| Concentration | | | | |
|---|---|---|---|---|
| Name | 50 μM | 30 μM | 10 μM | 1 μM |
| GO85 | 41.07 | 46.64 | 47.75 | 46.06 |
| GO88 | 45.55 | 44.41 | 47.80 | 49.89 |
| PBK-2024-003 | 25.59 | 33.59 | 36.90 | 37.13 |
| PBK-2024-010 | 35.57 | 40.39 | 35.87 | 36.14 |
| PBK-2024-026 | 31.61 | 38.08 | 36.04 | 39.45 |
| PBK-2025-016 | 34.95 | 35.52 | 35.91 | 41.25 |
| PBK-2025-017 | 24.98 | 31.61 | 35.50 | 35.93 |

TABLE 14
Relative cell viability (%)
Human Bladder cancer cell, T24

| Concentration | | | | |
|---|---|---|---|---|
| Name | 50 μM | 30 μM | 10 μM | 1 μM |
| GO78 | 29.32 | 29.73 | 30.42 | 28.34 |
| GO85 | 9.11 | 10.36 | 10.78 | 10.57 |
| GO86 | 29.34 | 30.42 | 31.17 | 28.15 |
| GO88 | 7.47 | 9.22 | 11.01 | 9.88 |
| GO89 | 8.75 | 9.57 | 9.29 | 7.92 |
| PBK-2024-010 | 9.46 | 9.53 | 10.08 | 10.04 |
| PBK-2024-021 | 6.92 | 7.58 | 7.06 | 7.59 |
| PBK-2024-026 | 6.64 | 6.90 | 7.79 | 7.49 |
| PBK-2025-002 | 9.04 | 9.95 | 10.98 | 9.91 |
| PBK-2025-017 | 7.23 | 7.55 | 7.63 | 8.87 |

TABLE 15
Relative cell viability (%)
Human Osteosarcoma, Saos-2

| Concentration | | | | |
|---|---|---|---|---|
| Name | 50 μM | 30 μM | 10 μM | 1 μM |
| GO78 | 2.60 | 19.77 | 33.69 | 28.36 |
| GO85 | −9.49 | 42.94 | 53.92 | 6.85 |
| GO86 | 36.73 | 16.00 | 24.98 | 36.73 |
| GO88 | 0.00 | 16.31 | 34.17 | 47.09 |

TABLE 16
Relative cell viability (%)
Human Head and Neck cancer, Fadu

| Concentration | | | | |
|---|---|---|---|---|
| Name | 50 μM | 30 μM | 10 μM | 1 μM |
| GO78 | 0.99 | 4.93 | 6.63 | 4.18 |
| GO86 | 4.78 | 8.51 | 9.68 | 2.08 |
| GO88 | 7.08 | 57.64 | 73.25 | 7.05 |
| GO89 | 9.44 | 41.53 | 29.27 | 4.15 |
| PBK-2024-003 | 2.79 | 8.66 | 11.26 | 31.30 |
| PBK-2024-004 | 3.31 | 2.30 | 0.22 | 29.18 |
| PBK-2025-001 | −0.70 | 2.94 | 5.89 | 1.22 |
| PBK-2025-002 | 18.18 | 59.98 | 74.11 | 29.84 |
| PBK-2025-011 | 5.02 | 7.34 | 19.91 | 10.04 |
| PBK-2025-012 | 31.37 | 26.70 | 28.87 | 32.04 |
| PBK-2025-014 | 7.05 | 23.42 | 26.55 | 20.78 |
| PBK-2025-015 | 7.69 | 10.56 | 17.30 | 24.93 |
| PBK-2025-017 | 9.56 | 27.92 | 39.97 | 37.43 |
| PBK-2025-018 | 18.65 | 32.41 | 33.03 | 24.97 |

TABLE 17
Relative cell viability (%)
Human multiple myeloma, KMS12PE

| Concentration | | | | |
|---|---|---|---|---|
| Name | 50 μM | 30 μM | 10 μM | 1 μM |
| GO15F | — | — | 41.72 | — |

TABLE 18
Relative cell viability (%)
Human multiple myeloma, KMS26

| Concentration | | | | |
|---|---|---|---|---|
| Name | 50 μM | 30 μM | 10 μM | 1 μM |
| GO15F | — | 43.15 | 44.43 | — |
| GO86 | — | — | — | 31.19 |

TABLE 19
Relative cell viability (%)
Human multiple myeloma, KMS34

| Concentration | | | | |
|---|---|---|---|---|
| Name | 50 μM | 30 μM | 10 μM | 1 μM |
| GO86 | — | — | — | 26.19 |

### Experiment Example 4: Apoptosis Experiment on Cancer Cell Lines

To confirm whether the compound of the present invention induces apoptosis in cancer cells, the following experiment was performed using human-derived B lymphoma cell line SU-DHL-8 (ATCC, Cat. #CRL-2961).

The cell line was cultured in RPMI-1640 medium containing 10% fetal bovine serum (FBS), 100 U/mL penicillin, and 100 μg/mL streptomycin at 37° C. under 5% CO₂ conditions using cells within 10-15 passages.

The cell culture medium was harvested, centrifuged at 1,300 rpm for 5 minutes, the supernatant was removed, and the cells were resuspended in the same medium.

The cells were then seeded into a 6-well plate at a concentration of 1.5 mL per well, adjusting to a final cell density of 1×10⁶ cells/well.

The compound of the present invention was added to each well at a final concentration of 30 M, and the control group was treated with 0.2% DMSO. The cells were then cultured at 37° C. and 5% CO₂ for 24 hours.

After the cells were cultured, they were collected and fluorescent staining was performed using the FITC Annexin Apoptosis Detection Kit I (BD Biosciences) according to the manufacturer's instructions.

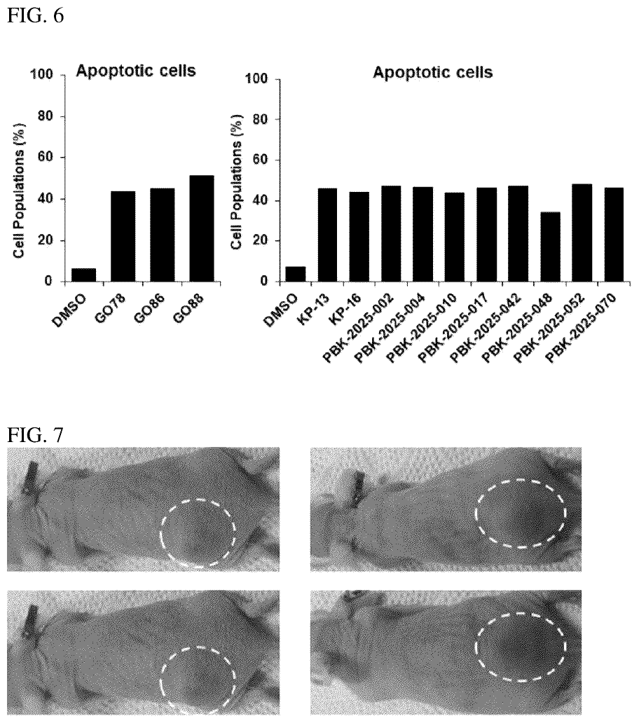

The stained cells were analyzed by flow cytometry and the results are shown in FIG. 6.

Annexin V binds to phosphatidylserine (PS), a phospholipid on the cell membrane, which is exposed to the extracellular space during the early stages of cell death. Propidium iodide (PI) is a dye that penetrates damaged cell membranes in late-stage apoptotic cells or necrotic cells and binds to nucleic acids.

Therefore, using Annexin V and PI together allows for the reliable differentiation of live cells, early-stage apoptotic cells, late-stage apoptotic cells, and necrotic cells, enabling a high-confidence distinction of cell death stages.

The extent of cell death was represented as a bar graph showing the sum of the percentages of cells located in the Annexin VHigh/PIHigh, Annexin VHigh/PILow, and Annexin VLow/PIHigh categories.

As a result, it was confirmed that the apoptosis rate in the group treated with the compound of the present invention was significantly increased compared to the control group, suggesting that the compound induces cell death in B lymphoma cells.

These results indicate that various compounds according to the present invention can be utilized as anticancer agents.

In other words, when the compounds according to the present invention were administered to lymphoma cancer cells, as confirmed in Example 1, the compounds inhibited the intracellular PDK1 (3-Phosphoinositide-dependent protein kinase-1) signaling pathway, thereby suppressing the cells' survival mechanisms and inducing apoptosis.

As a result, it was confirmed that early and late apoptosis, accompanied by the external exposure of phosphatidylserine and cell membrane damage, was effectively induced.

This suggests that the compounds according to the present invention selectively block the survival mechanism of lymphoma cells via PDK1, thereby performing an effective action for lymphoma treatment.

### Experimental Example 5: In Vivo Verification of Anticancer Efficacy Against B-Cell Lymphoma

To verify the anticancer efficacy of the compounds according to the present invention in vivo, an animal model (xenograft tumor model) was established by subcutaneously implanting human-derived B cell lymphoma cell line SU-DHL-8 (ATCC, Cat. #CRL-2961) into mice.

For this purpose, 7-week-old normal mice (BALB/c nude mice, female) were acclimatized in an animal facility for more than one week, followed by subcutaneous injection of 100 μL of PBS containing 8×10⁶ SU-DHL-8 cells into the back to induce tumors. Approximately 14 days later, the presence of tumors in the subcutaneous tissue was confirmed.

This is shown in FIG. 7. To verify the therapeutic efficacy of the compounds according to the present invention, tumor growth inhibition experiments were conducted using human-derived B lymphoma cell lines and the aforementioned animal model with SU-DHL-8 cells subcutaneously implanted.

From the point when the initial tumor volume of each animal model reached approximately 100 mm³, the compounds of the present invention, G048 (0.5 mg/kg), G088 (0.5 mg/kg), or the control substance (vehicle), were administered at a dose of 30 mg/kg once daily for 12 days via intratumoral injection.

Throughout the entire experimental period, body weight measurements were performed twice weekly on each mouse to assess toxicity, such as weight loss, caused by drug administration. Tumor size was measured using a vernier caliper in all directions and calculated using the formula “0.5× (width)×(length) (unit: mm³).”

As a result, the following common phenomena were observed in mice administered G048 and G088 in the subcutaneous tumor animal model.

First, mice administered the compound according to the present invention exhibited inhibited tumor growth at 14 days post-administration. In contrast, mice in the control group administered vehicle or no substance showed no therapeutic effect, with tumor growth continuing to increase.

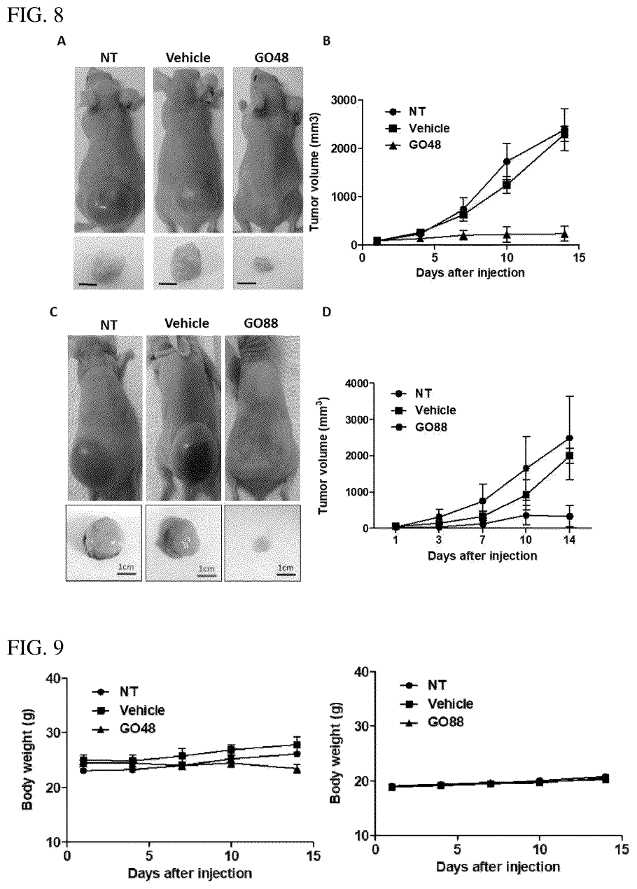

Photographs of these mice are shown in FIG. 8.

Additionally, the same pattern was confirmed through actual measurements using a Vernier caliper.

Furthermore, no weight loss was observed in mice administered the compounds according to the present invention until the end of the experiment, confirming that no weight changes due to drug toxicity occurred.

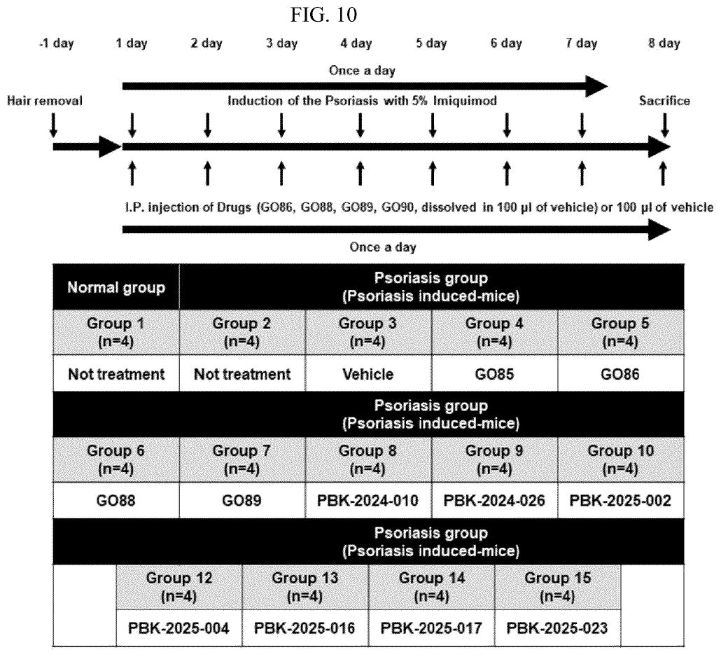

This is shown in FIG. 9.

Therefore, based on these results, it was verified that the compounds according to the present invention exhibit excellent anticancer efficacy in vivo when subcutaneous tumors are present.

In other words, based on the above results, it can be concluded that the compounds according to the present invention inhibit or antagonize the activity of PDK1 (3-Phosphoinositide-dependent protein kinase-1), thereby inhibiting the signaling pathway mediated by PDK1, and effectively suppressing cell proliferation, survival, and tumor formation.

Through these in vivo effects, the compounds demonstrated significant effects in inhibiting tumor growth and reducing tumor size, thereby confirming that the compounds according to the present invention possess excellent efficacy as anticancer therapeutic compositions.

### Experimental Example 6: In Vivo Treatment Effect Verification Experiment for Psoriasis

To verify the therapeutic effect of the compound according to the present invention on psoriasis, a psoriasis animal model was established as follows, and the compound according to the present invention was administered to evaluate the alleviation of skin lesions and changes in immune cell patterns.

For the establishment of the psoriasis animal model, BALB/c mice (7 weeks old, female, 20 g) were purchased from HanabioTech Co., Ltd. and acclimatized to the rearing environment for one week.

The schedule for establishing the psoriasis animal model is shown in FIG. 10.

Subsequently, the hair on the mouse back was shaved using a hair removal device for the first time, and then completely shaved using a depilatory cream (Nicrin Cream (80% thioglycolic acid, II Dong Pharmaceutical)) for the second time. The mice were then kept for 24 hours to confirm the absence of skin damage.

To verify the psoriasis prevention and therapeutic effects of the compounds according to the present invention, the following experimental groups were established: [Normal control group (Normal, Not Treatment), psoriasis-induced control group (Psoriasis, Not Treatment), psoriasis-induced vehicle control group (Psoriasis, Vehicle), experimental group (GO85, G086, G088, G089, PBK-2024-010, PBK-2024-026, PBK-2025-002, PBK-2025-004, PBK-2025-016, PBK-2025-017, PBK-2025-023)].

As shown in Table 20, psoriasis was induced in each group by applying Aldara cream (Imiquimod, 250 mg/dose) once daily at a dose of 62.5 mg per mouse for 7 consecutive days.

To compare and evaluate the psoriasis prevention and treatment effects of the compounds according to the present invention, for the drug-administered group, the compound was completely dissolved in DMSO at a concentration corresponding to 100 of the total dose, then diluted in a solvent mixture of Cremophor EL, PEG 400, and distilled water to achieve a final ratio ofDMSO:Cremophor EL:PEG 400:distilled water of 1:1:4:4 (v/v/v/v).

The prepared test solution was administered intraperitoneally at 100 μL per mouse once daily starting 24 hours after complete hair removal.

The solvent control group (Vehicle) received the same solvent (DMSO: Cremophor EL: PEG 400: distilled water=1:1:4:4 (v/v/v/v)) without the compound, administered intraperitoneally at 100 μL per mouse once daily.

TABLE 20

| | Imiquimod | | Number |
|---|---|---|---|
| Group | 62.5 mg | Intervention | of mouse |
| Normal | X | Depilation only | 4 |
| | | (No medication) | |
| Psoriasis_NT | ◯ | Depilation only | 4 |
| (Nottreatment) | | (No medication) | |
| Psoriasis_Vehicle | ◯ | solvent (DMSO:cremophorEL:PEG | 4 |
| | | 400:distilled water) = | |
| | | 1:1:4:4 (v/v/v/v) | |
| | | 100 μl/ once daily intraperitoneal | |
| | | injection | |
| Psoriasis_{—} | ◯ | solvent (DMSO:cremophorEL:PEG | 4 |
| GO85 | | 400:distilled water) = | |
| | | 1:1:4:4 (v/v/v/v) + 1 mg/kg GO85 | |
| | | 100 μl/ once daily intraperitoneal | |
| | | injection | |
| Psoriasis_{—} | ◯ | solvent (DMSO:cremophorEL:PEG | 4 |
| GO86 | | 400:distilled water) = | |
| | | 1:1:4:4 (v/v/v/v) + 2.5 mg/kg | |
| | | GO86 | |
| | | 100 μl/ once daily intraperitoneal | |
| | | injection | |
| Psoriasis_{—} | ◯ | solvent (DMSO:cremophorEL:PEG | 4 |
| GO88 | | 400:distilled water) = | |
| | | 1:1:4:4 (v/v/v/v) + 0.25 mg/kg | |
| | | GO88 | |
| | | 100 μl/ once daily intraperitoneal | |
| | | injection | |
| Psoriasis_{—} | ◯ | solvent (DMSO:cremophorEL:PEG | 4 |
| GO89 | | 400:distilled water) = | |
| | | 1:1:4:4 (v/v/v/v) + 2.5 mg/kg | |
| | | GO89 | |
| | | 100 μl/ once daily intraperitoneal | |
| | | injection | |
| Psoriasis_{—} | ◯ | solvent (DMSO:cremophorEL:PEG | 4 |
| PBK-2024-010 | | 400:distilled water) = | |
| | | 1:1:4:4 (v/v/v/v) + 0.5 mg/kg | |
| | | PBK-2024-010 | |
| | | 100 μl/ once daily intraperitoneal | |
| | | injection | |
| Psoriasis_{—} | ◯ | solvent (DMSO:cremophorEL:PEG | 4 |
| PBK-2024-026 | | 400:distilled water) = | |
| | | 1:1:4:4 (v/v/v/v) + 0.5 mg/kg | |
| | | PBK-2024-026 | |
| | | 100 μl/ once daily intraperitoneal | |
| | | injection | |
| Psoriasis_{—} | ◯ | solvent (DMSO:cremophorEL:PEG | 4 |
| PBK-2025-002 | | 400:distilled water) = | |
| | | 1:1:4:4 (v/v/v/v) + 0.5 mg/kg | |
| | | PBK-2025-002 | |
| | | 100 μl/ once daily intraperitoneal | |
| | | injection | |
| Psoriasis_{—} | ◯ | solvent (DMSO:cremophorEL:PEG | 4 |
| PBK-2025-004 | | 400:distilled water) = | |
| | | 1:1:4:4 (v/v/v/v) + 0.5 mg/kg | |
| | | PBK-2025-004 | |
| | | 100 μl/ once daily intraperitoneal | |
| | | injection | |
| Psoriasis_{—} | ◯ | solvent (DMSO:cremophorEL:PEG | 4 |
| PBK-2025-016 | | 400:distilled water) = | |
| | | 1:1:4:4 (v/v/v/v) + 0.5 mg/kg | |
| | | PBK-2025-016 | |
| | | 100 μl/ once daily intraperitoneal | |
| | | injection | |
| Psoriasis_{—} | ◯ | solvent (DMSO:cremophorEL:PEG | 4 |
| PBK-2025-017 | | 400:distilled water) = | |
| | | 1:1:4:4 (v/v/v/v) + 0.5 mg/kg | |
| | | PBK-2025-017 | |
| | | 100 μl/ once daily intraperitoneal | |
| | | injection | |
| Psoriasis_{—} | ◯ | solvent (DMSO:cremophorEL:PEG | 4 |
| PBK-2025-023 | | 400:distilled water) = | |
| | | 1:1:4:4 (v/v/v/v) + 0.5 mg/kg | |
| | | PBK-2025-023 | |
| | | 100 μl/ once daily intraperitoneal | |
| | | injection | |

Body weights of mice in each group were measured on days 1, 4, and 8 after the start of injection and psoriasis induction. From days 1 to 8, the extent of erythema, thickness, and scaling of the skin on the dorsal region of the mice were observed daily.

On day 8, mice were sacrificed, and skin tissue from the dorsal region was collected. This tissue was stained with H&E and subjected to histopathological analysis.

The results showed that in the group where psoriasis was induced alone (Psoriasis, Not Treatment) and the group that induced psoriasis and administered the vehicle solution (Psoriasis, Vehicle) showed significant hyperkeratosis, parakeratosis, irregular acanthosis, and infiltration of inflammatory cells, whereas the group administered the compound of the present invention exhibited mild or almost no such symptoms.

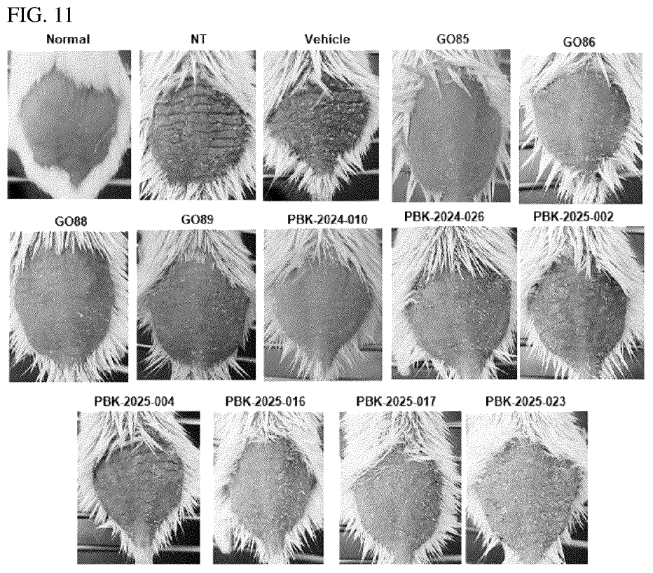

This is shown in FIG. 11.

Referring to FIG. 11, analysis of skin pathological tissues from mice with induced psoriasis using H&E staining revealed that the control group (Psoriasis, Not Treatment, and Vehicle) exhibited significant epidermal thickening, irregular acanthosis, and infiltration of inflammatory cells.

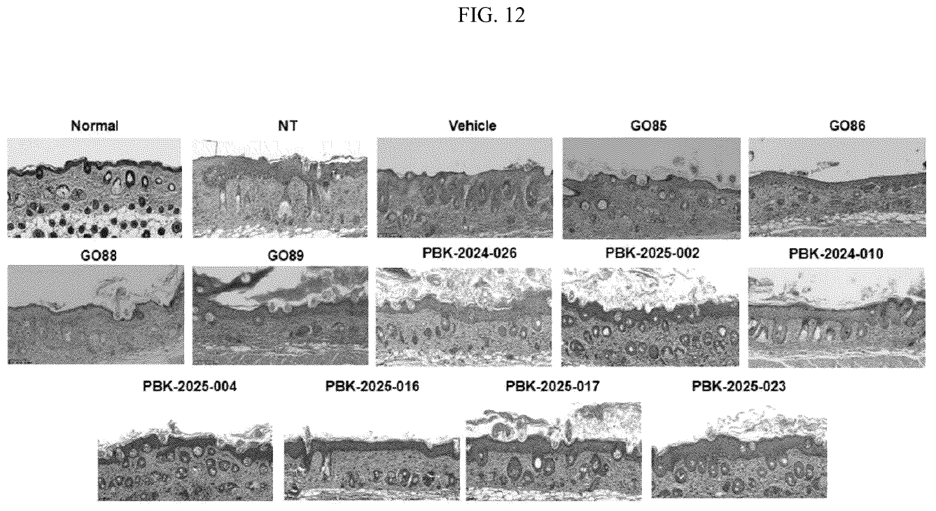

In contrast, as shown in FIG. 12, in the group treated with the compound of the present invention, the thickness of the epidermis decreased, and the degree of inflammatory cell infiltration and acanthosis were also alleviated.

FIG. 12 shows photographs of skin thickness taken at 8 days after administration of the compound of the present invention to assess the efficacy of psoriasis treatment in mice.

Based on these results, it is evident that the compound effectively suppresses chronic inflammation and epidermal hyperproliferation associated with psoriasis.

To evaluate the therapeutic effect in a psoriasis-induced mouse model, the PASI (Psoriasis Area and Severity Index) score was measured.

The PASI score is based on three criteria: erythema, scaling, and skin thickness, each scored from 0 (none) to 4 (severe). The scores for each criterion are summed to obtain a total score (12 points), which is used to compare treatment effects.

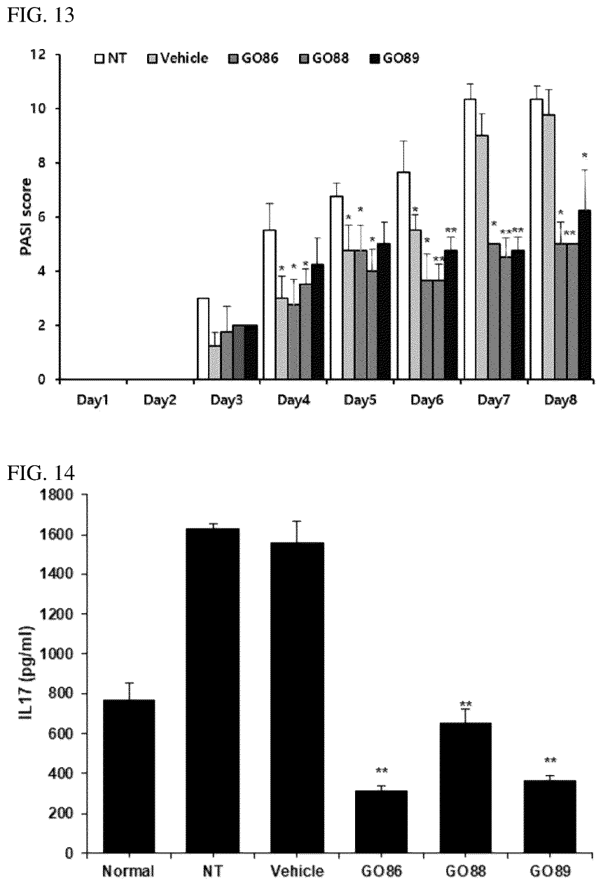

Observations were conducted daily at the same time, and the same observer visually assessed each criterion and recorded the scores, with the results presented in FIG. 13.

As shown in FIG. 13, in the group with psoriasis alone (Psoriasis, Not Treatment) and the group with psoriasis and vehicle injection (Psoriasis, Vehicle), the Psoriasis score increased continuously over time.

However, in the group treated with the compound of the present invention, the Psoriasis score increased up to day 5 and then remained stable, indicating that the compound has an effect of suppressing the worsening of psoriasis symptoms.

After the experiment (day 8), the mice were anesthetized by inhalation and blood was collected via the heart.

The collected blood was allowed to clot at room temperature, then centrifuged at 3,000 rpm for 10 minutes to separate the serum from the supernatant.

Using the collected serum, IL-17A concentrations were analyzed using the IL-17A Mouse ELISA kit (BioLegend) according to the manufacturer's manual in the GO86, GO88, and G089 treatment groups, which were evaluated to have excellent efficacy in improving dermatological symptoms.

The results are presented in FIG. 14.

All samples were measured in duplicate, and the absorbance was measured at 450 nm using a microplate reader.

As shown in FIG. 14, the IL-17A concentrations in the serum of the group induced with psoriasis only (Psoriasis, Not Treatment) and the group induced with psoriasis and administered with the injection solution solvent (Psoriasis, Vehicle) were significantly increased compared to the normal group.

On the other hand, the serum IL-17A concentrations in the experimental group administered with the composition of the present invention were significantly reduced (p<0.001).

This suggests that the compounds according to the present invention possess biological activity capable of inhibiting the secretion of IL-17A, a cytokine associated with psoriasis.

In other words, based on the above results, it was found that the compounds according to the present invention inhibit the expression of IL-17, a major cytokine that induces inflammatory responses in skin tissue, thereby regulating excessive immune responses and inflammatory responses and alleviating abnormal keratinization.

These results suggest that the compounds of the present invention exhibit excellent efficacy as therapeutic candidates capable of effectively inhibiting the progression of inflammatory skin diseases mediated by IL-17, particularly psoriasis.

### Experimental Example 7: Therapeutic Efficacy Against Systemic Lupus Erythematosus (SLE)

To verify the therapeutic efficacy of the compounds according to the present invention against systemic lupus erythematosus (SLE), a lupus animal model was established, and therapeutic efficacy verification experiments were conducted.

### (1) Establishment of a Lupus Animal Model and Changes in Spleen Size

The MRL/MpJ-Faslpr/J (hereinafter referred to as “MRL/lpr”) used in this experiment is one of the representative animal models for systemic lupus erythematosus (SLE), known to produce various autoantibodies, including antinuclear antibodies, due to the lpr (lymphoproliferation) gene.

Generally, female MRL/lpr develop systemic lupus erythematosus around 12 weeks of age, and approximately 50% of the animals die by 30 weeks of age.

In this invention, the period up to 9 weeks of age was considered the pre-disease stage, and the period after 23 weeks of age was considered the post-disease stage, and efficacy verification experiments using this model were conducted.

To obtain experimental animals, female MRL/lpr mice aged 6 weeks were imported from The Jackson Laboratory, and MRL/lpr mice were imported from The Jackson Laboratory and acclimatized for one week in an SPF (Specific Pathogen Free) facility maintained at a temperature of 21-24° C., humidity of 40-60%, and a 12-hour light-dark cycle (lights on at 7:00 AM, lights off at 7:00 PM).

Subsequently, female and male MRL/lpr mice were mated at a ratio of 2:1 in the same facility, and the female offspring were administered the compound G015F of the present invention or vehicle at a dose of 30 mg/kg once daily via intraperitoneal injection or oral administration from the age of 10 weeks until 23 weeks of age.

Urine samples were collected once a week from each individual and used for analytical experiments.

At 23 weeks of age, necropsy was performed on each MRL/lpr mouse, and serum, kidneys, and spleens were isolated and used for analytical experiments.

The size and weight of the separated spleens were measured, and it was observed that the spleens tended to enlarge due to the progression of lupus disease. In the group administered the compound according to the present invention, the size and weight of the spleens were relatively reduced.

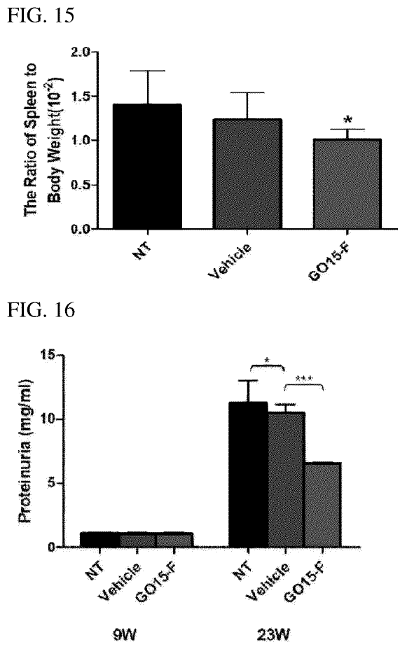

FIG. 15 is a graph showing the body weight of mice with lupus disease administered the compound according to the present invention, which was referenced.

This demonstrates that administering the compound according to the present invention regulates the overactivated autoimmune response in a lupus animal model, thereby normalizing the enlarged spleen size.

### (2) Proteinuria Measurement

The protein concentration in urine collected weekly from the control group or experimental group was analyzed using ‘Quick Start™ Bradford 1× Dye Reagent (Bio-Rad, Cat. #5000205).’

In each well of a 96-well plate, 5 μl of urine sample diluted with triple-distilled water and 250 μl of 1× dye reagent were added, reacted, and the absorbance was measured at 595 nm.

The results showed that the increase in protein concentration in urine was significantly higher in the MRL/lpr mice belonging to the control group, whereas the concentration in the group administered with G015F, the compound of the present invention, was significantly lower than that in the control group.

This is shown in FIG. 16.

These results confirm that the compound of the present invention has an effect of alleviating lupus-induced renal function deterioration and inhibiting proteinuria, and it is considered that it may also inhibit the progression of lupus nephritis.

### (3) Measurement of Anti-dsDNA IgG

The concentration of anti-nuclear antibodies (Anti-dsDNA Ab) was measured using the ‘Mouse Anti-dsDNA IgG ELISA Kit (Alpha Diagnostic International, Cat. #5120)’ on serum isolated at the time of necropsy in the lupus animal model.

Each mouse serum was diluted to a final concentration of 1/5000 with Working Sample Diluent (WSD) and Low NSB Sample Diluent (LNSD), followed by adding 100 μl of the diluted serum to wells coated with anti-dsDNA and incubating at room temperature for 60 minutes.

The wells were then washed four times with 200 μl of washing buffer, followed by the addition of 100 μl of Anti-Mouse IgG-HRP antibody, and the mixture was incubated at room temperature for 30 minutes.

Each well was washed four times with 200 μl of washing buffer, and 100 μl of 3,3′,5,5′-Tetramethylbenzidine (TMB) substrate solution was added to each well.

The degree of color change to blue in the wells containing the standard solution was checked, and after 15 minutes, 100 μl of stop solution was added to terminate the reaction.

After the reaction was complete, the optical density (OD) was measured at 450 nm.

The results showed that the concentration of anti-dsDNA IgG in the blood of mice was significantly lower in the experimental group treated with G015F compared to the control group (MRL/lpr).

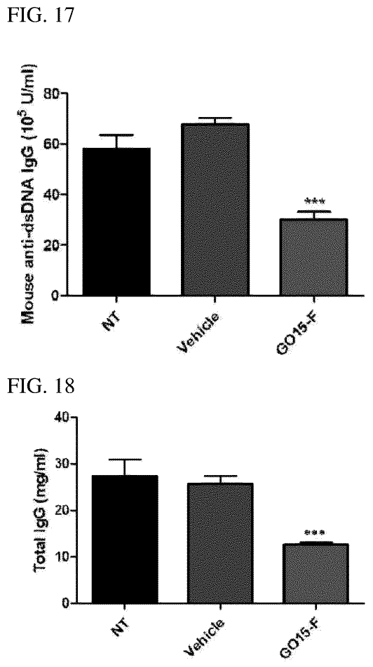

This is shown in FIG. 17.

Thus, it was confirmed that the compound according to the present invention exhibits a significant effect in inhibiting the production of anti-nuclear antibodies in a lupus animal model.

### (4) Measurement of Total IgG Concentration in Serum

The serum separated at the time of necropsy of the lupus animal model was analyzed using the ‘IgG (Total) Mouse Uncoated ELISA Kit (Invitrogen, Cat. #88-50400)’ to measure the concentration of total IgG.

Capture antibody (anti-mouse IgG) diluted 1/250 in coating buffer was added to each well of a 96-well plate (Nunc™ MaxiSorp™ ELISA Plates, Uncoated, BioLegend, Cat. #423501) at 100 μl per well, and the plate was incubated at 4° C. for 16 hours.

Each well was then washed twice with 400 μl of wash buffer, followed by 250 μl of blocking buffer, and incubated at room temperature. After 2 hours, each well was washed twice with 400 μl of wash buffer.

Add 90 μl of assay buffer, 10 μl of serum diluted in assay buffer, and 50 μl of detection antibody diluted 1:250 to each well in that order, then let it react at room temperature for 2 hours.

After that, each well was washed twice with 400 μl of washing solution, and 100 μl of TMB substrate solution was added to each well.

The degree of color change to blue in the wells containing the standard solution was checked, and after 15 minutes, 100 μl of stop solution was added to stop the reaction.

After the reaction was complete, the optical density (OD) was measured at 450 nm.

The results showed that the total IgG concentration in the blood of mice was significantly lower in the experimental group treated with G015F compared to the control group (MRL/lpr).

This is shown in FIG. 18.

This confirmed that the compound according to the present invention exhibits pharmacological efficacy in effectively inhibiting the excessive formation of total IgG in lupus, an autoimmune disease.

### (5) Measurement of Changes in the Ratio of Immune Cells in the Spleen of a Lupus Animal Model

To investigate the effect of G015F administration on changes in the ratio of immune cells in the spleen of a lupus animal model, the spleens of mice administered each compound were excised, cells were extracted, and the cells were suspended in MACS buffer (autoMACS Washing Solution, Miltenyi Biotec., Cat. #130-092-987).

For flow cytometry analysis, FACS tubes (5 mL round tubes, Falcon, Cat. #352052)were used. To prevent non-specific binding of antibodies, 1 μg/mL of anti-mouse CD16/CD32 (InVivoPlus anti-mouse CD16/CD32, BioXCell, Cat. #BP0307) for 30 minutes to prevent non-specific binding of the antibody, then centrifuge at 1,500 rpm for 5 minutes and resuspend in 100 μL of MACS buffer.

Subsequently, the experiment was conducted as follows depending on the type of immune cells to be analyzed.

For B cells, each FACS tube was stained with 1 μg/mL of FITC-conjugated anti-B220 antibody (eBioscience™, Cat. #11-0452-81) at 4° C. for 30 minutes, then washed with 2 mL of MACS buffer.

The tubes were then centrifuged at 1,500 rpm for 5 minutes, the supernatant was discarded, and the precipitated cells were fixed using 200 μL of 2% formaldehyde solution.

For T cells, each FACS tube was stained with 1 μg/mL PerCP-Cy5.5-conjugated anti-CD3 (Biolegend, Cat. #100218) at 4° C. for 30 minutes.

Each tube was then washed with 2 mL of MACS buffer, centrifuged at 1,500 rpm for 5 minutes, the supernatant was discarded, and the settled cells were fixed using 200 μL of 2% formaldehyde (Formaldehyde) solution.

For macrophages, each FACS tube was stained with 1 μg/mL of PE-conjugated anti-CD11b antibody (Biolegend, Cat. #101207) and PerCP-Cy5.5-conjugated anti-F4/80 (Biolegend, Cat. #123128) and incubated at 4° C. for 30 minutes. The tubes were then washed with 2 mL of MACS buffer.

Each tube was then centrifuged at 1,500 rpm for 5 minutes, the supernatant was discarded, and the settled cells were fixed using 200 μL of 2% formaldehyde solution.

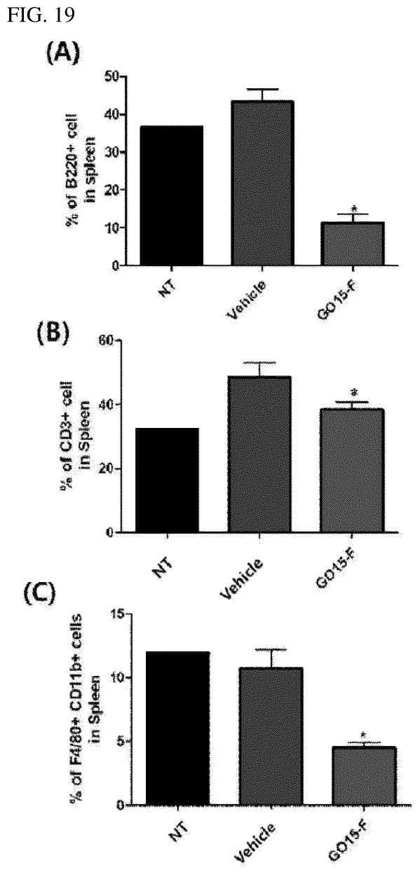

The results of flow cytometry analysis of the stained immune cells were shown in FIG. 19.

As shown in FIG. 19, the administration of G015F reduced the increased B cells, T cells, and macrophages in lupus, an autoimmune disease, due to disease progression.

This indicates that the administration of the compound according to the present invention inhibits PDK1, thereby regulating the overactivation of B cells, T cells, and macrophages in spleen cells of a lupus animal model, and is judged to possess excellent functionality as an autoimmune therapeutic agent.

### (6) Measurement of Changes in Cytokine Expression Levels in the Kidneys

To investigate the effects of compound G015F according to the present invention on systemic immune responses in a lupus animal model, gene expression analysis of cytokines in kidney tissue was performed as described in (1).

Each kidney tissue was used to isolate total RNA using TRIzol™ (Invitrozen, Cat. #15596018). RNA from each mouse was quantified at 1 g using a micro-quantitative spectrophotometer (ThermoFisher Scientific, ND2000) and cDNA was synthesized.

The synthesized cDNA from each mouse was quantified to a concentration of 50 ng using a micro-quantitative spectrophotometer, and qPCR was performed for the cytokines ‘IL-2, IL-6, IL-10, IL-17, TNF-α, IFN-γ′ using a SYBR green probe (CFX96, BIO-RAD).

The primers used for qPCR were designed as forward and reverse gene pairs that could bind complementarily to the nucleotide sequences of each mRNA.

Analysis of cytokine mRNA expression levels in kidney tissue from control and drug-treated groups revealed that the expression of mRNA for cytokines ‘IL-2, IL-6, IL-10, IL-17, TNF-α, and IFN-γ′ was significantly suppressed upon drug administration.

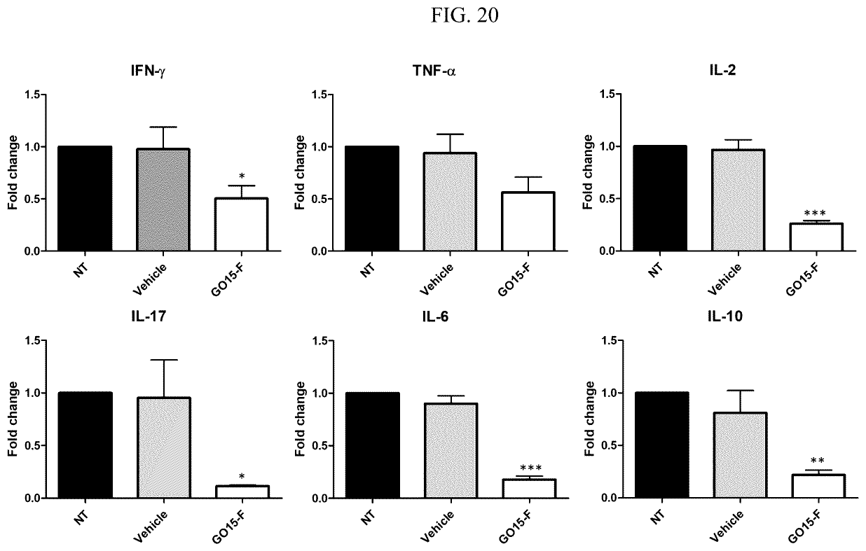

This is shown in FIG. 20.

Referring to FIG. 20, it was found that the administration of the compound according to the present invention suppressed the mRNA expression of ‘IL-2, IL-6, IL-10, IL-17, TNF-α, IFN-γ,’ which are major cytokines involved in inflammatory responses and disease progression in the kidney tissue of lupus animal models, thereby regulating excessive immune responses and inflammatory responses.

### (7) Renal Pathological Tissue Staining

To investigate the effects of drug administration on renal tissue in MRL/lpr mice, the tissue was fixed in 10% neutral buffered formalin at room temperature, followed by stepwise ethanol penetration for dehydration. The tissue was then embedded in xylene and paraffin.

The blocked tissues were sectioned into 4 m thick slices using a microtome, followed by deparaffinization, hydration, and staining.

Hematoxylin & Eosin staining, PAS staining, and Masson trichrome staining were performed for pathological evaluation of the tissues.

As a result, in the kidney tissue of MRL/lpr mice, which served as the control group, mesangial cells were proliferated, and the glomerular capillary loops were closed. In contrast, in mice administered G015F, the glomerular capillary loops were well maintained.

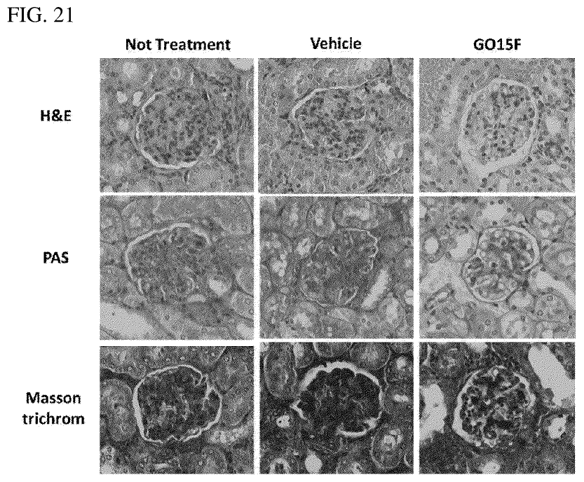

This is shown in FIG. 21.

These results confirm that the compound according to the present invention exhibits an effect of reducing glomerular inflammation in renal tissue in a lupus animal model.

### (8) IgG Immunofluorescence Staining of Renal Tissue

To determine the extent of IgG deposition in kidney tissue of MRL/lpr mice, immunofluorescence staining was performed using anti-IgG antibodies on paraffin-embedded kidney tissue sections, and the staining results were examined using a fluorescence microscope.

The results showed that IgG deposition was significantly higher in the kidney tissue of MRL/lpr mice in the control group, whereas the IgG deposition level was significantly lower in the group treated with G015F, the compound of the present invention.

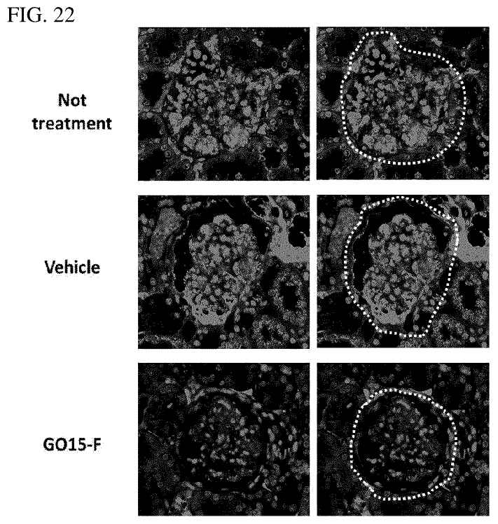

This is shown in FIG. 22.

These results confirm that administration of the compound according to the present invention effectively suppresses IgG deposition, which induces inflammatory responses in the glomeruli of kidney tissue, thereby ultimately leading to the alleviation of lupus symptoms.

Additionally, the compound of the present invention demonstrates therapeutic effects in a systemic lupus erythematosus animal model and exhibits pharmacological efficacy in inhibiting excessive proliferation of immune cells during this process.

### (9) Hair Loss Treatment

In a study involving 23-week-old female MRL/lpr mice exhibiting systemic lupus erythematosus and hair loss symptoms in the facial region, the compound GO15F of the present invention was administered intraperitoneally at a dose of 30 mg/kg once daily for repeated administration.

As a result, hair growth began on the facial area of the mice starting from 3 days after compound administration, and by 2 months after administration, significant hair regrowth was observed across the entire facial area.

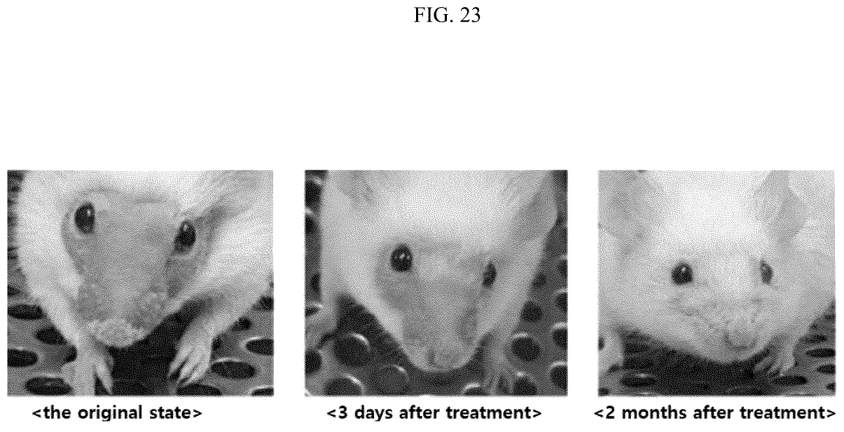

This is illustrated in FIG. 23.

Additionally, in 41-week-old female MRL/lpr mice, the compounds GO15F (30 mg/kg), G048 (1 mg/kg), and G088 (0.5 mg/kg) were administered intraperitoneally once daily. Over time, hair growth was confirmed on the facial region of these mice as well.

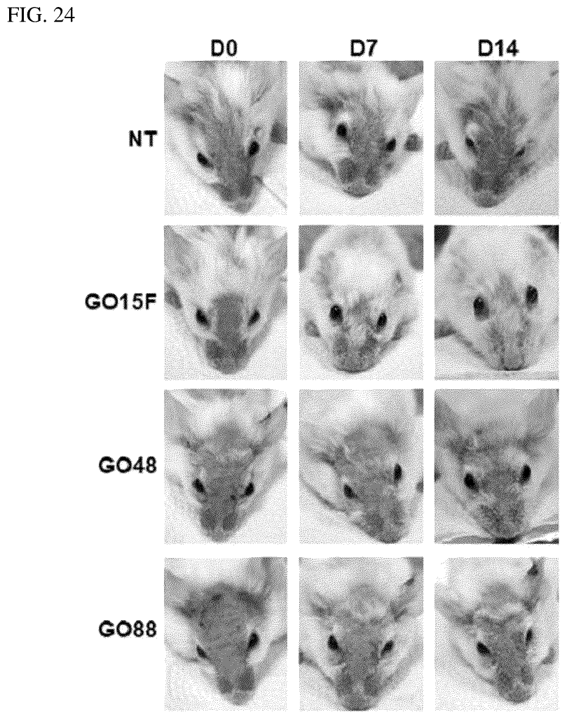

This is shown in FIG. 24.

### Confirmation of Hair Follicle Cell Proliferation

To confirm the proliferative activity of hair follicle cells in the skin tissue of these mice, the mice were sacrificed 14 days after administration of the compounds of the present invention, and Ki-67 immunohistochemistry (TIC) was performed.

For this purpose, the excised skin tissue was fixed in formalin, embedded in paraffin blocks, and stained with a Ki-67-specific antibody on tissue sections prepared as slides.

The results showed that, compared to the control group (NT), the Ki-67-positive reaction in cells surrounding the hair follicles was increased in the group treated with the compound of the present invention, indicating that the proliferation activity of hair follicle cells was significantly increased.

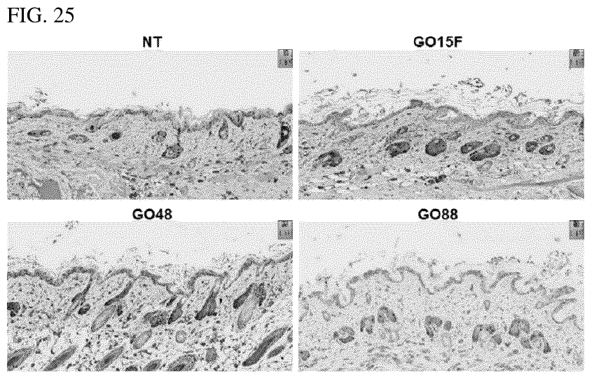

This is shown in FIG. 25.

Additionally, the number of hair follicles observed increased significantly alongside the increased Ki-67 positive response, suggesting that the compound contributes to hair follicle regeneration and proliferation activation.

In conclusion, despite the advanced age of the MRL/lpr mice (41 weeks old), the compound according to the present invention induced proliferation of hair follicle cells, increased the number of hair follicles, and promoted actual hair growth in skin where hair loss was induced by aging and inflammation, suggesting that it restores damaged hair follicle function or enhances cell proliferation activity.

Therefore, it is concluded that the compounds of the present invention are involved in the treatment of alopecia symptoms caused by inflammation in systemic lupus erythematosus, and based on the results observed in aged lupus mice, the compounds of the present invention are considered to have potential for use in anti-aging or rejuvenation therapy through the inhibition of PDK1.

Experimental Example 8: In vivo Verification of Therapeutic Effects on Rheumatoid Arthritis

To verify the therapeutic effect of the compounds of the present invention on rheumatoid arthritis, a rheumatoid arthritis animal model was established as follows, and the compounds of the present invention were administered to evaluate the reduction in paw swelling and joint damage.

To establish a rheumatoid arthritis mouse animal model, DBA/1 mice (7 weeks old, 20 g, male) were purchased from HanabioTech Co., Ltd. and acclimatized to the experimental environment for one week.

Subsequently, in mice excluding the Normal group, an ampoule containing a 1:1 mixture of Bovine type 2 collagen 2 mg/ml (Chondrex, #20022) and Complete Freund's Adjuvant (CFA) 1 mg/ml (Chondrex, #7008) was subcutaneously injected into the mouse tail at 100 μl to induce rheumatoid arthritis.

Three weeks later, an ampoule containing a 1:1 mixture of bovine type 2 collagen 2 mg/ml (Chondrex, #20022) and Incomplete Freund's Adjuvant (IFA) (Chondrex, #7002) was subcutaneously injected into the tails of the mice at a dose of 100 l.

After confirming that the paw swelling had increased to a certain extent, an experimental group was established to verify the rheumatoid treatment effect of the compound according to the present invention.

To compare the rheumatoid treatment effects of the compound according to the present invention, for the drug-treated group, the compound was completely dissolved in DMSO at 10% of the administered dose per mouse, and the final mixture was diluted with a Cremophor EL-PEG 400-distilled water mixture to achieve a ratio of DMSO:Cremophor EL:PEG 400:distilled water of 1:1:4:4 (v/v/v/v, volume ratio).

Details are summarized in Table 29 below.

The injection solution was administered intraperitoneally at a dose of 100 μL per mouse once daily when the paw swelling of the mice reached a certain level.

For the vehicle control group, the injection solution excluding the drug (DMSO:Cremophor EL:PEG 400:distilled water=1:1:4:4 (v/v/v/v)) was administered via intraperitoneal injection at 100 μL per day once daily.

TABLE 21

| | Induction of | | |
|---|---|---|---|
| | Rheumatoid | | Number |
| Group | arthritis | Intervention | of mouse |
| Normal | X | No medication | 4 |
| Rheumatoid | ◯ | No medication | 4 |
| arthritis_NT | | | |
| (Nottreatment) | | | |
| Rheumatoid | ◯ | Solvent(DMSO:cremophorEL:PEG | 4 |
| arthritis_{—} | | 400:Distilled Water) = | |
| Vehicle | | 1:1:4:4 (v/v/v/v) | |
| | | 100 μl/ once daily intraperitoneal | |
| | | injection | |
| Rheumatoid | ◯ | Solvent(DMSO:cremophorEL:PEG | 4 |
| arthritis_{—} | | 400:Distilled Water) = | |
| GO15F | | 1:1:4:4 (v/v/v/v) + 30 mg/kg GO15F | |
| | | 100 μl/ once daily intraperitoneal | |
| | | injection | |
| Rheumatoid | ◯ | Solvent(DMSO:cremophorEL:PEG | 4 |
| arthritis_{—} | | 400:Distilled Water) = | |
| GO48 | | 1:1:4:4 (v/v/v/v) + 0.5 mg/kg GO48 | |
| | | 100 μl/ once daily intraperitoneal | |
| | | injection | |
| Rheumatoid | ◯ | Solvent(DMSO:cremophorEL:PEG | 4 |
| arthritis_{—} | | 400:Distilled Water) = | |
| GO77 | | 1:1:4:4 (v/v/v/v) + 0.5 mg/kg GO77 | |
| | | 100 μl/ once daily intraperitoneal | |
| | | injection | |
| Rheumatoid | ◯ | Solvent(DMSO:cremophorEL:PEG | 4 |
| arthritis_{—} | | 400:Distilled Water) = | |
| GO78 | | 1:1:4:4 (v/v/v/v) + 1 mg/kg GO78 | |
| | | 100 μl/ once daily intraperitoneal | |
| | | injection | |
| Rheumatoid | ◯ | Solvent(DMSO:cremophorEL:PEG | 4 |
| arthritis_{—} | | 400:Distilled Water) = | |
| GO86 | | 1:1:4:4 (v/v/v/v) + 2.5 mg/kg GO86 | |
| | | 100 μl/ once daily intraperitoneal | |
| | | injection | |
| Rheumatoid | ◯ | Solvent(DMSO:cremophorEL:PEG | 4 |
| arthritis_{—} | | 400:Distilled Water) = | |
| GO88 | | 1:1:4:4 (v/v/v/v) + 0.5 mg/kg GO88 | |
| | | 100 μl/ once daily intraperitoneal | |
| | | injection | |

Twenty-one days after the start of injection, the thickness of the paws of mice in each group was measured.

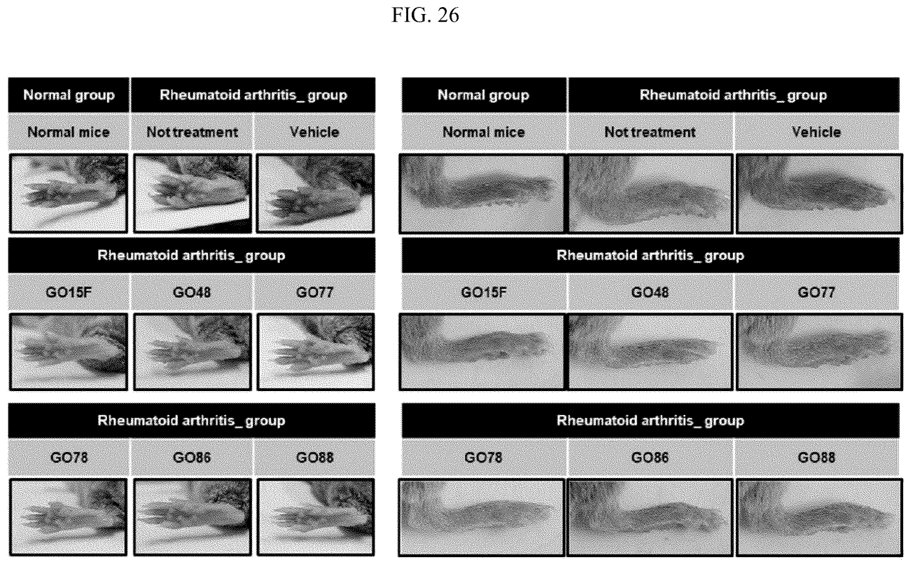

This is shown in FIG. 26.

The results showed that in the group induced with rheumatoid arthritis alone (CIA, Not Treatment) and the group where the injection solution solvent was administered into the peritoneal cavity simultaneously with inducing rheumatoid arthritis (CIA, Vehicle), severe paw swelling and significant infiltration of inflammatory cells were observed. However, in mice administered the compound according to the present invention, such symptoms were found to be mild.

Additionally, when measuring paw thickness using a Vernier caliper, a reduction in paw swelling was observed in the group treated with the compound of the present invention compared to the control groups (NT and Vehicle).

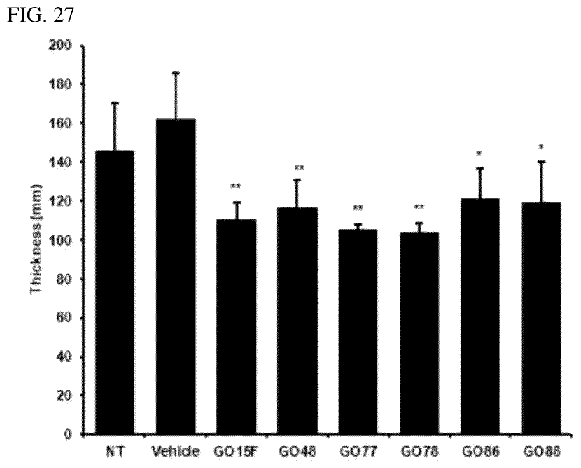

The measurement results are shown in FIG. 27.

### Joint Observation

After the experiment, the paw joint tissues of the experimental animals induced with rheumatoid arthritis were excised, fixed in 10% neutral formalin, undergo decalcification, and paraffin blocks were prepared. The blocks were sectioned at a thickness of 5 μm and stained with H&E and Safranin 0.

H&E staining results showed that in the control group (Not treat and Vehicle groups), the paw joint areas were hypertrophied compared to the Normal group, with numerous inflammatory cell infiltrates observed, and damage to cartilage and bone tissue was confirmed.

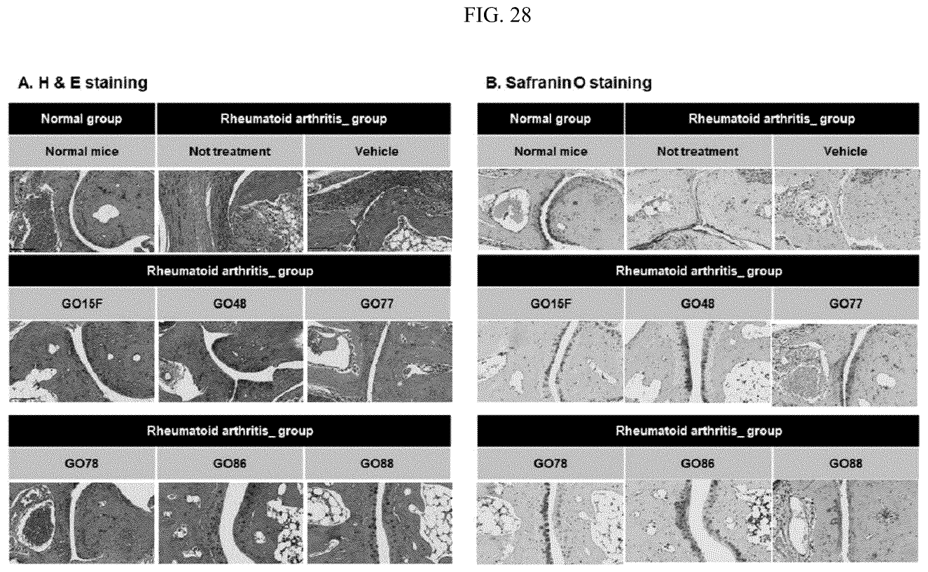

These results are shown in FIG. 28.

In contrast, in the group treated with the compound of the present invention, hypertrophy of cartilage tissue and inflammatory cell infiltration were significantly reduced, and damage to cartilage and bone tissue was minimized, showing a relatively well-preserved tissue appearance.

Additionally, Safranin O staining revealed that in the control group, staining intensity was significantly reduced or lost due to the loss of proteoglycans in the cartilage, whereas in the group treated with the composition of the present invention, strong red staining was maintained, and the cartilage layer structure was well preserved, confirming that proteoglycan content was preserved.

### Cytokine Inhibition Confirmation

After the experiment (day 8), mice were anesthetized by inhalation and blood was collected via the heart.

The collected blood was allowed to clot at room temperature, then centrifuged at 3,000 rpm for 10 minutes to separate the serum.

The collected serum was analyzed using a commercially available IL-17A, TNF-α Mouse ELISA kit (BioLegend, 436204, 430916) according to the manufacturer's manual.

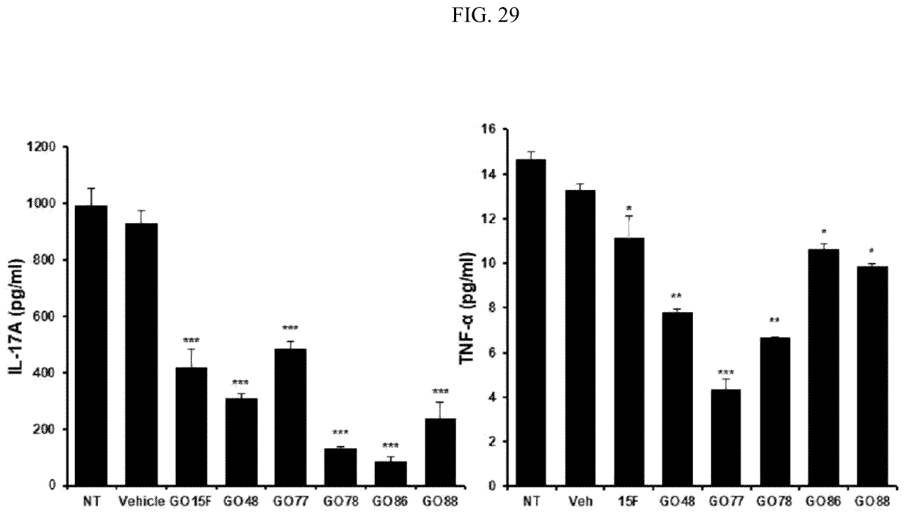

The analysis results are shown in FIG. 29.

All samples were measured in duplicate, and the absorbance was measured at 450 nm using a microplate reader.

The analysis results showed that the serum levels of IL-17A and TNF-α were elevated in the rheumatoid arthritis-induced group (Not Treatment) and the rheumatoid arthritis-induced group treated with the vehicle solution (Vehicle). the experimental group treated with the compound of the present invention showed a relative decrease (*: p<0.05, **: p<0.01, ***: p<0.001), suggesting that the compound of the present invention possesses physiological activity capable of inhibiting the secretion of IL-17A and TNF-α, which are rheumatoid arthritis-related cytokines.

This result demonstrates that the composition of the present invention effectively suppresses joint damage and inflammatory responses at the tissue level in an animal model of rheumatoid arthritis by reducing inflammatory cytokines, suggesting that the compound of the present invention may effectively mitigate the progression of rheumatoid arthritis.

These results provide evidence supporting the potential of the compounds of the present invention for use as pharmaceutical compositions for the prevention or treatment of rheumatoid arthritis.

### Experimental Example 9: Metastasis Inhibition Experiment on Colon Cancer Cell Lines

To evaluate the metastasis inhibitory efficacy of the compounds of the present invention on HCT-116 colon cancer cell lines, an Invasion Assay using Matrigel was performed to assess the cells' invasion ability.

First, Matrigel was thawed on ice in RPMI-1640 medium without FBS, then diluted with DMEM at a ratio of 1:20.

The diluted Matrigel mixture was applied to the upper chamber at 100 μl per well, and 750 μl of RPMI-1640 without FBS was added to the lower chamber.

The plates were then incubated at 37° C. for 1 hour to allow the Matrigel to solidify.

After coating, the Matrigel mixture in the upper chamber was removed using a pipette, and the chamber was washed with RPMI without FBS.

Human-derived colorectal cancer cell line HCT-116, obtained from the Korean Cell Bank, was cultured in RPMI-1640 medium containing 10% FBS.

Following stabilization, the cells were harvested using Trypsin-EDTA treatment, centrifuged at 1,200 rpm for 3 minutes, the supernatant was removed, and the cells were resuspended in the same medium. the medium in the lower chamber was replaced with 750 μl of RPMI-1640 supplemented with 10% FBS, and the cells were seeded into the upper chamber at a density of 5×104 cells/well.

The cells were cultured for 24 hours after attachment, and then the compounds according to the present invention (G078, G085, G086, G088, G089, PBK-2024-010, PBK-2025-010, PBK-2025-013, PBK-2025-016) and the control substance DMSO were diluted and added to the upper chamber after removing the medium.

Additionally, the medium in the lower chamber was replaced with fresh RPMI-1640 containing FBS, and the culture was continued.

After an additional 24 hours, the medium was removed, and 1 ml of methanol was added for 10 minutes to fix the cells.

Subsequently, 1 ml of Hematoxylin was added and reacted for 5 minutes, followed by 1 ml of Eosin, which was reacted for 30 seconds. The sample was then washed with distilled water (DW).

After staining was complete, any remaining cells in the upper chamber were removed. Finally, the infiltrated cells were observed under a microscope, photographed, and the degree of infiltration was analyzed.

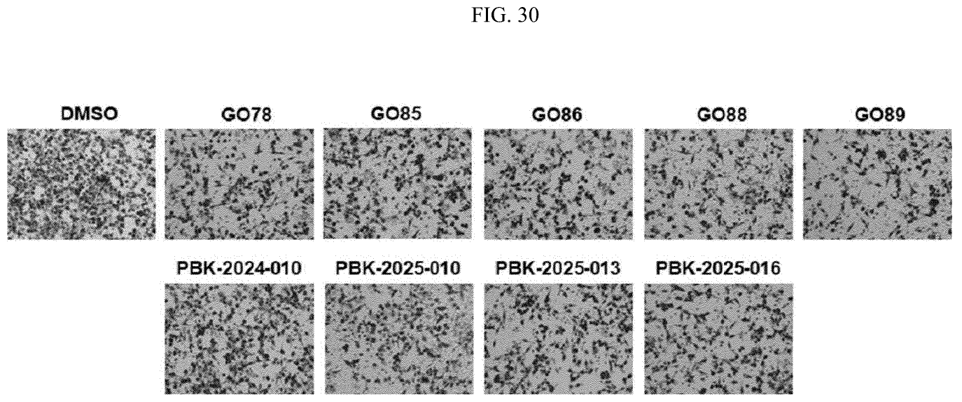

This image is shown in FIG. 30.

Referring to FIG. 30,

the results of staining the infiltrated cells show that the number of infiltrated cells in the treatment group (G078, G085, G086, G088, G089, PBK-2024-010, PBK-2025-010, PBK-2025-013, PBK-2025-016) showed a reduction in the number of infiltrated cells.

This result, confirmed through an invasion assay, demonstrates that the compounds of the present invention inhibit the invasive ability of cancer cells at the cellular level, thereby effectively reducing their invasiveness.

This indicates that the compounds of the present invention can be utilized as compositions for inhibiting cancer metastasis.

### Experimental Example 10: In Vivo Evaluation of Anticancer Efficacy Against Human-Derived Colorectal Cancer Cells

To verify the efficacy of the compounds according to the present invention, which have been validated for therapeutic efficacy at the cancer cell level, an animal model (xenograft tumor model) was established by subcutaneously implanting human-derived colorectal cancer cells, HCT116 (Korea Cell Line Bank; KCLB, Cat. #10247), into animals.

For this purpose, 7-week-old normal mice (BALB/c nude mice, female) were acclimatized in an animal facility for more than one week, followed by subcutaneous injection of 100 μL of PBS containing 5×106 HCT116 cells into the dorsal region to induce tumors. Approximately 14 days later, the presence of tumors in the subcutaneous tissue was confirmed.

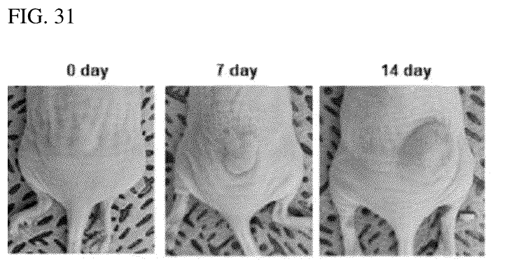

This is shown in FIG. 31.

### Verification of Tumor Growth Inhibition

To verify the therapeutic efficacy of the compounds according to the present invention, tumor growth inhibition (TGI) experiments were conducted using the aforementioned animal model in which human-derived colorectal cancer cells (HCT116) were subcutaneously implanted.

From the point when the initial tumor volume of each animal model reached approximately 100 mm³, the compounds of the present invention, G048 (0.5 mg/kg), G078 (1 mg/kg), and G086 (5 mg/kg), or the control substance (vehicle) were administered intraperitoneally once daily for 1 day.

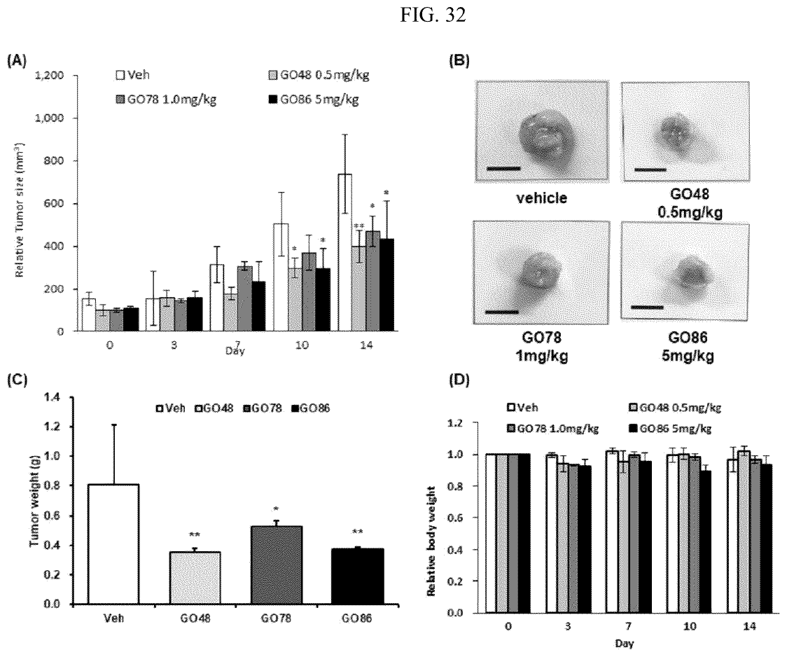

This is shown in FIG. 32.

FIG. 32 is a diagram showing the results of experiments to inhibit tumor growth in mice implanted with human-derived colorectal cancer cell lines treated with the compounds according to the present invention. During the entire experimental period, body weight measurements were performed twice weekly for each mouse to confirm toxicity such as weight loss caused by drug administration.

Tumor size was measured using a vernier caliper in each direction and calculated using the formula “0.5×(width)²×(length) (unit: mm3).”

Referring to FIG. 32, when G048 (0.5 mg/kg), G078 (1 mg/kg), and G086 (5 mg/kg) were administered to the subcutaneous tumor animal model, the following common phenomena were observed.

First, mice administered the compound according to the present invention showed inhibited tumor growth at 14 days post-administration, while mice in the control group administered vehicle showed continued tumor growth without therapeutic effects.

Additionally, the weight of the excised tumors was measured to quantitatively evaluate the tumor growth inhibitory effect, and it was confirmed that the size and weight of the excised tumors were reduced in the group administered the compounds of the present invention compared to the control group. Visual differences in size were also clearly observable through photography.

Furthermore, no weight loss was observed in mice administered the compound according to the present invention until the end of the experiment, confirming that no weight changes due to drug toxicity occurred.

Therefore, it is concluded that the compound according to the present invention has therapeutic effects against colorectal cancer.

### Experimental Example 11: Immunosuppressive Mechanism via a Sensitized Animal Model

To verify the antibody production inhibitory effect of the compound according to the present invention, a sensitized animal model was established as described below, and experimental groups were set as shown in Table 30 to evaluate the concentration of total IgG in serum and the changes in each immune cell in the spleen.

TABLE 22

| | Sensitization | |
|---|---|---|
| | animal model | |
| Group | induction | Dose and number of daily administrations |
| Normal | X | No administration of compound or control |
| | | substance |
| NT | ◯ | No administration of compound or control |
| | | substance |
| Vehicle | | Solvent 100 μl administered intraperitoneally |
| | | once daily |
| | | (Solvent: DMSO, Cremophor EL, PEG400, |
| | | DW mixed in a volume ratio of 1:1:4:4) |
| GO15F | | 30 mg/kg administered once daily by |
| | | intraperitoneal injection |
| | | (Dissolved in 100 μl of solvent with the same |
| | | composition as the vehicle and injected) |
| GO78 | | Administer 1 mg/kg once daily by |
| | | intraperitoneal injection |
| | | (Dissolve in 100 μl of the same solvent as the |
| | | vehicle and inject) |
| GO88 | | Administer 0.5 mg/kg once daily by |
| | | intraperitoneal injection |
| | | (Dissolve in 100 μl of the same solvent as the |
| | | vehicle and inject) |

To establish a sensitized mouse animal model, C57BL/6 and BALB/c mice (7 weeks old, 20 g, female) were purchased from HanabioTech Co., Ltd. and acclimatized to the experimental environment for one week.

After one week, spleen cells were isolated from BALB/c mice, suspended in PBS, and intraperitoneally injected into C57BL/6 mice at a dose of 1×107 cells per mouse. This procedure was repeated three times at two-week intervals to induce the sensitized animal model.

Six weeks after the first spleen cell injection, blood was collected from C57BL/6 mice, and the following method was used to measure the total IgG levels in serum to confirm the establishment of the mouse sensitization model.

Mouse blood was centrifuged at 4000 rpm for 10 minutes, and the supernatant was separated. The concentration of total IgG in the serum was measured using the IgG (Total) Mouse Uncoated ELISA Kit (Invitrogen, Cat. #88-50400).

Capture antibody (anti-mouse IgG) diluted 1/250 in coating buffer was added to each well of a 96-well plate (Nunc™ MaxiSorp™ ELISA Plates, Uncoated, BioLegend, Cat. #423501) at 100 μl per well, and the plate was incubated at 4° C. for 16 hours.

Each well was then washed twice with 400 μl of wash buffer, followed by 250 μl of blocking buffer, and incubated at room temperature. After 2 hours, each well was washed twice with 400 μl of wash buffer.

Add 90 μl of assay buffer, 10 μl of serum diluted in assay buffer, and 50 μl of detection antibody diluted 1/250 in each well in that order, then incubate at room temperature for 2 hours.

After that, each well was washed twice with 400 μl of washing solution, and 100 μl of TMB substrate solution was added to each well. The degree of color change to blue in the wells containing the standard solution was checked, and after 15 minutes, 100 μl of stop solution was added to terminate the reaction.

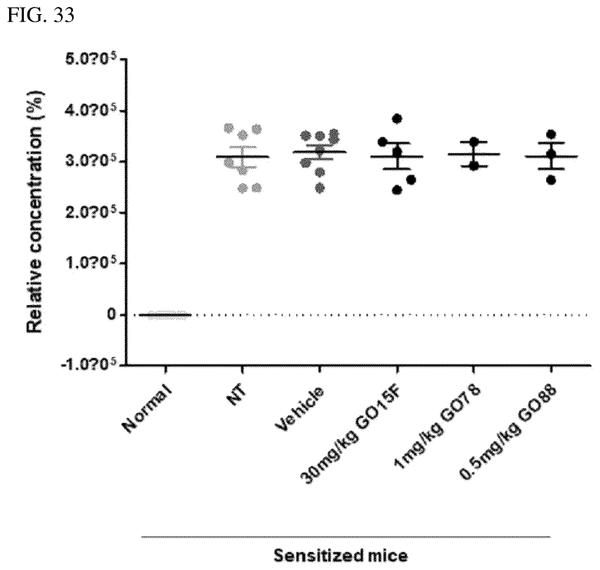

After the reaction was complete, the optical density (OD) was measured at 450 nm, and the results were presented in FIG. 33.

FIG. 33 shows a graph of the relative concentration values of total IgG concentrations in serum from normal mice and mice induced with a sensitized animal model.

Here, the relative concentration value represents the percentage of the IgG concentration in the sample serum relative to the IgG concentration in the serum of normal mice.

The IgG concentration in the serum of mice (sensitized mice) injected with the antigen (spleen cells from BalB/c mice) was approximately 3×105 times higher than that in mice (normal mice) not injected with the antigen, confirming that an adaptive immune induction animal model was successfully established.

Subsequently, the compound of the present invention or a control substance was administered intraperitoneally once daily to the sensitized animal model, and 8 days after the start of administration, the antigen (1×107 spleen cells from BalB/c mice) was injected into the peritoneal cavity to induce antibody production and immune cell activity. At 11 days after administration, all mice were necropsied, and serum and spleen were isolated for analysis.

To investigate the effect of the compound administered in this invention on changes in the ratio of immune cells in sensitized animal models, spleen cells were extracted from mice administered each compound and suspended in MACS buffer (autoMACS Washing Solution, Miltenyi Biotec., Cat. #130-092-987).

For flow cytometry analysis, FACS tubes (5 mL round tubes, Falcon, Cat. #352052)were used. To prevent non-specific binding of antibodies, 1 μg/mL of anti-mouse CD16/CD32 (InVivoPlus anti-mouse CD16/CD32, BioXCell, Cat. #BP0307)was added to each tube to prevent non-specific binding of the antibody. After incubating for 30 minutes, the tubes were centrifuged at 1,500 rpm for 5 minutes and resuspended in 100 μL of MACS buffer.

Subsequently, depending on the type of immune cells to be analyzed, the spleen cells were stained with antibodies specific for fluorescent markers labeled with different wavelengths, followed by flow cytometry analysis.

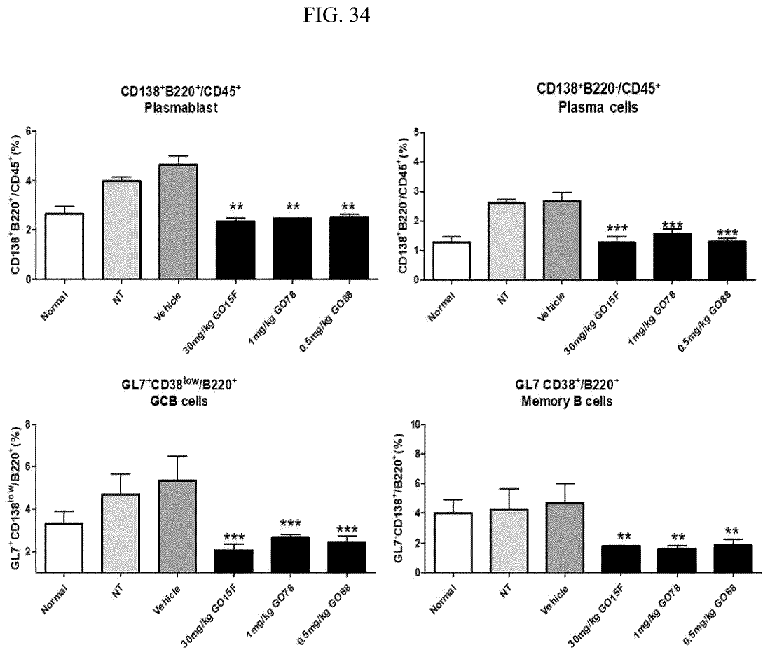

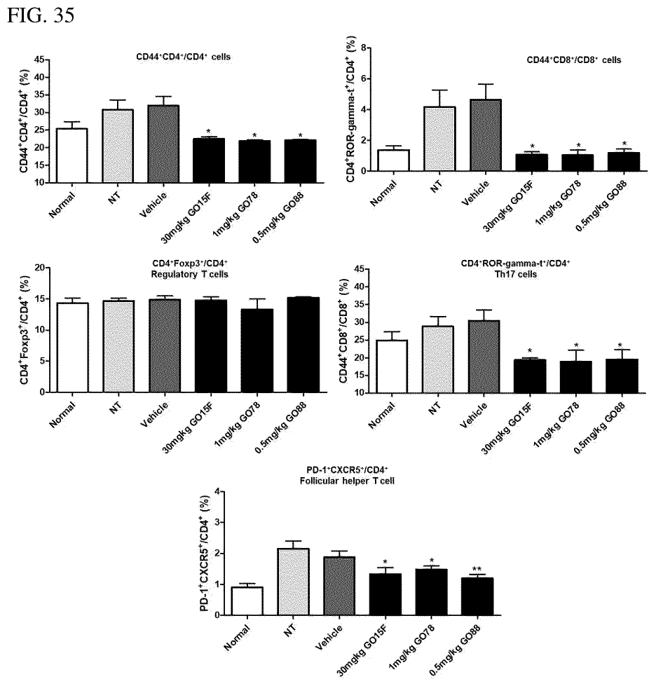

FIGS. 34 and 35 are graphs showing the results of analyzing changes in B cell and T cell subtypes within spleen cells following administration of the compound or control substance according to the present invention to a sensitized animal model.

Referring to the B cell analysis results in FIG. 34, the ratio of CD138+B220+ and CD138+B220—cells, which were selected based on CD45+, significantly decreased.

These cell populations correspond to differentiated plasma cells and their precursor cells, respectively, and are closely associated with the production of autoantibodies.

Additionally, GL7+CD38-/B220+ and GL7+CD38+/B220+ cells also showed a significant decrease. These cells are germinal center B cells associated with antigen-specific responses, antibody class switching, and the formation of memory B cells.

These results indicate that the compound of the present invention relatively reduces the number of B cell subtypes associated with pathological antibody production and B cell hyperactivity.

This suggests that the compound may serve as a therapeutic agent for regulating pathological B cell responses in conditions such as systemic lupus erythematosus (SLE), rheumatoid arthritis (RA), IgG4-related diseases, allograft rejection (AMR), and multiple myeloma, where autoantibodies and pathological antibodies are major pathological factors.

Additionally, as shown in FIG. 35, T cell analysis results indicate a decrease in CD44+CD4+/CD4+(memory helper T cells) and CD44+CD8+/CD8+(memory cytotoxic T cells).

Furthermore, CD4+RORγt+/CD4+ cells (Th17 cells) and PD-1+CXCR5+/CD4+ cells (T follicular helper cells) were also significantly reduced.

Th17 cells play a crucial role in autoimmune and inflammatory diseases by secreting inflammatory cytokines (e.g., IL-17), while Tfh cells are involved in inducing antibody production by B cells.

In contrast, CD4+Foxp3+/CD4+regulatory T cells (Tregs), which maintain immune homeostasis and suppress autoimmunity, did not show a significant decrease following treatment with the compound.

These results indicate that the compound of the present invention significantly reduces the proportion of T cell subtypes that play important roles in inflammatory and autoimmune pathologies.

Therefore, the compound is considered to have potential as an immunomodulatory agent that can selectively act on diseases caused by the overactivation of Th17 and Tfh cells, such as inflammatory bowel disease (IBD), multiple sclerosis (MS), psoriasis, rheumatoid arthritis (RA), and systemic lupus erythematosus (SLE).

### Measurement of Total IgG Concentration in Serum

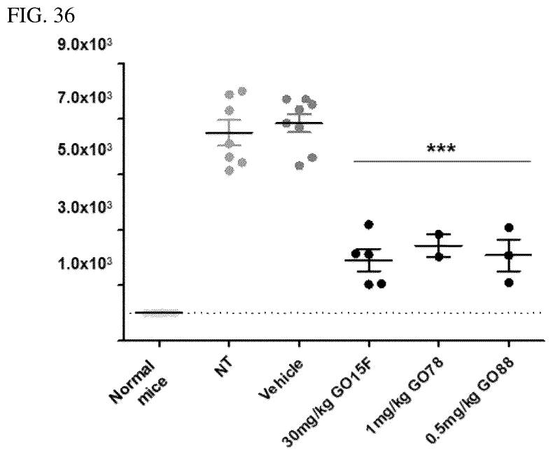

FIG. 36 shows a graph of the total IgG concentration in serum obtained from a sensitized animal model during necropsy and in serum from normal mice, expressed as relative concentration values.

Based on this, it was confirmed that the IgG concentration in serum from mice administered the compound according to the present invention was significantly lower than that in mice administered the control substance, indicating a reduction in actual antibody production.

Based on the above, it was confirmed that the compound according to the present invention inhibits the activity of specific immune cells involved in antibody formation, thereby ultimately inhibiting antibody production.

In other words, the compounds of the present invention selectively reduce pathologically activated cell populations among B cells and T cell subtypes, making them potentially effective as immune modulators for various immune-related diseases such as autoimmune diseases, inflammatory diseases, transplant rejection, GVHTD, and cancerous diseases.

Experimental Example 12: In vivo skin graft immune suppression efficacy experiment

To evaluate the immune-suppressive effects of the compounds according to the present invention, a skin graft animal model was established, and the efficacy of the compounds was analyzed in the model.

First, female BALB/c mice aged 7 weeks and weighing approximately 20 g were used as donor animals, and female C57BL/6 mice of the same age and weight were used as recipient animals. All experimental animals were supplied by HanabioTech Co., Ltd.

Prior to the experiment, the animals were acclimatized in SPF-certified facilities for one week and then used in the experiment. The housing conditions were maintained at a temperature of 22±2° C., relative humidity of 55+10%, and a 12-hour light-dark cycle.

To establish the skin graft model, a anesthetic solution consisting of ketamine, rumen, and PBS in a volume ratio of 1:1:3 was administered intraperitoneally to C57BL/6 mice to induce anesthesia.

The hair on the dorsal side of the anesthetized mice was removed and disinfected, followed by excision of the full-thickness skin using an 8 mm punch. Subsequently, skin of the same size was harvested from donor mice (BALB/c) and transplanted.

In the experimental group, the compounds G015F (20 mg/kg) and G088 (0.15 mg/kg) of the present invention were administered intraperitoneally twice daily at 12-hour intervals starting two days before transplantation, and the same administration was continued post-transplantation.

The control group received vehicle alone via the same method.

For immune cell analysis, mice from each group were sacrificed on day 9 post-transplantation, and cells were isolated from the spleen.

The isolated spleen cells were suspended in MACS buffer (Miltenyi Biotec., Cat. #130-092-987), and 5×105 cells were aliquoted into FACS tubes. To block non-specific antibody binding, the cells were incubated with anti-mouse CD16/CD32 (BioXCell, Cat. #BP0307) at a concentration of 1 μg/mL for 30 minutes.

Subsequently, the antibody PerCP/Cyanine5.5 anti-CD3 (Biolegend, Cat. #100218) was used to label T cells, and the antibody FITC anti-mouse B220 (eBioscience™, Cat. #11-0452-81) was used to label B cells.were added and incubated for 30 minutes. The cells were then centrifuged at 1,500 rpm for 5 minutes and resuspended in 100 μL of MACS buffer.

Subsequently, the proportions of CD3- and B220-positive cells were analyzed using a flow cytometer.

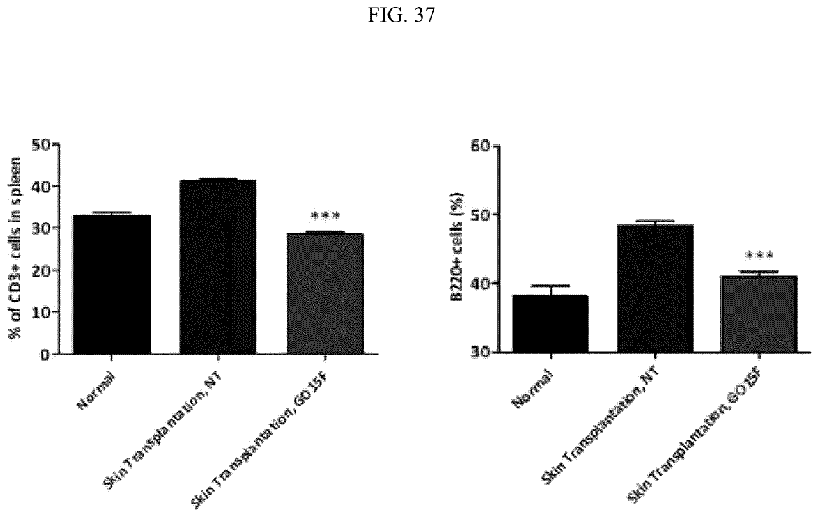

The results are shown in FIG. 37.

This allowed us to quantitatively evaluate the relative changes in the ratios of T cells and B cells following treatment with the compounds of the present invention.

FIG. 37 visualizes these results as a bar graph, where the vertical axis represents the percentage of T cells and B cells in the spleen cells, and the horizontal axis distinguishes each experimental group (e.g., NT, GO15F, etc.).

As a result, in the control group (not treated, NT) where only skin transplantation was performed, both the ratio of T cells (CD3+) and B cells (B220+) in the spleen increased.

In contrast, the groups administered the compound of the present invention showed a decrease in the ratio of T cells and B cells, confirming that the compound of the present invention exhibits an immunosuppressive effect in the transplanted animal model.

Additionally, among the compounds of the present invention, G088 administered at 0.15 mg/kg twice daily, three out of five animals showed graft pinkening and maintained good blood flow on day 10 post-transplantation, and one out of five animals showed the same on day 15 post-transplantation, indicating stable graft survival.

These results suggest that G088 is effective in delaying immune rejection responses against the graft.

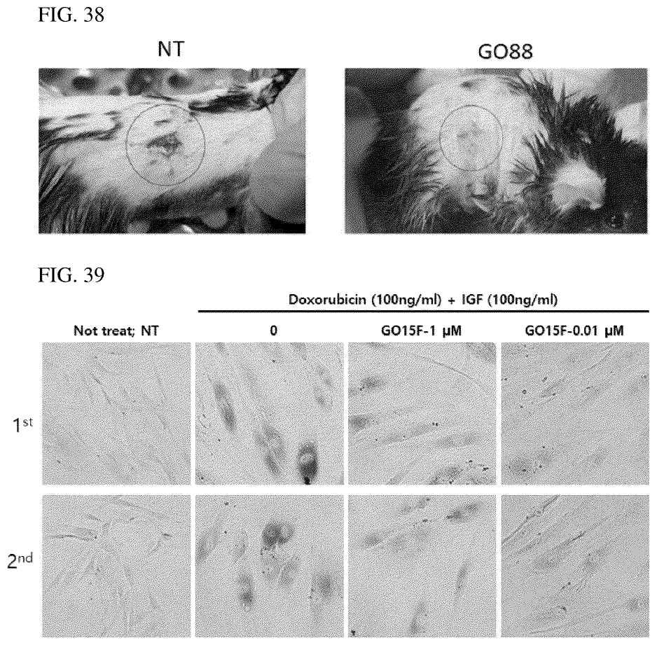

FIG. 38 shows photographs of skin grafts in mice treated with the compounds according to the present invention.

Based on the above results, it was confirmed that the immune response induced during biological transplantation is effectively regulated by the administration of the compound according to the present invention, thereby mitigating or preventing graft rejection.

In particular, the compounds of the present invention are believed to contribute to inhibiting the activation of immune cells such as T cells and B cells by inhibiting the phosphorylation of PDK1 or acting antagonistically against it.

This mechanism of action is considered to play a crucial role in promoting graft engraftment and extending the survival period of the graft by suppressing the excessive proliferation of immune cells.

Therefore, it can be concluded that the compounds of the present invention act as immunomodulatory agents that effectively control the activity of immune cells and mitigate immune rejection responses against grafts by inhibiting or antagonistically acting against the functional activity of PDK1.

It is therefore concluded that the compounds of the present invention, when administered at appropriate therapeutic doses, can be utilized as therapeutic agents to improve graft survival rates in situations requiring immune regulation, such as organ transplantation and tissue transplantation.

### Experimental Example 13: Anti-Aging and Rejuvenating Effects in NHDF Cells

To confirm the anti-aging and rejuvenating effects of the compound G015F of the present invention, a NHDF (Normal Human Dermal Fibroblast, Promocell) cell model was established by inducing aging with doxorubicin (Doxorubicin), and (3-galactosidase activity and Ki-67 expression were analyzed in the cells.

### Confirmation of Anti-Aging Efficacy

First, NHDF cells were seeded at 1×10⁴ cells per well in a 12-well plate and stabilized for 24 hours.

Following stabilization, the cells were treated with the compound GO15F (0.01, 1 μM) from the present invention and pre-incubated for 24 hours.

After pre-incubation, the medium was removed, and the cells were cultured for 7 days in medium containing Doxorubicin (100 ng/ml) and IGF-1 (100 ng/ml) with GO15F.

During this process, the medium was replaced with fresh medium every 2 days.

The cultured cells were washed with PBS, fixed with 1× fixation buffer for 15 minutes, washed again with PBS, and then 300 μL of 1× working SA-β-gal buffer was added to each well. The cells were incubated at 37° C. for 24 hours.

After the reaction was terminated, the cells were washed with PBS, and the fluorescence intensity of P-gal-positive cells stained blue was observed and photographed using a light microscope.

This is shown in FIG. 39.

As shown in FIG. 39, compared to the control group, where β-galactosidase activity increased due to Doxorubicin and IGF-1 treatment, the number of β-gal-positive cells decreased in the GO15F-treated group, suggesting that G015F may have an anti-aging effect by inhibiting cellular aging mechanisms.

### Increased Cell Proliferation Capacity

Additionally, to investigate the effect of the compound (GO15F) of the present invention on proliferation activity in aging-induced NHDF (Normal Human Dermal Fibroblast) cells, Ki-67 expression was measured via FACS analysis.

First, NHDF cells were seeded at 1×104 cells per well in a 12-well plate and stabilized for 24 hours. Subsequently, the cells were pre-incubated with G015F at a concentration of 0.01 μM for 24 hours. After pre-incubation, the medium was removed, and G015F (0.01 μM) was added to the medium containing Doxorubicin (100 ng/ml) and IGF-1 (100 ng/ml), followed by incubation for 7 days with fresh medium replaced every 2 days.

After culture completion, cells were dissociated with Trypsin-EDTA, washed once with PBS, and centrifuged at 1,500 rpm for 3 minutes.

Cells were fixed with 1× Fixation buffer for 15 minutes, washed with PBS, and incubated with blocking buffer for 1 hour.

Following blocking, the cells were incubated with Ki-67 antibody diluted 1:100 at room temperature for 1 hour, washed three times with PBS (1,500 rpm, 3 minutes), and then incubated with Alexa Fluor 488-conjugated secondary antibody at room temperature for 1 hour. The cells were washed three times with PBS.

Finally, the prepared cells were analyzed using a flow cytometer to measure the number of cells exhibiting Ki-67 fluorescence.

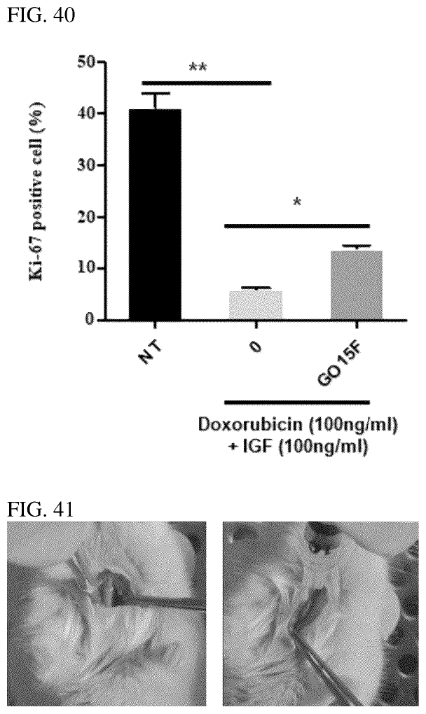

The results are shown in FIG. 40.

The results showed that, compared to the control group where Ki-67 expression was significantly reduced by doxorubicin and IGF-1, the number of cells exhibiting Ki-67 expression increased in the group treated with the compound of the present invention.

These results indicate that the compound of the present invention reduces the accumulation of β-galactosidase-positive cells in a cell aging-inducing environment, while simultaneously increasing Ki-67 expression to restore cell proliferation capacity, thereby exhibiting functions that inhibit cell aging and induce rejuvenation.

Based on these effects, the compound is considered to have potential for use as a PDK1 inhibitor for anti-aging or rejuvenation purposes.

Experimental Example 14: In vivo and in vitro efficacy experiments for inhibiting pulmonary fibrosis

To evaluate the therapeutic efficacy of the compound according to the present invention against pulmonary fibrosis, an animal model of pulmonary fibrosis induced by bleomycin intratracheal administration was established.

For this purpose, 8-week-old male Balb/c mice were subjected to general anesthesia using an inhalation anesthetic (2-3% isoflurane in oxygen), followed by disinfection of the neck region, skin incision, and exposure of the trachea.

Bleomycin (Sigma-Aldrich, USA)was directly administered into the trachea at a concentration of 5 mg/kg in a solution of 100 L.

This procedure was photographed and attached as FIG. 41.

Two days prior to Bleomycin administration, the compound of the present invention or the comparative drug was administered intraperitoneally at the specified concentration and dose, and the administration was continued once daily for a total of 14 days.

The experimental groups included Normal, Bleomycin alone (BLM), and BLM plus the compound of the present invention (BLM+G088, 0.2 mg/kg).

Ten days later, the mice were euthanized humanely using carbon dioxide gas, the trachea was incised, and PBS was injected into the trachea to remove blood from the lungs.

The lung tissue washed with PBS was excised, fixed in 4% paraformaldehyde solution for 24 hours, and then paraffin blocks and tissue sections were prepared. The prepared sections were subjected to pathological analysis using H&E, Masson's trichrome, and Sirius red staining.

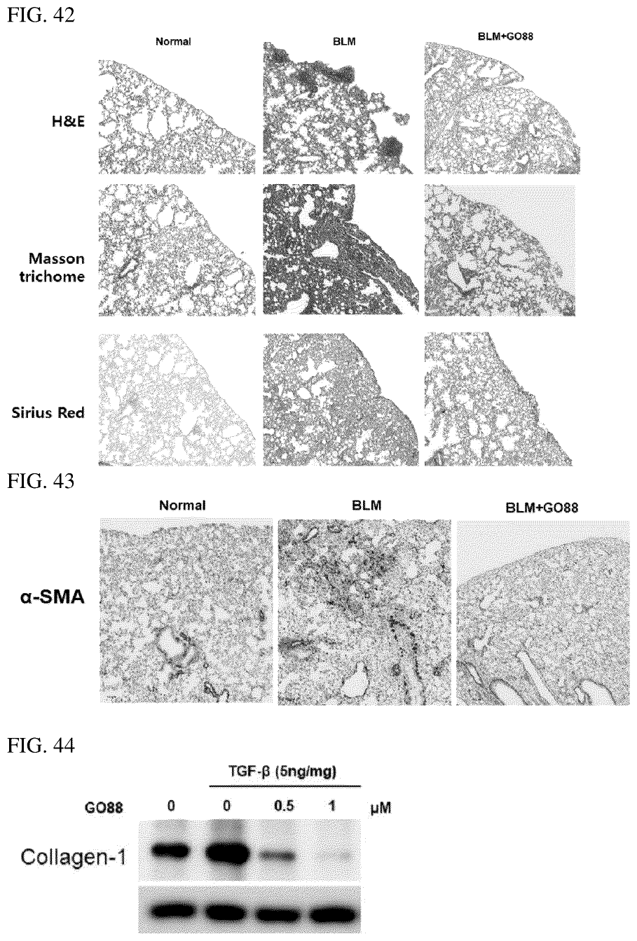

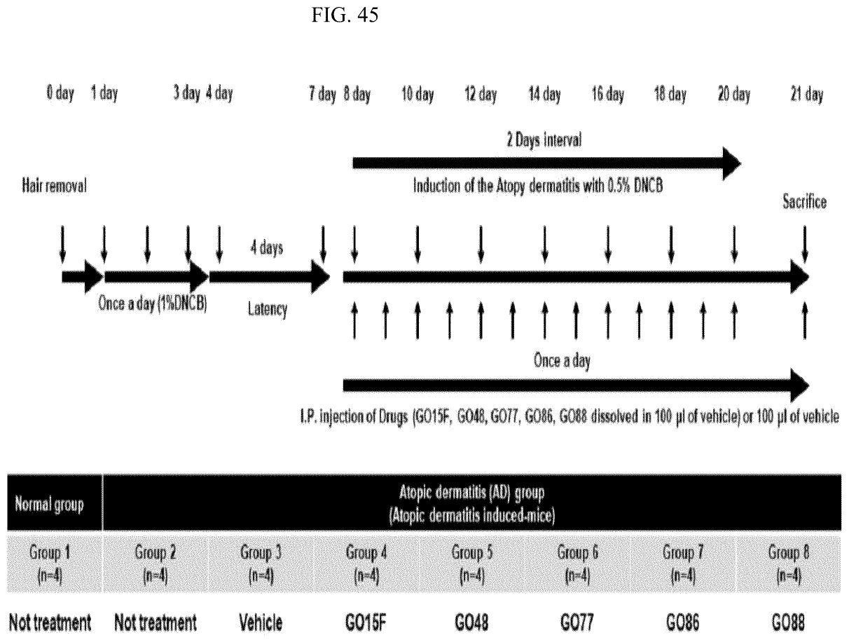

This is shown in FIG. 42.

FIG. 42 shows photographs of lungs extracted from mice with induced pulmonary fibrosis after administration of the compound according to the present invention, followed by staining, pathological analysis, and photography.

H&E staining revealed tissue damage such as loss of alveolar structure and infiltration of numerous inflammatory cells in the BLM-treated group.

In contrast, in the group treated with the compound of the present invention, the alveolar structure was relatively well preserved, and a reduction in inflammatory cell infiltration was confirmed.

Additionally, analysis of collagen deposition, a fibrosis marker, using Masson's trichrome and Sirius red staining revealed that the area of collagen fibers stained blue and red was significantly increased in the BLM-treated group.

However, in the G015F and G088-treated groups, such collagen deposition was significantly reduced.

### Confirmation of α-SMA Expression Inhibition

Additionally, immunohistochemical (IHC) staining was performed for α-SMA (α-smooth muscle actin), an indicator of lung fibroblast activation.

This is shown in FIG. 43.

FIG. 43 shows photographs of the lungs of mice with induced pulmonary fibrosis treated with the compounds according to the present invention, followed by IHC staining and analysis.

Referring to FIG. 43, in the BLM-treated group, the expression of α-SMA-positive cells in lung tissue was significantly increased, suggesting the activation of fibroblasts and the formation of myofibroblasts.

In contrast, in the G015F and G088-treated groups, the expression of α-SMA was significantly suppressed compared to the BLM-treated group.

### Confirmation of Direct Action on Fibrosis-Related Mechanisms

To confirm that the compound of the present invention directly acts on fibrosis-related mechanisms, in vitro experiments were performed using human lung fibroblasts MRC-5 cell lines (Korea Cell Bank).

First, MRC-5 cells were seeded at 5×10³ cells per well in a 6-well plate and stabilized for 24 hours.

Subsequently, experimental groups were established, including a control group, a group treated with TGF-β (5 ng/ml) alone, and groups treated with TGF-β and the compound G088 from the present invention at concentrations of 0.5 μM and 1 M, respectively.

After treating each group with the drugs for 24 hours, the cell medium was removed, and the cells were washed twice with cold PBS. Then, RIPA lysis buffer containing phosphatase inhibitor and protease inhibitor was added to each group to extract proteins.

The extracted proteins were incubated at 4° C. for 30 minutes, then centrifuged at 13,000 rpm for 15 minutes, and the supernatant was collected. The total protein concentration was measured using the BCA protein quantification method.

Subsequently, the proteins were separated by SDS-PAGE, transferred to a PVDF membrane, and Western blot analysis was performed.

The expression levels of Collagen I, a marker related to pulmonary fibrosis, were confirmed, with GAPDH used as an internal control.

This is shown in FIG. 44.

As shown in FIG. 44, the protein expression of Collagen I was significantly increased in the TGF-β-treated group compared to the untreated group, but in the group treated with G088, the protein expression of Collagen I was inhibited in a concentration-dependent manner.

These results suggest that the compound G088 of the present invention effectively inhibits the fibrosis mechanism induced by TGF-β, thereby demonstrating its potential as a preventive or therapeutic agent for pulmonary fibrosis.

Based on these results, it is concluded that the compound of the present invention (GO88) alleviates lung damage by inhibiting inflammatory and fibrotic responses in lung tissue, suppresses the activation of fibroblasts through reduced α-SMA expression, and prevents excessive accumulation of Collagen I in tissues.

Additionally, it has been confirmed that the compound effectively inhibits the increase in Collagen I expression in MRC-5 cells induced by TGF-β, suggesting the potential to regulate fibrotic mechanisms not only at the tissue level but also at the cellular level.

Therefore, the compound of the present invention can be useful as a pharmaceutical composition for the prevention or treatment of various fibrotic diseases, including pulmonary fibrosis.

### Experimental Example 15: Establishment of an Autoimmune Disease Model (Atopic Dermatitis) and Verification of Therapeutic Effects

To verify the therapeutic effects of the compound according to the present invention on atopic dermatitis (AD), an atopic animal model was established as follows, and the compound according to the present invention was administered to evaluate the alleviating effects on skin lesions.

To establish an atopic mouse animal model, BALB/c mice (7 weeks old, 20 g, female) were purchased from HanabioTech Co., Ltd. and acclimatized to the experimental environment for one week.

Subsequently, the hair on the back of the mice was shaved once using a hair removal device, followed by a second complete shaving using a depilatory cream (Nikrin Cream [80% thioglycolic acid, II Dong Pharmaceutical]) to ensure complete hair removal. The mice were then housed for 24 hours to observe any wounds on the back.

To verify the preventive and therapeutic effects of the compounds according to the present invention on atopic dermatitis, the following groups were established: a group without atopic dermatitis (Normal, Not Treated (NT)), a group with atopic dermatitis only (AD, NT), a group with atopic dermatitis and the solvent of the injection solution administered intraperitoneally (AD, Vehicle), and a group with atopic dermatitis and the compounds according to the present invention administered intraperitoneally (AD, the compound of the present invention (GO15F, G048, G077, GO86, GO88)).

To induce atopy in mice, 1% DNCB, 200 μl was applied topically to the skin once daily for three days to induce atopic lesions, followed by a four-day rest period. Then, 0.5% DNCB, 200 l, once every two days for a total of seven applications to induce atopic dermatitis.

Concurrently, to compare the preventive and therapeutic effects of the compound according to the present invention on atopic dermatitis, the drug treatment group was administered the compound at the set dose per mouse, dissolved in DMSO at 10% of the drug dose, and finally mixed with PEG 400:distilled water at a ratio of 1:1:4:4 (v/v/v/v) to form a solution.PEG 400:distilled water at a ratio of 1:1:4:4 (v/v/v/v) to obtain the final mixture.

This injection solution was administered intraperitoneally at a dose of 100 μL per mouse once daily for 7 days. For the vehicle group, the injection solution excluding the drug (DMSO: Cremophor EL: PEG 400: distilled water=1:1:4:4 (v/v/v/v)) was administered intraperitoneally at 100 μL once daily.

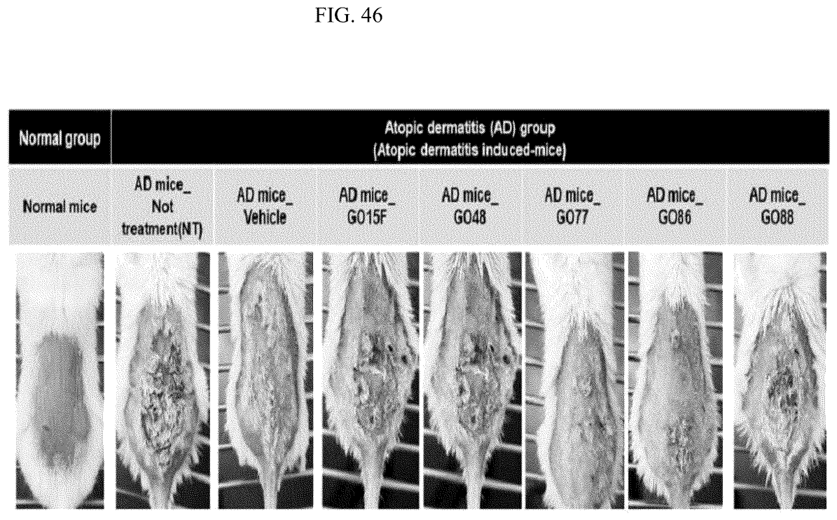

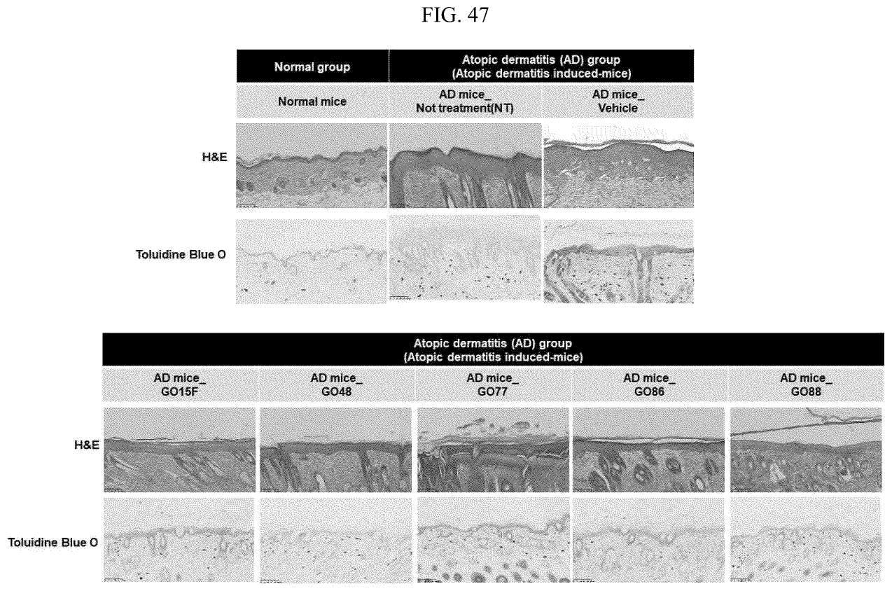

At 14 days after the start of injection, the severity of skin lesions on the back of each mouse in the corresponding group was observed, and pathological tissue analysis was performed using H&E and Toluidine Blue staining. The results are presented in FIGS. 46 and 47.

As shown in FIG. 46, in the group that induced atopic dermatitis alone (AD, NT) and the group that induced atopic dermatitis and simultaneously administered the injection solution solvent into the peritoneal cavity (AD, Vehicle), severe accumulation of hyperkeratosis was observed. However, in mice administered the compound according to the present invention, a reduction in hyperkeratosis accumulation was observed.

Referring to FIG. 47, H&E staining of skin tissue from atopic dermatitis-induced mice revealed that, compared to the normal control group (Normal), the control groups (NT and Vehicle) exhibited increased epidermal thickness and increased infiltration of inflammatory cells.

Additionally, Toluidine Blue staining showed a significant increase in the number of mast cells in the dermis.

In contrast, in the group treated with the compound according to the present invention, epidermal hyperplasia was significantly alleviated, and a reduction in mast cell accumulation was also observed in Toluidine Blue staining.

These results confirm that the compound according to the present invention inhibits inflammatory responses in atopic dermatitis and exhibits effects in alleviating hyperkeratosis and mast cell accumulation.

The compound is considered to have the potential to act as an effective substance for the treatment of atopic dermatitis.

Experimental Example 16: Binding affinity experiment with PDK1 using MST Microscale Thermophoresis (MST) analysis was performed to quantitatively measure the binding affinity between PDK1 protein and substrate peptides (PDKTide) or the company-developed low-molecular-weight compound (GO88).

The target proteins were the full-length (PDK1-FL) protein of human-derived PDK1 and the Protein Kinase Domain (PKD) fragment, which were expressed using a baculovirus expression system in insect cell lines (Sf9).

The harvested cells were lysed with a lysis buffer, followed by Ni-NTA affinity chromatography and Superdex 200 Increase 10/300 GL size exclusion chromatography (Cytiva) to purify the proteins to over 90% purity. The purity of the purified proteins was confirmed by SDS-PAGE and Coomassie Brilliant Blue staining.

The purified protein was labeled with a fluorescent label using the Monolith NT™ RED-NHS Protein Fluorescent Labeling Kit (NanoTemper).

Specifically, 20 μM of the protein was dissolved in PBS (pH 7.4) buffer, mixed with the fluorescent reagent, and reacted at room temperature under dark conditions for 30 minutes. The mixture was then buffered with MST analysis buffer (50 mM HEPES, 150 mM NaCl, 1 mM DTT, 0.05% Tween-20, pH 7.5).

The fluorescently labeled protein was diluted to a final concentration of 20 nM and mixed with ligands (GO88, 10 nM-1 mM) diluted to various concentrations. The mixture was reacted at room temperature for 30 minutes in the presence or absence of ATP or ADP (final concentration 1 mM).

The reaction samples were injected into an MST-specific capillary (NanoTemper) and analyzed using a Monolith NT.115 device (NanoTemper) under laser intensity conditions.

The laser exposure time was 30 seconds, and the total measurement time was approximately 40 seconds.

The measured data were analyzed using MO.Affinity Analysis software (NanoTemper), and the dissociation constant (Kd) was calculated from the binding curve.

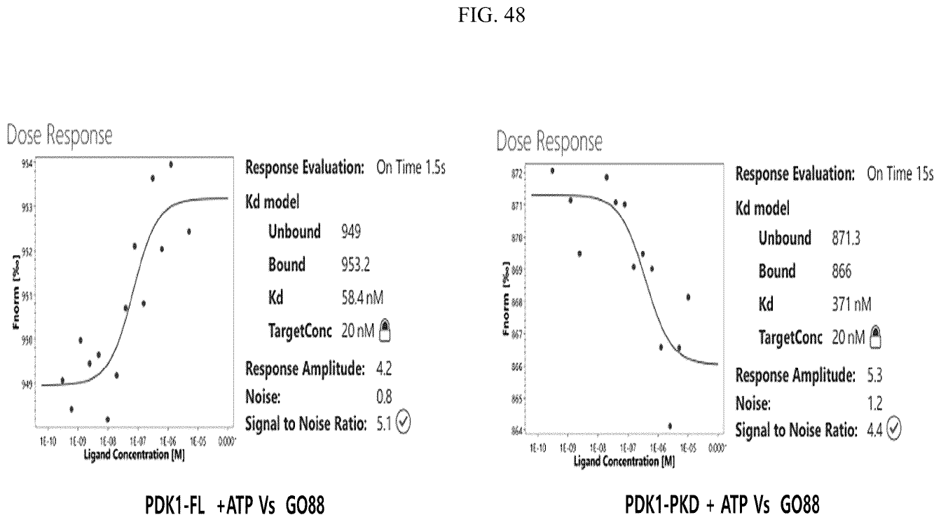

This is shown in FIG. 48.

FIG. 48 is a graph obtained through a comparative analysis of the binding affinity between PDK1 and G088 using Microscale Thermophoresis (MST) according to the invention.

As shown, the binding between PDK1-PKD protein and G088 was measured to have a dissociation constant (Kd) of approximately 371 nM in the presence of ATP, indicating an intermediate level of binding affinity.

In contrast, the binding between full-length PDK1 and G088 was measured to have a Kd of approximately 58.4 nM, showing approximately six times stronger binding affinity compared to the PDK fragment.

These results suggest that G088 may exhibit high binding affinity through interactions with specific domains of PDK1 (e.g., the PIF domain) or through binding at allosteric sites, and that it may act as a non-competitive inhibitor, as the binding is maintained even in the presence of ATP.

1. An anticancer compound characterized by comprising the compound represented by chemical formula 1 or its pharmaceutically acceptable salts: 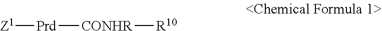
wherein Z¹ is —NH₂ (or —NHR1, wherein R1 is benzene or a benzene derivative), —SH, methylthio, benzoic acid, Benzyloxy group, halogen-substituted alkyl, benzimidazolyl group, —OH, —SH, halogen, —NO₂—, —CF₃, C1-C6 alkyl, C2-C6 alkenyl, C2-C6 alkynyl, C3-C10 arylalkyl (Ar—(CH₂)n—), C5-C10 aryl (Ar—), C3-C10 alkylaryl ((CH₂)n—Ar—), C3-C10 cycloalkyl, C3-C10 heteroaryl, or C3-C10 heterocycloalkyl, or unsubstituted C5-C10 aryl or C3-C10 heteroaryl, and
Prd is pyridine or benzene,
—CONH— is an amide bond,
R¹⁰ is C8-C14 alkyl, C8-C14 alkenyl, or C8-C14 alkynyl, wherein one, two, or three hydrogen atoms of the terminal carbon of the alkyl, alkenyl, or alkynyl group are substituted with halogen (fluorine, chlorine, bromine, iodine), morpholine or its analogues, piperazine or its analogues, thiomorpholine or its analogs, pyrrolidine or its analogs, piperidine or its analogs, 3,4-dihydro-2H-benzo[b][1,4]oxazine or its analogs, pyrimidine or its analogs, pyrazine or its analogs, or a functional group listed in Table 1,
TABLE 1

| Derivative | NO. | Functional group |
|---|---|---|
| Morpholine | M1 | 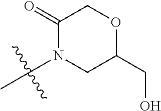 [SMILES: *N1CC(CO)OCC1=O] |
| | M2 | 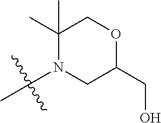 [SMILES: *N1CC(CO)OCC1(C)C] |
| | M3 | 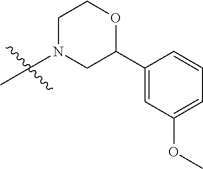 [SMILES: *N1CCOC(c2cccc(OC)c2)C1] |
| | M4 | 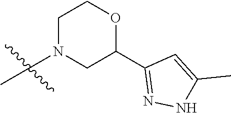 [SMILES: *N1CCOC(c2cc(C)[nH]n2)C1] |
| | M5 | 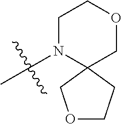 [SMILES: *N1CCOCC12CCOC2] |
| | M6 | 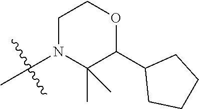 [SMILES: *N1CCOC(C2CCCC2)C1(C)C] |
| | M7 | 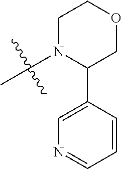 [SMILES: *N1CCOCC1c1cccnc1] |
| | M8 | 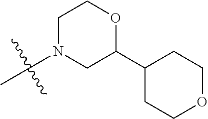 [SMILES: *N1CCOC(C2CCOCC2)C1] |
| | M9 | 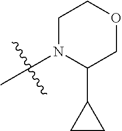 [SMILES: *N1CCOCC1C1CC1] |
| | M10 | 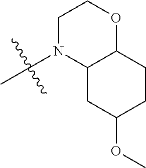 [SMILES: *N1CCOC2CCC(OC)CC21] |
| | M11 | 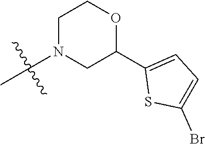 [SMILES: *N1CCOC(c2ccc(Br)s2)C1] |
| | M12 | 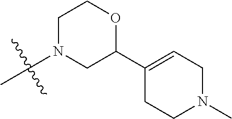 [SMILES: *N1CCOC(C2=CCN(C)CC2)C1] |
| | M13 | 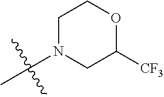 [SMILES: *N1CCOC(C(F)(F)F)C1] |
| | M14 | 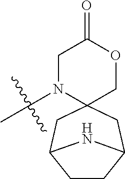 [SMILES: *N1CC(=O)OCC12CC1CCC(C2)N1] |
| | M15 | 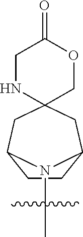 [SMILES: *N1C2CCC1CC1(COC(=O)CN1)C2] |
| | M16 | 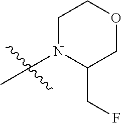 [SMILES: *N1CCOCC1CF] |
| | m17 | 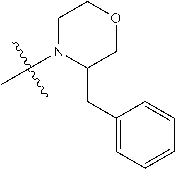 [SMILES: *N1CCOCC1Cc1ccccc1] |
| | M18 | 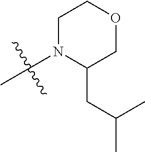 [SMILES: *N1CCOCC1CC(C)C] |
| | M19 | 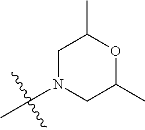 [SMILES: *N1CC(C)OC(C)C1] |
| | M20 | 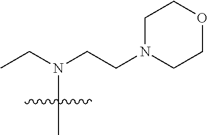 [SMILES: *N(CC)CCN1CCOCC1] |
| | M21 | 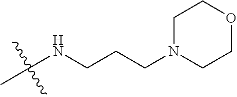 [SMILES: *NCCCN1CCOCC1] |
| | M22 | 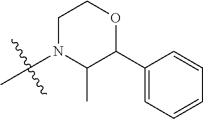 [SMILES: *N1CCOC(c2ccccc2)C1C] |
| | M23 | 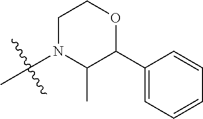 [SMILES: *N1CCOC(c2ccccc2)C1C] |
| | M24 | 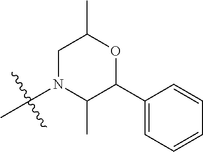 [SMILES: *N1CC(C)OC(c2ccccc2)C1C] |
| | M25 | 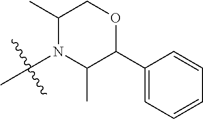 [SMILES: *N1C(C)COC(c2ccccc2)C1C] |
| | M26 | 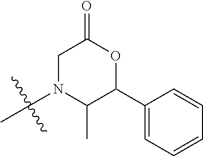 [SMILES: *N1CC(=O)OC(c2ccccc2)C1C] |
| | M27 | 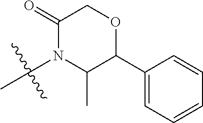 [SMILES: *N1C(=O)COC(c2ccccc2)C1C] |
| | M28 | 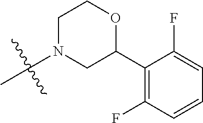 [SMILES: *N1CCOC(c2c(F)cccc2F)C1] |
| | M29 | 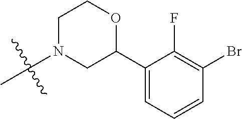 [SMILES: *N1CCOC(c2cccc(Br)c2F)C1] |
| | M30 | 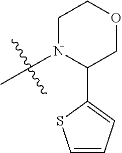 [SMILES: *N1CCOCC1c1cccs1] |
| | M31 | 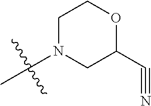 [SMILES: *N1CCOC(C#N)C1] |
| | M32 | 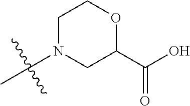 [SMILES: *N1CCOC(C(=O)O)C1] |
| | M33 | 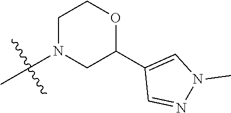 [SMILES: *N1CCOC(c2cnn(C)c2)C1] |
| | M34 | 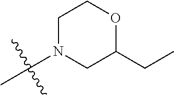 [SMILES: *N1CCOC(CC)C1] |
| | M35 | 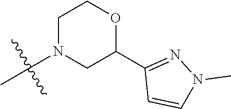 [SMILES: *N1CCOC(c2ccn(C)n2)C1] |
| Piperazine | Pi1 | 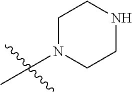 [SMILES: *N1CCNCC1] |
| | Pi2 | 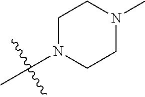 [SMILES: *N1CCN(C)CC1] |
| | Pi3 | 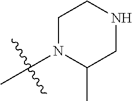 [SMILES: *N1CCNCC1C] |
| | Pi4 | 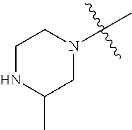 [SMILES: *N1CCNC(C)C1] |
| | Pi5 | 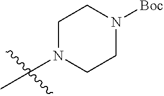 [SMILES: *N1CCN(C(=O)OC(C)(C)C)CC1] |
| | Pi6 | 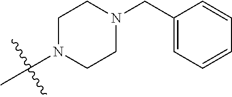 [SMILES: *N1CCN(Cc2ccccc2)CC1] |
| | Pi7 | 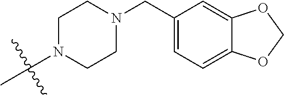 [SMILES: *N1CCN(Cc2ccc3c(c2)OCO3)CC1] |
| | Pi8 | 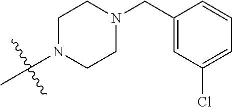 [SMILES: *N1CCN(Cc2cccc(Cl)c2)CC1] |
| | Pi9 | 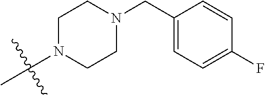 [SMILES: *N1CCN(Cc2ccc(F)cc2)CC1] |
| | Pi10 | 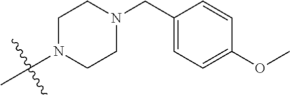 [SMILES: *N1CCN(Cc2ccc(OC)cc2)CC1] |
| | Pi11 | 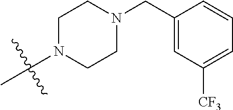 [SMILES: *N1CCN(Cc2cccc(C(F)(F)F)c2)CC1] |
| | Pi12 | 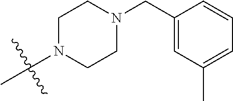 [SMILES: *N1CCN(Cc2cccc(C)c2)CC1] |
| | Pi13 | 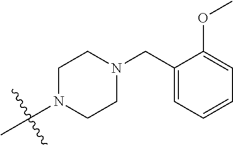 [SMILES: *N1CCN(Cc2ccccc2OC)CC1] |
| | Pi14 | 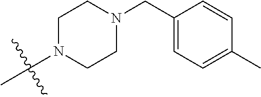 [SMILES: *N1CCN(Cc2ccc(C)cc2)CC1] |
| | Pi15 | 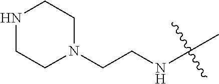 [SMILES: *NCCN1CCNCC1] |
| | Pi16 | 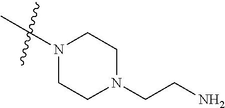 [SMILES: *N1CCN(CCN)CC1] |
| | Pi17 | 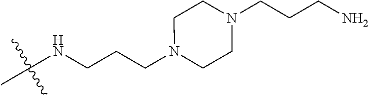 [SMILES: *NCCCN1CCN(CCCN)CC1] |
| | Pi18 | 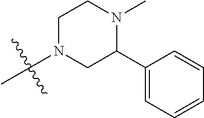 [SMILES: *N1CCN(C)C(c2ccccc2)C1] |
| | Pi19 | 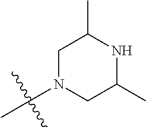 [SMILES: *N1CC(C)NC(C)C1] |
| | Pi20 | 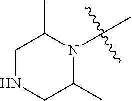 [SMILES: *N1C(C)CNCC1C] |
| | Pi21 | 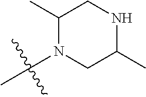 [SMILES: *N1CC(C)NCC1C] |
| | Pi22 | 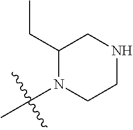 [SMILES: *N1CCNCC1CC] |
| | Pi23 | 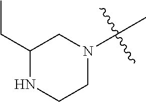 [SMILES: *N1CCNC(CC)C1] |
| | Pi24 | 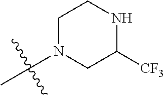 [SMILES: *N1CCNC(C(F)(F)F)C1] |
| | Pi25 | 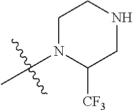 [SMILES: *N1CCNCC1C(F)(F)F] |
| | Pi26 | 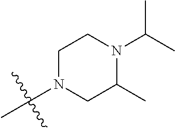 [SMILES: *N1CCN(C(C)C)C(C)C1] |
| | Pi27 | 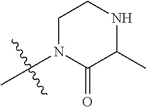 [SMILES: *N1CCNC(C)C1=O] |
| | Pi28 | 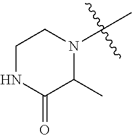 [SMILES: *N1CCNC(=O)C1C] |
| | Pi29 | 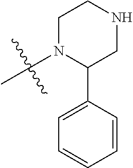 [SMILES: *N1CCNCC1c1ccccc1] |
| | Pi30 | 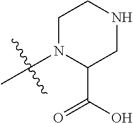 [SMILES: *N1CCNCC1C(=O)O] |
| | Pi31 | 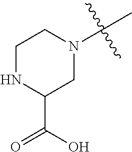 [SMILES: *N1CCNC(C(=O)O)C1] |
| | Pi32 | 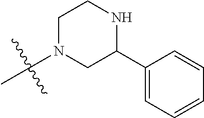 [SMILES: *N1CCNC(c2ccccc2)C1] |
| | Pi33 | 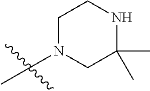 [SMILES: *N1CCNC(C)(C)C1] |
| | Pi34 | 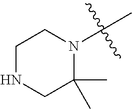 [SMILES: *N1CCNCC1(C)C] |
| | Pi35 | 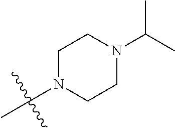 [SMILES: *N1CCN(C(C)C)CC1] |
| | Pi36 | 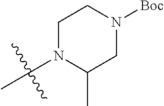 [SMILES: *N1CCN(C(=O)OC(C)(C)C)CC1C] |
| | Pi37 | 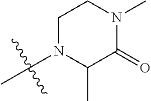 [SMILES: *N1CCN(C)C(=O)C1C] |
| | Pi38 | 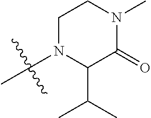 [SMILES: *N1CCN(C)C(=O)C1C(C)C] |
| | Pi39 | 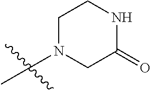 [SMILES: *N1CCNC(=O)C1] |
| | Pi40 | 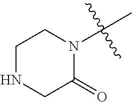 [SMILES: *N1CCNCC1=O] |
| | Pi41 | 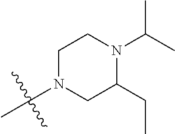 [SMILES: *N1CCN(C(C)C)C(CC)C1] |
| | Pi42 | 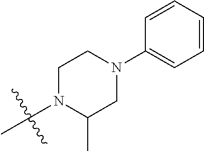 [SMILES: *N1CCN(c2ccccc2)CC1C] |
| | Pi43 | 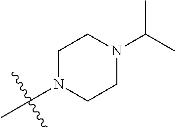 [SMILES: *N1CCN(C(C)C)CC1] |
| Thiomorpholine | Th1 | 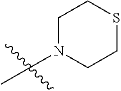 [SMILES: *N1CCSCC1] |
| | Th2 | 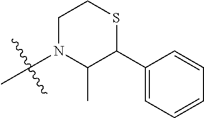 [SMILES: *N1CCSC(c2ccccc2)C1C] |
| | Th3 | 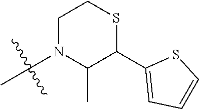 [SMILES: *N1CCSC(c2cccs2)C1C] |
| pyrrolidine | Py1 | 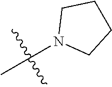 [SMILES: *N1CCCC1] |
| | Py2 | 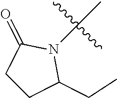 [SMILES: *N1C(=O)CCC1CC] |
| | Py3 | 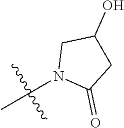 [SMILES: *N1CC(O)CC1=O] |
| | Py4 | 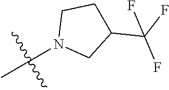 [SMILES: *N1CCC(C(F)(F)F)C1] |
| | Py5 | 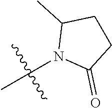 [SMILES: *N1C(=O)CCC1C] |
| | Py6 | 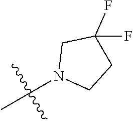 [SMILES: *N1CCC(F)(F)C1] |
| | Py7 | 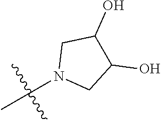 [SMILES: *N1CC(O)C(O)C1] |
| | Py8 | 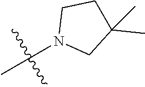 [SMILES: *N1CCC(C)(C)C1] |
| | Py9 | 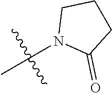 [SMILES: *N1CCCC1=O] |
| | Py10 | 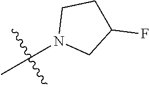 [SMILES: *N1CCC(F)C1] |
| | Py11 | 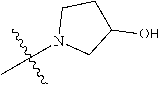 [SMILES: *N1CCC(O)C1] |
| | py12 | 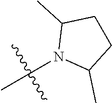 [SMILES: *N1C(C)CCC1C] |
| | py13 | 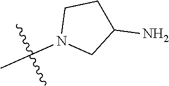 [SMILES: *N1CCC(N)C1] |
| | py14 | 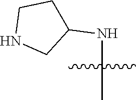 [SMILES: *NC1CCNC1] |
| | Py15 | 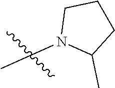 [SMILES: *N1CCCC1C] |
| | Py16 | 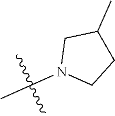 [SMILES: *N1CCC(C)C1] |
| | py17 | 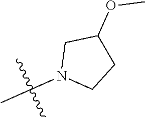 [SMILES: *N1CCC(OC)C1] |
| | py18 | 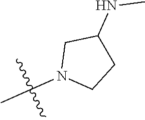 [SMILES: *N1CCC(NC)C1] |
| | py19 | 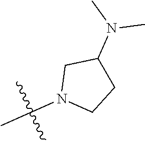 [SMILES: *N1CCC(N(C)C)C1] |
| | Py20 | 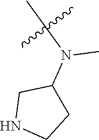 [SMILES: *N(C)C1CCNC1] |
| | Py21 | 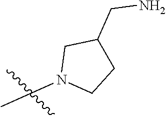 [SMILES: *N1CCC(CN)C1] |
| | Py22 | 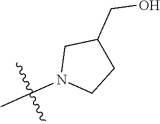 [SMILES: *N1CCC(CO)C1] |
| | Py23 | 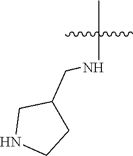 [SMILES: *NCC1CCNC1] |
| | Py24 | 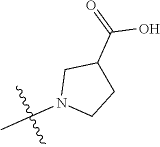 [SMILES: *N1CCC(C(=O)O)C1] |
| | Py25 | 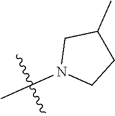 [SMILES: *N1CCC(C)C1] |
| | py26 | 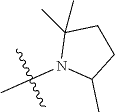 [SMILES: *N1C(C)CCC1(C)C] |
| piperidine | Pd1 | 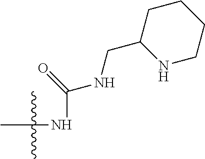 [SMILES: *NC(=O)NCC1CCCCN1] |
| | Pd2 | 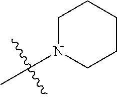 [SMILES: *N1CCCCC1] |
| | Pd3 | 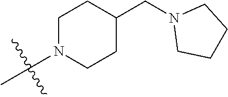 [SMILES: *N1CCC(CN2CCCC2)CC1] |
| | Pd4 | 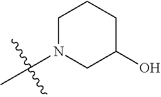 [SMILES: *N1CCCC(O)C1] |
| | Pd5 | 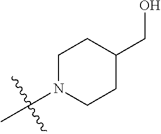 [SMILES: *N1CCC(CO)CC1] |
| | Pd6 | 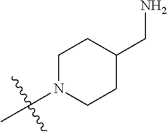 [SMILES: *N1CCC(CN)CC1] |
| | Pd7 | 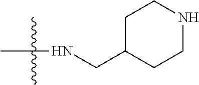 [SMILES: *NCC1CCNCC1] |
| | Pd8 | 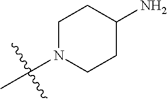 [SMILES: *N1CCC(N)CC1] |
| | Pd9 | 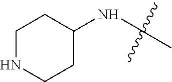 [SMILES: *NC1CCNCC1] |
| | Pd10 | 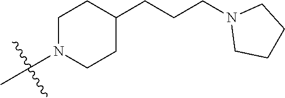 [SMILES: *N1CCC(CCCN2CCCC2)CC1] |
| | Pd11 | 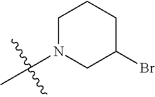 [SMILES: *N1CCCC(Br)C1] |
| | Pd12 | 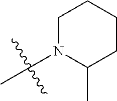 [SMILES: *N1CCCCC1C] |
| | Pd13 | 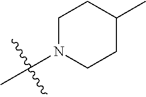 [SMILES: *N1CCC(C)CC1] |
| | Pd14 | 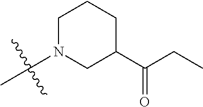 [SMILES: *N1CCCC(C(=O)CC)C1] |
| | Pd15 | 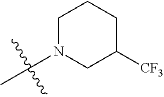 [SMILES: *N1CCCC(C(F)(F)F)C1] |
| | Pd16 | 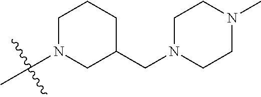 [SMILES: *N1CCCC(CN2CCN(C)CC2)C1] |
| | Pd17 | 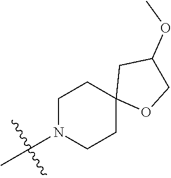 [SMILES: *N1CCC2(CC1)CC(OC)CO2] |
| | Pd18 | 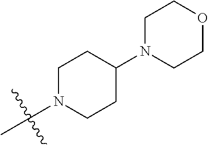 [SMILES: *N1CCC(N2CCOCC2)CC1] |
| | Pd19 | 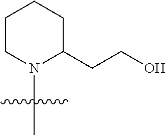 [SMILES: *N1CCCCC1CCO] |
| | Pd20 | 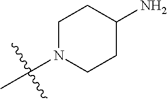 [SMILES: *N1CCC(N)CC1] |
| | Pd21 | 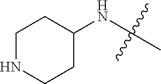 [SMILES: *NC1CCNCC1] |
| | Pd22 | 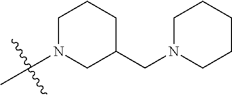 [SMILES: *N1CCCC(CN2CCCCC2)C1] |
| | Pd23 | 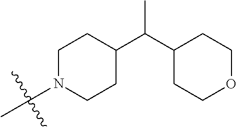 [SMILES: *N1CCC(C(C)C2CCOCC2)CC1] |
| | Pd24 | 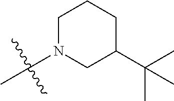 [SMILES: *N1CCCC(C(C)(C)C)C1] |
| | Pd25 | 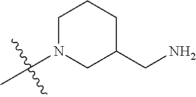 [SMILES: *N1CCCC(CN)C1] |
| | Pd26 | 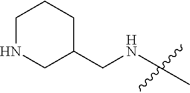 [SMILES: *NCC1CCCNC1] |
| | Pd27 | 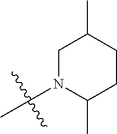 [SMILES: *N1CC(C)CCC1C] |
| | Pd28 | 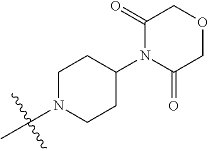 [SMILES: *N1CCC(N2C(=O)COCC2=O)CC1] |
| 3,4-dihydro-2H- benzo[b][1,4]oxazine | Di1 | 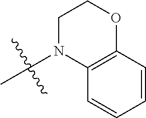 [SMILES: *N1CCOc2ccccc21] |
| | Di2 | 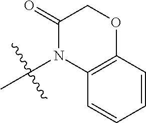 [SMILES: *N1C(=O)COc2ccccc21] |
| | Di3 | 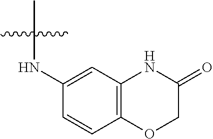 [SMILES: *Nc1ccc2c(c1)NC(=O)CO2] |
| | Di4 | 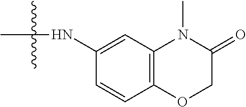 [SMILES: *Nc1ccc2c(c1)N(C)C(=O)CO2] |
| | Di5 | 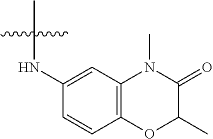 [SMILES: *Nc1ccc2c(c1)N(C)C(=O)C(C)O2] |
| | Di6 | 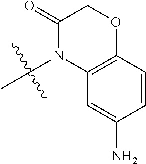 [SMILES: *N1C(=O)COc2ccc(N)cc21] |
| | Di7 | 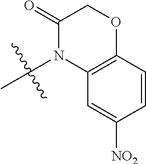 [SMILES: *N1C(=O)COc2ccc([N+](=O)[O-])cc21] |
| Pyrimidine | Pyd1 | 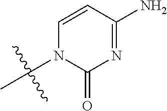 [SMILES: *n1ccc(N)nc1=O] |
| | Pyd2 | 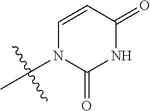 [SMILES: *n1ccc(=O)[nH]c1=O] |
| | Pyd3 | 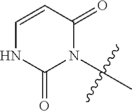 [SMILES: *n1c(=O)cc[nH]c1=O] |
| | Pyd4 | 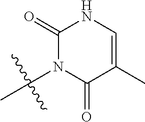 [SMILES: *n1c(=O)[nH]cc(C)c1=O] |
| | Pyd5 | 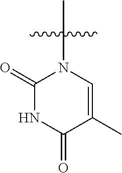 [SMILES: *n1cc(C)c(=O)[nH]c1=O] |
| | Pyd6 | 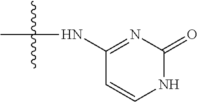 [SMILES: *Nc1cc[nH]c(=O)n1] |
| Pyrazine | Pyz1 | 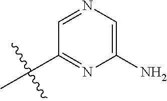 [SMILES: *c1cncc(N)n1] |
| | Pyz2 | 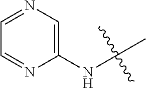 [SMILES: *Nc1cnccn1] |
| | Pyz3 | 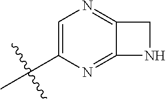 [SMILES: *c1cnc2c(n1)NC2] |
| | Pyz4 | 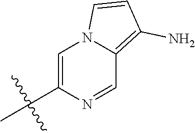 [SMILES: *c1cn2ccc(N)c2cn1] |
| | Pyz5 | 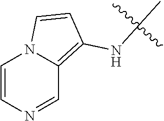 [SMILES: *Nc1ccn2ccncc12] |
| | Pyz6 | 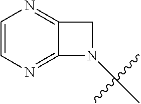 [SMILES: *N1Cc2nccnc21] |
| | Pyz7 | 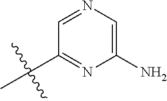 [SMILES: *c1cncc(N)n1] |
| Other | O1 | 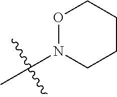 [SMILES: *N1CCCCO1] |
| | O2 | 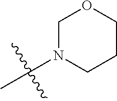 [SMILES: *N1CCCOC1] |
| | O3 | 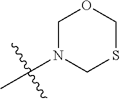 [SMILES: *N1COCSC1] |
| | O4 | 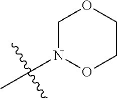 [SMILES: *N1COCCO1] |
| | O5 | 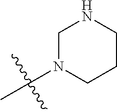 [SMILES: *N1CCCNC1] |
| | O6 | 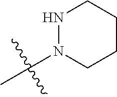 [SMILES: *N1CCCCN1] |
| | O7 | 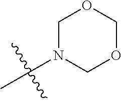 [SMILES: *N1COCOC1] |
| | O8 | 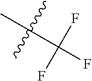 [SMILES: *C(F)(F)F] |
| | O9 | 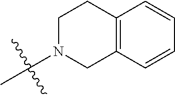 [SMILES: *N1CCc2ccccc2C1] |
| | O10 | 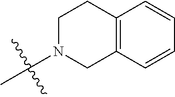 [SMILES: *N1CCc2ccccc2C1] |
| | O11 | 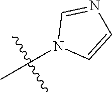 [SMILES: *n1ccnc1] |
| | O12 | 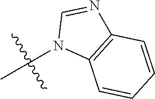 [SMILES: *n1cnc2ccccc21] |
| | O13 | 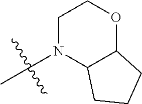 [SMILES: *N1CCOC2CCCC21] |
| | O14 | 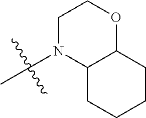 [SMILES: *N1CCOC2CCCCC21] |
| | O15 | 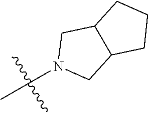 [SMILES: *N1CC2CCCC2C1] |
| | O16 | 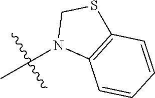 [SMILES: *N1CSc2ccccc21] |
| | O17 | 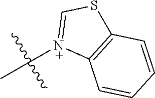 [SMILES: *[n+]1csc2ccccc21] |
| | O18 | 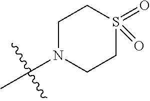 [SMILES: *N1CCS(=O)(=O)CC1] |
| | O19 | 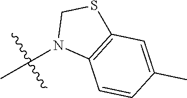 [SMILES: *N1CSc2cc(C)ccc21] |
| | O20 | 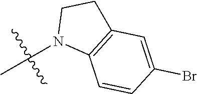 [SMILES: *N1CCc2cc(Br)ccc21] |
| | O21 | 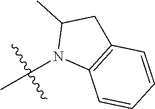 [SMILES: *N1c2ccccc2CC1C] |
| | O22 | 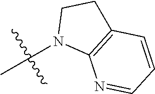 [SMILES: *N1CCc2cccnc21] |
| | O23 | 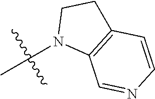 [SMILES: *N1CCc2ccncc21] |
| | O24 | 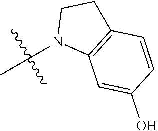 [SMILES: *N1CCc2ccc(O)cc21] |
| | O25 | 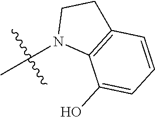 [SMILES: *N1CCc2cccc(O)c21] |
| | O26 | 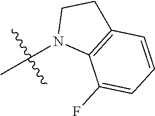 [SMILES: *N1CCc2cccc(F)c21] |
| | O27 | 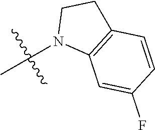 [SMILES: *N1CCc2ccc(F)cc21] |
| | O28 | 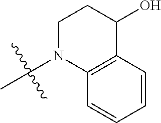 [SMILES: *N1CCC(O)c2ccccc21] |
| | O29 | 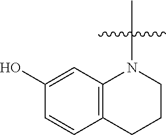 [SMILES: *N1CCCc2ccc(O)cc21] |
| | O30 | 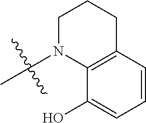 [SMILES: *N1CCCc2cccc(O)c21] |
| | O31 | 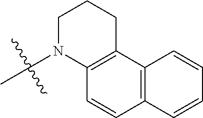 [SMILES: *N1CCCc2c1ccc1ccccc21] |
| | O32 | 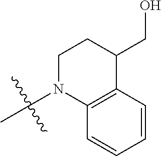 [SMILES: *N1CCC(CO)c2ccccc21] |
| | O33 | 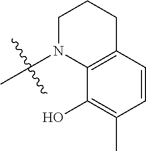 [SMILES: *N1CCCc2ccc(C)c(O)c21] |
| | O34 | 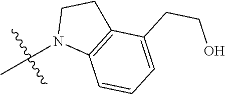 [SMILES: *N1CCc2c(CCO)cccc21] |
| | O35 | 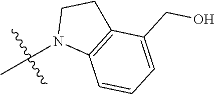 [SMILES: *N1CCc2c(CO)cccc21] |
| | O36 | 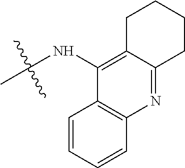 [SMILES: *Nc1c2c(nc3ccccc13)CCCC2] |
| | O37 | 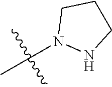 [SMILES: *N1CCCN1] |
| | O38 | 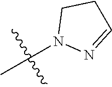 [SMILES: *N1CCC=N1] |
| | O39 | 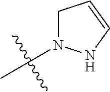 [SMILES: *N1CC=CN1] |
| | O40 | 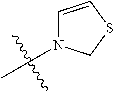 [SMILES: *N1C=CSC1] |
| | O41 | 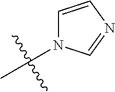 [SMILES: *n1ccnc1] |
| | O42 | 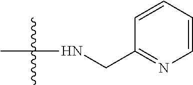 [SMILES: *NCc1ccccn1] |
| | O43 | 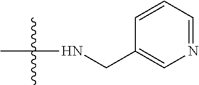 [SMILES: *NCc1cccnc1] |
| | O44 | 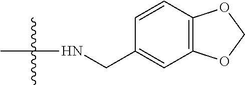 [SMILES: *NCc1ccc2c(c1)OCO2] |
| | O45 | 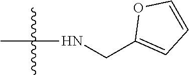 [SMILES: *NCc1ccco1] |
| | O46 | 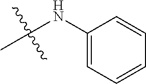 [SMILES: *Nc1ccccc1] |
| | O47 | 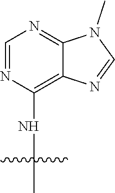 [SMILES: *Nc1ncnc2c1ncn2C] |
| | O48 | 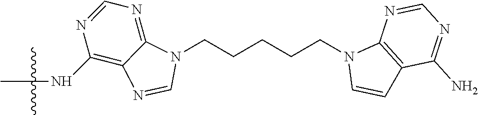 [SMILES: *Nc1ncnc2c1ncn2CCCCCn1ccc2c(N)ncnc21] |
| | O49 | 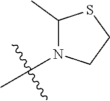 [SMILES: *N1CCSC1C] |
| | O50 | 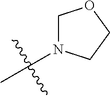 [SMILES: *N1CCOC1] |
| | O51 | 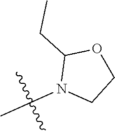 [SMILES: *N1CCOC1CC] |
| | O52 | 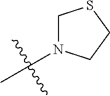 [SMILES: *N1CCSC1] |
| | O53 | 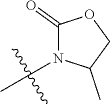 [SMILES: *N1C(=O)OCC1C] |
| | O54 | 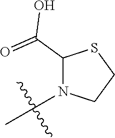 [SMILES: *N1CCSC1C(=O)O] |
| | O55 | 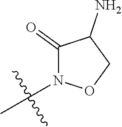 [SMILES: *N1OCC(N)C1=O] |
| | O56 | 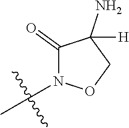 [SMILES: *N1OCC(N)C1=O] |
| | O57 | 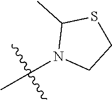 [SMILES: *N1CCSC1C] |
| | O58 | 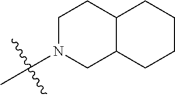 [SMILES: *N1CCC2CCCCC2C1] |
| | O59 | 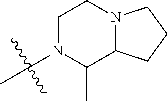 [SMILES: *N1CCN2CCCC2C1C] |
| | O60 | 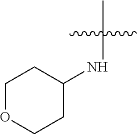 [SMILES: *NC1CCOCC1] |
| | O61 | 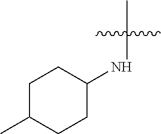 [SMILES: *NC1CCC(C)CC1] |
| | O62 | 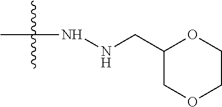 [SMILES: *NNCC1COCCO1] |
| | O63 | 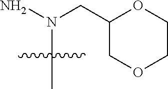 [SMILES: *N(N)CC1COCCO1] |
| | O64 | 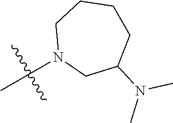 [SMILES: *N1CCCCC(N(C)C)C1] |
| | O65 | 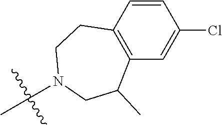 [SMILES: *N1CCc2ccc(Cl)cc2C(C)C1] |
| | O66 | 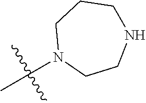 [SMILES: *N1CCCNCC1] |
| | O67 | 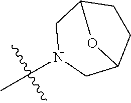 [SMILES: *N1CC2CCC(C1)O2] |
| | O68 | 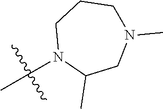 [SMILES: *N1CCCN(C)CC1C] |
| | O69 | 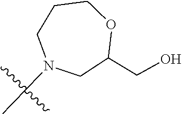 [SMILES: *N1CCCOC(CO)C1] |
| | O70 | 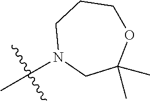 [SMILES: *N1CCCOC(C)(C)C1] |
| | O71 | 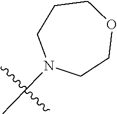 [SMILES: *N1CCCOCC1] |
| | O72 | 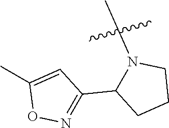 [SMILES: *N1CCCC1c1cc(C)on1] |
| | O73 | 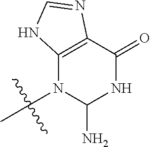 [SMILES: *N1c2[nH]cnc2C(=O)NC1N] |
| | O74 | 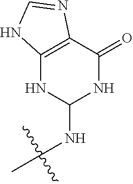 [SMILES: *NC1NC(=O)c2nc[nH]c2N1] |
| | O75 | 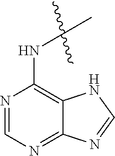 [SMILES: *Nc1ncnc2nc[nH]c12] |
| | O76 | 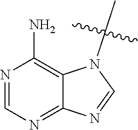 [SMILES: *n1cnc2ncnc(N)c21] |
| | O77 | 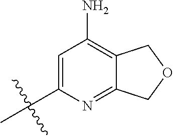 [SMILES: *c1cc(N)c2c(n1)COC2] |
| | O78 | 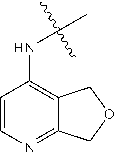 [SMILES: *Nc1ccnc2c1COC2] |
| | O79 | 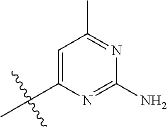 [SMILES: *c1cc(C)nc(N)n1] |
| | O80 | 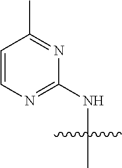 [SMILES: *Nc1nccc(C)n1] |
| | O81 | 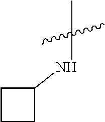 [SMILES: *NC1CCC1] |
| | O82 | 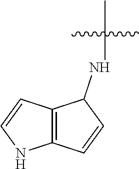 [SMILES: *NC1C=Cc2[nH]ccc21] |
| | O83 | 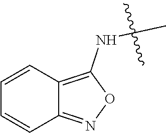 [SMILES: *Nc1onc2ccccc12] |
| | O84 | 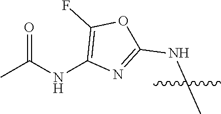 [SMILES: *Nc1nc(NC(C)=O)c(F)o1] |
| | O85 | 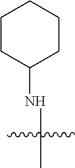 [SMILES: *NC1CCCCC1] |
| | O86 | 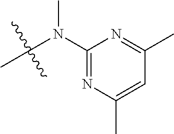 [SMILES: *N(C)c1nc(C)cc(C)n1] |
| | O87 | 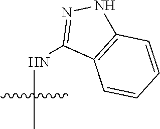 [SMILES: *Nc1n[nH]c2ccccc12] |
| | O88 | 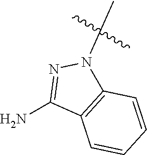 [SMILES: *n1nc(N)c2ccccc21] |

or a functional group selected from the group consisting of

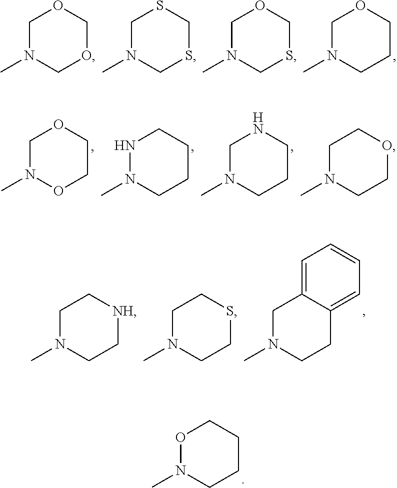

2. The anticancer compound according to claim 1, wherein the alkyl, alkenyl, or alkynyl of R¹⁰ has a carbon number of 8 to 14, characterized in that it exhibits a non-competitive inhibitory effect on the phosphorylation of PDK1.

3. The anticancer compound according to claim 1, wherein the carbon number is 7 or less, making it difficult to exhibit an immunosuppressive effect, and when the carbon number is 15 or more, it becomes difficult to form other stereochemical bonds or to be absorbed in the body.

4. The anticancer compound according to claim 1, wherein the compound is characterized by containing at least one of the following substances: 2-amino-N-(12-fluoro-dodecyl)-nicotinamide, 4′-methyl-biphenyl-2-carboxylic acid (12-fluoro-dodecyl)-amide, 2-amino-N-dodecylnicotinamide, 2-Bromo-N-(12-fluoro-dodecyl)-nicotinamide, 2-Amino-N-[12-(3,4-dihydro-1H-isoquinolin-2-yl)-dodecyl]-nicotinamide, N-(12-fluoro-dodecyl)-3-(3-trifluoromethyl-phenylamino)-isonicotinamide, 4-chloro-N-(12-fluoro-dodecyl)-nicotinamide, 2-amino-N-(12-morpholin-4-yl-dodecyl)-nicotinamide, 2-amino-N-(8-fluoro-octyl)-nicotinamide, N-(12-fluoro-dodecyl)-2-mercapto-nicotinamide, 2-amino-N-(10-morpholin-4-yl-decyl)-nicotinamide, Biphenyl-2-carboxylic acid (12-fluoro-dodecyl)-amide, 1H-Indole-4-carboxylic acid (12-fluoro-dodecyl)-amide, 3-Hydroxy-pyridine-2-carboxylic acid (12-fluoro-dodecyl)-amide, 3-benzoyl-pyridine-2-carboxylic acid (12-fluoro-dodecyl)-amide, isoquinoline-1-carboxylic acid (12-fluoro-dodecyl)-amide, 2-(4-chloro-phenoxy)-N-(12-fluoro-dodecyl)-nicotinamide, N-(12-fluoro-dodecyl)-2-methylsulfonyl-nicotinamide, 2-amino-N-(10-fluoro-decyl)-nicotinamide, 3-(12-fluoro-dodecylcarbamoyl)-pyridine-2-yl-ammonium chloride, 2-Amino-N-(11-fluoro-undecyl)-nicotinamide, 3-(11-fluoro-undecylcarbamoyl)-pyridin-2-yl-ammonium chloride, N-(12-fluoro-dodecyl)-2-hydroxy-nicotinamide, 4-Amino-N-(12-fluoro-dodecyl)-nicotinamide, 3-Amino-N-(12-fluoro-dodecyl)-isonicotinamide, N-(12-fluoro-dodecyl)-2-trifluoromethyl-nicotinamide, N-(12-fluoro-dodecyl)-3-iodoisonicotinamide, 2-amino-N-(12-thiophenyl-4-yl-dodecyl)nicotinamide, and 2-amino-N-(12-(piperazin-1-yl)dodecyl)nicotinamide.

5. The anticancer compound according to claim 1, wherein the compound is characterized by being selected from at least one of the compounds selected from the group consisting of chemical formulas 2 to 20,

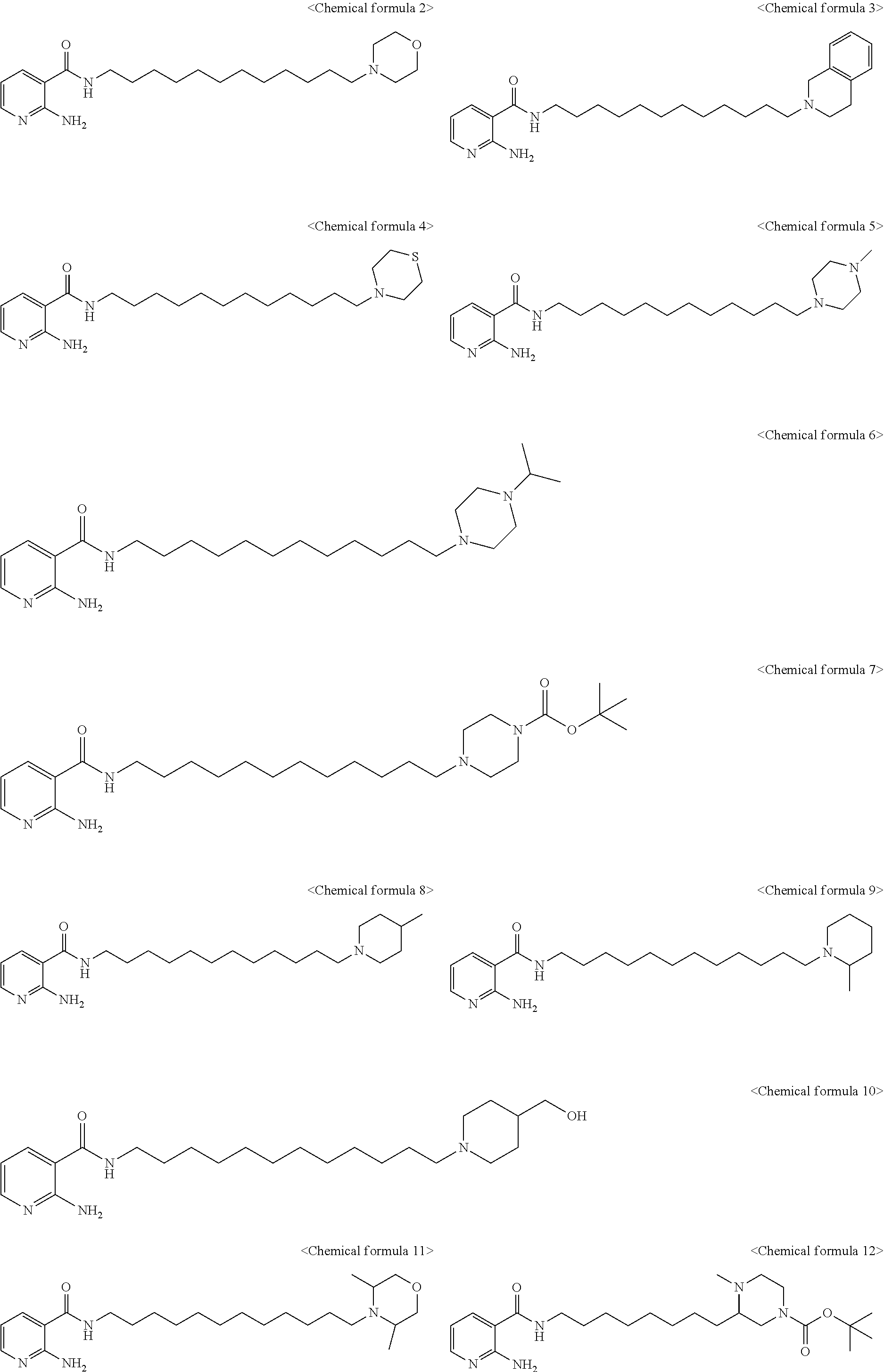

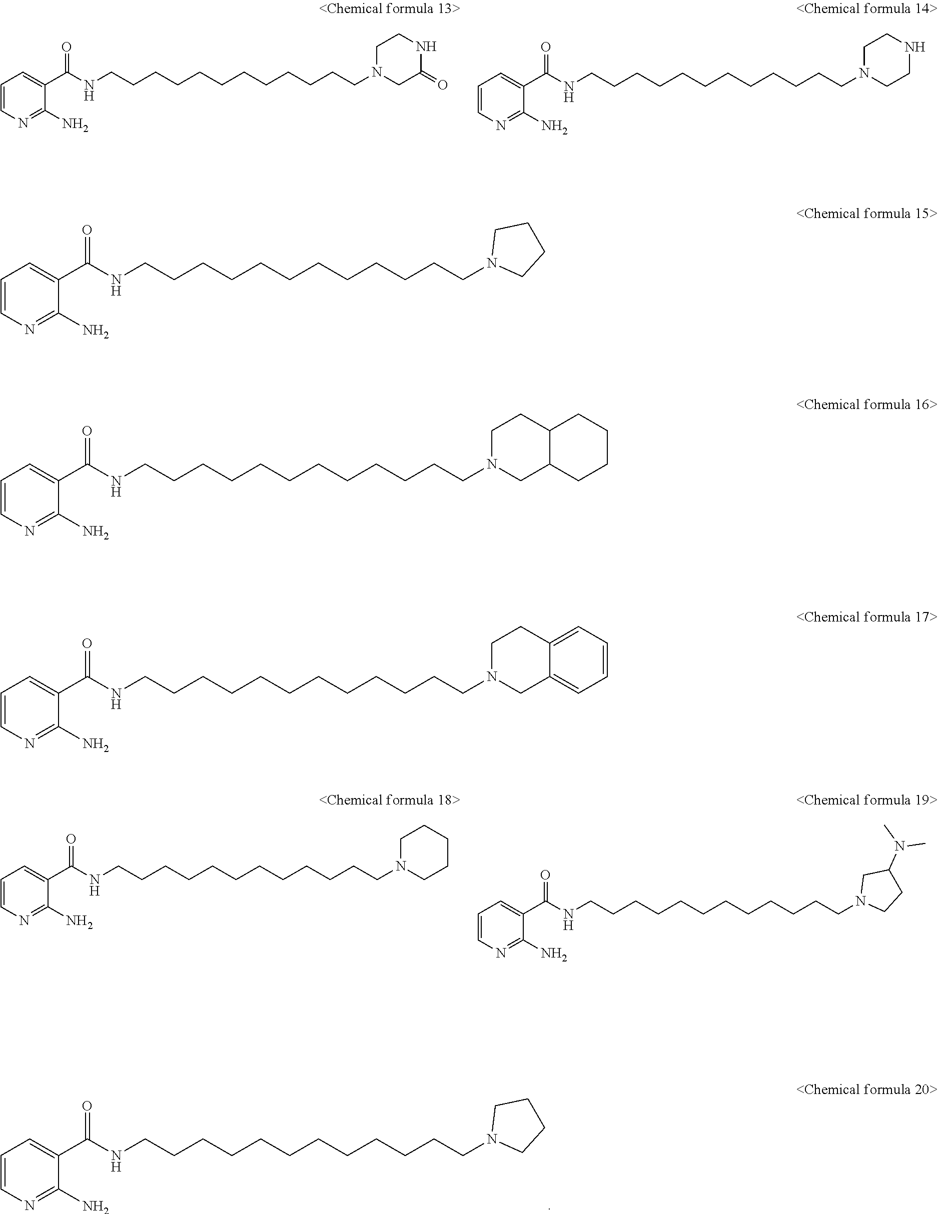

6. The anticancer compound according to claim 1, wherein the compound is characterized by being selected from at least one of the compounds selected from the group consisting of chemical formulas 21 to 39,

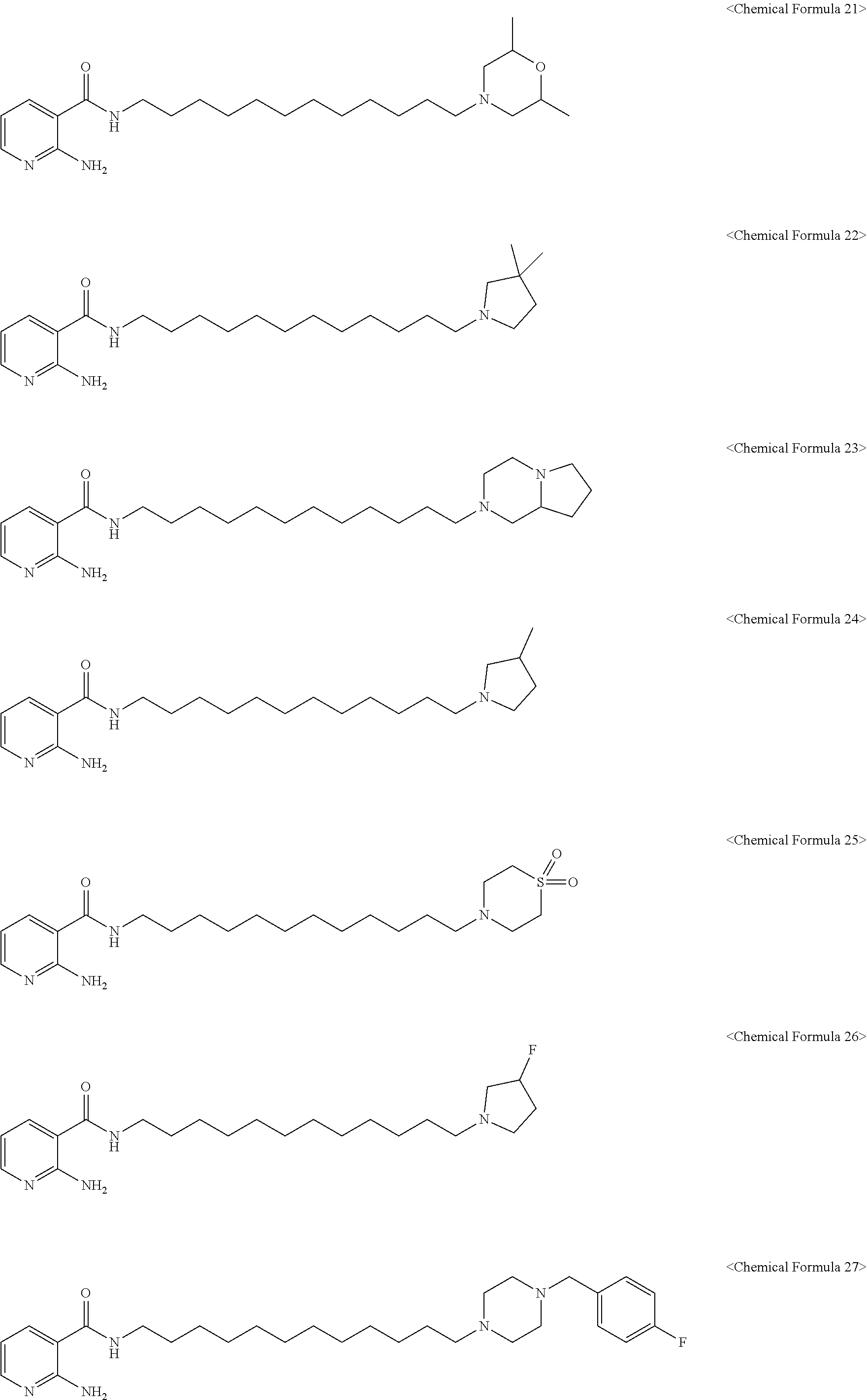

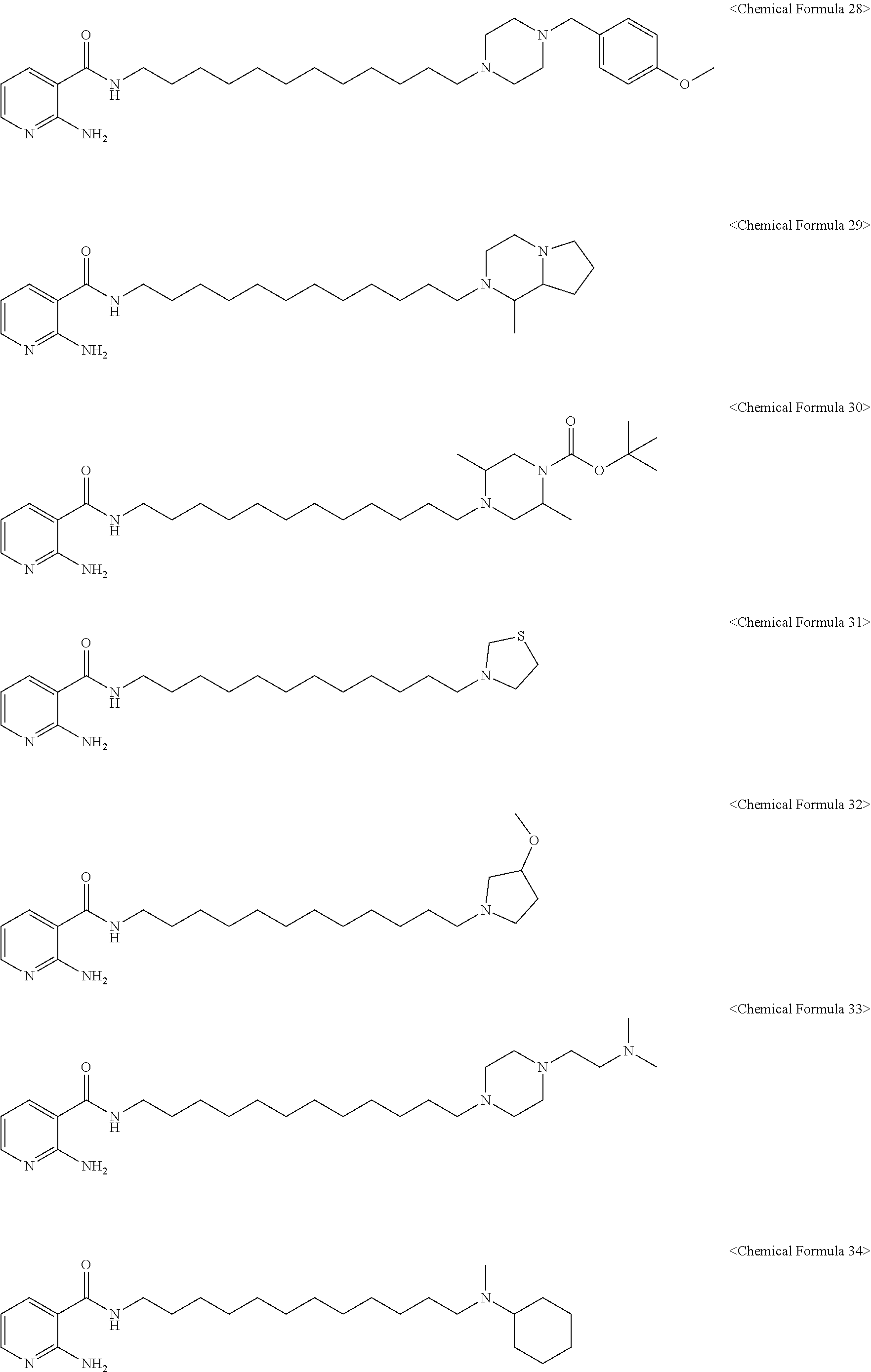

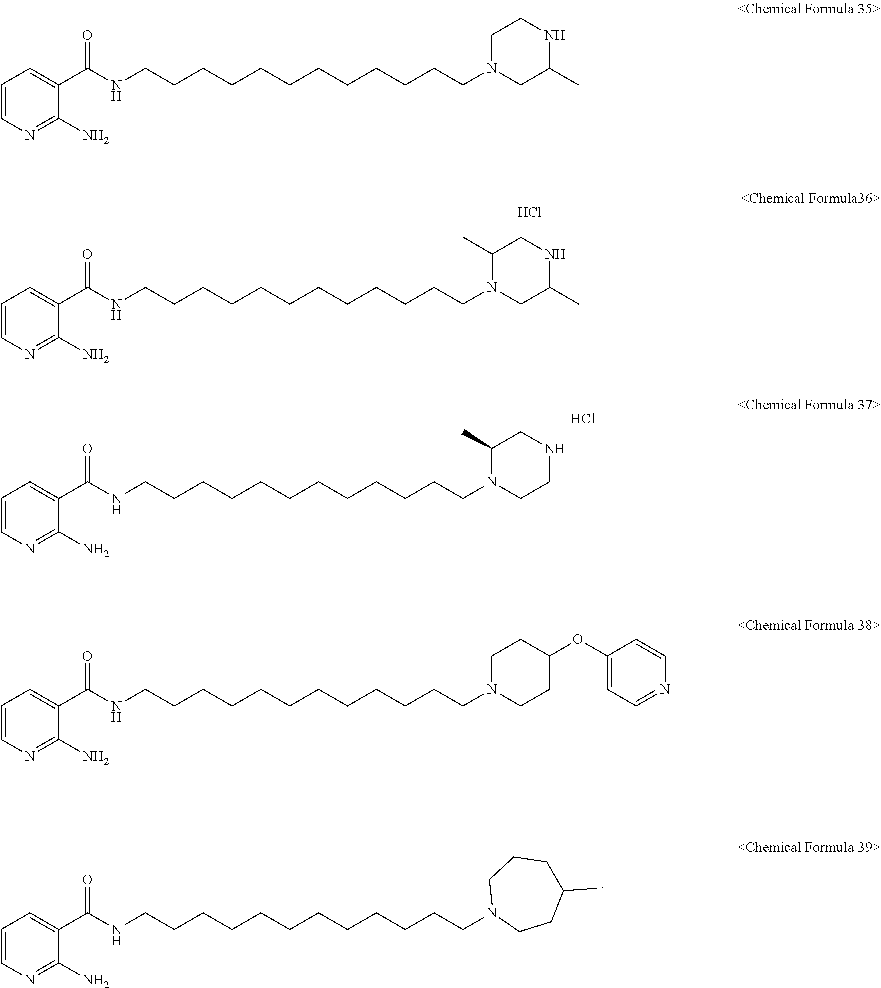

7. The anticancer compound according to claim 1, wherein the compound is characterized by being selected from at least one of the group consisting of compounds represented by chemical formulas 43 to 60,

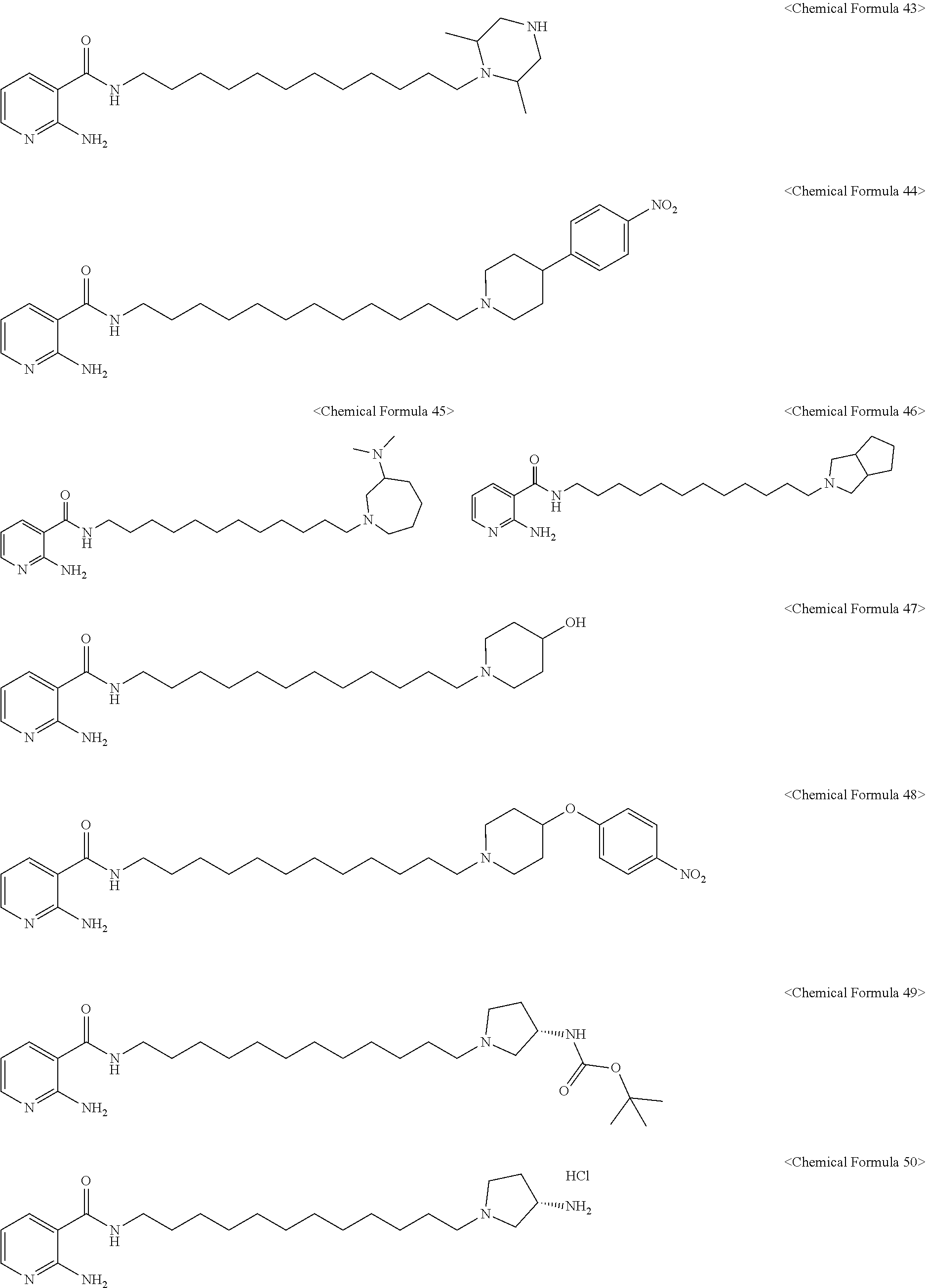

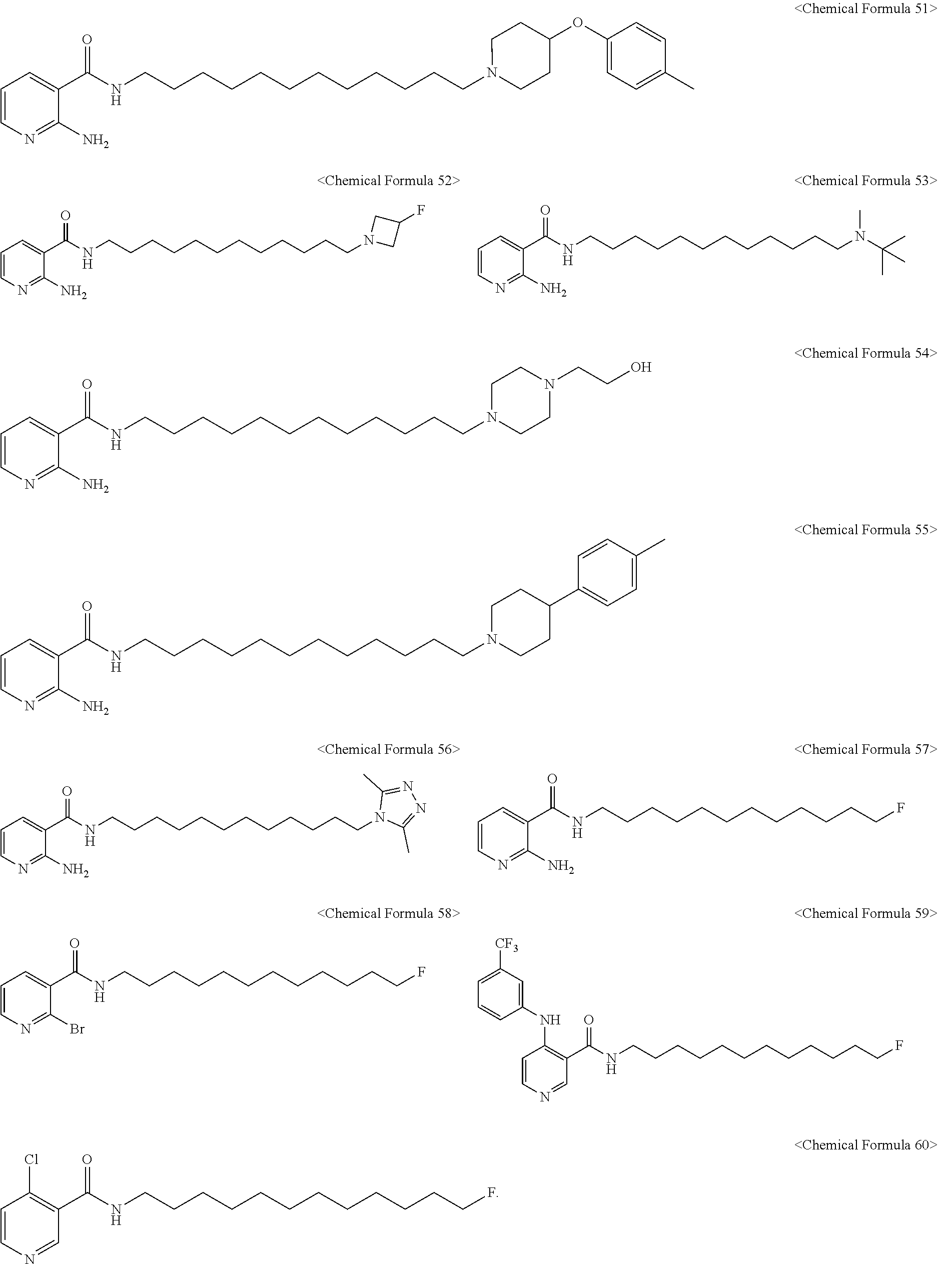

8. The anticancer compound according to claim 1, wherein the compound is characterized by being selected from at least one of the group consisting of chemical formulas 61 to 64, 67 to 71, 73, and 76,

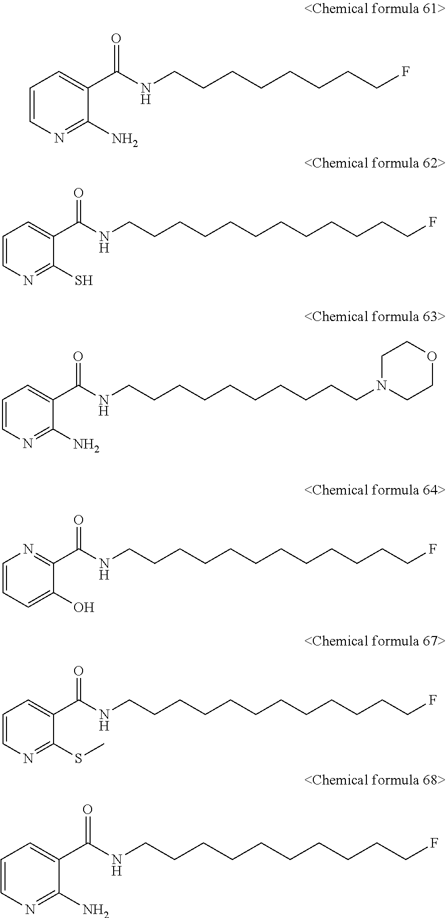 [SMILES: Nc1ncccc1C(=O)NCCCCCCCCF ; O=C(NCCCCCCCCCCCCF)c1cccnc1S ; Nc1ncccc1C(=O)NCCCCCCCCCCN1CCOCC1 ; O=C(NCCCCCCCCCCCCF)c1ncccc1O ; CSc1ncccc1C(=O)NCCCCCCCCCCCCF ; Nc1ncccc1C(=O)NCCCCCCCCCCF]

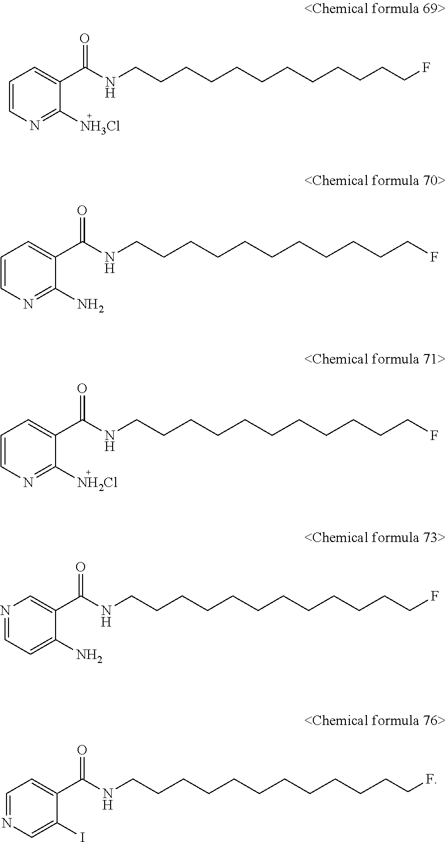

9. The anticancer compound according to claim 1, wherein the compound is characterized by being selected from at least one of the group consisting of chemical formulas 88 to 99,

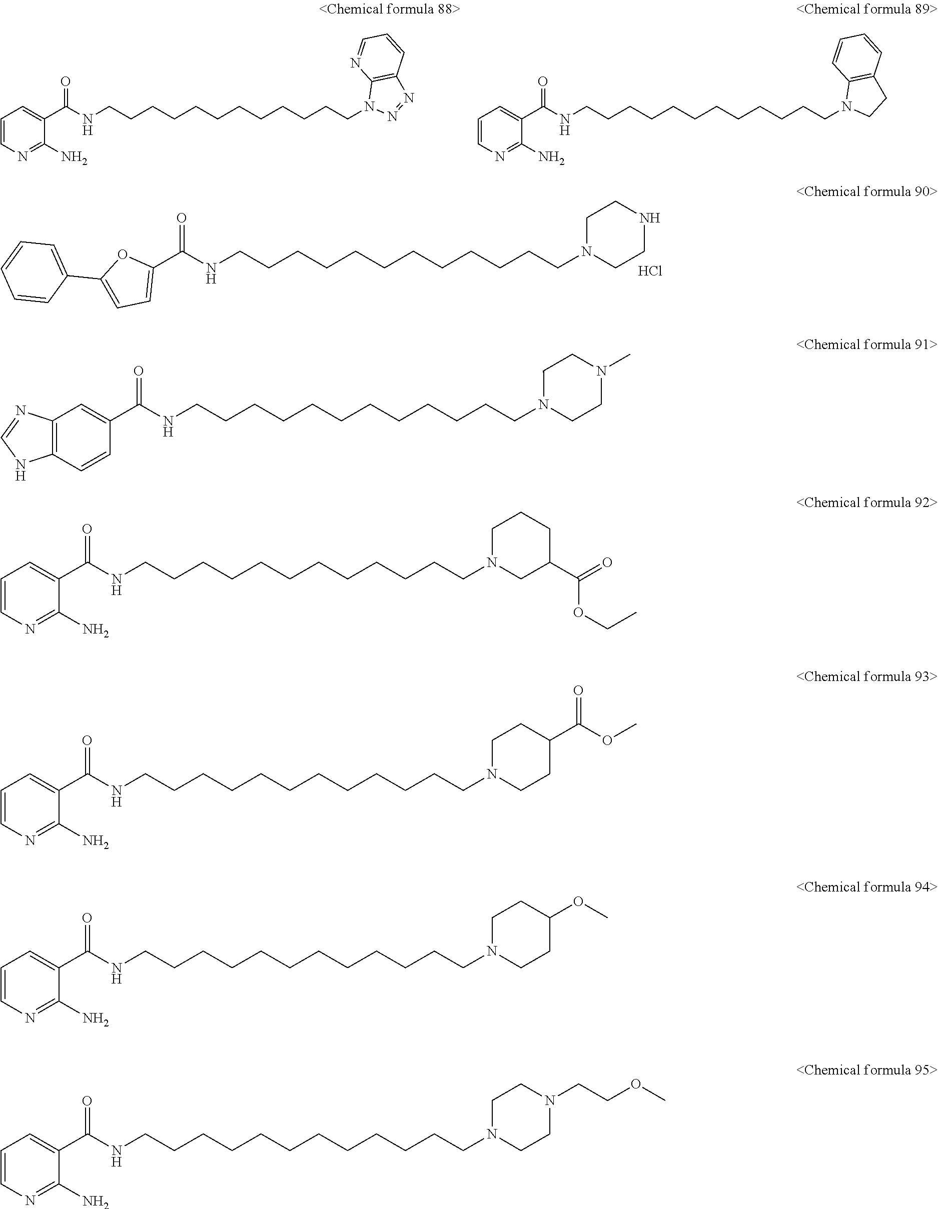

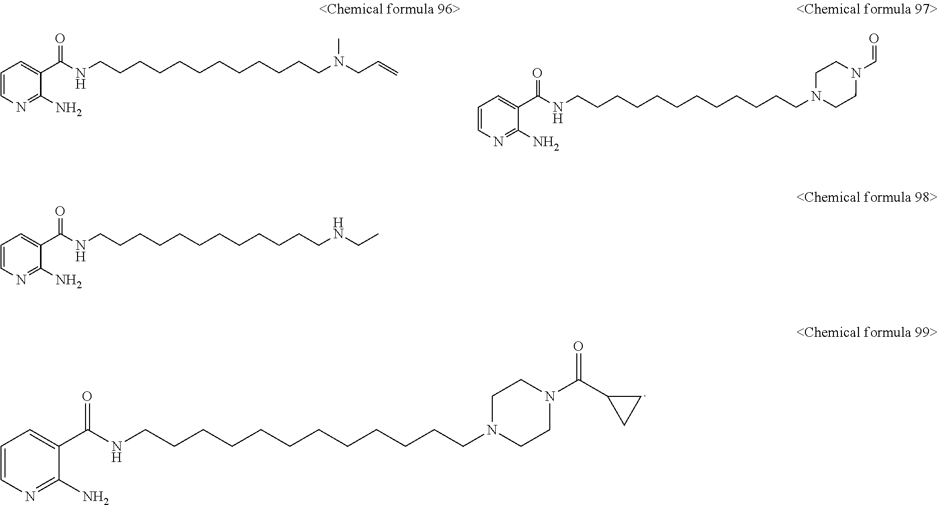 [SMILES: C=CCN(C)CCCCCCCCCCCCNC(=O)c1cccnc1N ; Nc1ncccc1C(=O)NCCCCCCCCCCCCN1CCN(C=O)CC1 ; CCNCCCCCCCCCCCCNC(=O)c1cccnc1N ; Nc1ncccc1C(=O)NCCCCCCCCCCCCN1CCN(C(=O)C2CC2)CC1]

10. The anticancer compound according to claim 1, wherein the compound is characterized by being selected from at least one of the group consisting of chemical formulae 101 to 111 and 113 to 118,

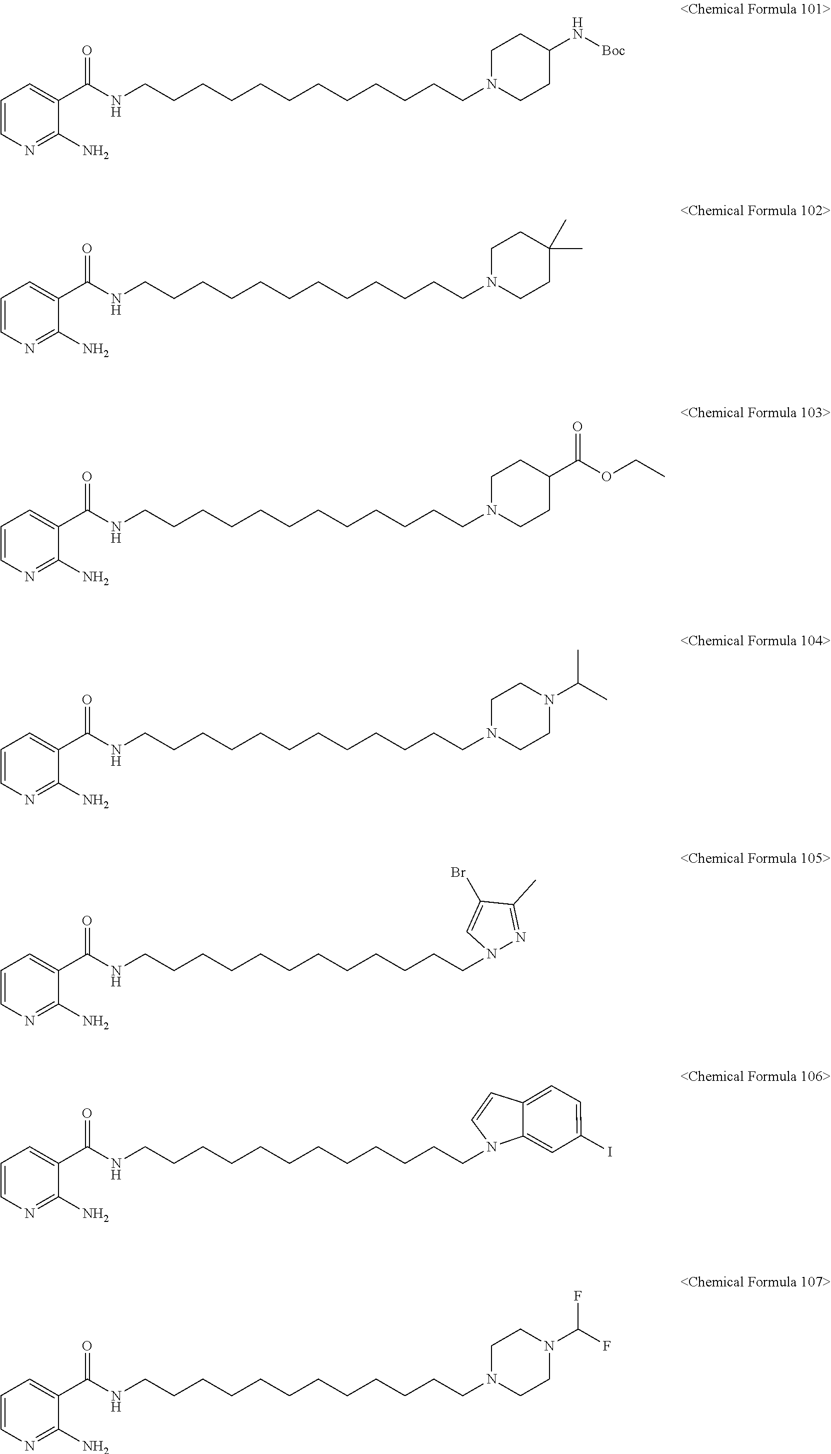

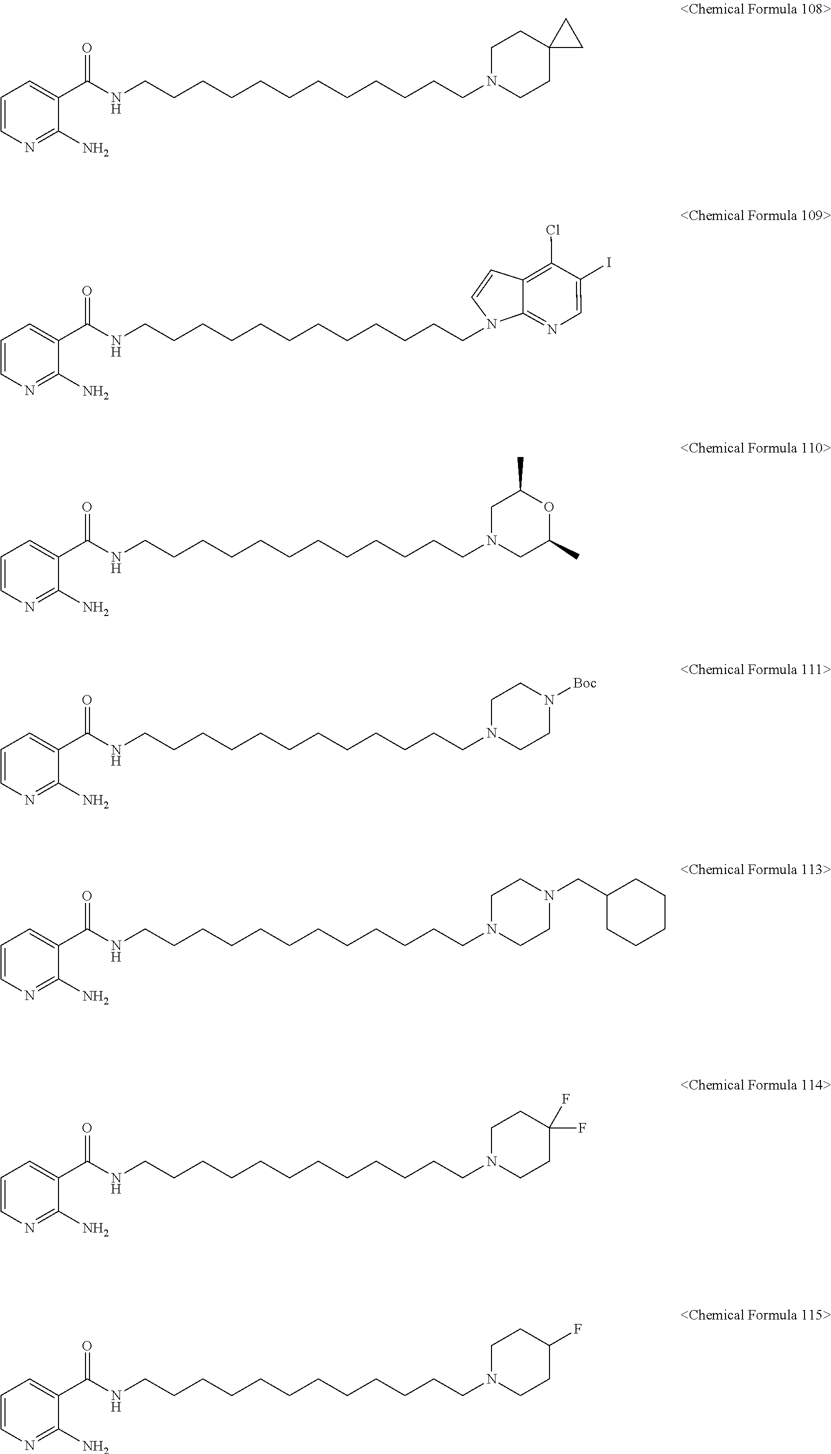

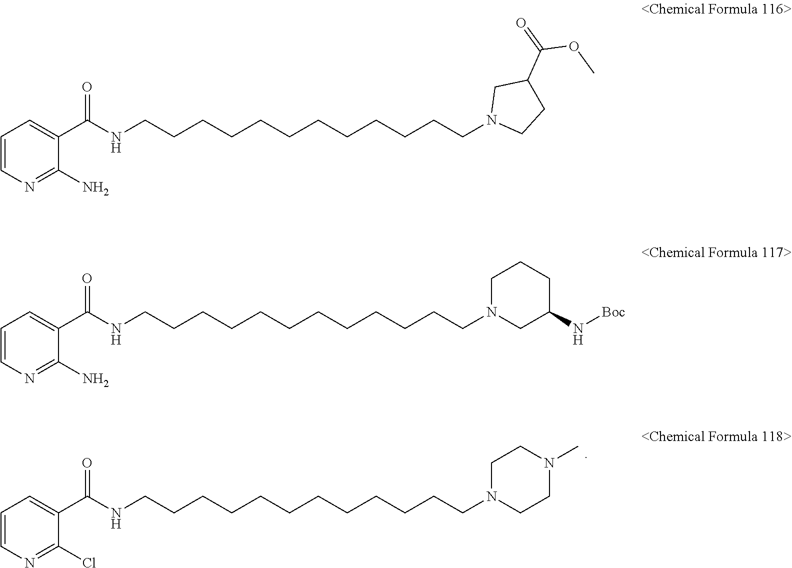 [SMILES: COC(=O)C1CCN(CCCCCCCCCCCCNC(=O)c2cccnc2N)C1 ; CC(C)(C)OC(=O)N[C@@H]1CCCN(CCCCCCCCCCCCNC(=O)c2cccnc2N)C1 ; CN1CCN(CCCCCCCCCCCCNC(=O)c2cccnc2Cl)CC1]

11. The anticancer compound according to claim 1, wherein the compound is characterized by comprising at least one selected from the group consisting of compounds having chemical formulas 121 to 134,

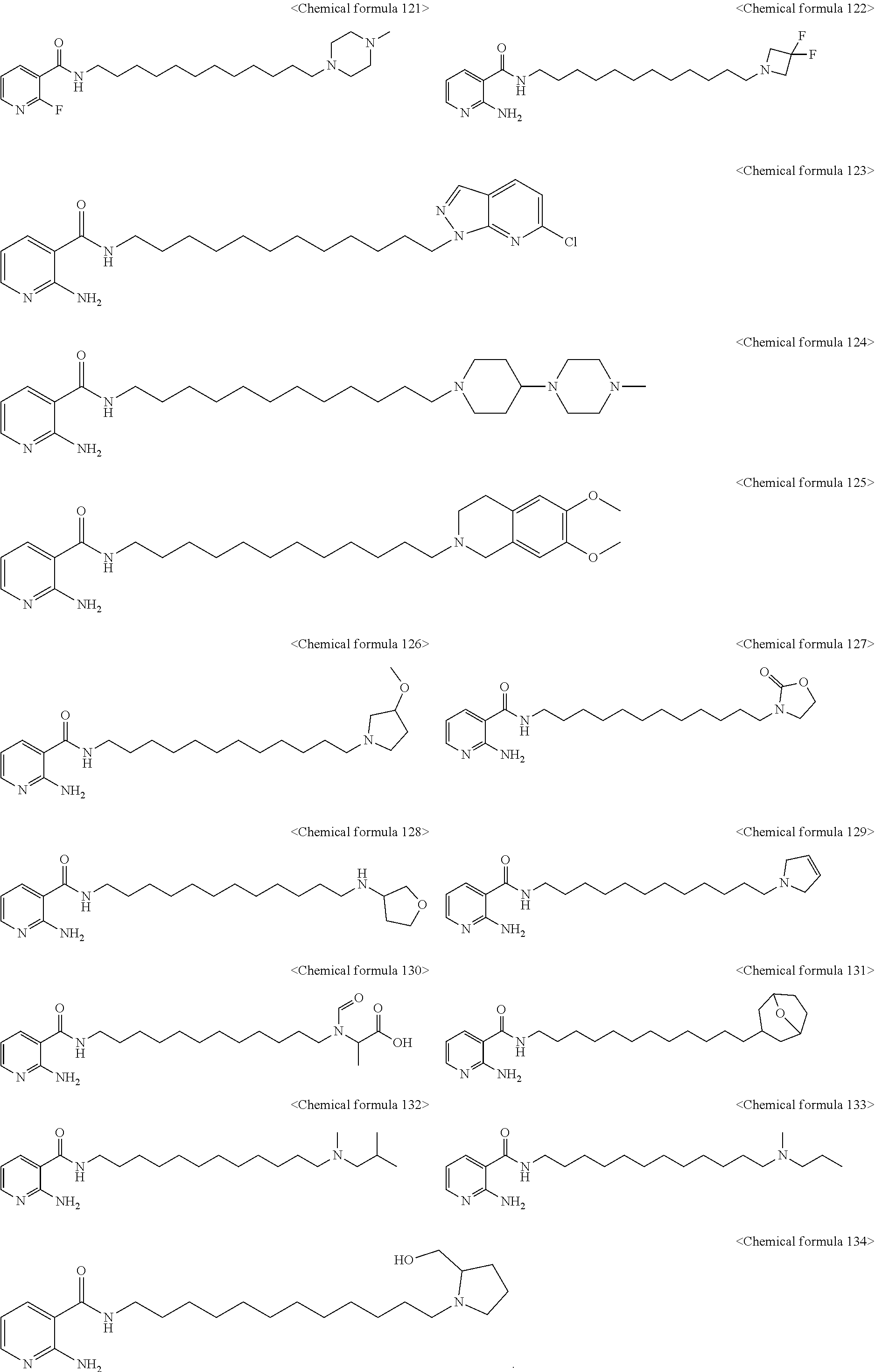

12. The anticancer compound according to claim 1, wherein the compound is characterized by comprising at least one selected from the group consisting of compounds having chemical formulas 147 to 160,

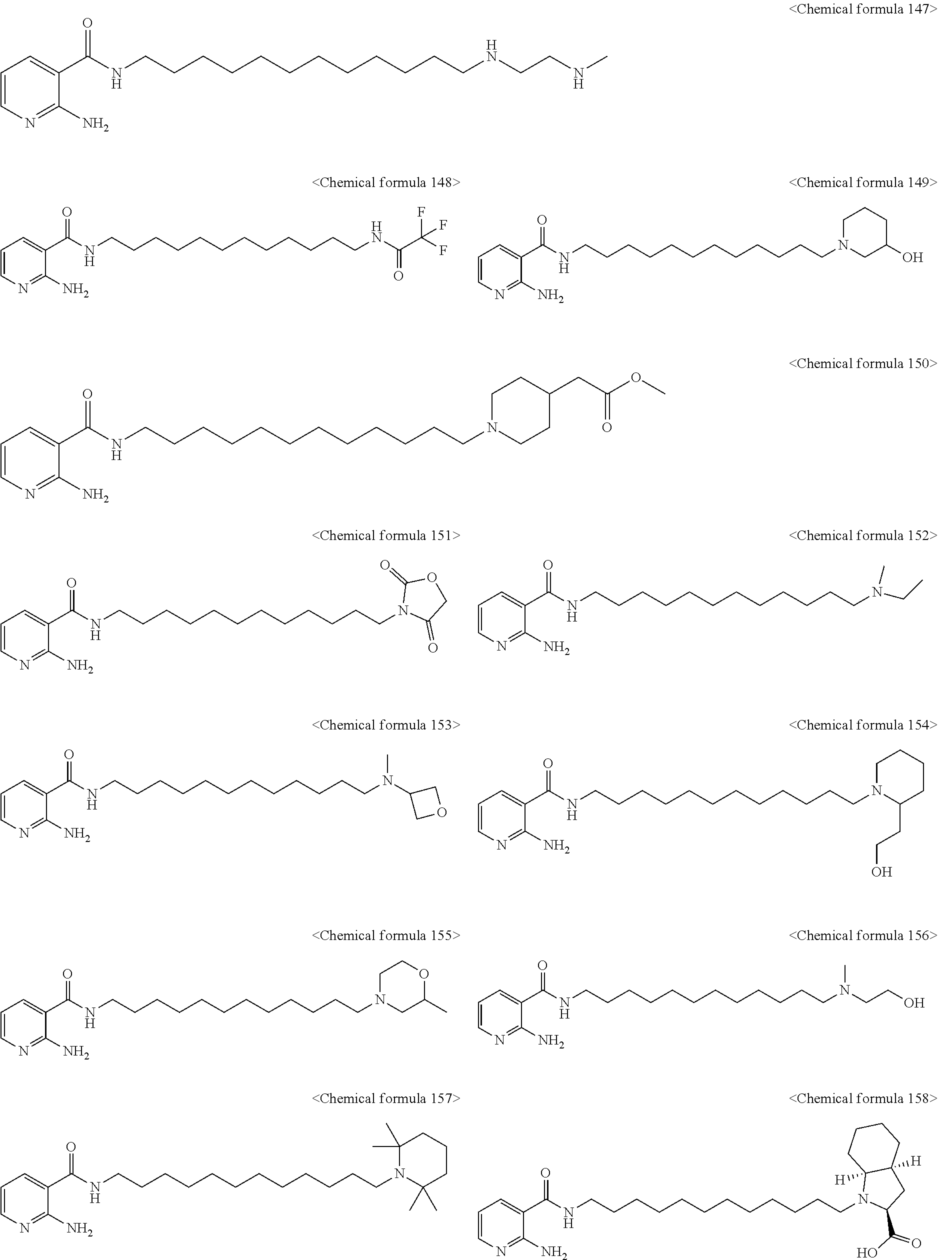

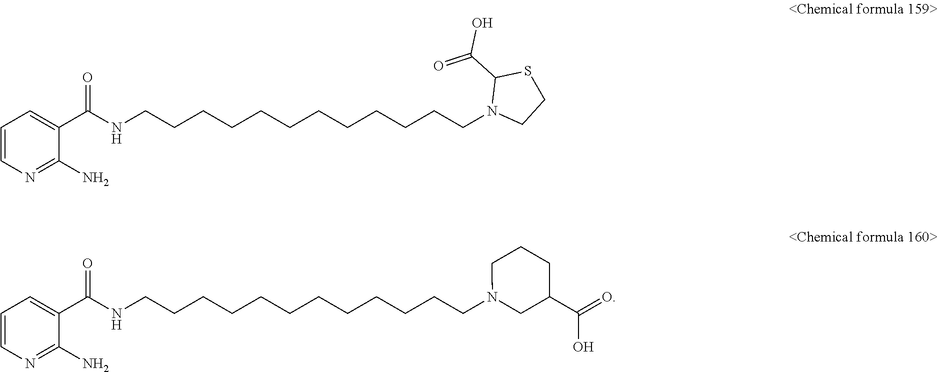 [SMILES: Nc1ncccc1C(=O)NCCCCCCCCCCCCN1CCSC1C(=O)O ; Nc1ncccc1C(=O)NCCCCCCCCCCCCN1CCCC(C(=O)O)C1]

13. The anticancer compound according to claim 1, wherein the compound is characterized by being selected from at least one of the compounds selected from the group consisting of chemical formulae 161 to 180,

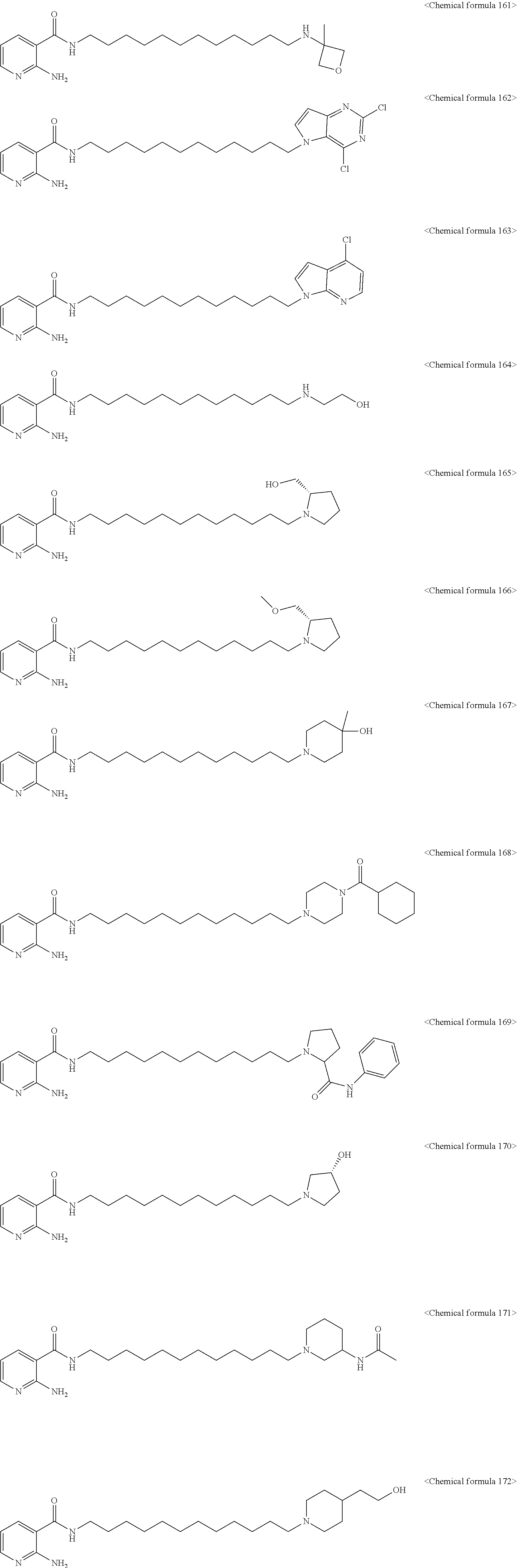

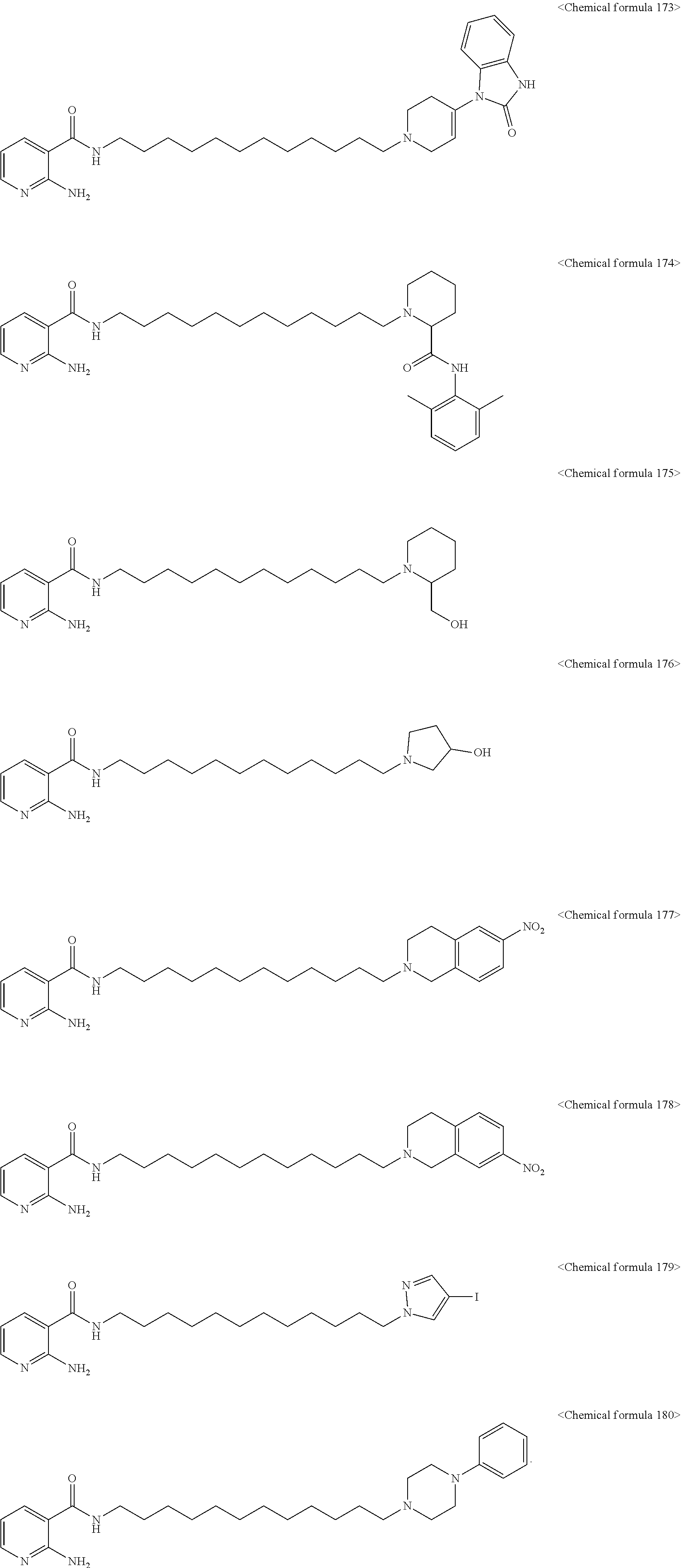

14. The anticancer compound according to claim 1, wherein the compound is characterized by being selected from at least one of the compounds selected from the group consisting of chemical formulae 181 to 190,

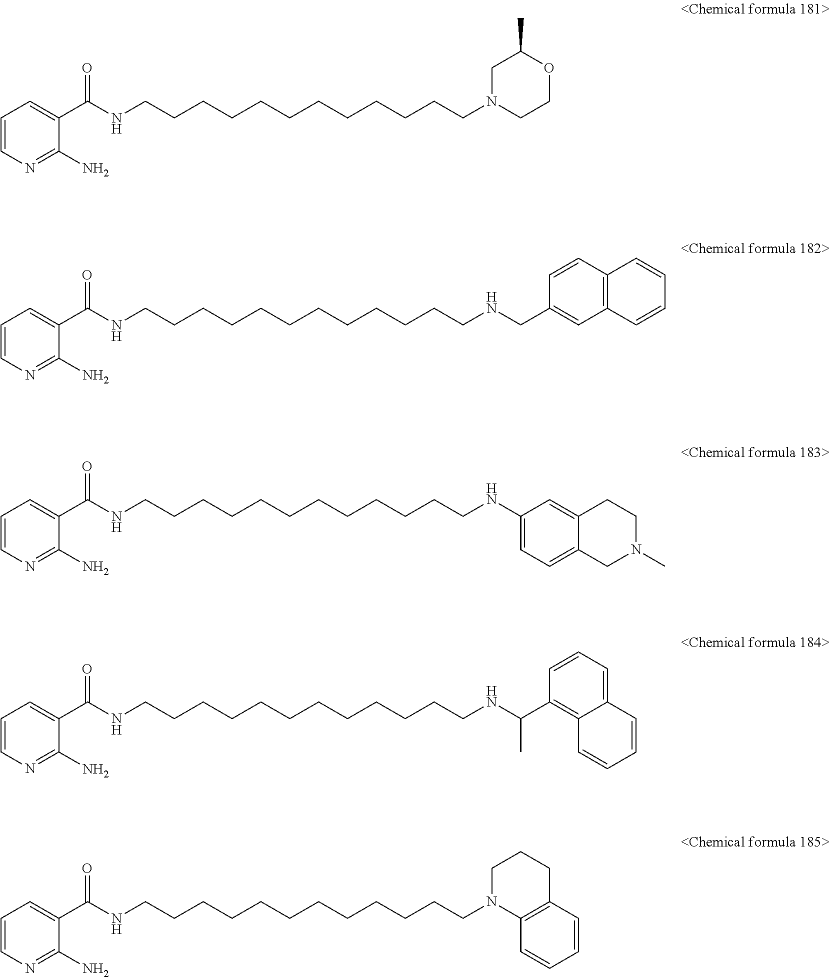 [SMILES: C[C@@H]1CN(CCCCCCCCCCCCNC(=O)c2cccnc2N)CCO1 ; Nc1ncccc1C(=O)NCCCCCCCCCCCCNCc1ccc2ccccc2c1 ; CN1CCc2cc(NCCCCCCCCCCCCNC(=O)c3cccnc3N)ccc2C1 ; CC(NCCCCCCCCCCCCNC(=O)c1cccnc1N)c1cccc2ccccc12 ; Nc1ncccc1C(=O)NCCCCCCCCCCCCN1CCCc2ccccc21]

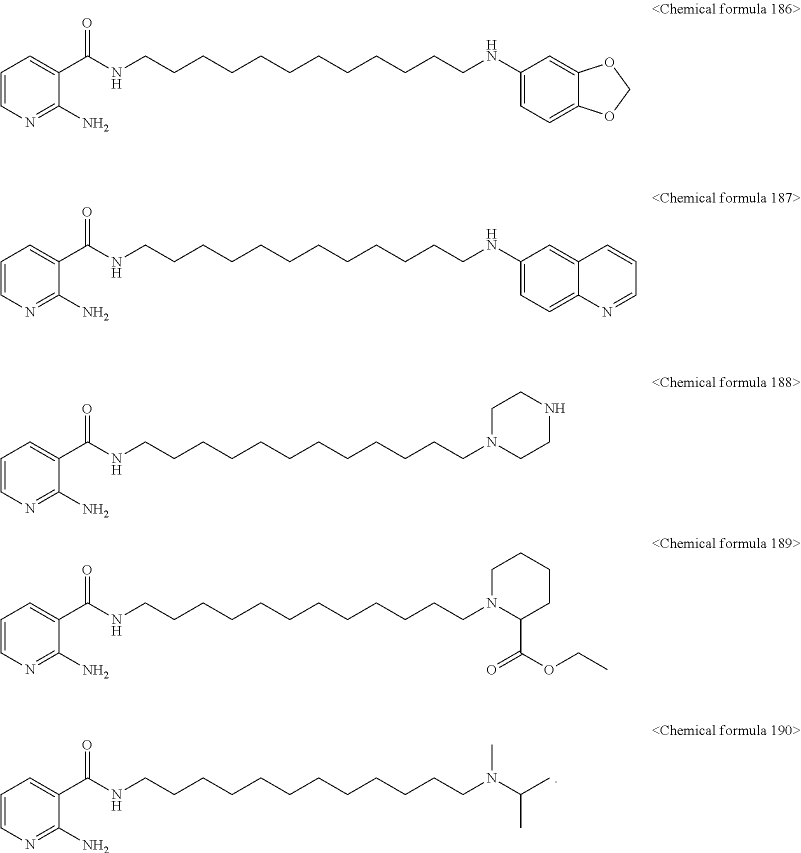 [SMILES: Nc1ncccc1C(=O)NCCCCCCCCCCCCNc1ccc2c(c1)OCO2 ; Nc1ncccc1C(=O)NCCCCCCCCCCCCNc1ccc2ncccc2c1 ; Nc1ncccc1C(=O)NCCCCCCCCCCCCN1CCNCC1 ; CCOC(=O)C1CCCCN1CCCCCCCCCCCCNC(=O)c1cccnc1N ; CC(C)N(C)CCCCCCCCCCCCNC(=O)c1cccnc1N]

15. The anticancer compound according to claim 1, wherein the compound is characterized in that it is at least one selected from the group consisting of chemical formulae 191 to 204, 207 to 214, 216 to 225,

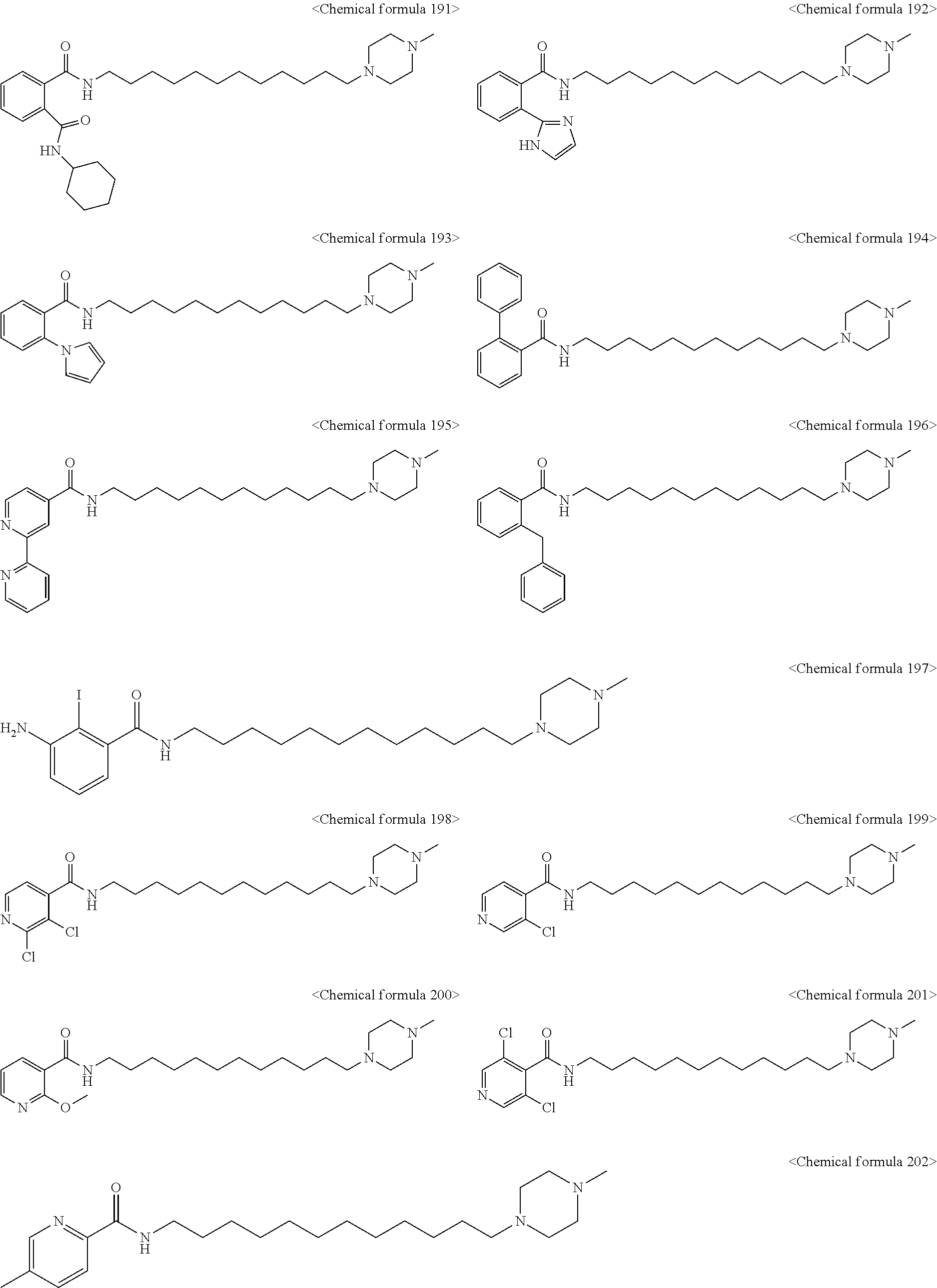

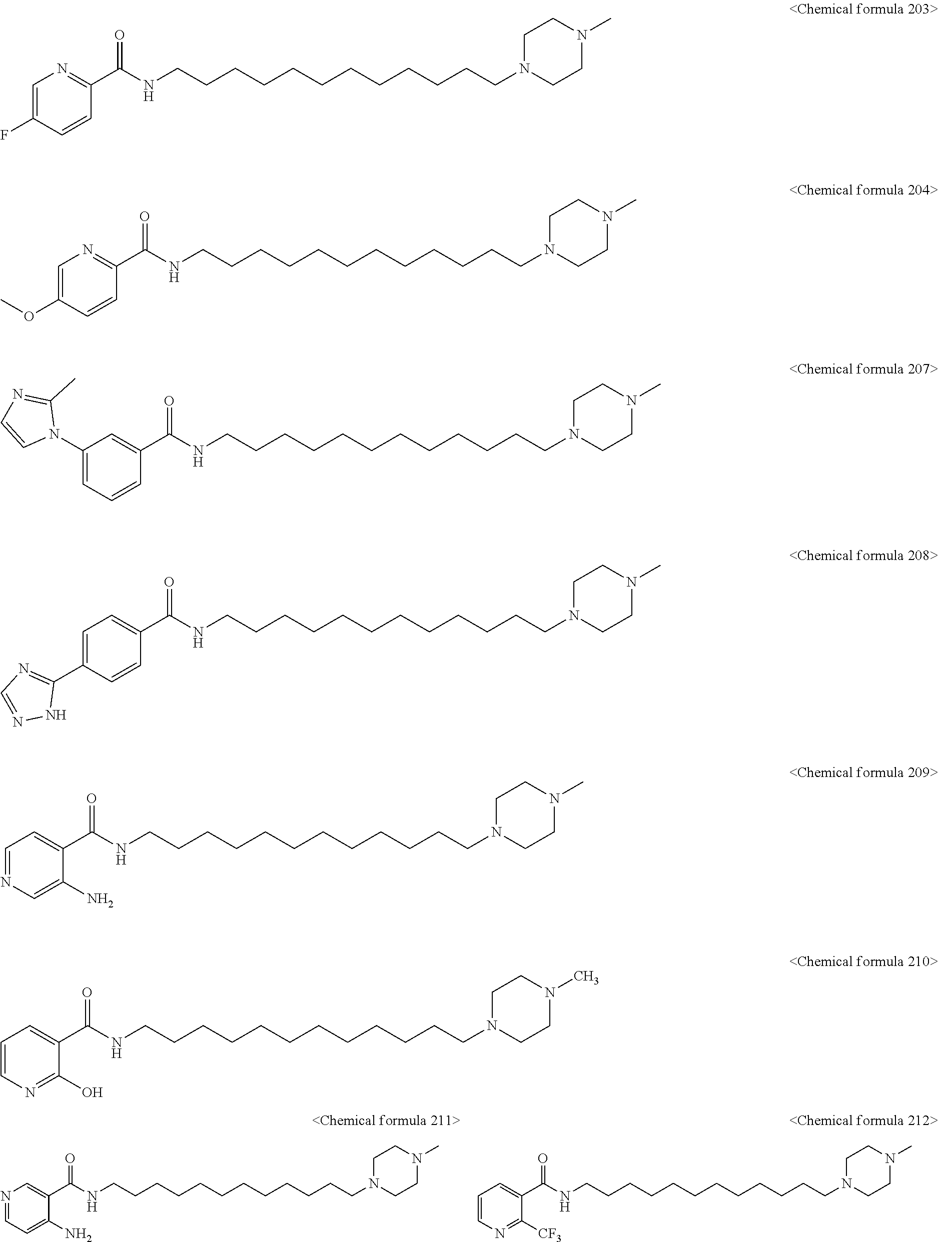

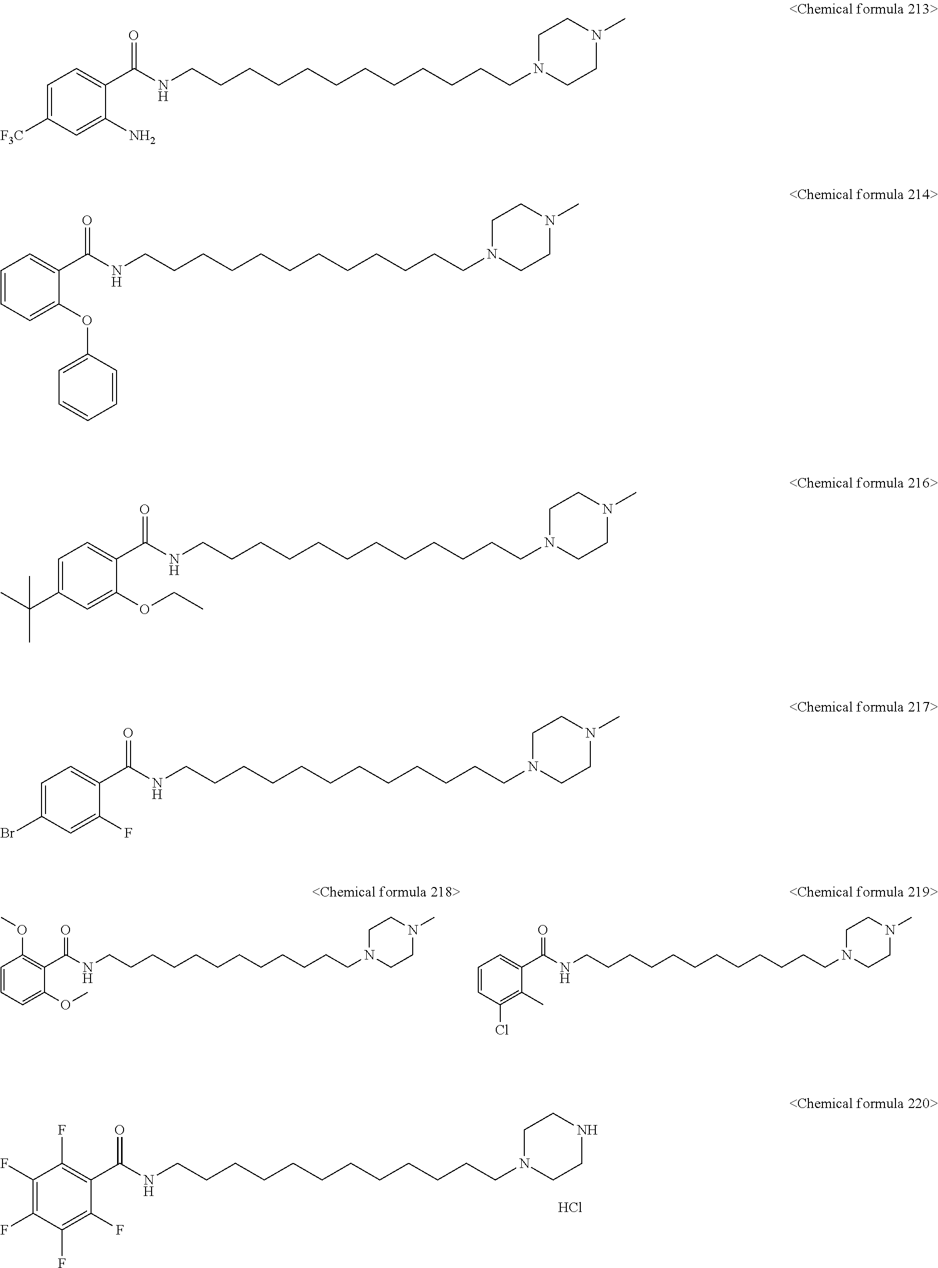

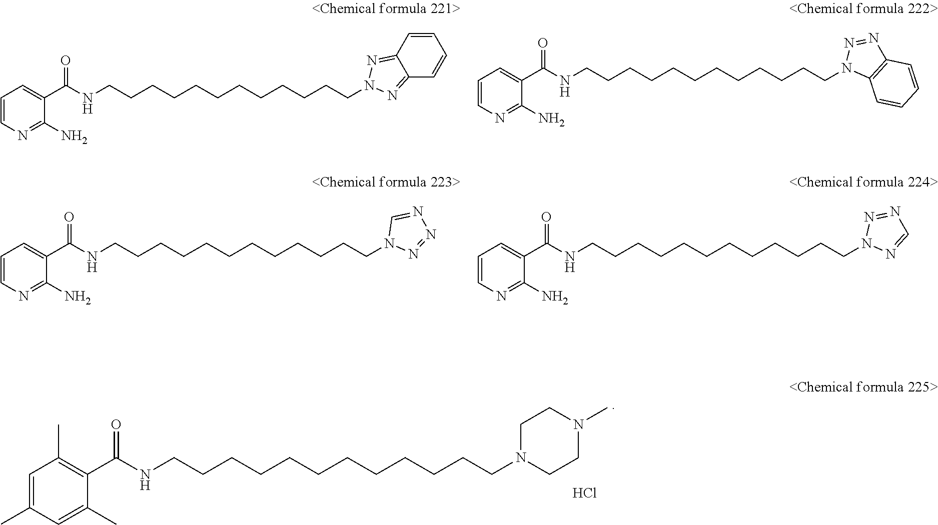

16. The compound or its pharmaceutically acceptable salts according to claim 1, characterized by being used for inhibiting, antagonizing, or regulating PDK1(3-phosphoinositide-dependent protein kinase 1).

17. A method for inhibiting PDK1, comprising the step of contacting an effective amount of the compound or its pharmaceutically acceptable salts according to claim 1 with a target containing PDK1 to inhibit PDK1.

18. The method for inhibiting PDK1 according to claim 17, wherein the target is characterized by being handled in vivo or in vitro.

19. A method for inhibiting PDK1, characterized by comprising the step of contacting an effective amount of the compound or its pharmaceutically acceptable salts according to claim 1 with cells containing PDK1 to inhibit PDK1.

20. A method for antagonizing PDK1, characterized by comprising the step of contacting an effective amount of the compound or its pharmaceutically acceptable salts according to claim 1 with a target containing PDK1 to antagonize PDK1.

21. A method for treating cancer, comprising administering a therapeutic effective amount of the compound or its pharmaceutically acceptable salts according to claim 1, which inhibits, antagonizes, or regulates PDK1 (3-phosphoinositide-dependent protein kinase 1), to a cancer-bearing subject.

22. An anticancer pharmaceutical composition characterized by comprising as an active ingredient of the compound or its pharmaceutically acceptable salts according to claim 1.

23. The anticancer pharmaceutical composition according to claim 22, wherein the pharmaceutical composition is characterized by non-competitively regulating PDK1 activity.

24. The anticancer pharmaceutical composition according to claim 23, wherein the PDK1 regulation is characterized by being achieved through allosteric binding.

25. The anticancer pharmaceutical composition according to claim 22, wherein the pharmaceutical composition is characterized by inhibiting the metastasis of cancer cells.

26. The anticancer pharmaceutical composition according to claim 22, wherein the pharmaceutical composition is characterized by being for the purpose of inhibiting or antagonizing PDK1 activity.

27. The anticancer pharmaceutical composition according to claim 22, wherein the pharmaceutical composition is characterized by being for the purpose of treating cancer or solid tumors.

28. The anticancer pharmaceutical composition according to claim 27, wherein the solid tumor cancer is glioma, glioblastoma, medulloblastoma, meningioma, pituitary adenoma, optic glioma, spinal cord tumor, Schwannoma, Brain metastases, Primary CNS lymphoma, Non-small cell lung cancer, Small cell lung cancer, Lung adenocarcinoma, Lung squamous cell carcinoma, Large cell carcinoma, Malignant mesothelioma, Tracheal cancer, Laryngeal cancer, Pharyngeal cancer, Nasal cavity cancer, Oral cancer, Tongue cancer, Salivary gland cancer, Laryngeal cancer, Pharyngeal cancer, Nasal cavity cancer, Paranasal sinus cancer, Thyroid cancer, Parathyroid cancer, Olfactory neuroblastoma, Invasive ductal carcinoma, Invasive lobular carcinoma, Ductal carcinoma in situ, Inflammatory breast cancer, Triple-negative breast cancer, Papillary carcinoma, Mucinous carcinoma, Tubular carcinoma, Medullary carcinoma, Metastatic breast cancer, Esophageal cancer, Gastric cancer, Small intestine cancer, Colon cancer, Rectal cancer, Hepatocellular carcinoma, Cholangiocarcinoma, Gallbladder cancer, Pancreatic cancer, Anal cancer, Renal cell carcinoma, Urothelial carcinoma, Bladder cancer, Ureteral cancer, Urethral cancer, Prostate cancer, Testicular cancer, Penile cancer, Adrenocortical carcinoma, Pheochromocytoma, Endometrial cancer, Cervical cancer, Uterine sarcoma, Ovarian cancer, Fallopian tube cancer, Vaginal cancer, Vulvar cancer, Hydatidiform mole, Choriocarcinoma, Germinoma, Testicular cancer, Penile cancer, Prostate cancer, Epididymal cancer, Seminal vesicle cancer, Urethral cancer, Germinoma, Choriocarcinoma, Teratoma, Leydig cell tumor, Osteosarcoma, Chondrosarcoma, Ewing sarcoma, Fibrosarcoma, Synovial sarcoma, Liposarcoma, Rhabdomyosarcoma, Leiomyosarcoma, Desmoid tumor, Giant cell tumor of bone, Skin cancer, Melanoma, Basal cell carcinoma, Squamous cell carcinoma, Kaposi's sarcoma, Neuroendocrine tumor, Gastrointestinal stromal tumor (GIST), GIST), retinoblastoma, Wilms tumor, or neuroblastoma.

29. The anticancer pharmaceutical composition according to claim 22, wherein the pharmaceutical composition is characterized by being used for the treatment of blood cancer.

30. The anticancer pharmaceutical composition according to claim 29, wherein the blood cancer is characterized by Acute lymphoblastic leukemia (ALL), Acute myeloid leukemia (AML), Chronic lymphocytic leukemia (CLL), Chronic myeloid leukemia (CML), non-Hodgkin lymphoma (NHL), Hodgkin lymphoma (HL), peripheral T-cell lymphoma (PTCL), anaplastic large cell lymphoma, cutaneous T-cell lymphoma (CTCL), CTCL), Plasmablastic lymphoma, Burkitt lymphoma, Multiple myeloma, Myelodysplastic syndromes (MDS), or Myeloproliferative neoplasms (MPN).
